(19) United States
(12) Patent Application Publication
(10) Pub. No.: US 2024/0158488 A1
(43) Pub. Date: May 16, 2024

(54) METHODS OF RESTORING FUNCTIONAL CAPACITY AND LINEAGE COMPOSITION OF AN AGING BLOOD AND VASCULAR SYSTEM

(71) Applicant: Hackensack Meridian Health Center for Discovery and Innovation, Nutley, NJ (US)

(72) Inventors: Jason Mathew Butler, Hackensack, NJ (US); Pradeep Ramalingam, Hackensack, NJ (US)

(21) Appl. No.: 17/918,675

(22) PCT Filed: Apr. 15, 2021
(86) PCT No.: PCT/US2021/027551

§ 371 Date: Oct. 13, 2022

Related U.S. Application Data
(60) Provisional application No. 63/011,815, filed on Apr. 17, 2020.

(51) Int. Cl. C07K 16/20; A61K 35/28; A61K 35/44; A61P 7/00; C12N 15/113

(52) U.S. Cl. CPC C07K 16/205; A61K 35/28; A61K 35/44; A61P 7/00; C12N 15/113; C07K 2317/76; C12N 2310/14

(57) ABSTRACT

The described invention provides a method for rejuvenating an aging blood and vascular system comprising aging-associated hematopoietic defects in an aging hematopoietic microenvironment of bone marrow including deteriorating vascular integrity, reduced hematopoietic stem cell function, or both. The method includes administering to a subject a pharmaceutical composition comprising an inhibitor of a pro-aging angiocrine factor, a splice variant, or a fragment thereof, and a pharmaceutically acceptable carrier. The described invention has identified thrombospondin-1 as a candidate pro-aging factor.

### CROSS-REFERENCE TO RELATED APPLICATIONS

This application claims the benefit of priority to U.S. Provisional Application No. 63/011,815, filed Apr. 17, 2020, entitled “Methods of Restoring Functional Capacity and Lineage Composition of An Aging Blood and Vascular System”. The entire contents of the aforementioned application is incorporated by reference herein in its entirety.

### STATEMENT OF GOVERNMENT FUNDING

This invention was made with Government support under contract HL133021 awarded by the National Institutes of Health. The Government has certain rights in this invention.

### SEQUENCE LISTING

The instant application contains a Sequence Listing which has been submitted electronically in ASCII format and is hereby incorporated by reference in its entirety. Said ASCII copy, created on Apr. 15, 2021, is named 128533-02620_SL.txt and is 2,833 bytes in size.

### FIELD OF THE INVENTION

The described invention relates to compositions and methods for reversing age-related deficiencies in hematopoiesis.

### BACKGROUND OF THE INVENTION

### Homeostasis

Homeostasis is a property of cells, tissues and organisms that allows the maintenance and regulation of the stability and constancy needed for proper body function. It is maintained by the constant adjustment of biochemical and physiological pathways despite changes in environment, position, and activity level. This adjusting of physiological systems within the body is termed homeostatic regulation. The homeostatic regulation of biologic tissue requires an orchestrated interplay between osteogenesis, angiogenesis/vasculogenesis, and hematopoiesis, which is thought to be mediated by endothelial cells (ECs). (Kenswil, K. J. G., et. al., (2018). Characterization of Endothelial Cells Associated with Hematopoietic Niche Formation in Humans Identifies IL-33 As an Anabolic Factor. Cell Reports, 22(3), 666-678; Rafii, S., Butler, J. M., & Ding, B.-S. (2016). Angiocrine functions of organ-specific endothelial cells. Nature, 529(7586), 316-325).

Endothelial cells lining blood vessel capillaries were thought of as passive conduits with a responsibility for delivering blood, oxygen and nutrients, modulating the coagulation of blood, regulating the transportation of inflammatory cells and serving as gatekeepers of cellular metabolism. However, these cells also perform other necessary physiological tasks, such as sustaining the homeostasis of resident stem cells and guiding the regeneration or repair of adult bone/bone marrow (osteogenesis), blood systems (hematopoiesis), and vasculature (angiogenesis/vasculogenesis). (Rafii, S., Butler, J. M., & Ding, B.-S. (2016). Angiocrine functions of organ-specific endothelial cells. Nature, 529(7586), 316-325).

### Vascular Integrity

The integrity of blood vessels is critical to vascular homeostasis. (Murakami, M., and Simons, M. J. Mol. Med. (Berl) (2009) 87 (6): 571-82). Maintenance of the vasculature is an active biological process that requires continuous, basal cellular signaling. Failure of this system results in serious consequences, such as hemorrhage, edema, inflammation, and tissue ischemia.

Studies in various animal models as well as mouse and human genetic studies have identified a number of factors that play critical roles in active maintenance of blood vessel integrity during embryonic vessel development or in the adult vasculature. These factors work coordinately over many steps of vascular stabilization and maintenance in an orchestrated manner. During the process of vascular formation, after nascent vessels are assembled, endothelial cells develop cell-cell junctions to establish an effective barrier, a process in which Ang1-Tie2 and FGF systems play pivotal roles (Id., citing Fiedler, U, Augustin, HG, Trends Immunol. (2006) 27: 552-58; Murakami, M., Simons, M.; Curr. Opin. Hematol. (2008) 15: 215-220). Concomitantly, while mesenchymal progenitor cells differentiate into pericytes or smooth muscle cells through the action of TGF-β, PDGF-BB derived from endothelial tip cells promotes pericyte recruitment and proliferation (Id., citing Pepper, MS, Cytokine Growth Factor Rev. (1997) 8: 21-43); Betsholtz, C. Cytokine Growth Factor Rev. (2004) 15: 215-228; Andrrae, J. et al., Genes Dev. (2008) 22: 1276-1312). Throughout this process, integrins mediate extracellular matrix (ECM)-cell signaling that further directs vessel stabilization (Id., citing Hynes, R O, J. Thromb. Haemost. (2007) 5 (Suppl. 1): 32-40).

Vascular integrity is tightly regulated by a number of factors that ensure proper functions of various components of the blood vessel wall. One of the early hallmarks of deteriorating vascular integrity is increased permeability, which is predominantly controlled by endothelial junction stability. Selective regulation of vascular permeability is achieved by regulation of the size and state of paracellular gaps and control of the transcellular transport. The normal vasculature demonstrates a certain level of basal permeability that varies from bed to bed. Early studies have revealed constitutively open junctions in a subset of vascular beds (Id., citing Simionescu, N. et. al., J. Cell Biol. (1978) 79: 27-44). Under normal conditions, about 30% of endothelial cell-cell junctions in postcapillary venules, where active permeability regulation occurs, are open and permeable to ˜60 Å molecules. (Id.) Upon stimulation with either histamine or 5-HT (5-hydroxytriptamine), cellular junctions in postcapillary venules selectively open up and allow passage of larger molecules; however, outflow through venular junctions is limited and restricted to the perivascular spaces ((Id., citing Simionescu, N. et al., J. Cell Biol. (1978) 79: 27-44), which suggests the existence of an external barrier in the perivascular tissue. (Id.)

Increased endothelial permeability, elicited by physiological and pathological stimuli, is usually reversible and does not permanently deteriorate vascular integrity. Interference with endothelial junctional components can, however, leads to severe impairment of vascular integrity. In this scenario, junctional disruption is usually accompanied by eventual endothelial detachment from the vessel wall followed by thrombus formation. Although the sequence of events in this process is not well understood, it is possible that duration of permeability-inducing stimuli may influence the outcome. (Id.) Unlike a transient increase of vascular permeability, in which endothelial cells can quickly restore the barrier function by reestablishing VE-cadherin-based junctions, prolonged stimuli may leads to a more profound effect such as accumulation of reactive oxygen species (ROS). Excessive amounts of ROS, known to exert a number of adverse effects on endothelial function, may mediate such a scenario. In fact ROS can irreversibly inactivate protein tyrosine phosphatases (PTPs) by oxidizing a Cys residue in the active site, thereby affecting tyrosine phosphorylation-dependent signaling events (Id., citing Tonks, N K, Nat. Rev. Mol. CVell Biol. (2006) 7: 833-846).

### Endothelial Junctions

In endothelial cells, among the three types of intercellular junctions, namely adherens-, tight- and gap junction, adherens and tight junctions contribute to the structural integrity of the endothelium (Id., citing Dejana, E., Nat. Rev. Mol. Cell Biol. (2004) 5: 261-270). Although it is difficult to precisely delineate the functional difference between these two types of junctions, it has been shown that assembly of tight junctions is dependent on prior formation of adherens junctions, and it is generally considered that adherens junctions are primarily important for the control of endothelial permeability, whereas tight junctions are implicated in blocking the movement of lipids and integral membrane proteins between the apical and basolateral surfaces of the cell (molecular fence) (Id., citing Dejana, E., Nature Rev. Mol. Cell Biol. (2004) 5:261-270; Taddei, A., et al., Nat. Cell Biol. (2008) 10: 923-34).

Each type of junction possesses a distinct set of proteins. Cadherins are a family of transmembrane proteins that constitute adherens junctions and mediate cell-cell contacts in a calcium-dependent manner through trans-homophilic interactions. In endothelial cells, VE-cadherin localizes at sites of cellular contacts, regulating the formation of adherens junctions and connecting the site of the junction to the actin cytoskeleton.

Stability of VE-cadherin at adherens junctions, which is controlled by binding to catenins, especially to p120-catenin, is critical to the maintenance of endothelial permeability and integrity. Src family kinases have been known to play an important role in VEGF-induced increase in endothelial permeability, and VE-cadherin phosphorylation via Src triggers disruption of cell-cell contacts, leading to VE-cadherin internalization (Id., citing Weis, S M, Chesh, D A, Nature (2005) 437: 497-504). This process is thought to be important for the establishment of endothelial motility and the angiogenic phenotype of “activated” endothelial cells. Thus, endothelial junctions are dynamic structures that actively assemble and disassemble even in the quiescent monolayer, suggesting that the balance of action controlling net VE-cadherin dynamics determines the endothelial behavior.

### Stem Cell Niches

Effective functioning of the body's tissues and organs depends upon innate regenerative processes that maintain proper cell numbers (homeostasis) and replace damaged cells after injury (repair). In many, though not all tissues, regenerative potential is determined by the presence and functionality of a dedicated population of stem and progenitor cells, which respond to exogenous cues to produce replacement cells when needed. (Wagers, A. J. The stem cell niche in regenerative medicine. Cell stem cell 10, 362-369, doi:10.1016/j.stem.2012.02.018 (2012)). These cells exist in a specialized environment, termed a “stem cell niche”, which provides spatial, temporal, and structural boundaries sufficient to protect these cells from damage or loss while maintaining communication with their surroundings to ensure appropriate responsiveness to physiological cues for cell replacement and repair. (Wagers, A. J. The stem cell niche in regenerative medicine. Cell stem cell 10, 362-369, doi:10.1016/j.stem.2012.02.018 (2012)).

Stem cell niches have been identified and characterized in many tissues, including the germline, bone marrow, digestive and respiratory systems, skeletal muscle, skin, hair follicle, mammary gland, and central and peripheral nervous systems. (Wagers, A. J. The stem cell niche in regenerative medicine. Cell stem cell 10, 362-369, doi:10.1016/j.stem.2012.02.018 (2012)).

Stem cell niche environments are composed of cellular and environmental components that are critical to their function and maintenance. Cell-cell interactions provide structural support, regulate adhesive interactions, and produce soluble signals that control stem cell function. Environmental components include physical forces such as pressure, structure, and chemical signals, and temperature, as well as physiological parameters, such as interaction with the extracellular matrix (ECM). (Id.).

Heterologous cell-cell interactions in stem cell niches exhibit complex, bidirectional signaling that is dependent on tight regulation and often cell-to-cell contact. Stem cell niches contain tissue specific and generic cell populations, each of which have specialized roles. (Lane, S. W., Williams, D. A. & Watt, F. M. Modulating the stem cell niche for tissue regeneration. Nature biotechnology 32, 795-803, doi:10.1038/nbt.2978 (2014)).

The hematopoietic microenvironment localized in the marrow space in adult bone and comprises a range of different cell types that distinctly define the hematopoietic stem cell (HSC) niche, including osteoblastic, vascular, and neural cells, megakaryocytes, macrophages and immune cells. Secreted and membrane-bound factors, including Wnt, SCF, Notch and chemokines directly bind surface receptors on stem cells to regulate cell fate, self-renewal and polarity. (Lane, S. W., Williams, D. A. & Watt, F. M. Modulating the stem cell niche for tissue regeneration. Nature biotechnology 32, 795-803, doi:10.1038/nbt.2978 (2014)).

The close association of many stem cell types with the vasculature and nervous system allows for modulation of stem cell responses by metabolic cues and circadian rhythms, and provides a conduit through which inflammatory and immune cells, as well as humoral factors, can be delivered to the niche. (Wagers, A. J. The stem cell niche in regenerative medicine. Cell stem cell 10, 362-369, doi:10.1016/j.stem.2012.02.018 (2012)). Immunological cells provide dynamic regulation of niches during inflammation and tissue damage, which is tightly regulated through the presence of “immune privilege” (referring to the observation that tissue grafts placed in certain anatomical sites, including the brain and eye, can survive for extended periods of time) and evasion from this privilege. (Lane, S. W., Williams, D. A. & Watt, F. M. Modulating the stem cell niche for tissue regeneration. Nature biotechnology 32, 795-803, doi:10.1038/nbt.2978 (2014)).

Extracellular matrix (ECM) proteins and stem cell interactions with the ECM provide retention cues, as well as mechanical signals, based in part on substrate rigidity, which allow stem cells to respond to external physical forces. ECM proteins are critical for orientation and structural maintenance of the niche and provide instructive signals through ligand interaction with integrins expressed on stem cells. (Lane, S. W., Williams, D. A. & Watt, F. M. Modulating the stem cell niche for tissue regeneration. Nature biotechnology 32, 795-803, doi:10.1038/nbt.2978 (2014)). In addition, the ECM can sequester or concentrate growth factors, chemokines, and other stem cell regulatory molecules by binding both locally and systematically produced factors within the niche. (Wagers, A. J. The stem cell niche in regenerative medicine. Cell stem cell 10, 362-369, doi:10.1016/j.stem.2012.02.018 (2012)).

Physical parameters, such as topography, rigidity/elasticity, shear forces, temperature, oxygen tension, and blood flow direct stem cell maintenance and differentiation. Further, many stem cell niches have altered environmental characteristics and require tight metabolic regulation to maintain the long-term quiescence and self-renewal of stem cell populations. (Lane, S. W., Williams, D. A. & Watt, F. M. Modulating the stem cell niche for tissue regeneration. Nature biotechnology 32, 795-803, doi:10.1038/nbt.2978 (2014)).

While the specific components that constitute a particular stem cell niche may vary in different tissues under distinct physiological contexts, in all cases, the signals provided by these cellular and acellular components appear to be integrated by stem cells to inform their fate decisions, including choices between quiescence or proliferation, self-renewal or differentiation, migration or retention, and cell death or survival. (Wagers, A. J. The stem cell niche in regenerative medicine. Cell stem cell 10, 362-369, doi:10.1016/j.stem.2012.02.018 (2012)).

### HSC Niche Development

The hematopoietic system supplies the human body with >100 billion mature blood cells every day that carry out functions such as oxygen transport, immunity, and tissue remodeling. It consists of various populations of highly specialized cells that have unique functions, such as oxygen transport and immune defense. It is estimated that an adult human generates ˜4-5×10¹¹ haematopoietic cells per day. The continuous production of many blood cell types requires a highly regulated, yet highly responsive, system. Within the mammalian haematopoietic organization, rare haematopoietic stem cells (HSCs) sit at the top of the hierarchy. (Pinho, S., Frenette, P. S. Haematopoietic stem cell activity and interactions with the niche. Nat Rev Mol Cell Biol 20, 303-320 (2019) doi:10.1038/s41580-019-0103-9).

During development, HSCs traffic between niches in order to establish hematopoiesis. Primitive hematopoiesis takes place in the yolk sac approximately on embryonic day 7.0 (E7.0) when immature precursors give rise to erythrocytes that will supply oxygen to the developing embryo. The presence of the first definitive HSC known to be able to fully reconstitute the hematopoietic system upon transplantation is found in the aorta-gonad-mesonephros in mice and humans. However, some studies have suggested that yolk sac cells from E9.0 to E10.0 can mature into definitive HSCs when transplanted into a newborn rather than an adult mouse. In addition, the placenta represents a significant reservoir of HSCs during development. Once the vasculature is developed, HSCs migrate to the fetal liver on or near E12.0 where they expand and differentiate. Fetal liver HSCs are actively cycling in contrast to their bone marrow counterparts and can also out-compete adult bone marrow HSCs when transplanted into irradiated recipients. During HSC expansion in the fetal liver, chondrocytes and osteoblasts are produced within mesenchymal condensations to create cartilage and bone. Skeletal remodeling is associated with bone vascularization, which allows homing of HSCs and colonization of the fetal bone marrow by E17.5. This process is mediated through CXCL12 production by bone marrow stromal cells, which attract HSCs expressing CXCR4 and specific adhesion molecules expressed on bone marrow endothelium. (Boulais, P. E., & Frenette, P. S. (2015). Making sense of hematopoietic stem cell niches. Blood, 125(17), 2621-2629. doi:10.1182/blood-2014-09-570192).

### HSC Niche and the Bone Marrow Microenvironment

In adult bones, HSCs are essentially kept in the G0 phase of the cell cycle in a stage of metabolic dormancy or quiescence, which preserves their function by limiting damage associated with cell replication. However, quiescent HSCs can quickly respond to a broad range of niche or systemic signals by entering the cell cycle and proliferating. These instructive cues are therefore essential for tailoring HSC differentiation and adjusting blood production to the needs of the organism. HSCs can also leave the BM niche upon receiving mobilization signals and enter the bloodstream to ensure immune surveillance of peripheral tissues and engraft distant BM sites. Thus, HSCs critically depend on short- and long-range instructive cues from the BM niche for many aspects of their biology, including cell cycle and trafficking activity, due to the dynamic regulation of the switch between quiescence/proliferation and anchoring/mobilization.

Resident Niche Cells. The HSC stem cell niche contains a variety of cell types, each with a distinct function, such as osteoblastic, vascular, and neural cells, megakaryocytes, macrophages and immune cells each have important roles and can be considered to define distinct HSC niches. It further comprises other specialized niches, for example, the osteoblastic and perivascular niches. Research is conflicting whether these two niches have distinct, specialized roles or whether there is coordinated regulation of HSCs and therefore functional overlap. For example, NG2+ peri-arteriolar cells regulate quiescence within long-term HSCs, and this quiescence appears essential for HSC function. Other cells, such as endosteal macrophages, retain HSCs within the niche, and loss of these cells causes mobilization of HSCs out of their supportive microenvironment. (Lane, S. W., Williams, D. A. & Watt, F. M. Modulating the stem cell niche for tissue regeneration. Nature biotechnology 32, 795-803, doi:10.1038/nbt.2978 (2014)).

Direct cell contact. Direct cell contact can be mediated by a range of receptors, such as cell-cell adhesion molecules and receptors with membrane bound ligands. For example, in bone marrow, Notch ligands expressed by sinusoidal cells are essential for HSC self-renewal during recovery from myeloablative injury. (Lane, S. W., Williams, D. A. & Watt, F. M. Modulating the stem cell niche for tissue regeneration. Nature biotechnology 32, 795-803, doi:10.1038/nbt.2978 (2014)).

Secreted Factors. Indirect communication between stem cells and niche cells is mediated by secreted factors. Mobilization of HSCs from their niche, for example, by using cytokines such as granulocyte colony-stimulating factor (G-CSF) or granulocyte-macrophage colony-stimulating factor (GM-CSF), is widely used to support treatment of hematological malignancy, bone marrow failure and rare genetic disorders. These factors act in a variety of ways, including promoting expansion of HSCs and release of HSC-niche adhesion. (Lane, S. W., Williams, D. A. & Watt, F. M. Modulating the stem cell niche for tissue regeneration. Nature biotechnology 32, 795-803, doi:10.1038/nbt.2978 (2014)). Specifically, secreted factors like stem cell factor (SCF), transforming growth factor beta-1 (TGF-b1), platelet factor 4 (PF4 or CXCL4), angiopoietin 1 (ANGPT1), and thrombopoietin (TPO) are all critical enforcers of HSC quiescence. Alongside the essential chemokine stromal-derived factor 1 (SDF1a or CXCL12) and its C—X—C chemokine receptor type 4 (CXCR4), adhesion molecules such as vascular cell adhesion protein 1 (VCAM-1), various selectins, and extracellular matrix (ECM) proteins like fibronectin or hyaluronic acid are all essential regulators of HSC homing and anchoring in the niche.

The decision to remain at a quiescent state or to enter into an actively proliferating state is controlled by a number of factors through both cell intrinsic and extrinsic mechanisms. In response to extrinsic soluble factors; inflammatory cytokines such as interferon (IFN)-α and IFN-γ; growth factors such as granulocyte colony stimulating factor (GCSF), stem cell factor (SCF), and thrombopoietin (TPO); cytokines such as transforming growth factor (TGF)-β and tumor necrosis factor (TNF)-α; and chemokines such as the stromal cell derived factor (SDF)-1, HSCs can either enter dormancy or the cell cycle. Intrinsic factors that regulate HSC quiescence include: cell cycle inhibitors such as p21 and p57; transcription factors (TFs) such as Gfi1, Egr1, FOXOs, and PBX1; and ubiquitin ligases such as c-Cbl, Itch, Fbxw7, and A20. A harmony between intrinsic and extrinsic factors is essential for proper maintenance of HSCs in the bone marrow niche. (Nakagawa, M. M., Chen, H., & Rathinam, C. V. (2018). Constitutive Activation of NF-κB Pathway in Hematopoietic Stem Cells Causes Loss of Quiescence and Deregulated Transcription Factor Networks. Frontiers in cell and developmental biology, 6, 143).

Bone Microenvironment. The bone marrow can be subdivided into a hematopoietic cell compartment (the paranchyma) and the stroma, which is mainly composed of fibroblasts, adipocytes, nerves, and the bone marrow's vascular system. (Kopp, et. al. “The Bone Marrow Vascular Niche: Home of HSC Differentiation and Mobilization.” PHYSIOLOGY 20: 349-356, 2005; 10.1152/physiol.00025.2005).

Arterial vessels enter the marrow through foramina nutricia and then divide into several arterioles. Small arterioles and capillaries from these vessels span throughout the bone marrow and supply sinusoids, which are interconnected by intersinusoidal capillaries. The sinusoids are radially distributed around the draining central sinus, which measures ˜100 μm in diameter. The bone marrow sinusoids are unique and are not to be compared with regular veins. The sinusoidal wall consists of a single layer of endothelial cells and is devoid of supporting cells. The endothelial cells have no connective tissue covering, but are rather in direct contact with the parenchymal cells. The surrounding hematopoietic marrow is the major cellular moiety that supports reconstruction and remodeling of the sinusoidal microcirculation.

The rapid induction of marrow hypocellularity with cytotoxic agents or radiation is followed by a marked dilatation and collapse of the sinusoids and the central sinus. The lack of a regular vessel wall in sinusoids is reflected by a high level of permeability. The bone marrow microenvironment houses HSCs and hematopoietic progenitor cells (HPCs), where the bone microanatomic environment composed of neighboring stromal cells supports and instructs the stem cells. It has been postulated that the stromal environment itself might determine the quality of hematopoiesis. (Kopp, et. al. “The Bone Marrow Vascular Niche: Home of HSC Differentiation and Mobilization.” PHYSIOLOGY 20: 349-356, 2005; 10.1152/physiol.00025.2005).

HSCs and HPCs HSCs and HPCs predominantly reside either in direct contact or in close proximity to the vascular endothelium throughout their lifespan, termed the perivascular niche. From the moment of HSC embryonic specification from hemogenic endothelium, endothelial cells act as a critical cellular-hub that regulates a vast repertoire of biological processes crucial for HSC maintenance throughout its lifespan (Ramalingam P, Poulos M G, Butler J M. Regulation of the hematopoietic stem cell lifecycle by the endothelial niche. Curr Opin Hematol. 2017; 24(4):289-99. doi: 10.1097/MOH.0000000000000350. PubMed PMID: 28594660; PMCID: 5554937).

The bone marrow microenvironment houses not only the perivascular HSC niche, but also the osteoblastic niche, where each is defined by the role they play in the localization of stem cells. The osteoblastic niche in the bone marrow provides signals for the maintenance of lymphopoiesis, whereas the perivascular niche regulates the quiescence and maintenance of the HSC and its progeny. These spatial differences in hematopoietic tissues do not reflect or translate into the properties of the harbored stem cells themselves. Further research showed that stromal structures like the bone marrow sinusoidal vessels could serve as alternative cellular scaffolds upon which hematopoietic cells could reside and mature.

To delineate the bone marrow sinusoidal and arteriole network as a separate anatomic and functional entity from the endosteal zone, the name “vascular niche” is employed. Whereas the endosteal niche is thought to favor differentiation of the HSC, the centrally located vascular niche serves as a location that allows quiescence, maintenance, and differentiation of the HSC and ultimately mobilization to the peripheral circulation. In vivo genetic functional studies have demonstrated that HSCs have a close association with the bone marrow microvasculature in the vascular niche. Furthermore, nearly all mature megakaryocytes were found to be located adjacent to the thin-walled sinusoids, and whole megakaryocytes where shown to be able to transmigrate through intact endothelial cells. This observation is not limited to thrombopoiesis but can be applied to erythroid and B-lymphoid progenitors, as these lineages have also been reported to reside in defined niches within the marrow. These findings point to HSPC-endothelial cell interactions as being critical determinants in the maturation process, further reinforcing the idea of stem cell niches as microanatomic structures that are both permissive and instructive for stem cell maintenance and differentiation. Further, bone marrow endothelial cells (BMECs) were found to have adhesive properties, to interact with angiogenic and chemokinetic factors, and to contribute to supporting HSC self-renewal and differentiation thereby demonstrating the interdependence of the bone marrow parenchyma and the vascular niche. (Crane, G M, et al., “Adult haematopoietic stem cell niches,” Nat. Rev. Immunol. (2017) 17(9): 573-90; Ramalingam, P. et al., “Regulation of the hematopoietic stem cell lifecycle by the endothelial niche,” Curr. Opin. Hematol. (2017) 24(4): 289-99; Yu, V W, and Scadden, D T, “Heterogeneity of the bone marrow niche,” Curr. Opin. Hematol. (2016) 23(4): 331-38.

### Angiocrine Factors in Bone Tissue Cell Signaling

There are numerous angiocrine growth factors that play multiple roles in bone tissue cell signaling in the bone microenvironment. Table 1 below describes such angiocrine factors and their crosstalk with tissue cells in bone. (Sivan U, De Angelis J, Kusumbe A P. 2019 Role of angiocrine signals in bone development, homeostasis and disease. Open Biol. 9: 190144. http://dx.doi.org/10.1098/rsob.190144).

TABLE 1
Angiogenic factors in the bone microenvironment

| Angiocrine Factor | Source | Target Cell | Function |
|---|---|---|---|
| OPG | endothelial cell | osteoclasts | inhibit osteoclastogenesis |
| SEMA-III | endothelial cells | osteoclasts | bone remodeling |
| IL-33 | CD105+ endothelial | osteoblasts | osteogenesis, haematopoiesis |
| | cells | | |
| BMP-2 | endothelial cells | chondrocytes | endochondral bone formation, |
| | | | fracture repair |
| matrix | type H endothelial | chondrocytes | cartilage resorption, |
| metalloproteinases: | cells | | directional bone elongation |
| Mmp2, Mmp9, Mmp14 | | | |
| Timp1, Timp2, | type H endothelial | chondrocytes | bone resorption and remodeling |
| Timp3, Timp4 | cells | | |
| SCF | type H, arterial and | HSCs | HSC maintenance |
| | sinusoidal | | |
| | endothelial cells | | |
| nidogen-1 | sinusoidal and | pro-B cells | pro-B cell maintenance |
| | perivascular stromal | | |
| | cells | | |
| IL-7 | endothelial cells and | pro-B cells | pro-B cell maintenance |
| | perivascular stromal | | |
| | cells | | |
| CXCL12 | endothelial cells and | HSCs | HSC maintenance |
| | mesenchymal stem cells | | |
| tenascin-C | endothelial cells | HSCs | HSC survival |
| FGF-2 | endothelial cells | HSPCs | HSPC expansion |
| Jag-1 | endothelial cells | HSCs | HSC regeneration, lymphoma |
| | | | cell proliferation |
| NOS2 | endothelial cells | osteoblast | negative regulation of |
| | | | osteoblast differentiation |
| PDGF | endothelial cells | osteoprogenitor | osteoprogenitor proliferation |
| | | | and survival |
| TGF | endothelial cells | osteoprogenitor | osteoprogenitor survival |
| FGF1 | endothelial cells | osteoblast and | osteoprogenitor survival |
| | | osteoprogenitor | |
| Noggin | endothelial cells | osteoblast and | bone growth, mineralization |
| | | osteoprogenitor | and chondrocyte maturation |
| BMP-4 | endothelial cells | HSPC | expansion of HSPC |
| angiopoietin-1 | endothelial cells | HSPC | protection of HSPC |
| VCAM-1 | endothelial cells | osteoclasts, | leucocytes trafficking, |
| | | leucocytes and | protection of DTCs |
| | | fibroblasts | |
| E-selectin | endothelial cells | osteoclasts, | trafficking leucocytes, |
| | | leucocytes | cancer metastasis |
| von Willebrand | endothelial cells | Disseminated | protection of DTCs |
| factor | | tumour cells | |
| thrombospondin-1 | endothelial cells | disseminated | quiescence of DTCs |
| | | tumour cells | |
| IGFBP2 | endothelial cells | HSPC | expansion of HSPCs |
| ICAM-1 | endothelial cells | leucocytes and | leucocytes trafficking |
| | | fibroblasts | |

The link between hematopoietic and endothelial cells was found to be the hemangioblast, a common precursor for endothelial cells and hematopoietic cells. There is a strong interdependence of HSCs/HPCs and endothelial cells embryologically, which extends to the adult. (Kopp, et. al. “The Bone Marrow Vascular Niche: Home of HSC Differentiation and Mobilization.” PHYSIOLOGY 20: 349-356, 2005; 10.1152/physiol.00025.2005; Ramalingam P, Poulos M G, Butler J M. Regulation of the hematopoietic stem cell lifecycle by the endothelial niche. Curr Opin Hematol. (2017) 24(4):289-99. doi: 10.1097/MOH.0000000000000350. PubMed PMID: 28594660; PMCID: 5554937).

### Bone Marrow Vascular Niche

The bone marrow (BM) sinusoidal and arteriole network is a separate anatomic and functional entity from the endosteal zone. It consists of a network of thin-walled and fenestrated sinusoidal vessels whose integrity is maintained and supported by surrounding hematopoietic cells. However, this dependence is highly reciprocal in that the bone marrow vasculature provides not only a conduit for mature hematopoietic cells to the peripheral circulation, but also a site where the HSC pool is maintained in a quiescent state and hematopoietic progenitors, especially megakaryocytes, differentiate and set the stage for full reconstitution of hematopoiesis. (Kopp, et. al. “The Bone Marrow Vascular Niche: Home of HSC Differentiation and Mobilization.” Physiology (2005) 20: 349-356. 10.1152/physiol.00025.2005).

The blood vessels of the BM not only constitute the wall that separates the hematopoietic compartment from the peripheral circulation but are able to regulate hematopoiesis as well as stem cell mobilization and homing. Id.

The closeness between sinusoidal endothelial cells and HSCs is very important for their maintenance and their lineage specific differentiation [Ramalingam P, Poulos M G, Butler J M. Regulation of the hematopoietic stem cell lifecycle by the endothelial niche. Curr Opin Hematol. 2017; 24(4):289-99. doi: 10.1097/MOH.0000000000000350. PubMed PMID: 28594660; PMCID: 5554937]. The link between hematopoiesis and the THPO/cMpl system, and in particular its role in promoting the proliferation of megakaryocytic progenitors, is well known [Kopp, et. al. “The Bone Marrow Vascular Niche: Home of HSC Differentiation and Mobilization.” Physiology (2005) 20: 349-356. 10.1152/physiol.00025.2005, citing Nagasawa, T., Microenvironmental niches in the bone marrow required for B-cell development. Nat. Rev. Immunol. (2006) 6:107-116]. Despite the absence of the THPO/cMpl system, megakaryocyte maturation and platelet production may take place if megakaryocyte progenitors are close to BM sinusoids [Id., citing Avecilla, S T, et al., Chemokine-mediated interaction of hematopoietic progenitors with the bone marrow vascular niche is required for thrombopoiesis. Nat. Med. (2003) 10:64-71]. In THPO and cMpl KO mice where thrombopoiesis is compromised, CXCL12 and fibroblast growth factor-4 (FGF-4) restore the normal platelet through a mechanism that induces the expression of adhesion molecules, including very late antigen (VLA)-4 on megakaryocytes and VCAM-1 on endothelial cells [Id., citing Yoon, C H et al, Characterization of two types of endothelial progenitor cells (EPC) Korean Circ. J. (2004) 34:304-313; Maher, PA, Modulation of the epidermal growth factor receptor by basic fibroblast growth factor. J. Cell. Physiol. (1993) 154:350-358]. FGF may be considered an important factor in mediating the crosstalk between the vascular and the endosteal niche [Id., citing De Haan, G. et al., In vitro generation of long-term repopulating hematopoietic stem cells by fibroblast growth factor-1. Dev. Cell. (2003) 4:241-251]. It is thought that fibroblastic growth factor (FGF) forms a gradient between the two BM niches, important in the recruitment of HSCs and their progenitors to the vascular niche, where high expression of FGF receptors has been found [Id., citing Wright, DE, Physiological migration of hematopoietic stem and progenitor cells. Science. (2001) 294:1933-1936].

### Endothelial Cells (ECs) and the Endothelial Microniche

Each organ is arborized by an extensive network of specialized capillaries. Within each organ, the capillaries assume unique structural, phenotypic, functional and angiocrine attributes. In the hematopoietic organs such as bone marrow, stem and progenitor cells are in direct cellular contact with arterial and fenestrated specialized sinusoidal vessels demarcated by VEGFR3⁺VEGFR2⁺VEcad⁺CD31⁺ ECs. Tissue-specific stem and progenitor cells are strategically positioned in close proximity to homotypic capillary ECs. This intimate cellular interaction facilitates the delivery of membrane-bound and soluble angiocrine factors from specialized ECs to the recipient cells, which are located on the basolateral surface of blood vessels. Moreover, the luminal surface of ECs can serve as a signaling platform for stem and immune cells that navigate through the circulation. Tissue-resident parenchymal and stem cells regulate the activation state and response of ECs to regenerative stimuli through the production of angiogenic factors such as vascular endothelial growth factor (VEGF)-A, fibroblast growth factor (FGF)-2, stromal-cell-derived factor (SDF-1; also known as CXCL12), angiopoietins and thrombospondin-1 (TSP-1). Thus, the capillary network—without the influence of pericytes and mesenchymal cells—provides an adaptive platform that has the functional plasticity to integrate and relay these intravascular and extravascular cues to both resting and regenerating organs. (Rafii, S., Butler, J. M., & Ding, B.-S. (2016). Angiocrine functions of organ-specific endothelial cells. Nature, 529(7586), 316-325).

Accumulating evidence indicates that on pathophysiological stress (e.g., exposure to ionizing radiation, chemical injury, or hypoxic conditions) or loss of tissue mass, activated ECs relay inflammatory and injury-induced angiocrine signals to quiescent tissue-specific stem cells, which drives regeneration and enforces developmental set points to re-establish homeostatic conditions. Microvascular ECs therefore fulfill the criteria for professional niche cells that choreograph tissue regeneration by cradling and nurturing stem cells with physiological levels and proper stoichiometry of angiocrine factors. (Rafii, S., Butler, J. M., & Ding, B.-S. (2016). Angiocrine functions of organ-specific endothelial cells. Nature, 529(7586), 316-325).

ECs express inhibitory and stimulatory angiocrine factors that regulate the quiescence and proliferation of HSCs and HSPCs. Co-culture studies have also been used to demonstrate that bone-marrow sinusoidal ECs that are positive for VEGFR-3, VEGFR-2, VE-cadherin and CD31 stimulate the self-renewal of HSPCs by expressing soluble and membrane-bound angiocrine factors. (Id., citing Kobayashi, H. et al., Angiocrine factors from Akt-activated endothelial cells balance self-renewal and differentiation of hematopoietic stem cells. Nature Cell Biol. (2010) 12: 1046-56; Butler, J M, et al, Endothelial cells are essential for the self-renewal and repopulation of Notch-dependent hematopoietic stem cells. Cell Stem Cell (2010) 6: 251-64; Hooper, A T, et al, Engraftment and reconstitution of hematopoiesis is dependent on VEGFR2-mediated regeneration of sinusoidal endothelial cells. Cell Stem Cell (2009) 4: 263-74), including bone morphogenic protein (BMP)2 and BMP4, insulin growth factor binding protein (IGFBP)2, SDF-1, Desert hedgehog (Dhh) protein, Notch ligands, Wingless-type MMTV integration site (Wnt)5a, and Kit ligand. Bone-marrow sinusoidal ECs also drive the lineage-specific differentiation of HSPCs by producing granulocyte macrophage colony-stimulating factor (GM-CSF), interleukin (IL)-6, IL-8, granulocyte colony-stimulating factor (G-CSF), IL-1, tumour necrosis factor (TNF), chemokines and metalloproteinases (Id., citing Kobayashi, H. et al., Angiocrine factors from Akt-activated endothelial cells balance self-renewal and differentiation of haematopoietic stem cells. Nature cell biology. (2010) 12:1046-1056). ECs that are transitioning through various activation states also produce inhibitory factors, such as transforming growth factor (TGF)-β1 (Id, citing Brenet, F. et al., TGFbeta restores hematopoietic homeostasis after myelosuppressive chemotherapy. J Exp Med. (2013) 210:623-639), dickkopf-related protein (DKK)1 and DKK3, which block WNT signaling, and Noggin, which interferes with BMP signaling (Id., citing Kobayashi, H. et al., Angiocrine factors from Akt-activated endothelial cells balance self-renewal and differentiation of haematopoietic stem cells. Nature cell biology. (2010) 12:1046-1056).

ECs cultured under serum-free conditions were shown to supply angiocrine factors at physiological levels that increase the self-renewal of repopulating authentic mouse haematopoietic stem cells by 150-fold (Id., citing Butler, J M et al, Endothelial cells are essential for the self-renewal and repopulation of Notch-dependent hematopoietic stem cells. Cell Stem Cell. (2010) 6:251-264). and of human cord blood severe combined immunodeficiency repopulating cells by 8-fold (Id., citing Butler, J M, et al., Development of a vascular niche platform for expansion of repopulating human cord blood stem and progenitor cells. Blood. (2012) 120:1344-1347). Studies have shown that direct contact between haematopoietic cells and ECs is essential for the self-renewal and differentiation of HSPCs (Id., citing Kobayashi, H. et al., Angiocrine factors from Akt-activated endothelial cells balance self-renewal and differentiation of hematopoietic stem cells. Nature Cell Biol. (2010) 12: 1046-56; Butler, J M, et al, Endothelial cells are essential for the self-renewal and repopulation of Notch-dependent hematopoietic stem cells. Cell Stem Cell (2010) 6: 251-64; Hooper, A T, et al, Engraftment and reconstitution of hematopoiesis is dependent on VEGFR2-mediated regeneration of sinusoidal endothelial cells. Cell Stem Cell (2009) 4: 263-74). Compared with mesenchymal cells, ECs are more efficient at expanding umbilical cord blood-derived HSPCs (Id., citing Raynaud, C M, et al., Endothelial cells provide a niche for placental hematopoietic stem/progenitor cell expansion through broad transcriptomic modification. Stem cell research. (2013) 11:1074-1090). Other angiocrine factors, such as prostaglandin E2 (PGE2) (Id., citing North, T E, et al, Prostaglandin E2 regulates vertebrate haematopoietic stem cell homeostasis. Nature (2007) 447:1007-1011; Hoggatt, J. et al., Differential stem- and progenitor-cell trafficking by prostaglandin E2. Nature. (2013) 495:365-369), pleiotrophin (Id., citing Himburg, H A, et al., Pleiotrophin regulates the retention and self-renewal of hematopoietic stem cells in the bone marrow vascular niche. Cell reports. (2012) 2:964-975) and epidermal growth factor (EGF) (Id., citing Doan, P L et al, Epidermal growth factor regulates hematopoietic regeneration after radiation injury. Nat Med. (2013) 19:295-304), drive haematopoietic reconstitution, which establishes ECs as a physiological repository of HSPC-supportive factors.

The endothelial niche is essential not only for sustaining the self-renewal of HSCs, but also for multi-lineage reconstitution. The first in vivo evidence to support the role of the endothelial niche in haematopoiesis came from a study of mice that are unable to produce soluble Kit ligand, an essential regulator of haematopoietic stem-cell biology. It demonstrated that compartmentalized—yet interactive—stromal and endothelial niche cells regulate the regeneration of HSPCs. In response to physiological stress, the activation of matrix metalloproteinase (MMP)-9 leads to the release of soluble Kit ligand from cells in the niche, which stimulates the regeneration and proper transportation of HSPCs. Follow-up studies showed that phenotypically marked stem cells reside in close proximity to the endothelial niche (Id., citing Kiel, M J et al., SLAM Family Receptors Distinguish Hematopoietic Stem and Progenitor Cells and Reveal Endothelial Niches for Stem Cells. Cell. (2005) 121:1109-1121). Further evidence indicated that haematopoietic regeneration and thrombopoiesis after chemotherapy or irradiation is impaired by the conditional deletion of VEGFR-2 in ECs of adult mice (Id., citing Hooper, A T et al, Engraftment and reconstitution of hematopoiesis is dependent on VEGFR2-mediated regeneration of sinusoidal endothelial cells. Cell Stem Cell. (2009) 4:263-274) and by the targeting of VE-cadherin to disrupt reconstitution of the endothelial niche (Id., citing Avecilla, S T, et al. Chemokine-mediated interaction of hematopoietic progenitors with the bone marrow vascular niche is required for thrombopoiesis. Nat Med. (2004) 10:64-71; Hamada, T. et al., Transendothelial migration of megakaryocytes in response to stromal cell-derived factor 1 (SDF-1) enhances platelet formation. J Exp Med. (1998) 188:539-548).

The equilibrium between AKT and MAPK activation regulates multi-lineage haematopoietic recovery. Haematopoietic regeneration is orchestrated by the differential production of angiocrine factors that are induced by signaling pathways activated within ECs (Id., citing Kobayashi, H. et al, Angiocrine factors from Akt-activated endothelial cells balance self-renewal and differentiation of haematopoietic stem cells. Nature cell biology. (2010) 12:1046-1056). After myeloablative stress, angiogenic factors such as VEGF-A, VEGF-C, FGF-2 and the angiopoietins upregulate other angiocrine factors, including Jagged-1, through activation of AKT (also known as protein kinase B). Conditional deletion of Jagged-1 in ECs impairs haematopoietic recovery (Id., citing Pooulos, M G, et al., Endothelial jagged-1 is necessary for homeostatic and regenerative hematopoiesis. Cell reports. (2013) 4:1022-1034), which has been interpreted to suggest that Notch activation prevents the exhaustion of HSPCs. During the angiogenic phase of regeneration, AKT phosphorylation is accompanied by the activation of p42/p44 mitogen-activated protein kinase (MAPK). This triggers the secretion of G-CSF, macrophage colony-stimulating factor (M-CSF), GM-CSF and IL-6 to expand populations of myeloid, megakaryocytic and lymphoid progenitor cells and aid haematopoietic reconstitution (Id., citing Kobayashi, H. et al, Angiocrine factors from Akt-activated endothelial cells balance self-renewal and differentiation of haematopoietic stem cells. Nature cell biology. (2010) 12:1046-1056). In turn, maturing haematopoietic cells produce inhibitory angiogenic factors that prevent excessive sprouting of regenerating sinusoidal vessels. For example, mature megakaryocytes produce TSP-1, which decelerates angiogenesis and shuts off the production of activating angiocrine factors to restore homeostasis (Id., citing Nolan, D J, et al., Nolan D J, et al. Molecular signatures of tissue-specific microvascular endothelial cell heterogeneity in organ maintenance and regeneration. Dev Cell. (2013) 26:204-219; Kopp, H G, et al., Thrombospondins deployed by thrombopoietic cells determine angiogenic switch and extent of revascularization. J Clin Invest. (2006) 116:3277-3291) AKT-activated bone marrow ECs, which emulate some of the functions of in vivo angiogenic ECs, expand long-term repopulating haematopoietic stem cells under serum-free culture conditions, whereas bone-marrow-derived stromal cells direct stem-cell attrition (Id., citing Poulos, M G et al., Vascular Platform to Define Hematopoietic Stem Cell Factors and Enhance Regenerative Hematopoiesis. Stem cell reports. (2015) 8(5): 881-94. Moreover, protection of the haematopoietic microenvironment through transplantation of AKT-activated bone marrow ECs, but not mesenchymal ones, accelerates haematopoietic recovery after lethal irradiation (Id., citing Poulos, M G, et al. Vascular Platform to Define Hematopoietic Stem Cell Factors and Enhance Regenerative Hematopoiesis. Stem cell reports. (2015) 8(5): 881-94).

The contribution of the endothelial niche to steady-state hematopoiesis was unraveled by studies in which selective deletion in ECs of SDF-1, Kit ligand or Jagged-1 impaired the maintenance of HSCs and HSPCs (Id., citing Poulos, M G et al, Vascular Platform to Define Hematopoietic Stem Cell Factors and Enhance Regenerative Hematopoiesis. Stem cell reports. (2015) Ding, L., et al., Endothelial and perivascular cells maintain haematopoietic stem cells. Nature. (2012) 481:457-462; Ding, L., Morrison, SJ, Haematopoietic stem cells and early lymphoid progenitors occupy distinct bone marrow niches. Nature (2013) 495 (7440): 231-236; Inra, C N, et al., A perisinusoidal niche for extramedullary haematopoiesis in the spleen. Nature. (2015) Kimura, Y. et. al., c-Kit-Mediated Functional Positioning of Stem Cells to Their Niches Is Essential for Maintenance and Regeneration of Adult Hematopoiesis. PLoS One. (2011) 6:e26918).

Several studies have also scrutinized the relative contribution of bone marrow perivascular cells to the homeostasis of HSPCs (Id., citing Kunisaki, Y. et al., Arteriolar niches maintain haematopoietic stem cell quiescence. Nature. (2013) 502:637-643, Morrison, SJ, Scadden, DT. The bone marrow niche for haematopoietic stem cells. Nature. (2014) 505:327-334. Acar, M. et al., Deep imaging of bone marrow shows non-dividing stem cells are mainly perisinusoidal. Nature. (2015) 526:126-130). Because the functions and structural stability of endothelial and non-vascular cells is mutually dependent, the deletion of factors in one niche has the potential to perturb the constituents of the neighboring one. Therefore, genetic manipulations within the intimately associated endothelial niche and accompanying perivascular cells could have off-target effects, which must be taken into consideration. Nonetheless, the findings of these in vivo and reductionist in vitro studies suggest that, irrespective of the localization of HSPCs, angiocrine factors that are presented by either arteriolar or sinusoidal endothelial niches have executive functions and serve as ‘rheostats’ that choreograph haematopoietic stem-cell self-renewal and differentiation during homeostasis and recovery after haematopoietic suppression. Furthermore, these studies demonstrate that some, but not all, heterotypic ECs can support HSPC expansion, which confirms that each organotypic vascular bed is endowed with unique angiocrine attributes that are suitable for stem-cell homeostasis and reconstitution (Id., citing Nolan, D J et al, Molecular signatures of tissue-specific microvascular endothelial cell heterogeneity in organ maintenance and regeneration. Dev Cell. (2013) 26:204-219; Poulos, M G et al., Stem Cell Repts (2015) 8(5): 881-94; Butler, J M et al., Cell Stem Cell. (2010) 6: 251-64; Hooper, A T, et al, Cell Stem Cell (2009) 4: 263-74).

During fetal development, inductive signals from ECs specify the development of haemogenic ECs. (Id., citing Nguyen, P D et al, “Haematopoietic stem cell induction by somite-derived endothelial cells controlled by meox1. Nature (2014) 512: 314-18; Medvinsky, a., Dzierzak, E. Efinitive hematopoiesis is automously initiated by the AGM region. Cell (1996) 86: 897-906; Chen, M J et al. Runx1 is required for the endothelial to haematopoietic cell transition but not thereafter. Nature (2009) 457: 887-91). Thus, an endothelial niche could induce the direct conversion of all ECs into haemogenic ones, which give rise to definitive hematopoietic stem cells. Adult ECs were transduced with the transcription factors FosB, Gfi1, Runx1 and Spi1 (collectively termed FGRS). (Id.) However, FGRS-transduced ECs failed to convert to engraftable haematopoietic cells unless they were co-cultured in direct contact with ECs (Id., citing Sandler, V M, et al., Reprogramming human endothelial cells to haematopoietic cells requires vascular induction. Nature. (2014) 511:312-318. Moreover, co-culture of haematopoietic cells that were derived from mouse and non-human primate pluripotent stem cells and an endothelial niche enhanced the engraftment of putative haematopoietic cells, in part through the deployment of Notch ligands Id., citing Gori, J L, et al., Vascular niche promotes hematopoietic multipotent progenitor formation from pluripotent stem cells. J Clin Invest. (2015) 125:1243-1254; Hadland, B K et al., Endothelium and NOTCH specify and amplify aorta-gonad-mesonephros-derived hematopoietic stem cells. J Clin Invest. (2015) 125:2032-2045. Thus, angiocrine signals from ECs participate in the specification, development, homeostasis, self-renewal and differentiation of haematopoietic stem cells.

### Vasculogenesis/Angiogenesis

In addition to its role in the hematopoietic process, bone marrow is involved in the process of vasculogenesis. Vasculogenesis (meaning the process of new blood vessel formation) happens through migration, proliferation, and differentiation of endothelial progenitors to form new vessels followed by stabilizing and vascular maturation steps. It is an essential step in organ regeneration, wound healing, inflammation as well as tumor growth [Rohban, R., et al., Crosstalk between stem and progenitor cellular mediators with special emphasis on vasculogenesis,” Transfus. Med. Hemother. (2017) 44(3); 174-82; citing Segura, I. et al, Inhibition of programmed cell death impairs in vitro vascular-like structure formation and reduces in vivo angiogenesis. FASEB J. (2002) 16:833-841; Elmore, S., Apoptosis: a review of programmed cell death. Toxicol Pathol. (2007) 35:495-516; Krysko, D V, Vandenabeele, P. From regulation of dying cell engulfment to development of anti-cancer therapy. Cell Death Differ. (2008) 15:29-38].

Vascularization consists of migration and replication of endothelial progenitor cells (EPCs) or endothelial colony forming cells (ECFCs) as the backbone of newly formed vessels and mesenchymal stem and progenitor cells (MSPCs) as pericytes which serve as vessel supporters and maintain microvessel stability [Id., citing Reinisch, A. et al, Humanized system to propagate cord blood-derived multipotent mesenchymal stromal cells for clinical application. Regen Med. (2007) 2:371-382; Schallmoser, K., et al., Human platelet lysate can replace fetal bovine serum for clinical-scale expansion of functional mesenchymal stromal cells. Transfusion. (2007) 47:1436-1446; Reinisch, A., et al., Humanized large-scale expanded endothelial colony-forming cells function in vitro and in vivo. Blood. (2009) 113:6716-6725; Hofmann, N A, et al., Endothelial colony-forming progenitor cell isolation and expansion. Methods Mol Biol. (2012) 879:381-387]. Studies have shown that HSCs are able to deliver specific vasculogenic factors that facilitate contribution of EPCs into newly forming vessels [Id., citing Rafii, S., Lyden, D. Therapeutic stem and progenitor cell transplantation for organ vascularization and regeneration. Nat Med. (2003) 9:702-712]. EPCs and hematopoietic stem cells (HSCs) derived from adult tissues such as bone marrow (BM) have been shown to contribute to vasculogenesis during embryonic and postnatal physiological processes.

BM-derived endothelial cells (BMECs) are involved in indirectly (Tamma, R., & Ribatti, D. (2017). Bone Niches, Hematopoietic Stem Cells, and Vessel Formation. International Journal of Molecular Sciences, 18(1), 151), citing Yang L., et al. Expansion of myeloid immune suppressor Gr+CD11b+ cells in tumor-bearing host directly promotes tumor angiogenesis. Cancer Cell. (2004) 6:409-421). promoting vascular growth through the expression of angiogenic factors at the site where the neovascularization occurs. (Id., citing Ziegelhoeffer T. Bone marrow-derived cells do not incorporate into the adult growing vasculature. Circ. Res. (2004) 94:230-238) CXCR4 is highly expressed by BMECs and is involved in their mobilization and homing. (Id., citing Burger J. A. CXCR4: A key receptor in the crosstalk between tumor cells and their microenvironment. Blood. (2006) 107:1761-1767; Ruiz de Almodovar C., Luttun A., Carmeliet P. An SDF-1 trap for myeloid cells stimulates angiogenesis. Cell. (2006) 124:18-21) CXCL12 expression is also directly regulated by vascular endothelial growth factor (VEGF). The role of VEGF in BMECs' recruitment has been studied, inducing its expression in a transgenic system without other stimuli such as hypoxia. VEGF stimulates the expression of CXCL12 in perivascular cells and the latter attracts CXCR4+ circulating cells. Blocking VEGF receptor-1 (VEGFR-1) reduces the number of recruited perivascular cells in tumors (Id., citing Hattori K., et al. Vascular endothelial growth factor and angiopoietin-1 stimulate postnatal hematopoiesis by recruitment of vasculogenic and hematopoietic stem cells. J. Exp. Med. (2001) 193:1005-1014), suggesting that the effect of VEGF could involve VEGFR-1. Overall, these data confirm that VEGF is a pro-angiogenic growth factor that stimulates BMECs through the recruitment of perivascular cells and the activation of VEGFR-1.

A mechanism proposed for the release of BMECs from BM involves matrix metalloproteinase-9 (MMP-9). In hypoxic conditions, VEGF-A and CXCL12 are upregulated and induce the release of activated MMP-9 within the BM cell niches, which activates soluble kit-ligand, resulting in the release of BMECs into the peripheral blood. (Id., citing Seandel M., et al. A catalytic role for proangiogenic marrow-derived cells in tumor neovascularization. Cancer Cell. (2008) 13:181-183). Studies on mice mutant for 03 phosphorylation sites (DiYF) showed the presence of a high number of circulating CXCR4+BMECs as well as the loss of the ability of BMECs derived from DiYF mice to transmigrate through the endothelial monolayer (Id., citing Feng W., et al. The angiogenic response is dictated by β 3 integrin on bone marrow-derived cells. J. Cell Biol. (2008) 183:1145-1157), suggesting that the presence of complete β3 integrin activity is crucial for the recruitment of BMECs from the circulation into target tissues.

EPCs are bone-marrow-derived cells, functionally and phenotypically distinct from mature endothelial cells, with the ability to differentiate in endothelial cells in vitro and contribute to new blood vessel formation. (Id., citing Thijssen D. H. J. The role of endothelial progenitor and cardiac stem cells in the cardiovascular adaptations to age and exercise. Front. Biosci. (2009) 14:4685-4702, Khakoo A. Y., Finkel T. Endothelial progenitor cells. Ann. Rev. Med. (2005) 56:79-101). EPCs directly form new vessels and are a rich source of pro-angiogenic factors. (Id., citing Rehman J. Peripheral blood “endothelial progenitor cells” are derived from monocyte/macrophages and secrete angiogenic growth factors. Circulation. (2003) 107:1164-1169). Primitive EPCs expressing CD133, CD34, and VEGFR-2 differentiate in a mature form that loses CD133 expression. (Id., citing Yoon C. H., Seo J. B., et al. Characterization of two types of endothelial progenitor cells (EPC) Korean Circ. J. (2004) 34:304-313). VEGF is a strong inducer of EPC mobilization (Id, citing Carmeliet P. Mechanisms of angiogenesis and arteriogenesis. Nat. Med. (2000) 6:389-395) both in vitro and in vivo, and mobilization of EPCs into the peripheral circulation is increased after human recombinant VEGF administration in vivo. (Id., citing Asahara T., et al. VEGF contributes to postnatal neovascularization by mobilizing bone marrow-derived endothelial progenitor cells. EMBO J. (1999) 18:3964-3972) Also, GM-CSF participates in EPCs' mobilization from BM (Id., citing Takahashi et al. Ischemia- and cytokine-induced mobilization of bone marrow-derived endothelial progenitor cells for neovascularization. Nat. Med. (1999) 5:434-438), and osteoblast progenitors respond to hypoxia or insulin-like growth factor-1 (IGF-1), augmenting hypoxia inducible factor (HIF) signaling that results in HSC niche expansion associated with selective expansion of the erythroid lineage. (Id., citing Akeno N., et al. Induction of vascular endothelial growth factor by IGF-I in osteoblast-like cells is mediated by the PI3K signaling pathway through the hypoxia-inducible factor-2a Endocrinology. (2002) 143:420-425) The effect of erythroid lineage seems to be directly related to erythropoietin (EPO) expression in osteoblasts. (Id., citing Rankin E. B., et al. The HIF signaling pathway in osteoblasts directly modulates erythropoiesis through the production of EPO. Cell. (2012) 149:63-74) EPO is a key molecule in the process of vascular repair and neoangiogenesis and affects EPCs activity by increasing mobility and enhancing their ability to form tubes. (Id., citing Sautina L., et al. Induction of nitric oxide by erythropoietin is mediated by the common receptor and requires interaction with VEGF receptor 2. Blood. (2009) 115:896-905; Bahlmann F. H. Erythropoietin regulates endothelial progenitor cells. Blood. (2003) 103:921-926; Aicher A., et al. Essential role of endothelial nitric oxide synthase for mobilization of stem and progenitor cells. Nat. Med. (2003) 9:1370-1376).

In both vasculogenesis and angiogenesis, bi-directional signaling between ECs and the surrounding mesenchymal cells is critical. (Suda, T., & Takakurab, N. (2001). Role of hematopoietic stem cells in angiogenesis. International Journal of Hematology, 74(3), 266-71), citing Folkman, J. et al., Blood vessel formation: what is its molecular basis? Cell (1996) 87: 1153-55). Many molecules have been isolated that regulate the processes of vasculo-angiogenesis and are involved in maintaining the integrity of vessels by recruitment and formation of the periendothelial layer (a layer of pericytes that envelope ECs in adult vessels) or by mediating interactions between arteries and veins. (Id., citing Folkman, J. et al., Blood vessel formation: what is its molecular basis? Cell (1996) 87: 1153-55; Hanahan, D. Signaling vascular morphogenesis and maintenance. Science (1997) 277: 48-50; Wang, H U et al. Molecular distinction and angiogenic interaction between embryonic arteries and veins revealed by ephrin-B2 and its receptor Eph-B4. Cell (1998) 93: 741-53; Gale, NW, Yancopoulos, GD. Growth factors acting via endothelial cell-specific receptor tyrosine kinases: VEGFs, Angiopoietins, and ephrins in vascular development. Genes Dev. (1999) 13: 1055-66). Among them, two receptor tyrosine kinase subfamilies are characterized by their largely endothelial-specific expression. One family includes Flt-1/VEGFR1, Flk-1/KDR/VEGFR2, and Flt-4/VEGFR3, all of which are members of the vascular endothelial growth factor (VEGF) receptor family. (Id., citing Shalaby, F., et al. Failure of blood-islant formation and vasculogenesis in Flk-1 deficient mice. Nature (1995) 376: 62-6; Fong, G-H, et al. Role of the Flt-1 receptor tyrosine kinase in regulating the assembly of vascular endothelium. Nature (1995) 376: 66-70; Dumont, D J, et al, Cardiovascular failure in mouse embryos deficient in VEGF receptor-3. Science (1998) 282: 946-49; Ferrara, N. et al. Heterozygous embryonic lethality induced by targeted inactivation of the VEGF gene. Nature (1996) 380: 439-442). The other family includes TIE1/TIE and TIE2/TEK; the onset of embryonic expression of these receptors appears to follow that of the VERGF receptors (Id., citing Dumont, D J, et al. Vascularization of the mouse embryo: a study of flk-1, tek, tie, and vascular endothelial growth factor expression during development. Dev. Dyn. (1995) 203: 80-92). Embryos deficient in VEGF- or VEGFR show an early defect in vasculogenesis; while mice lacking TIE2 or TIE1 exhibit later defects in angiogenesis and vascular remodeling as well as in vascular integrity.

In the adult, ECs are already enveloped by pericytes in normal vessels. A necessary first step in angiogenesis in the adult vessel is the dissociation of pericytes tightly adhering to ECs. The balance between adhesion and dissociation of ECs and pericytes depends on angiopoietins, the ligands for TIE2. (Id., citing Davis, S. et al. Isolation of angiopoietin-1, a ligand for the TIE2 receptor, by secretion-trap expression cloning. Cell (1996) 87: 1171-80). Vessel sprouting begins with dissociation of pericytes from ECs, a process mediated by inactivation of Ang1 signaling. Subsequently, ECs may sprout toward Ang1-producing tissues. As recruitment of pericytes is followed by migration of ECs, Ang1-producing cells may promote vessel sprouting at a distance.

While, it has been shown that ECs migrate toward Ang1-producing HSCs, the basic mechanism of how HSCs migrate from the intraluminal cavity into parenchymal cells at a restricted point of a vessel is unclear. Id. It has been reported that peripheral CD34+ hematopoietic progenitors express high levels of matrix metallo-proteinases (MMP)-2 and -9 (Id., citing Janowska-Wieczorek, A. et al., Growth factors and cytokines upregulate gelatinase expression in bone marrow CD34+ cells and their transmigration through reconstituted basement membrane. Blood (1999) 93: 3379-90). Further embryonic HSCs (CD45+c-Kit+CD34+ cells) strongly express MMP-9. Moreover, these HSCs express TIE2 and adhere to fibronectin (FN) following stimulation by Ang1. (Id., citing Talakura, N. et al. Critical role of the TIE2 endothelial cell receptor in the development of definitive hematopoiesis. Immunity (1998) 9: 677-86). Taken together, these findings suggest that HSCs adhere to FN on ECs near the ischemic region, digest the matrix, and transmigrate through the basement membrane of capillary ECs into parenchymal cells. Therefore, it has been hypothesized that FN production on the intraluminal surface of ECs is the initial step in migration of HSCs and ECs. (Id.).

### Ossification/Osteogenesis

New vessels formed through angiogenesis during endochondral ossification are a source of perivascular osteoprogenitor cells and osteoclasts, important for modeling and remodeling processes that ensure correct skeletal development and growth. (Tamma, R., & Ribatti, D. (2017). Bone Niches, Hematopoietic Stem Cells, and Vessel Formation. International Journal of Molecular Sciences, 18(1), 151, citing Brandi M. L., Collin-Osdoby P. Vascular biology and the skeleton. J. Bone Miner. Res. (2006) 21:183-192). Bone cells secrete pro-angiogenic factors such as VEGF, which interacts with VEGFR-expressing cells including the endothelial cells, chondrocytes, osteoblasts, and osteoclasts. In the same way, endothelial cells release factors that regulate chondrocytes and cells of the osteoblast lineage. (Id., citing Kusumbe A. P., et al. Coupling of angiogenesis and osteogenesis by a specific vessel subtype in bone. Nature. (2014) 507:323-328) During angiogenesis, which occurs in endochondral ossification, endothelial cells of the advancing capillaries directly and indirectly influence the matrix resorption by producing proteases and regulatory molecules and by recruiting osteoclast precursors from the circulation. Moreover, endothelial cells release VEGF in response to the secretion of HIF1-α by hypoxic chondrocytes [Id., citing Manalo D. J. Transcriptional regulation of vascular endothelial cell responses to hypoxia by HIF-1. Blood. (2005) 105:659-669], and produce BMP-2 and BMP-4, stimulators of osteoblast differentiation. (Id., citing Sorescu G. P., et al. Bone morphogenic protein 4 produced in endothelial cells by oscillatory shear stress stimulates an inflammatory response. J. Biol. Chem. (2003) 278:31128-31135)).

Endothelin-1 (ET-1), expressed by endothelial cells, regulates angiogenesis directly by promoting endothelial cell migration, proliferation, and differentiation, or indirectly by inducing VEGF production in endothelial cells. The stimulation of ET-1 receptors in osteoblasts induces both their differentiation and the VEGF expression. (Id., citing Epstein F. H., Levin E. R. Endothelins. N. Engl. J. Med. (1995) 333:356-363.).

Osteoclasts are also involved in the angiogenesis process. Some authors have indicated that VEGF constitutes a chemoattractant factor for osteoclast precursors (Id., citing Henriksen K., et al. RANKL and vascular endothelial growth factor (VEGF) induce osteoclast chemotaxis through an ERK1/2-dependent mechanism. J. Biol. Chem. (2003) 278:48745-48753), and an autocrine/paracrine action of VEGF in osteoclasts has been found. Osteoclasts express VEGF in response to HIF1α, increased by the RANKL activation during osteoclast differentiation. (Id., citing Trebec-Reynolds D. P., et al. VEGF-A expression in osteoclasts is regulated by NF-κB induction of HIF-1α J. Cell. Biochem. (2010) 110:343-351) During bone resorption, the TGFβ1 released from the bone matrix induces VEGF expression in the bone-resorbing compartment, which consequently stimulates endothelial activity and supports angiogenesis.

Also, osteocytes contribute to angiogenesis. It is thought that during bone damage, the osteocytes that undergo apoptosis express VEGF. (Id., citing Cheung W.-Y., et al. Osteocyte apoptosis is mechanically regulated and induces angiogenesis in vitro. J. Orthop. Res. (2010) 29:523-530) Moreover, it has been found that the pulsatile fluid shear stress stimulation of MLOY4 osteocytes induces the secretion of VEGF. (Id., citing Juffer P., et al. Expression of muscle anabolic and metabolic factors in mechanically loaded MLO-Y4 osteocytes. AJP. (2011) 302:E389-E395)

Notch/Dll4 signaling is involved in angiogenesis in adult long bones. Arteries express D114 and JAG1, the latter also in perivascular osteoprogenitor cells. The role of the Notch/Dll4 system is to stimulate vessel growth and endothelial proliferation by regulating VEGFR expression. Moreover, mice with an impaired Notch/D114 pathway showed a reduction in long bone development and an increased number of immature osteoblasts. (Id., citing Ramasamy S. K., et al. Endothelial notch activity promotes angiogenesis and osteogenesis in bone. Nature. (2014) 507:376-380).

Pleiotrophin (PTN), a heparin binding growth factor (Id., citing Himburg H. A., et al. Pleiotrophin regulates the retention and self-renewal of hematopoietic stem cells in the bone marrow vascular niche. Cell Rep. (2012) 2:1774) is another local bone factor differentially expressed and secreted by BM sinusoidal endothelial cells within the vascular niche involved in angiogenesis in vivo and in vitro (d., citing Papadimitriou E., et al. Roles of pleiotrophin in tumor growth and angiogenesis. Eur. Cytokine Netw. (2009) 20:180-190). PTN exerts chemotaxis of pro-angiogenic early EPCs in a NOS-dependent manner (Id., citing Heiss C., et al. Pleiotrophin induces nitric oxide dependent migration of endothelial progenitor cells. J. Cell. Physiol. (2008) 215:366-373), and stimulates both osteoblast proliferation and bone matrix deposition. (Id., citing Tare R. S., et al. Pleiotrophin/osteoblast-stimulating factor 1: Dissecting its diverse functions in bone formation. J. Bone Miner. Res. (2002) 17:2009-2020).

### Angiocrine Factor Governing Vasculogenesis and Vascular Maturations

Molecular mediators governing vasculogenesis and vascular maturation can be grouped into three categories: 1) molecules that mediate mural-endothelial and endothelial-endothelial cell interactions; 2) molecules involved in cell-matrix interactions, and 3) molecules involved in signaling pathways. (Rohban, R., et al., Crosstalk between stem and progenitor cellular mediators with special emphasis on vasculogenesis,” Transfus. Med. Hemother. (2017) 44(3); 174-82).

Category I; Molecules that Mediate Mural-Endothelial and Endothelial-Endothelial Cell Interactions

VE-cadherin is an important mediator for endothelial-endothelial cell junctions, whereas neural cadherin (N-cadherin) mostly mediates the EC-mural cell junction in the process of vasculogenesis [Rohban, R., et al., Crosstalk between stem and progenitor cellular mediators with special emphasis on vasculogenesis,” Transfus. Med. Hemother. (2017) 44(3); 174-82, citing Dejana, E. et al, The control of vascular integrity by endothelial cell junctions: molecular basis and pathological implications. Dev Cell. (2009) 16:209-221]. Cellular communication between supporting stromal cells can also be promoted through N-cadherin molecules. The gap junction components, connexins (Cx37, Cx40 and Cx43), promote communication between endothelial cells and perivascular cells. Furthermore, the endothelial/leukocyte surface marker CD31 has been shown to provide permeability to endothelial-endothelial cell junctions [Id., citing Carmeliet, P., Jain, RK, Molecular mechanisms and clinical applications of angiogenesis. Nature. (2011) 473:298-307]. Tight junction molecules such as occludin, claudin, zonula occludens molecules (ZO-1, 2, 3) and the endothelial surface molecule CD148 are responsible for forming tight junctions in the blood brain barrier (BBB) and retinal microvessels in order to regulate endothelial-mural cell interaction in these sites.

While it has been shown that mechanical forces during growth and expansion of the vascular network serve as a stimulator to many of the cellular and molecular interactions in the process of vasculogenesis and vascular maturation [Id., citing Jain, RK, Molecular regulation of vessel maturation. Nat Med. (2003) 9:685-693], a limited number of studies that discuss the mechanical parameters that regulate the cellular junctions resulting in concise vasculogenesis pattern are available.

### Category II; Molecules Involved in Cell-Matrix Interactions

The extracellular matrix (ECM) serves as a pool enriched for different growth factors and enzymes contributing to vasculogenesis. Studies on integrins have provided information about the effect of various extracellular matrix components on the survival and migration of endothelial cells. It has been shown previously that the receptor α5β1 fibronectin, collagen I and collagen receptors α1β1 and α2β1 are involved in promoting vasculogenesis and inhibiting endothelial cell apoptosis, whereas thrombospondin 1 and 2 (Tsp1 and Tsp2) have been shown to block vasculogenesis through integrins and proteases [Id., citing Lawler, J., The functions of thrombospondin-1 and -2. Curr Opin Cell Biol. (2000) 12:634-640]. Paradoxically, some studies reported that suppressing the genes encoding integrin αvβ3 and integrin αvβ5 (integrins binding fibronectin, fibrinogen, endostatin and von Willebrand factor) does not inhibit vasculogenesis [Id., citing Hynes, R O, A reevaluation of integrins as regulators of angiogenesis. Nat Med. (2002) 8:918-921; Stupack, D G, Cheresh, D A, Get a ligand, get a life: integrins, signaling and cell survival. J Cell Sci. (2002) 115:3729-3738]. On the other hand, proteases released from endothelial and mural cells can cleave the matrix and plasma proteins into components that mediate endothelial cell apoptosis (e.g. angiostatin that results from plasminogen cleavage) whereas protease inhibitors in the matrix sustain vascular stability [Id., citing Jain, R K, Molecular regulation of vessel maturation. Nat Med. (2003) 9:685-693]. The precise role of cell-matrix interactions in vasculogenesis and vascular stability processes is not understood.

### Category III; Molecules Involved in Signaling Pathways

Cellular activities are governed by a series of molecular events resulting in cellular crosstalk. This regulatory cell signaling implicates protein-protein interactions as the major cell process regulators as well as cell-microenvironment interactions which are necessary for differentiation, tissue homeostasis and repair [Id., citing Kolch, W., Pitt, A. Functional proteomics to dissect tyrosine kinase signaling pathways in cancer. Nat Rev Cancer. (2010); 10:618-629; Gadbois, D M, et al, Multiple kinase arrest points in the G1 phase of nontransformed mammalian cells are absent in transformed cells. Proc Natl Acad Sci USA. (1992) 89:8626-8630]. Aberrant cell signaling could result in cell malfunction or diseases such as cancer and diabetes [Id., citing Kolch W, Pitt A. Functional proteomics to dissect tyrosine kinase signaling pathways in cancer. Nat Rev (Cancer). 2010; 10:618-629].

Although cell signaling has been studied intensively within the single cell types, it may also happen between two different cell types e.g. when the surface embryonic cells are being attached and the embryo is implanted to the endometrial tissue. This process is known to be mediated by β-catenin signaling molecule in the process of Wingless (WNT) signaling [Id., citing Mohamed, O A, et al., Uterine Wnt/beta-catenin signaling is required for implantation. Proc Natl Acad Sci USA. (2005); 102:8579-8584].

Cellular components that are involved in vasculogenesis are dependent on a potent, well-orchestrated and regulated system of cellular crosstalk catalyzed by different classes of signaling molecules. Several signaling pathways have been identified to regulate endothelial progenitor-mesenchymal stem and progenitor crosstalk during vasculogenesis, such as calcium calmodulin and focal adhesion protein kinase signaling pathway [Id., citing Rohban, R. et al., Identification of an effective early signaling signature during neo-vasculogenesis in vivo by ex vivo proteomic profiling. PLoS One (2013) 8:e66909]. Other signaling events have been studied through which hematopoietic stem and endothelial progenitor cells communicate within the vascular microenvironment e.g. SDF-1 (CXCL12)/CXCR4 signaling [Id., citing HO, T K, et al, Stromal-cell-derived factor-1 (SDF-1)/CXCL12 as potential target of therapeutic angiogenesis in critical leg ischaemia. Cardiol Res Pract. (2012) 2012:7; Petit, I. et al, The SDF-1-CXCR4 signaling pathway: a molecular hub modulating neo-angiogenesis. Trends Immunol. (2007) 28:299-307; Burger, J A, et al, CXCR4 chemokine receptors (CD184) and alpha4beta1 integrins mediate spontaneous migration of human CD34+ progenitors and acute myeloid leukemia cells beneath marrow stromal cells (pseudoemperipolesis) Br J Haematol. (2003) 122:579-589; De Clercq, E., Potential clinical applications of the CXCR4 antagonist bicyclam AMD3100. Mini Rev Med Chem. (2005) 5:805-824], vascular endothelial growth factor signaling (VEGF) [Id., citing Ferrara, N., VEGF-A: a critical regulator of blood vessel growth. Eur Cytokine Netw. (2009) 20:158-163; Nagy, J A et al, VEGF-A and the induction of pathological angiogenesis. Annu Rev Pathol. (2007) 2:251-275; Phang, LK, Gerhardt, H. Angiogenesis: a team effort coordinated by notch. Dev Cell. (2009) 16:196-208; Tvorogov, D. et al., Effective suppression of vascular network formation by combination of antibodies blocking VEGFR ligand binding and receptor dimerization. Cancer Cell. (2010); 18:630-640], Tie2/Ang-1 signaling [Id., citing Maisonpierre, P. C. et al., Angiopoietin-2, a natural antagonist for Tie2 that disrupts in vivo angiogenesis. Science. (1997) 277:55-60, Uemura, A., et al., Recombinant angiopoietin-1 restores higher-order architecture of growing blood vessels in mice in the absence of mural cells. J Clin Invest. (2002) 110:1619-1628], hedgehog [Id., citing Guo, W. et al., Activation of SHH signaling pathway promotes vasculogenesis in post-myocardial ischemic-reperfusion injury. Int J Clin Exp Pathol. (2015) 8:12464-12472; Williams, C. et al. Hedgehog signaling induces arterial endothelial cell formation by repressing venous cell fate. Dev Biol. (2010) 341:196-204; Lawson, N D, et al., sonic hedgehog and vascular endothelial growth factor act upstream of the Notch pathway during arterial endothelial differentiation. Dev Cell. (2002) 3:127-136] and Notch signaling [Id., Lawson, N D et al., Notch signaling is required for arterial-venous differentiation during embryonic vascular development. Development. (2001) 128:3675-3683; Fischer, A., et al., The Notch target genes Hey1 and Hey2 are required for embryonic vascular development. Genes Dev. (2004) 18:901-911], as well as Wingless (Wnt) signaling [Id., citing Gore, A V, et al, Rspo1/Wnt signaling promotes angiogenesis via Vegfc/Vegfr3. Development. (2011) 138:4875-4886; Li, R. et al., Shear stress-activated Wnt-angiopoietin-2 signaling recapitulates vascular repair in zebrafish embryos. Arterioscler Thromb Vasc Biol. (2014) 34:2268-2275; Chen, Y. et al., Inhibition of Wnt inhibitory factor 1 under hypoxic condition in human umbilical vein endothelial cells promoted angiogenesis in vitro. Reprod Sci. (2016) 23:1348-1358; Zhang, Z. et al., Wnt/beta-catenin signaling determines the vasculogenic fate of postnatal mesenchymal stem cells. Stem Cells. (2016) 34:1576-1587].

Communication paths between hematopoietic stem cells and endothelial progenitor cells that create effective cellular crosstalks during vasculogenesis include the following.

SDF-1-CXCR4 Signaling Pathway

Studies have revealed that SDF-1 (also known as CXCL12) has a crucial impact on recruitment of CXCR4+BM cells to vascular microenvironment, resulting in revascularization of injured tissues and tumor growth [Id., citing Ho, T K et al., Stromal-cell-derived factor-1 (SDF-1)/CXCL12 as potential target of therapeutic angiogenesis in critical leg ischaemia. Cardiol Res Pract. (2012) 2012:7; Petit, I. et al. The SDF-1-CXCR4 signaling pathway: a molecular hub modulating neo-angiogenesis. Trends Immunol. (2007) 28:299-307]. The precise mechanism by which activation of CXCR4 modulates vasculogenesis has not been unraveled yet. SDF-1 has been also shown to promote mobilization of pro-vasculogenic CXCR4+ VEGFR-1+ hematopoietic cells, thereby supporting revascularization of injured and ischemic organs [Id., citing Petit, I. et al., The SDF-1-CXCR4 signaling pathway: a molecular hub modulating neo-angiogenesis. Trends Immunol. (2007) 28:299-307]. During migration, HSCs have been found to express CXCR4, the receptor for the chemokine SDF-1, and integrin α4β1. In BM stromal microenvironment, osteoblasts and endothelial cells release SDF-1 while CXCR4 is expressed by hematopoietic progenitor cells, thus supporting successful BM engraftment in vivo [Id., Burger, J A et al., CXCR4 chemokine receptors (CD184) and alpha4beta1 integrins mediate spontaneous migration of human CD34+ progenitors and acute myeloid leukaemia cells beneath marrow stromal cells (pseudoemperipolesis) Br J Haematol. (2003) 122:579-589; De Clercq, E., Potential clinical applications of the CXCR4 antagonist bicyclam AMD3100. Mini Rev Med Chem. (2005) 5:805-824].

VEGF Signaling

VEGF family mediators like VEGF-A play an important role not only during initiation of vasculogenesis through VEGFR-2 (FLK-1), but also during vessel maturation that ultimately results in establishment of arteries (arteriogenesis) [Id., citing Ferrara, N., VEGF-A: a critical regulator of blood vessel growth. Eur Cytokine Netw. (2009) 20:158-163; Nagy, J A, VEGF-A and the induction of pathological angiogenesis. Annu Rev Pathol. (2007) 2:251-275]. Neurophilins (NRP1 and NRP2) serve as independent VEGF receptors that increase VEGFR-2 activity. VEGFR-2 deficiency and severe decrease in VEGF expression hampers vascular development [Id., citing Carmeliet, P. Angiogenesis in health and disease. Nat Med. (2003) 9:653-660]. Mutations and polymorphism in the gene encoding VEGFR-2 result in vascular tumor formation as well as in abnormal and/or pathological vasculogenesis patterns [Id., citing Jain, R K, et al, Biomarkers of response and resistance to antiangiogenic therapy. Nat Rev Clin Oncol. (2009) 6:327-338].

In perfused vessels, an endothelial tip cell is activated by VEGF-C, a ligand for VEGFR-2 and VEGFR-3, to direct the vessel growth in the presence of VEGF receptors and Notch ligands like DLL4 and JAGGED1. Upregulation of DLL4 and activation of Notch signaling in stalk cells result in VEGFR-2 downregulation and cause the stalk cell to be less responsive to VEGF, thus ensuring a guiding role for the tip cell in the process of vascular development [Id., citing Phng, LK, Gerhardt, H., Angiogenesis: a team effort coordinated by notch. Dev Cell. (2009) 16:196-208; Tvorogov, D., et al., Effective suppression of vascular network formation by combination of antibodies blocking VEGFR ligand binding and receptor dimerization. Cancer Cell. (2010) 18:630-640]. Paracrine VEGF secreted from cancer cells, myeloid cells, or pericytes promotes vascular branching, whereas vasculature homeostasis is sustained by autocrine VEGF secretion [Id., citing Stockmann, C. et al., Deletion of vascular endothelial growth factor in myeloid cells accelerates tumorigenesis. Nature. (2008) 456:814-818; Lee, S. et al., Autocrine VEGF signaling is required for vascular homeostasis. Cell. (2007) 130:691-703].

VEGFR-3 signaling plays a crucial role in prenatal venous-derived vasculogenesis as well as in lymphatic vessel remodeling from the pre-existing ones. A study in zebrafish revealed that vessel formation through sprouting of venous endothelial cells is prohibited by VEGF-2, whereas VEGF-3 facilitates the sprouting of the venous-fated endothelial cells resulting in vein development [Id., citing Herbert, S P et al., Arterial-venous segregation by selective cell sprouting: an alternative mode of blood vessel formation. Science. (2009) 326:294-298].

VEGF-B is another member of the VEGF family that shows vasculogenic potential only in some particular tissues like heart tissue, facilitating cardiac vascular development without additional effects on permeability of the vessels [Bry, M. et al., Vascular endothelial growth factor-B acts as a coronary growth factor in transgenic rats without inducing angiogenesis, vascular leak, or inflammation. Circulation. (2010) 122:1725-1733].

VEGFR-1—also known as FLT-1—possesses weak tyrosine kinase activity but can trap extra amounts of free VEGF to maintain VEGFR-2 activity in a normal state. VEGFR-1 blockade and/or deficiency lead to vessel overgrowth [Fischer, C. et al., FLT1 and its ligands VEGFB and PlGF: drug targets for anti-angiogenic therapy? Nat Rev Cancer. (2008) 8:942-956]. In contrast, the endothelial and stromal VEGFR-1 signaling cascade has been shown to promote pathological vasculogenesis by providing a higher growth rate for VEGFR-1+ cancer cells and by increasing matrix metalloproteinase 9 expression in endothelial cells at a metastatic state [Id., citing Schwartz, J D et al., Vascular endothelial growth factor receptor-1 in human cancer: concise review and rationale for development of IMC-18F1 (human antibody targeting vascular endothelial growth factor receptor-1) Cancer. (2010) 116:1027-1032].

It has been reported that the loss of a single VEGF allele in mice leads to severe vascular impairment and mortality before HSC specification [Id., citing Carmeliet, P., Abnormal blood vessel development and lethality in embryos lacking a single VEGF allele. Nature. (1996) 380:435-439]. Other studies have reported that VEGF-A is crucial for HSC formation: Longer VEGF-A isoforms are essential for HSC specification [Id., citing Kim, A D, et al., Cell signaling pathways involved in hematopoietic stem cell specification. Exp Cell Res. (2014) 329:227-233]. Also, VEGF signaling has been shown to have crucial impact in formation of HSCs from endothelial precursors [Kim, A D et al., Cell signaling pathways involved in hematopoietic stem cell specification. Exp Cell Res. (2014) 329:227-233].

Notch Signaling

The Notch signaling pathway is required for determining the arterial program of both endothelial and smooth muscle cells; however, it is simultaneously involved in the generation of HSCs, which will give rise to hematopoietic cells. Notch signaling also regulates the function of EPCs, which are BM-derived cells able to differentiate into endothelial cells that could be considered the adult correlate of the angioblast. In addition, Notch signaling has been reported to control sprouting angiogenesis during blood vessels formation in adults [Id., citing Caolo, V. et al., Notch regulation of hematopoiesis, endothelial precursor cells, and blood vessel formation: orchestrating the vasculature. Stem Cells Int. (2012) 2012:805602].

Notch signaling is involved in cell fate decisions during murine vascular development and hematopoiesis in BM microenvironment. In order to elucidate the relation between HSCs and human EPCs in the BM niche, impact of Notch signals (Jagged-1 and delta-like ligand 1 (Dll-1)) on proliferation and differentiation of human CD133+ EPCs needs to be studied [Id., citing Caolo, V. et al., Notch regulation of hematopoiesis, endothelial precursor cells, and blood vessel formation: orchestrating the vasculature. Stem Cells Int. (2012) 2012:805602].

To investigate the vasculogenic properties of human Jagged-1- and human Dll-1-stimulated EPC in vivo, these cells were transplanted into the ischemic limb of nude mouse. The result indicated that transplantation of EPCs stimulated by human Jagged-1, but not human Dll-1, elevated density of microvessels in ischemic limb muscles, suggesting that human Notch signaling affects EPC proliferation and differentiation in the BM niche. Human Jagged-1 has been shown to induce proliferation and differentiation of CD133+ cord blood progenitor cells compared to human Dll-1; therefore, human Jagged-1 signaling in the BM microenvironment can be used to expand EPCs for therapeutic and regenerative vasculogenic interventions. Moreover, it has been revealed that Jagged-1 signaling in the BM microenvironment supports proliferation and expansion of EPCs and promotes commitment of CD133+ human umbilical cord blood cells during vasculogenesis [Id., citing Ishige-Wada, et al., Jagged-1 signaling in the bone marrow microenvironment promotes endothelial progenitor cell expansion and commitment of CD133+ human cord blood cells for postnatal vasculogenesis. PLoS One. (2016) 11:e0166660].

In the vessel branching model, it has been shown that the tip cells of the vessel migrate while stalk cells proliferate. It has been hypothesized that this might be a result of Notch signaling in this model [Id., citing Phng, LK, Gerhardt, H., Angiogenesis: a team effort coordinated by notch. Dev Cell. (2009) 16:196-208]. VEGFR-2 is activated in response to VEGF and causes expression of DLL4 in the tip cells; consequently, DLL4 activates Notch in the stalk cells that suppresses VEGFR-2 while upregulating VEGFR-1, resulting in less sprouting and branching but more vessel formation. JAGGED1 is another Notch ligand that is mainly expressed by stalk cells and contributes to DLL4 in order to select the tip cell [Id., citing Benedito, R. et al., The notch ligands D114 and Jagged1 have opposing effects on angiogenesis. Cell. (2009) 137:1124-1135]. However, as Notch signaling itself activates its inhibitor Notch-regulated ankyrin protein over time, this signaling cascade varies in the vascular microenvironment [Id., citing Phng, L K, et al., Nrarp coordinates endothelial Notch and Wnt signaling to control vessel density in angiogenesis. Dev Cell. (2009) 16:70-82].

Notch signaling mediators play an important role in the development of arterio-venous and venous endothelial structure in the establishing vessel [Id., citing Lawson, N D, et al., Notch signaling is required for arterial-venous differentiation during embryonic vascular development. Development. (2001) 128:3675-3683; Shawber, C J, Kitajewski, J., Notch function in the vasculature: insights from zebrafish, mouse and man. Bioessays. (2004) 26:225-234]. In endothelial cells, activation of Notch signaling leads to induction of many arterial markers including ephrin B2 and CD44, and suppression of venous markers such as ephrin type B receptor 4 [Id., citing Lawson, N D, et al., Notch signaling is required for arterial-venous differentiation during embryonic vascular development. Development. (2001) 128:3675-3683; Fischer, A., et al., The Notch target genes Hey1 and Hey2 are required for embryonic vascular development. Genes Dev. (2004) 18:901-911].

Hedgehog Signaling

Signaling by the hedgehog (hh) family molecules, including sonic hedgehog (shh) and Indian hedgehog (ihh), have a regulatory role in Notch expression, thus contributing to vascular structure and arterial formation in the embryo [Id., citing Swift. M R, Weinstein, BM., Arterial-venous specification during development. Circ Res. (2009) 104:576-588]. It has been revealed that hh signaling is genetically upstream of VEGF cascade, which governs Notch activation in the endothelium [Id., citing Lawson, N D et al., sonic hedgehog and vascular endothelial growth factor act upstream of the Notch pathway during arterial endothelial differentiation. Dev Cell. (2002) 3:127-136]. It can be concluded that hh signaling is a key regulator of a crucial crosstalk which governs accurate pattern of vascular formation and endothelium generation [Id., citing Lawson, N D et al., sonic hedgehog and vascular endothelial growth factor act upstream of the Notch pathway during arterial endothelial differentiation. Dev Cell. (2002) 3:127-136; Kim, A D, et al., Cell signaling pathways involved in hematopoietic stem cell specification. Exp Cell Res. (2014) 329:227-233]. A study aiming to investigate the impact of shh expression in vasculogenesis in the course of myocardial injury revealed that upon application of shh, vasculogenic components such as VEGF and fibroblast growth factor were significantly upregulated [Id., citing Guo, W., et al., Activation of SHH signaling pathway promotes vasculogenesis in post-myocardial ischemic-reperfusion injury. Int J Clin Exp Pathol. (2015) 8:12464-12472].

It has been shown that arterial endothelial cell formation is induced by hh signaling through repressing venous cell fate in zebrafish models; upregulation of hh signaling elevates arterial cellular population hampers venous cell fate during vasculogenesis [Id., citing Williams, C. et al., Hedgehog signaling induces arterial endothelial cell formation by repressing venous cell fate. Dev Biol. (2010) 341:196-204]. This evidence, together with several reports on the role of hh signaling in vascularity of tumors such as breast cancerous tumors [Id., citing Harris, L G et al., Increased vascularity and spontaneous metastasis of breast cancer by hedgehog signaling mediated upregulation of cyr61. Oncogene. (2012) 31:3370-3380], is unraveling the involvement of hh signaling in vascular formation and development.

Wnt Signaling

Wnt signaling has been shown to govern specification and homeostasis of several tissues. Wnt signaling pathway consists of 19 ligands that associate with frizzled (FZD) receptors on the surface of several cell types [Id., citing Bhanot, P. et al, A new member of the frizzled family from Drosophila functions as a Wingless receptor. Nature. (1996) 382:225-230; Yang-snyder, J., et al., A frizzled homolog functions in a vertebrate Wnt signaling pathway. Curr Biol. (1996) 6:1302-1306]. When there is no ligand binding, β-catenin is degraded [Id., citing Gao, Z H, et al., Casein kinase I phosphorylates and destabilizes the beta-catenin degradation complex. Proc Natl Acad Sci USA. (2002) 99:1182-1187]. However, activation of Wnt receptors through induction of the ligand blocks β-catenin degradation, thus allowing for translocation of this molecule to the nucleus and activation of target gene transcription [Id., citing Angers, S., Moon, R T, Proximal events in Wnt signal transduction. Nat Rev Mol Cell Biol. (2009) 10:468-477]. It has been revealed that β-catenin deletion in the endothelium results in hematopoietic deficiencies [Id., citing Kim, A D et al., Traver D. Cell signaling pathways involved in hematopoietic stem cell specification. Exp Cell Res. (2014) 329:227-233]. These findings indicate that Wnt signaling plays a crucial role in HSC and artery fate.

It has also been shown that endothelial cells express Wnt ligands and their FZD receptor that control endothelial cell proliferation. As vessel branching takes place, Wnt signaling is activated by Notch in stalk cells [Id., citing Phng, L K, et al., Nrarp coordinates endothelial Notch and Wnt signaling to control vessel density in angiogenesis. Dev Cell. (2009); 16:70-829]. Suppression of some of Wnt and FZD genes (Wnt2, Wnt5a, FZD4, and FZD5) in mouse resulted in defective vascular structures. Inactivation of WNT7a and WNT7b has been shown to result in an impaired BBB vessel formation [Id., citing Dejana, E., The role of Wnt signaling in physiological and pathological angiogenesis. Circ Res. (2010) 107:943-952].

### Hematopoiesis

The term “hematopoiesis” as used herein refers to the process by which the cellular constituents of blood are continually replenished throughout the lifetime of an organism by differentiating from hematopoietic stem cells (HSCs) to mature, functional cell types of the blood lineages.

The hematopoietic cell lineage is organized such that HSCs sit atop the hierarchy and give rise to committed progenitor cells, which in turn give rise to mature, differentiated cells. There are two major differences between HSCs and committed progenitors: HSCs are multipotent and they have the ability to self-renew indefinitely. LT-HSCs are a rare, quiescent population in bone marrow and have full long-term (>3˜4 months) reconstitution capacity, whereas ST-HSCs only have a short-term (mostly <1 month) reconstitution ability [Chang, H. et al. Protein & Cell (2020) 11: 34-44]. LT-HSCs differentiate into ST-HSCs, and subsequently, ST-HSCs differentiate into multipotent progenitors (MPPs), which have no detectable self-renewal ability [Id., citing Yang, L. et al. Blood (2005) 105: 2717-23]. The poorly self-renewing short-term HSC (ST-HSC) and other multipotent progenitors (MPP) nevertheless are fully multipotent at the single cell level [Weiskopf, K. et al, Microbiol. Spectr. (2016) 4 (5): doi:10.1128/microbiolspec.MCHD-0031-2016, citing Morrison, S J et al. Development (1997) 124 (10): 1929-39]. Downstream of MPP are committed progenitors. Committed progenitors are oligopotent (i.e., they are further restricted than MPPs in their capacity to differentiate) and have limited ability to self-renew. Therefore, when cells progress through hematopoiesis they become more differentiated and more frequent in number. They also lose their capacity to self-renew, become more restricted in their differentiation potential, and gain expression of molecules required for functional specialization. Differentiation occurs in one direction with restriction towards a particular lineage, with no significant evidence of transdifferentiation between hematopoietic lineages under normal conditions.

The hematopoietic lineage is divided into two main branches: the myeloid arm and the lymphoid arm. The Common Myeloid Progenitor (CMP) gives rise to the myeloid arm, which can give rise to all myeloid cells. The Common Lymphoid Progenitor (CLP) gives rise to the lymphoid arm, which can give rise to all lymphoid cells.

The hematopoietic stem cell (HSC) is a multipotent stem cell that resides in the bone marrow and has the ability to form all the cells of the blood and immune system. It has the ability to self-replicate and differentiate into progeny of multiple lineages. Human HSC activity resides in CD34Thy-1− populations. [Weiskopf, K. et al., “Myeloid cell origins, differentiation, and clinical implications,” Microbiol. Spectr. (2016) 4(5): 10.1128/microbiolspec. MCHD-0031-2016]. The CD90+CD45RA− population contains the true long-term HSC (“LT-HSC”) in humans, while the CD90−CD45RA− population represents an intermediate downstream multipotent progenitor (MPP). [Id]. The lin−CD34+CD38+ population of human bone marrow has limited ability to self-renew and exhibits a high proportion of myeloid-biased differentiation [Id., citing Manz, M G, et al, “Prospective isolation of human clonogenic common myeloid progenitors.” Proc. Natl Acad. Sci. USA (2002) 99 (18): 11872-77]. Expression of CD45RA and IL-3Rα further subdivide this population, yielding three distinct subpopulations: IL-3RαloCD45RA−, IL-3RαloCD45RA+, and IL-3Rα−CD45RA− cells. In vitro, the IL-3RαloCD45RA− population gave rise to the full range of the myeloid lineage, including mixed colonies, suggesting this population represented the human common myeloid progenitor (CMP) [Id., citing Manz, M G, et al, “Prospective isolation of human clonogenic common myeloid progenitors.” Proc. Natl Acad. Sci. USA (2002) 99 (18): 11872-77]. On the other hand, the IL-3RαloCD45RA+ population only gave rise to cells of the granulocyte and macrophage lineages, and the IL-3Rα−CD45RA− population predominantly gave rise to cells of the erythroid and megakaryocyte lineage; thereby indicating these populations represented the granulocyte/macrophage lineage-restricted progenitor (GMP) and the megakaryocyte/erythrocyte lineage-restricted progenitor (MEP), respectively [Id., citing Manz, M G, et al, “Prospective isolation of human clonogenic common myeloid progenitors.” Proc. Natl Acad. Sci. USA (2002) 99 (18): 11872-77].

Within the human MEP population, fractionation studies helped define the unipotent human erythrocyte progenitor (EP) as CD71intermediate(int)/+CD105+, and when sorted to purity, gave rise exclusively to erythrocytes in vitro with no megakaryocyte potential [Id., citing Mori, Y. et al., “Prospective isolation of human erythroid lineage-committed progenitors,” Proc. Natl Acad. Sci. USA (2015) 112 (31): 9638-43]. Additionally, an erythrocyte-biased MEP (E-MEP) was identified as CD71+CD105 that was an intermediate between the MEP and the EP [Id]. Downstream stages of human erythropoiesis have also been isolated to purity, including the primitive erythroid progenitor cells (burst-forming unit-erythroid or BFU-E) and later-stage colony-forming-unit-erythroid (CFU-E). These populations were principally distinguished as IL-3R−CD34+CD36− and IL-3R−CD34−CD36+, respectively [Id., citing Li, J. et al, “Isolation and transcriptome analyses of human erythroid progenitors: BFU-E and CFU-E.” Blood (2014) 124 (24): 3636-45].

Bone marrow endothelial cells (BMECs) are key to a mechanistic understanding of the blood cell-producing capability of the bone marrow (i.e., hematopoiesis). Studies on other endothelial cell types like human umbilical vein endothelial cells (HUVECs) showed that transendothelial trafficking was dependent on the expression of surface receptors or adhesion molecules, which were inducible by inflammatory cytokines. Therefore, it was thought that the release of mature blood cells as well as HSC/HPC mobilization and homing were likely to be regulated by similar mechanisms. BMECs were found to support the proliferation and differentiation of hematopoietic progenitors in vitro via production of various cytokines and also possibly via physical contact. Coculturing megakaryocytes and BMECs resulted in survival prolongation of BMECs, probably because megakaryocytes secrete the endothelial cell survival factor VEGF-A. [Kopp, et. al. “The Bone Marrow Vascular Niche: Home of HSC Differentiation and Mobilization.” PHYSIOLOGY 20: 349-356, 2005; 10.1152/physiol.00025.2005].

The sufficiency of the vasculature of the bone marrow microenvironment is also key to hematopoiesis. Indeed, a function of the vascular niche interaction with BMECs is to provide a cellular platform conducive to HSC support, however the molecular mechanism by which the proper structural integrity of endothelial cells leads to this development is still unclear. [Kopp, et. al. “The Bone Marrow Vascular Niche: Home of HSC Differentiation and Mobilization.” PHYSIOLOGY 20: 349-356, 2005; 10.1152/physiol.00025.2005].

mTOR Signaling in Hematopoiesis

HSCs need to coordinate proliferation and differentiation with their available essential nutrients and metabolic demands. [Wang, X., Chu, Y., Wang, W., & Yuan, W. (2016). mTORC signaling in hematopoiesis. International Journal of Hematology, 103(5), 510-518]. Mammalian target of rapamycin (mTOR) signaling acts as an important integrator of nutrient-sensing pathways for metabolism and plays essential roles in regulating hematopoiesis during embryonic development and adulthood. [Id.]

mTOR belongs to the phosphatidylinositol-3 kinase related-kinase (PI3KK) family of serine/threonine (Ser/Thr) protein kinases; it works as a sensor of cellular growth and metabolism in response to nutrient and hormonal cues. [Id.] mTOR forms two complexes, mTOR complex 1 (mTORC1) and mTOR complex 2 (mTORC2). They share the same catalytic mTOR subunit and another three known complex components, mLST8, DEPTOR, and Tti1/Tel2. [Id., citing Kim, D H, et al., mTOR interacts with raptor to form a nutrient-sensitive complex that signals to the cell growth machinery. Cell. (2002) 110(2):163-75; Jacinto, E. et al., Mammalian TOR complex 2 controls the actin cytoskeleton and is rapamycin insensitive. Nat Cell Biol. (2004) 6(11):1122-8; Peterson, T R, et al., DEPTOR is an mTOR inhibitor frequently overexpressed in multiple myeloma cells and required for their survival. Cell. (2009) 137(5):873-86; Kaizuka, T. et al., Tti1 and Tel2 are critical factors in mammalian target of rapamycin complex assembly. J Biol Chem. (2010) 285(26):20109-16]. Additionally, mTORC1 has unique subunits of regulatory-associated protein of mammalian target of rapamycin (raptor) and PRAS40; mTORC2 has rapamycininsensitive companion of mTOR (rictor), mSin1, and protor1/2 as its specific components. [Wang, X., et al. (2016). mTORC signaling in hematopoiesis. International Journal of Hematology, 103(5), 510-518; citing Hara, K. et al., Raptor, a binding partner of target of rapamycin (TOR), mediates TOR action. Cell. (2002) 110(2):177-89; Jacinto, E. et al., SIN1/MIP1 maintains rictor-mTOR complex integrity and regulates Akt phosphorylation and substrate specificity. Cell. (2006) 127(1):125-371; Pearse, L R, et al., Identification of protor as a novel rictor-binding component of mTOR complex-2. Biochem J. (2007) 405(3):513-22 Sancak, Y., et al., PRAS40 is an insulin-regulated inhibitor of the mTORC1 protein kinase. Mol Cell. (2007) 25(6):903-15].

Genetic studies of important regulatory molecules associated with mTOR signaling demonstrate the central role of the mTOR pathway in both embryonic and adult hematopoiesis. mTOR and its complex components such as Raptor, Rictor, and mSin1 play important roles during embryonic development; mice lacking mTOR, Rictor, or Raptor die early in development and display functional abnormality in various organs. [Id., citing Guertin, D A, et al., Ablation in mice of the mTORC components raptor, rictor, or mLST8 reveals that mTORC2 is required for signaling to Akt-FOXO and PKCalpha, but not S6K1. Dev Cell. (2006) 11(6):859-71; Gangloff Y G, et al., Disruption of the mouse mTOR gene leads to early post-implantation lethality and prohibits embryonic stem cell development. Mol Cell Biol. (2004) 24(21):9508-16; Murakami M, et al. mTOR is essential for growth and proliferation in early mouse embryos and embryonic stem cells. Mol Cell Biol. (2004) 24(15):6710-8; Shiota, C. et al., Multiallelic disruption of the rictor gene in mice reveals that mTOR complex 2 is essential for fetal growth and viability. Dev Cell. (2006) 11(4):583-9]. Conditional mTOR deletion results in the loss of quiescence for hematopoietic stem cells, leading to a transient increase but a long-term exhaustion of HSCs and the defective engraftment of HSCs in lethally irradiated recipient mice. [Id., citing Guo, F. et al., Mouse gene targeting reveals an essential role of mTOR in hematopoietic stem cell engraftment and hematopoiesis. Haematologica. (2013) 98(9):1353-8]. These results demonstrate that mTOR is essential for hematopoietic stem cell engraftment and multi-lineage hematopoiesis. [Wang, X., et al. (2016). mTORC signaling in hematopoiesis. International Journal of Hematology, 103(5), 510-518)]

The over-activation of mTOR also drives HSCs from quiescence into more active cell cycling. For example, mTOR over-activation increased mitochondrial biogenesis and caused the accumulation of a much higher level of reactive oxygen species (ROS). [Id., citing Chen, C. et al., The axis of mTOR-mitochondria-ROS and stemness of the hematopoietic stem cells. Cell Cycle. 2009; 8(8):1158-60; Chen, C. et al., TSC-mTOR maintains quiescence and function of hematopoietic stem cells by repressing mitochondrial biogenesis and reactive oxygen species. J Exp Med. 2008; 205(10):2397-408]. The removal of ROS rescued HSC defects associated with hyper-activated mTOR. [Id., citing Chen, C. et al., Cell Cycle (2009) 8(8):1158-60]. Moreover, the hematopoietic lineage deletion of TSC Complex Subunit 1 (TSC1, part of the hamartin-tuberin complex with TSC2 that negatively regulates mTORC1 signaling) reduced the self-renewal of HSCs, as revealed by serial and competitive bone marrow transplantation. In vivo treatment with an ROS antagonist restored HSC numbers and functions. These data demonstrated that the TSC-mTOR pathway is key to HSC quiescence and maintains the quiescence of HSCs by repressing ROS production. [Chen, C. et al., TSC-mTOR maintains quiescence and function of hematopoietic stem cells by repressing mitochondrial biogenesis and reactive oxygen species. J Exp Med. (2008) 205(10):2397-408; Gan B, et al. mTORC1-dependent and -independent regulation of stem cell renewal, differentiation, and mobilization. Proc Natl Acad Sci. (2008) 105(49):19384-9].

Additionally, other studies showed increased mTOR activity in HSCs from older mice compared with those from young mice. Conditional TSC1 deletion reduced mTORC1 activity and impaired HSCs' regenerative capacity. The phenotypes of TSC1-deficient HSCs are similar in several ways to those of HSCs derived from wild type aged mice. The data indicate that mTOR signaling is important for HSC aging. [Id., citing Chen C, et al. mTOR regulation and therapeutic rejuvenation of aging hematopoietic stem cells. Sci Signal. (2009) 2(98):ra75].

Deficiency in Raptor, an essential component of mTORC1, leads to the expansion of CD48+CD150− and CD48+CD150+ LSK populations and promotes more ST-HSCs to transition from G0 phase to G1 phase. [Id.] Furthermore, Raptor-deficient BM cells cannot reconstitute hematopoiesis in lethally irradiated recipient mice. [Id., citing Kalaitzidis, D. et al., mTOR complex 1 plays critical roles in hematopoiesis and Pten-loss-evoked leukemogenesis. Cell Stem Cell. (2012) 11(3):429-39]. The deletion of Rictor, the regulatory-associated protein of mTORC2 in the hematopoietic system, did not affect HSC number or its function. Rictor-deficient bone marrow cells achieved long-term multilineage reconstitution of all recipient mice for at least 16 weeks after transplantation, although there was reduced B cell development due to the blocking of B cell development at an immature stage. [Id., citing Kentsis, A., Look, A T, Distinct and dynamic requirements for mTOR signaling in hematopoiesis and leukemogenesis. Cell Stem Cell. (2012) 11(3):281-2; Magee, J A, et. al., Temporal changes in PTEN and mTORC2 regulation of hematopoietic stem cell self-renewal and leukemia suppression. Cell Stem Cell. (2012) 11(3):415-28; Zhang, Y. et al., Rictor is required for early B cell development in bone marrow. PLoS One. (2014) 9(8):e103970].

mTORC1 and mTORC2 also play a role in HSC homeostasis. Although evidence indicates that HSCs reside in a low perfusion and low-nutrient niche, how cellular metabolism regulates stem cell function is poorly understood. Several studies have demonstrated that several nutrient-sensing pathways contribute to HSC homeostasis. For example, Huang et al. reported that the suppression of the mTOR pathway, an established nutrient sensor, combined with the activation of canonical Wnt-β-catenin signaling, allows for the ex vivo maintenance of human and mouse long-term HSCs under cytokine-free conditions. [Id., citing Huang, J. et al., Maintenance of hematopoietic stem cells through regulation of Wnt and mTOR pathways. Nat Med. (2012) 18(12):1778-85]. They also showed that the combination of CHIR99021 (a GSK-3 inhibitor) and rapamycin (an mTOR inhibitor) activates Wnt-β-catenin, inhibits mTOR signaling, and increases the absolute number of long-term HSCs (LT-HSCs) in vivo. [Id., citing Huang J, et al. Nat Med. (2012) 18(12):1778-85]. In addition, GSK-3 regulates both HSC Wnt and mTOR signaling in mice and thus promotes HSC self-renewal and lineage commitment; GSK-3 inhibition in the presence of rapamycin expanded the HSC pool in vivo. [Id., citing Huang, J. et al., Pivotal role for glycogen synthase kinase-3 in hematopoietic stem cell homeostasis in mice. J Clin Invest. (2009) 119(12):3519-29]. Furthermore, mTOR and p38 mitogen-activated protein kinase (MAPK) signaling pathways were always activated in the HSC population with higher ROS levels. However, this population was exhausted more quickly than the HSC population with lower ROS levels. Treatment with an mTOR inhibitor or a p38 inhibitor can restore HSC function in vivo. [Id., citing Jang, YY, Sharkis, SJ, A low level of reactive oxygen species elects for primitive hematopoietic stem cells that may reside in the low-oxygenic niche. Blood. 2007; 110(8):3056-63].

HSCs and Hematopoietic Challenges

All hematopoietic and immune cells are continuously generated by HSCs and HPCs through a highly organized process of stepwise lineage commitment. [(Kovtonyuk, L. V., Fritsch, K., Feng, X., Manz, M. G., & Takizawa, H. (2016). Inflamm-Aging of Hematopoiesis, Hematopoietic Stem Cells, and the Bone Marrow Microenvironment. Frontiers in Immunology, 7) In the steady state, HSCs are mostly quiescent, while HPCs are actively proliferating and contributing to daily hematopoiesis.

In response to hematopoietic challenges, e.g., life-threatening blood loss, infection, and inflammation, HSCs can be activated to proliferate and engage in blood formation. ((Id.))

### Inflammation

Inflammatory signals play key roles during diverse processes including embryonic specification of the hematopoietic stem cell (HSC) during development, emergency granulopoiesis during infections, and hematopoietic regeneration following transplantation. [See Baldridge, M. T., King, K. Y. & Goodell, M. A. Inflammatory signals regulate hematopoietic stem cells. Trends in immunology (2011) 32, 57-65, doi:10.1016/j.it.2010.12.003; Zhao, J. L. & Baltimore, D. Regulation of stress-induced hematopoiesis. Current opinion in hematology (2015) 22, 286-292, doi:10.1097/moh.0000000000000149; Boettcher, S. & Manz, M. G. Regulation of Inflammation- and Infection-Driven Hematopoiesis. Trends in immunology (2017) 38, 345-357, doi:10.1016/j.it.2017.01.004; Espin-Palazon, R., Weijts, B., Mulero, V. & Traver, D. Proinflammatory Signals as Fuel for the Fire of Hematopoietic Stem Cell Emergence. Trends in cell biology (2018) 28, 58-66, doi:10.1016/j.tcb.2017.08.003; Bowers, E. et al. Granulocyte-derived TNFalpha promotes vascular and hematopoietic regeneration in the bone marrow. Nature medicine (2018) 24, 95-102, doi:10.1038/nm.4448].

Although every stem cell niche is dynamic and exhibits cell turnover, it is useful to distinguish between niche cells that are ‘permanent residents’ (such as endothelial cells, nerve cells and connective tissue fibroblasts) and cells that occupy the niche in a transient fashion. (such as immune cells and cells that respond to tissue damage, for example, to protect against pathogens or to promote healing). In contrast to resident niche cells, many cells of the innate and adaptive immune system migrate into and out of tissues. The function of immune cells can be modulated to promote stem cell function. [Lane, S. W., et al. Modulating the stem cell niche for tissue regeneration. Nature biotechnology (2014) 32, 795-803].

Proinflammatory mediators that regulate HSC development include toll-like receptors (TLR), cytokines, and eicosanoids, each of which activates the immune system to fight insult. Tissue disruption due to injury or pathogenic agents leads to the release of proinflammatory cytokines that result in classical inflammation. Briefly, myeloid cells (such as macrophages and neutrophils) are armed with toll-like receptors (TLRs) and nucleotide-binding oligomerization domain (NOD)-like receptors (NLRs), which on recognition of damage-associated molecular patterns (DAMPs) and pathogen-associated molecular patterns (PAMPs), induce the proliferation of proinflammatory cytokines and eicosanoids. TLRs promote the induction of gene expression and the intracellular accumulation of major proinflammatory cytokines interleukin (IL)-1β and IL-18 via the master inflammation/immune transcription factor nuclear factor kappa B (NF-κB). Subsequently, recognition of PAMPs/DAMPs in the cytosolid compartment by NLRs promote caspase-1 mediated proteolytic cleavage and release of proinflammatory cytokines and cytosolic phospholipase A2-mediated eicosanoid biosynthesis. Proinflammatory cytokines and eicosanoids then activate immune cells to eliminate the cause of infection and restore healthy tissue. [Espin-Palazon, R., et al. Proinflammatory Signals as Fuel for the Fire of Hematopoietic Stem Cell Emergence. Trends in cell biology (2018) 28, 58-66].

HSCs are believed to sense immune or tissue insults by both cell intrinsic and extrinsic mechanisms; they respond dynamically to locally produced cytokines (niche/microenvironment) and distally (injury or infection) produced cytokines, including pro-inflammatory cytokines, chemokines and PAMPs. HSCs and hematopoietic stem and progenitor cells (HSPCs) indirectly (through proinflammatory cytokines or DAMPs) or directly (through PAMPs) sense the immune or tissue insult. Typically, HSCs respond to proinflammatory signals by skewing normal hematopoiesis towards myelopoiesis, often at the expense of lymphopoiesis and erythropoiesis, which is thought to occur to replenish the number of myeloid cells as the existing cells have been recruited to the site of infection. [Espin-Palazon, R., et al. Proinflammatory Signals as Fuel for the Fire of Hematopoietic Stem Cell Emergence. Trends in cell biology (2018) 28, 58-66].

Similar to differentiated immune cells, HSCs recognize insults through their expression of TLRs. Ligation of TLR signals in HSCs leads to proliferation and differentiation. A cell extrinsic mode of recognition of a tissue or immune insult by HSCs involves signaling through receptors for pro-inflammatory cytokines. [Nakagawa, M. M., et al. Constitutive Activation of NF-κB Pathway in Hematopoietic Stem Cells Causes Loss of Quiescence and Deregulated Transcription Factor Networks. Frontiers in cell and developmental biology (2018) 6: 143].

A spectrum of pro-inflammatory cytokines and chemokines, including IL-1, IL-6, IL-8, TNF, CC-Chemokine ligand 2 (CCL2), IFN-α and IFN-γ has been shown to influence HSCs. Indeed, the stimulation of agonists for TLR2, TLR7, and TLR8 in vitro has been shown to induce cytokine production, e.g., IL-1b, IL-6, IL-8, TNF-α, and GM-CSF, as well as cell differentiation of the myeloid lineage. [Kovtonyuk, L. V., et al. Inflamm-Aging of Hematopoiesis, Hematopoietic Stem Cells, and the Bone Marrow Microenvironment. Frontiers in immunology (2016) 7: 502, doi:10.3389/fimmu.2016.00502, citing Sioud M, et al. Signaling through toll-like receptor 7/8 induces the differentiation of human bone marrow CD34+ progenitor cells along the myeloid lineage. J Mol Biol (2006) 364:945-54; Sioud M, Floisand Y. TLR agonists induce the differentiation of human bone marrow CD34+ progenitors into CD11c+ CD80/86+DC capable of inducing a Th1-type response. Eur J Immunol (2007) 37:2834-46] The exposure of human CD34+ HSPCs to IFN-γ has been shown to produce drastic transcriptional changes in genes involved in pro-apoptotic processes, immune responses, and myeloid proliferation that results in an increased number of viable cells. [Id., citing Caux C, et al. Interferon-gamma enhances factor-dependent myeloid proliferation of human CD34+ hematopoietic progenitor cells. Blood (1992) 79:2628-35, Zeng W, et al. Interferon-gamma-induced gene expression in CD34 cells: identification of pathologic cytokine-specific signature profiles. Blood (2006) 107:167-75] While some transcriptional changes are specific to HSPCs, others, e.g., cell growth and signal transduction, generally occur in stromal cells incubated with IFN-γ. [Id., citing Zeng W, et al. Interferon-gamma-induced gene expression in CD34 cells: identification of pathologic cytokine-specific signature profiles. Blood (2006) 107:167-75] In contrast, studies have shown that in vitro stimulation with IFN-γ and TNF severely compromised the ability of HSPCs to undergo multi-lineage reconstitution in xenografted mice. [Id., citing Dybedal I, et al. Tumor necrosis factor (TNF)-mediated activation of the p55 TNF receptor negatively regulates maintenance of cycling reconstituting human hematopoietic stem cells. Blood (2001) 98:1782-91, Yang L, et al. IFN-gamma negatively modulates self-renewal of repopulating human hemopoietic stem cells. J Immunol (2005) 174:752-7]. Membrane-anchored TNF-α has been found to enhance the engraftment of purified HSCs in allogeneic and syngeneic recipients. [Espin-Palazon, R., et al. Trends in cell biology (2018) 28: 58-66].

Prolonged exposure of HSCs to pro-inflammatory cytokines causes diminished self-renewal and quiescence. The activation of quiescent ECs to generate the pro-inflammatory response is typically driven by transcription factor nuclear factor κB (NF-κB), which not only activates the transcription of pro-inflammatory genes, including TNF-α, interleukin-1 (IL-1), E-selectin, vascular cell adhesion molecule 1 (VCAM-1), and intercellular adhesion molecule 1 (ICAM-1), but also renders ECs more susceptible to apoptosis [Jin, Z., et al., Int. J. Mol. Sci. (2019) 20(1): 172; citing Pober, J S, Sessa, W C, Nat. Rev. Immunol. (2007) 7: 803-815; Aoki, M. et al., Hypertension (2001) 38: 48-55; Kempe, S. et al., Nucleic Acids Res. (2005) 33: 5308-5319].

The role of proinflammatory immunomodulators is not limited to adult HSC function. Studies have found that proinflammatory pathways, such as the prototypical proinflammatory transcription factor NF-κB, are linked to the formation of the hematopoietic system during embryogenesis in both vertebrates and invertebrates. Other immunomodulators also have an effect on HSC specification (identity), emergence and maintenance during hemopoietic system formation. For example, IL-3, a cytokine that regulates the function, proliferation, and differentiation of immune cells, has been found to promote survival of HSCs in the murine aorta-gonad-mesonephors (AGM) region, which is the site of HSC specification, by acting downstream of Runx1, an essential transcription factor in HSC specification. In another example, IL-1, a regulator of inflammation, plays an active role in HSC development by enhancing HSC expansion. Prostaglandin E2 (PGE2), a major regulator of inflammation, has also been shown to be a potent inducer for HSC emergence or expansion by controlling Wnt autonomously in HSCs at the level of beta-catenin degradation through cAMP/PKA mediated stabilizing phosphorylation events. [Espin-Palazon, R., et al., Trends in cell biology (2018) 28, 58-66].

HSC fate decisions have been linked to proinflammatory cytokines TNFα, IFNγ, and IL-10 where in addition to TLR4 signaling, these cytokines are each a key determinant of HSC specification. TNFα acts through TNF receptor 2 (TNFR2) to specify HSCs from hemogenic endothelial cells (HEs), a specialized subset of developing vascular endothelium that acquires hematopoietic potential and can give rise to multilineage hematopoietic stem and progenitor cells during a narrow developmental window in e.g., the extraembryonic yolk sac and embryonic aorta-gonad-mesonephros. [Griz, E. “Specification and function of hemogenic endothelium during embryogenesis,” Cell Mol. Life Sci. (2016) 73: 1547-67]. Action of TNFR2 is required for the expression of jag1a, a Notch ligand essential for HSC specification in the dorsal aorta. Expression of Jag1 signals to the Notch1a receptor on adjacent hemogenic endothelium cells (i.e., specialized endothelial cells from which HSPCs originate) to help establish the HSC fate. Proinflammatory transcription factor NF-κB has been found to be active in nascent HSCs. TNFR2, NF-κB member p65 and TLR4 are all upregulated in HSCs. Moreover, TLR4, IL-1β and TNFα are required for HSC specification by acting upstream of NF-κB and Notch. It was demonstrated that HSCs, but not endothelial cells, rapidly respond to IFNs. IFN-α4 and IFN-γ are also needed for HSC specification across vertebrates through IFNαR1 and IFNγR1, respectively. Unlike TNF-α and TLR4 signaling, IFN-γ acts downstream of Notch signaling and blood flow by activating Stat3. IFN-γ signaling acts autonomously in the HE. [Espin-Palazon, R., et al., Trends in cell biology (2018) 28, 58-66, doi:10.1016/j.tcb.2017.08.003].

Cellular sources of proinflammatory cytokines during hematopoietic system formation are not well known. Such cytokines are known to have an influence on HSC differentiation. In steady state, platelet-biased HSCs are at the top of the hematopoietic hierarchy and are able to generate myeloid-biased and lymphoid-biased HSCs. [Kovtonyuk, L. V., et al. Inflamm-Aging of Hematopoiesis, Hematopoietic Stem Cells, and the Bone Marrow Microenvironment. Frontiers in immunology (2016) 7: 502, doi:10.3389/fimmu.2016.00502], Myeloid-biased HSCs can generate both balanced- and lymphoid-biased HSCs, whereas lymphoid-biased HSCs do not generate their myeloid-biased counterparts. [Id.] Platelet-biased HSCs have the potential to repopulate platelet populations faster than other HSC subsets. Myeloid-biased HSCs preferentially give rise to myeloid lineage cells through myeloid committed progenitors. Balanced HSCs make equal contributions to both myeloid and lymphoid lineages. [Id.] Lymphoid-biased HSCs predominantly generate lymphoid over myeloid lineage cells through lymphoid-committed progenitors. Inflammation, specifically chronic inflammation, enhances myeloid lineage production, including myeloid progenitors and mature myeloid cells, leading to myeloid bias in hematopoiesis. [Id.]

Growing evidence indicates that the crosstalk between hematopoietic cells and their supportive niche cells initiates and sustains chronic inflammation within the bone marrow (BM), although their precise contributions in this process remain unclear. [Ramalingam, P. et al., Chronic activation of endothelial MAPK disrupts hematopoiesis via NFKB dependent inflammatory stress reversible by SCGF. Nature Communic. (2020) 11: 666, citing Kovtonyuk, L. V., et al. Inflamm-Aging of Hematopoiesis, Hematopoietic Stem Cells, and the Bone Marrow Microenvironment. Frontiers in immunology (2016) 7, 502, doi:10.3389/fimmu.2016.0050); Pietras, E. M. et al. Chronic interleukin-1 exposure drives haematopoietic stem cells towards precocious myeloid differentiation at the expense of self-renewal. Nature cell biology (2016) 18, 607-618; Lussana, F. & Rambaldi, A. Inflammation and myeloproliferative neoplasms. Journal of autoimmunity (2017) 85, 58-63; Pietras, E. M. Inflammation: a key regulator of hematopoietic stem cell fate in health and disease. Blood (2017) 130, 1693-1698].

Within the BM microenvironment, endothelial cells (ECs) have been established as an integral component of the HSC-supportive perivascular niche, as illustrated by their expression of a diverse array of HSC-regulatory paracrine factors [Id., citing Hooper, A. T. et al. Engraftment and reconstitution of hematopoiesis is dependent on VEGFR2-mediated regeneration of sinusoidal endothelial cells. Cell stem cell (2009) 4, 263-274, doi:10.1016/j.stem.2009.01.006; Butler, J. M. et al. Endothelial cells are essential for the self-renewal and repopulation of Notch-dependent hematopoietic stem cells. Cell stem cell (2010) 6, 251-264, doi:10.1016/j.stem.2010.02.001; Kobayashi, H. et al. Angiocrine factors from Akt-activated endothelial cells balance self-renewal and differentiation of haematopoietic stem cells. Nature cell biology (2010) 12, 1046-1056, doi:10.1038/ncb2108; Winkler, I. G. et al. Vascular niche E-selectin regulates hematopoietic stem cell dormancy, self-renewal and chemoresistance. Nature medicine (2012) 18, 1651-1657, doi:10.1038/nm.2969; Ding, L., et al., Endothelial and perivascular cells maintain haematopoietic stem cells. Nature (2012) 481, 457-462, doi:10.1038/nature10783; Poulos, M. G. et al. Endothelial jagged-1 is necessary for homeostatic and regenerative 947 hematopoiesis. Cell reports (2013) 4, 1022-1034, doi:10.1016/j.celrep.2013.07.048; Greenbaum, A. et al. CXCL12 in early mesenchymal progenitors is required for haematopoietic stem-cell maintenance. Nature (2013) 495, 227-230, doi:10.1038/naturel 1926; Doan, P. L. et al. Epidermal growth factor regulates hematopoietic regeneration after radiation injury. Nature medicine (2013) 19, 295-304, doi:10.1038/nm.3070; Poulos, M. G. et al. Endothelial-specific inhibition of NF-kappaB enhances functional haematopoiesis. Nat Commun (2016) 7, 13829, doi:10.1038/ncomms13829; Kusumbe, A. P. et al. Age-dependent modulation of vascular niches for haematopoietic stem cells. Nature (2016) 532, 380-384, doi:10.1038/nature17638; Morrison, S. J. & Scadden, D. T. The bone marrow niche for haematopoietic stem cells. Nature (2014) 505, 327-334, doi:10.1038/nature12984; Rafii, S., Butler, J. M. & Ding, B. S. Angiocrine functions of organ-specific endothelial cells. Nature (2016) 529, 316-325, doi:10.1038/nature17040]. Modulation of signaling pathways within the endothelium has also been shown to directly impact niche activity, thereby regulating HSC self-renewal and lineage commitment decisions [Id., citing Kobayashi, H. et al. Angiocrine factors from Akt-activated endothelial cells balance self-renewal and differentiation of haematopoietic stem cells. Nature cell biology (2010) 12, 1046-1056, doi:10.1038/ncb2108 (2010); Poulos, M. G. et al. Endothelial-specific inhibition of NF-kappaB enhances functional haematopoiesis. Nat Commun (2016) 7, 13829, doi:10.1038/ncomms13829; Kusumbe, A. P. et al. Age-dependent modulation of vascular niches for haematopoietic stem cells. Nature (2016) 532, 380-384, doi:10.1038/nature17638]. In addition to serving as tissue-specific niche cells, ECs play a key role during chronic inflammation [Id., citing Rafii, S., Butler, J. M. & Ding, B. S. Angiocrine functions of organ-specific endothelial cells. Nature (2016) 529, 316-325, doi:10.1038/nature17040; Pober, J. S. & Sessa, W. C. Evolving functions of endothelial cells in inflammation. Nature reviews. Immunology (2007) 7, 803-815, doi:10.1038/nri2171] and have emerged as an important source of niche-derived inflammatory signals within the BM, including IL-1 and G-CSF, which drive myelopoiesis during response to acute demands [Id., citing Pietras, E. M. et al. Chronic interleukin-1 exposure drives haematopoietic stem cells towards precocious myeloid differentiation at the expense of self-renewal. Nature cell biology (2016) 18, 607-618, doi:10.1038/ncb3346; Boettcher, S. et al. Endothelial cells translate pathogen signals into G-CSF-driven emergency granulopoiesis. Blood (2014) 124, 1393-1403, doi:10.1182/blood-2014-04-570762]. Sustained endothelial inflammation has been implicated in the initiation of myeloproliferative diseases through the expression of G-CSF and TNFα [Id., citing Wang, L. et al. Notch-dependent repression of miR-155 in the bone marrow niche regulates hematopoiesis in an NF-kappaB-dependent manner. Cell stem cell (2014) 15, 51-65, doi:10.1016/j.stem.2014.04.021]. However, signaling pathways mediating chronic endothelial inflammation within the BM microenvironment that impact niche activity and HSC function remain poorly understood.

NF-κB and MAPK are the principal signaling pathways regulating chronic inflammatory responses within endothelial cells [Id., citing Pober, J. S. & Sessa, W. C. Evolving functions of endothelial cells in inflammation. Nature reviews. Immunology (2007) 7, 803-815, doi:10.1038/nri2171]. However, their role in modulating inflammation within the BM endothelial niche and the concomitant impact on HSC function remains unexplored. Prior research has shown that suppression of NF-κB signaling within the endothelium enhances steady state hematopoiesis as well as regeneration following myelosuppression, in part by decreasing pro-inflammatory cytokines [Id., citing Poulos, M. G. et al. Endothelial-specific inhibition of NF-kappaB enhances functional haematopoiesis. Nat Commun (2016) 7, 13829, doi:10.1038/ncomms13829]. Recent reports suggest that endothelial MAPK plays essential roles during inflammatory processes including LPS-induced granulopoiesis and chronic vascular inflammation associated atherosclerosis [Id., citing Sanchez, A. et al. Map3k8 controls granulocyte colony-stimulating factor production and neutrophil precursor proliferation in lipopolysaccharide-induced emergency granulopoiesis. Sci Rep (2017) 7, 5010, doi:10.1038/s41598-017-04538-3; Roth Flach, R. J. et al. Endothelial protein kinase MAP4K4 promotes vascular inflammation and atherosclerosis. Nat Commun (2015) 6, 8995, doi:10.1038/ncomms9995]. Utilizing an ex vivo niche model system, it has been demonstrated that endothelial MAPK activation drives myeloid-biased differentiation of co-cultured HSCs at the expense of their self-renewal, features that are suggestive of an inflammatory stress [Id., citing Kobayashi, H. et al. Angiocrine factors from Akt-activated endothelial cells balance self-renewal and differentiation of haematopoietic stem cells. Nature cell biology (2010) 12, 1046-1056, doi:10.1038/ncb2108].

Hematopoietic Changes During Aging.

Aging of the hematopoietic system is represented by functional declines in both the adaptive and the innate immune system, an immunosenescence that leads to high susceptibility to infections, low efficacy of vaccinations, and increased vulnerability to the development of autoimmunity and hematologic malignancies. [Kovtonyuk, L V, et al., Inflamm-Aging of Hematopoiesis, Hematopoietic stem cells, and the bone marrow microenvironment. Front. Immunol. (2016) 7: 502. Doi: 10/3389/Immu.2016.00502, citing Dorshkind, K. et al, The ageing immune system: is it ever too old to become young again?Nat Rev Immunol (2009) 9:57-62.10.1038/nri2471; Haq K, McElhaney J E. Ageing and respiratory infections: the airway of ageing. Immunol Lett (2014) 162:323-8.10.1016/j.imlet.2014.06.009 5]. B cell production decreases significantly with advancing age, i.e., the naïve B cell pool diminishes, while the memory B cell pool expands. Diversity of the B cell repertoire also decreases in association with lowered antibody affinity and impaired class switching. B cells are prone to produce auto-antibodies increasing the incidence of spontaneous autoimmunity. [Id., citing Frasca D, et al. Mechanisms for decreased function of B cells in aged mice and humans. J Immunol (2008) 180:2741-6, Linton P J, Dorshkind K. Age-related changes in lymphocyte development and function. Nat Immunol (2004) 5:133-9]. De novo T cell production also declines with aging partially due to thymic involution. CD8+ T cells undergo oligoclonal expansion and their repertoire is skewed toward previously encountered antigens, as niches for naïve T cells in peripheral lymphoid tissues become occupied by terminally differentiated cells. [Id., citing Akbar A N, Fletcher J M. Memory T cell homeostasis and senescence during aging. Curr Opin Immunol (2005) 17:480-5]. NK cells show diminished cytotoxicity and cytokine secretion. Although myeloid cells increase in number, their functionality is decreased, e.g., neutrophils migrate less in response to stimuli, and macrophages have reduced phagocytic activity and decreased oxidative burst. [Id., citing Kuranda K, et al. Age-related changes in human hematopoietic stem/progenitor cells. Aging Cell (2011) 10:542-6; Ogata K, et al. Natural killer cells in the late decades of human life. Clin Immunol Immunopathol (1997) 84:269-75; Plowden J, Renshaw-Hoelscher M, Engleman C, Katz J, Sambhara S. Innate immunity in aging: impact on macrophage function. Aging Cell (2004) 3:161-7]. Finally erythropoiesis also declines in the elderly causing frequent anemia [Id., citing Berliner N. Anemia in the elderly. Trans Am Clin Climatol Assoc (2013) 124:230-7]. The thrombolytic (platelet) lineage has not, to date, been reported to be significantly affected by aging.

### HSC Functional Alteration During Aging.

Sustained inflammation has been proposed as a key driver of aging-associated hematopoietic defects, including loss of HSC self-renewal ability, myeloid-biased differentiation and a predisposition towards leukemias [Ramalingam, P. et al., “Chronic activation of endothelial MAPK disrupts hematopoiesis via NFKB dependent inflammatory stress reversible by SCGF. Nature Communic. (2020) 11: 666, citing Kovtonyuk, L. V., et al. Inflamm-Aging of Hematopoiesis, Hematopoietic Stem Cells, and the Bone Marrow Microenvironment. Frontiers in immunology (2016) 7, 502, doi:10.3389/fimmu.2016.0050); Pietras, E. M. et al. Chronic interleukin-1 exposure drives haematopoietic stem cells towards precocious myeloid differentiation at the expense of self-renewal. Nature cell biology (2016) 18, 607-618, doi:10.1038/ncb3346; Lussana, F. & Rambaldi, A. Inflammation and myeloproliferative neoplasms. Journal of autoimmunity (2017) 85, 58-63, doi:10.1016/j.jaut.2017.06.010; Pietras, E. M. Inflammation: a key regulator of hematopoietic stem cell fate in health and disease. Blood (2017) 130, 1693-1698, doi:10.1182/blood-2017-06-780882].

Since multiple blood lineages change during the aging process, it is possible that hematopoietic aging is in part due to functional changes in early hematopoietic compartments that repopulate the affected lineages, including HSCs [Kovtonyuk, L. V., et al. Inflamm-Aging of Hematopoiesis, Hematopoietic Stem Cells, and the Bone Marrow Microenvironment. Frontiers in immunology (2016) 7, 502, doi:10.3389/fimmu.2016.0050]. Single-cell and limiting dilution transplantations have demonstrated that the self-renewal capacity of HSCs is apparently reduced on a per-cell basis during aging, as the frequency of phenotypically defined HSCs does not correlate with that of functionally defined HSCs in aged BM [Id., citing Yamamoto, R. et al., Clonal analysis unveils self-renewing lineage-restricted progenitors generated directly from hematopoietic stem cells. Cell (2013) 154:1112-26.10.1016/j.cell.2013.08.007, Chambers, S M et al., Aging hematopoietic stem cells decline in function and exhibit epigenetic dysregulation. PLoS Biol (2007) 5:e201.10.1371/journal.pbio.0050201; Dykstra, B. et al., Clonal analysis reveals multiple functional defects of aged murine hematopoietic stem cells. J Exp Med (2011) 208:2691-703.10.1084/jem.20111490; Harrison, D E, Astle, C M, Loss of stem cell repopulating ability upon transplantation. Effects of donor age, cell number, and transplantation procedure. J Exp Med (1982) 156:1767-79; Morrison, S J et al., The aging of hematopoietic stem cells. Nat Med (1996) 2:1011-6; Rossi, D J et al., Cell intrinsic alterations underlie hematopoietic stem cell aging. Proc Natl Acad Sci USA (2005) 102:9194-9; Sudo, K. et al., Age-associated characteristics of murine hematopoietic stem cells. J Exp Med (2000) 192:1273-80]. It was also shown that phenotypic HSCs (LKS CD34−Flt3−) upregulate CD150 expression [Id., citing Challen, G A et al., Distinct hematopoietic stem cell subtypes are differentially regulated by TGF-beta1. Cell Stem Cell (2010) 6:265-78, Rossi, D J et al., Cell intrinsic alterations underlie hematopoietic stem cell aging. Proc Natl Acad Sci USA (2005) 102:9194-9, Beerman, I et al., Functionally distinct hematopoietic stem cells modulate hematopoietic lineage potential during aging by a mechanism of clonal expansion. Proc Natl Acad Sci USA (2010) 107:5465-70], resulting in expansion of the myeloid-biased HSC population and the domination of this fraction over the entire aged HSC pool [Id., citing Beerman, I et al., Functionally distinct hematopoietic stem cells modulate hematopoietic lineage potential during aging by a mechanism of clonal expansion. Proc Natl Acad Sci USA (2010) 107:5465-70], with advancing age. Consistent with the phenotypic characterization, hematopoietic repopulation after transplantation is biased toward myeloid cell production, and this change in differentiation potential persists over the course of serial transplantations, indicative of aging-associated cell-autonomous alterations in HSCs. Based on these observations, two possible theories for age-associated myeloid bias can be proposed: (a) clonal evolution within the aged HSC population, in which lymphoid-biased HSC clones turn into myeloid-biased or platelet-biased HSC clones via cell-intrinsic changes [Id., citing Waterstrat A, Van Zant G. Effects of aging on hematopoietic stem and progenitor cells. Curr Opin Immunol (2009) 21:408-13]; (b) clonal composition shift, in which subsets of myeloid-biased or platelet-biased HSC clones dominate the entire HSC pool via clonal expansion and/or selection [Id., citing Morita Y, et al. Heterogeneity and hierarchy within the most primitive hematopoietic stem cell compartment. J Exp Med (2010) 207:1173-82, Challen, G A et al., Distinct hematopoietic stem cell subtypes are differentially regulated by TGF-beta1. Cell Stem Cell (2010) 6:265-78, Dykstra, B. et al., Clonal analysis reveals multiple functional defects of aged murine hematopoietic stem cells. J Exp Med (2011) 208:2691-703, Beerman, I et al., Functionally distinct hematopoietic stem cells modulate hematopoietic lineage potential during aging by a mechanism of clonal expansion. Proc Natl Acad Sci USA (2010) 107:5465-70, Cho, R H et al., A new mechanism for the aging of hematopoietic stem cells: aging changes the clonal composition of the stem cell compartment but not individual stem cells. Blood (2008) 111:5553-61; Goto M. Inflammaging (inflammation+aging): a driving force for human aging based on an evolutionarily antagonistic pleiotropy theory? Biosci Trends (2008) 2:218-30; Muller-Sieburg C E, Sieburg H B. Clonal diversity of the stem cell compartment. Curr Opin Hematol (2006) 13:243-8]. Aging-associated myeloid lineage skewing may also involve disturbance in the composition of committed progenitors: aged mice show a decreased frequency of common lymphoid progenitors, while frequencies of GMPs are increased [Id., citing Rossi, D J et al., Cell intrinsic alterations underlie hematopoietic stem cell aging. Proc Natl Acad Sci USA (2005) 102:9194-9]. These findings are accompanied by decreased B cell lymphopoiesis and diminished fitness of lymphoid progenitors, coinciding with altered receptor-associated kinase signaling [Id., citing Henry, C J et al., Declining lymphoid progenitor fitness promotes aging-associated leukemogenesis. Proc Natl Acad Sci USA (2010) 107:21713-8]. Which level of the hematopoietic hierarchy is affected by aging is not understood.

The BM homing efficiency of aged HSCs is significantly reduced when transplanted intravenously into irradiated recipients [Id., citing Dykstra, B., et al., Cell Stem Cell (2007) 1:218-29], although similar mobilizing efficacies are observed in aged and young HSCs released into the circulation in response to granulocyte colony-stimulating factor (G-CSF) treatment [Id., citing Verovskaya, E. et al., Asymmetry in skeletal distribution of mouse hematopoietic stem cell clones and their equilibration by mobilizing cytokines. J Exp Med (2014) 211:487-97]. Transcriptome profiling of aged versus young HSCs has provided molecular insights into potential mechanisms of HSC aging [Id., citing Chambers, S M et al., Aging hematopoietic stem cells decline in function and exhibit epigenetic dysregulation. PLoS Biol (2007) 5:e201.10.1371/journal.pbio.0050201; Tremaroli V, Bickhed F. Functional interactions between the gut microbiota and host metabolism. Nature (2012) 489:242-9.10.1038/nature11552]: aged HSCs show dysregulation of intracellular homeostasis, e.g., upregulated stress responses, increased pro-inflammatory signaling, protein misfolding, downregulated DNA repair machinery, and aberrant chromatin modification [Id., citing Challen, G A et al., Cell Stem Cell (2010) 6:265-78, Chambers, S M et al., Aging hematopoietic stem cells decline in function and exhibit epigenetic dysregulation. PLoS Biol (2007) 5:e201.10.1371/journal.pbio.0050201, Rossi, D J et al., Proc Natl Acad Sci USA (2005) 102:9194-9]. Further investigations have demonstrated that aged HSCs accumulate more DNA damage possibly due to higher levels of intracellular reactive oxygen species (ROS) and naturally produced genotoxic metabolites [Id., citing Ito, K. et al., Reactive oxygen species act through p38 MAPK to limit the lifespan of hematopoietic stem cells. Nat Med (2006) 12:446-51; Rossi, D J et al, Nature (2007) 447:725-9; Rü̅be, C E et al., Accumulation of DNA damage in hematopoietic stem and progenitor cells during human aging. PLoS One (2011) 6:e17487.10.1371/journal.pone.0017487], but these cells are still able to efficiently repair the damage upon cell cycle induction [Id., citing Beerman, I. et al., Cell Stem Cell (2014) 15:37-50]. Other studies have indicated that accumulation of proliferative stress in aged HSCs causes inefficient DNA replication and transcriptional repression [Id., citing Flach, J. et al., Replication stress is a potent driver of functional decline in ageing haematopoietic stem cells. Nature (2014) 512:198-202]. Aged HSCs also exhibit activation of the mammalian target of rapamycin (mTOR) [Id., citing Chen, C. et al., mTOR regulation and therapeutic rejuvenation of aging hematopoietic stem cells. Sci Signal (2009) 2:ra75.10.1126/scisignal.20005593], autophagy-dependent survival [Id., citing Warr, M R, et al. FOXO3A directs a protective autophagy program in haematopoietic stem cells. Nature (2013) 494:323-7], dysregulated DNA methylation, specifically at the site of genes controlling myeloid and lymphoid balancing [Id., citing Beerman, I. et al., Cell Stem Cell (2014) 15:37-50], impaired histone modification [Id., citing Sun, D. et al. Epigenomic profiling of young and aged HSCs reveals concerted changes during aging that reinforce self-renewal. Cell Stem Cell (2014) 14:673-8855], and disturbed cell polarity [Id., citing Florian, M C, et al., Cdc42 activity regulates hematopoietic stem cell aging and rejuvenation. Cell Stem Cell (2012) 10:520-30]. These characteristics of HSC aging can, in part, be experimentally recapitulated by increasing the proliferative history of HSCs or stressing them with multiple injections of myeloablative chemotherapeutic regimens [Id., citing Beerman, I. et al., Proliferation-dependent alterations of the DNA methylation landscape underlie hematopoietic stem cell aging. Cell Stem Cell (2013) 12:413-25], or by conducting serial trans-plantations (“experimental aging” [Id., citing Dykstra, B. et al, J Exp Med (2011) 208:2691-703; Harrison, DE, Astle, CM, J Exp Med (1982) 156:1767-79]. As this indicates that proliferative history might be associated with the aging process, several groups have compared the cycling activity of young versus aged HSCs. The results are, however, controversial: some data indicate that aged HSCs have increased cycling activity [Id., citing Morrison, S J et al., Nat Med (1996) 2:1011-6], whereas others suggest no difference in cell cycle status [Id., citing Chambers, S M et al., PLoS Biol (2007) 5:e201.10.1371/journal.pbio.0050201; Sudo, K., et al., Age-associated characteristics of murine hematopoietic stem cells. J Exp Med (2000) 192:1273-80.10.1084/jem.192.9.1273], or more quiescent HSCs in aged as compared to young BM [Id., citing Takizawa, H. et al., Dynamic variation in cycling of hematopoietic stem cells in steady state and inflammation. J Exp Med (2011) 208:273-84; Chen, C. et al., mTOR regulation and therapeutic rejuvenation of aging hematopoietic stem cells. Sci Signal (2009) 2:ra75.10.1126/scisignal.2000559].

Aging-Associated Changes in BM Niche Hematopoietic stem cell homeostasis is preserved in the BM microenvironment, the so-called HSC niche that supplies these cells with pivotal factors for their own maintenance. [Id., citing Nakamura-Ishizu, A. et al., Development (2014) 141:4656-66, Morrison S J, Scadden D T. The bone marrow niche for haematopoietic stem cells. Nature (2014) 505:327-34] Recent research on the BM niche has revealed a perivascular HSC niche comprised mesenchymal stromal cells (MSCs) and endothelial cells (ECs) as major cellular components, reflecting hierarchic HSC function and the effects exerted by aging. [Id., citing Morrison S J, Scadden D T. The bone marrow niche for haematopoietic stem cells. Nature (2014) 505:327-34; Kusumbe, A P et al. Age-dependent modulation of vascular niches for haematopoietic stem cells. Nature (2016) 532:380-4.10.1038/nature17638]. MSCs are characterized by plastic adherence, high growth potential, and mesenchymal immunophenotypes, as well as differentiation into mesenchymal lineages, such as osteocytes, adipocytes, chondrocytes, fibroblasts, and epithelial cells. [Id., citing Dominici, M. et al., Minimal criteria for defining multipotent mesenchymal stromal cells. The International Society for Cellular Therapy position statement. Cytotherapy (2006) 8:315-7; Lin, Z-J et al., Trafficking and differentiation of mesenchymal stem cells. J Cell Biochem (2009) 106:984-911]. Aged MSCs exhibit reduced clonogenic and proliferative capacity, and differentiation potentials are skewed toward adipogenesis at the expense of osteogenesis. [Id., citing Singh, L. et al. Aging alters bone-fat reciprocity by shifting in vivo mesenchymal precursor cell fate towards an adipogenic lineage. Bone (2016) 85:29-36; Tuljapurkar, S R et al. Changes in human bone marrow fat content associated with changes in hematopoietic stem cell numbers and cytokine levels with aging. J Anat (2011) 219:574-81; Walenda, T. et al. Co-culture with mesenchymal stromal cells increases proliferation and maintenance of haematopoietic progenitor cells. J Cell Mol Med (2010) 14:337-50)]. These cells also show enlargement, telomere shortening, or p53/p21-mediated DNA damage accumulation, impaired DNA methylation or histone acetylation, and increased levels of ROS and nitric oxide (NO). [Id., citing Behrens, A. et al. Impact of genomic damage and ageing on stem cell function. Nat Cell Biol (2014) 16:201-7; Fernandez, L. et al., Tumor necrosis factor-alpha and endothelial cells modulate Notch signaling in the bone marrow microenvironment during inflammation. Exp Hematol (2008) 36:545-58; Komicka, K. et al., The effect of age on osteogenic and adipogenic differentiation potential of human adipose derived stromal stem cells (hASCs) and the impact of stress factors in the course of the differentiation process. Oxid Med Cell Longev (2015) 2015:309169.10.1155/2015/309169; Zhang, D-Y, et al., Wnt/0-catenin signaling induces the aging of mesenchymal stem cells through the DNA damage response and the p53/p21 pathway. PLoS One (2011) 6:e21397.10.1371/journal.pone.0021397; Zheng, Y. et al., H3K9me-enhanced DNA hypermethylation of the p16INK4a gene: an epigenetic signature for spontaneous transformation of rat mesenchymal stem cells. Stem Cells Dev (2013) 22:256-6765-69] Although age-dependent mechanisms underlying adipogenesis-favoring MSC differentiation are not fully understood, possible molecular changes have been reported, including activation of peroxisome proliferator-activated receptor gamma 2 [PPPARγ2] and CCAAT/enhancer binding protein. [Id., citing Shockley, KR, e al., PPARgamma2 nuclear receptor controls multiple regulatory pathways of osteoblast differentiation from marrow mesenchymal stem cells. J Cell Biochem (2009) 106:232-46; Takeshita, S. et al. Age-related marrow adipogenesis is linked to increased expression of RANKL. J Biol Chem (2014) 289:16699-710]. It has been suggested that adipogenesis enhancement in aged BM [Id., citing Justesen, J. et al., Adipocyte tissue volume in bone marrow is increased with aging and in patients with osteoporosis. Biogerontology (2001) 2:165-71] might be linked to dysregulation of insulin growth factor signaling [Id., citing Linton P J, Dorshkind K. Age-related changes in lymphocyte development and function. Nat Immunol (2004) 5:133-9], changes in extracellular matrix composition, and decreased bone formation [Id., citing Bellantuono, I. et al., Aging of marrow stromal (skeletal) stem cells and their contribution to age-related bone loss. Biochim Biophys Acta (2009) 1792:364-70, Wagner, W. et al., Aging and replicative senescence have related effects on human stem and progenitor cells. PLoS One (2009) 4:e5846.10.1371/journal.pone.0005846], based on the reasoning that since adipocytes are shown to negatively regulate HSC function and B-lymphopoiesis [Id., citing Kennedy D E, Knight K L. Inhibition of B lymphopoiesis by adipocytes and IL-1-producing myeloid-derived suppressor cells. J Immunol (2015) 195:2666-74.10.4049/jimmunol.1500957; Naveiras, O. et al., Bone-marrow adipocytes as negative regulators of the haematopoietic microenvironment. Nature (2009) 460:259-63], adipogenesis enhanced in aged BM might promote myelopoiesis over lymphopoiesis as well as impair HSC function, and the observation that young HSCs in the aged environment reportedly tend to produce slightly more myeloid cells than in a young environment. [Id., citing Rossi, D J et al., Proc Natl Acad Sci USA (2005) 102:9194-97; Ergen, A V et al., Rantes/Ccl5 influences hematopoietic stem cell subtypes and causes myeloid skewing. Blood (2012) 119:2500-9).

Endothelial cells are another niche cell component that secrete HSC maintenance and retention factors, such as stem cell factor and CXC motif ligand (CXCL)12 [Id., citing Morrison S J, Scadden D T. The bone marrow niche for haematopoietic stem cells. Nature (2014) 505:327-34, Nombela-Arrieta C, et al., Quantitative imaging of haematopoietic stem and progenitor cell localization and hypoxic status in the bone marrow microenvironment. Nat Cell Biol (2013) 15:533-43]. Aging involves decreases in CD31^{hi}Emcn^{hi} EC-associated osteoprogenitors [Id., citing Kusumbe A P, et al. Coupling of angiogenesis and osteogenesis by a specific vessel subtype in bone. Nature (2014) 507:323-8], fewer PDGFRβ+/NG2+ perivascular cells, arterioles, and ECs, thereby resulting in reduced stem cell factor production. [Id., citing Kusumbe A P, et al. Age-dependent modulation of vascular niches for haematopoietic stem cells. Nature (2016) 532:380-4] Activation of endothelial Notch signaling can reverse these age-dependent vascular niche alterations, without affecting aged HSC function. Additionally, vascular endothelial function declines with aging, due to reduced NO which in turn induces vasodilation, elevated oxidative stress causing genomic instability, and increased ROS levels associated with impaired proangiogenic functions of EC. [Id., citing Groleau J, et al. Essential role of copper-zinc superoxide dismutase for ischemia-induced neovascularization via modulation of bone marrow-derived endothelial progenitor cells. Arterioscler Thromb Vasc Biol (2010) 30:2173-81] As it has been suggested that NO production regulates CXCL12-mediated HSC mobilization [Id., citing Gur-Cohen S, et al. PAR1 signaling regulates the retention and recruitment of EPCR-expressing bone marrow hematopoietic stem cells. Nat Med (2015) 21:1307-17], it has been hypothesized that aging-related reductions of EC-derived NO and the enhancement of angiogenic function in the BM niche might be involved in aberrant HSC maintenance and/or retention in aged BM. [Id.]

### Hematopoietic Aging in Humans

Most of the data on aging of the hematopoietic system was obtained employing a mouse system. However, a few pioneering studies have indicated similar tendencies in the human hematopoietic system. HSCs containing fractions such as Lin−CD34+CD38− [Id., citing Kuranda K, et al. Age-related changes in human hematopoietic stem/progenitor cells. Aging Cell (2011) 10:542-6], Lin−CD34+CD38−CD90+CD45RA− [Id., citing Pang W W, et al. Human bone marrow hematopoietic stem cells are increased in frequency and myeloid-biased with age. Proc Natl Acad Sci USA (2011) 108:20012-7], or Lin−CD34+CD 10−CD123−CD45RA−CD90+ [Id., citing Rundberg Nilsson A, et al. Human and murine hematopoietic stem cell aging is associated with functional impairments and intrinsic megakaryocytic/erythroid bias. PLoS One (2016) 11:e0158369.10.1371/journal.pone.0158369] increase with age. While granulocyte-monocyte progenitors (GMPs) appear to be retained at the same frequency [Id., citing Kuranda K, et al. Age-related changes in human hematopoietic stem/progenitor cells. Aging Cell (2011) 10:542-6, Pang W W, et al. Human bone marrow hematopoietic stem cells are increased in frequency and myeloid-biased with age. Proc Natl Acad Sci USA (2011) 108:20012-7, Rundberg Nilsson A, Soneji S, Adolfsson S, Bryder D, Pronk C J. Human and murine hematopoietic stem cell aging is associated with functional impairments and intrinsic megakaryocytic/erythroid bias. PLoS One (2016) 11:e0158369.10.1371/journal.pone.0158369], early B cell progenitors and common lymphoid progenitors (CLPs) decrease with advancing age [Id., citing Pang W W, et al. Human bone marrow hematopoietic stem cells are increased in frequency and myeloid-biased with age. Proc Natl Acad Sci USA (2011) 108:20012-7, Rundberg Nilsson A, Soneji S, Adolfsson S, Bryder D, Pronk C J. Human and murine hematopoietic stem cell aging is associated with functional impairments and intrinsic megakaryocytic/erythroid bias. PLoS One (2016) 11:e0158369.10.1371/journal.pone.0158369]. The functionality and differentiation bias of HSCs remain unclear: one study, using xenograft mouse models, indicated no change in immunodeficient NSG-repopulating cell frequency and decreased myeloid lineage repopulation of aged HSCs, [Id., citing Kuranda K, et al. Age-related changes in human hematopoietic stem/progenitor cells. Aging Cell (2011) 10:542-6], while another [Id., citing Pang W W, et al. Human bone marrow hematopoietic stem cells are increased in frequency and myeloid-biased with age. Proc Natl Acad Sci USA (2011) 108:20012-7] showed a two-fold decreased engraftment with significant myeloid lineage dominance. Further molecular analyses indicated upregulations of myeloid and megakaryocyte-associated genes and downregulations of lymphoid differentiation genes. [Id., citing Pang W W, et al. Human bone marrow hematopoietic stem cells are increased in frequency and myeloid-biased with age. Proc Natl Acad Sci USA (2011) 108:20012-7, Rundberg Nilsson A, Soneji S, Adolfsson S, Bryder D, Pronk C J. Human and murine hematopoietic stem cell aging is associated with functional impairments and intrinsic megakaryocytic/erythroid bias. PLoS One (2016) 11:e0158369.10.1371/journal.pone.0158369]. These findings indicate major aging-associated changes in hematopoiesis to be conserved among species.

### Myoablative Therapy

In patients that require reconstitution of their hematopoietic system, preparative or conditioning regimens are administered as part of the procedure to achieve two goals: to provide sufficient immunoablation to prevent host rejection, and to provide tumor cytoreduction/disease eradication. There are variations of conditioning regimens as the intensity can vary based on disease-related factors such as diagnosis and remission status, as well as patient-related factors including age, donor availability, and presence of comorbid conditions. Conditioning regimens have been classified as high-dose (myeloablative), reduced-intensity, and nonmyeloablative therapy. [Gyurkocza, Boglarka, and Brenda M Sandmaier. “Conditioning regimens for hematopoietic cell transplantation: one size does not fit all.” Blood (2014) vol. 124, 3: 344-53]. Myeloablative therapy (MBT) refers to the treatment of a patient with high-dose chemotherapy (HDC) or HDC with total body radiation (TBI) to eradicate the immune and hematopoietic systems as well as all malignant cells within the body. Typically, patients who receive MBT have done so in preparation for bone marrow transplantation, stem cell transplantation, or hematopoietic cell transplantation (referred to herein as “stem cell rescue” or “SCR”); however, as seen in Table 2 below, MBT can also be used as a treatment type for various types of malignancies in which SCR has not been shown to be beneficial. [Riley, et al., “Hematologic Aspects of Myeloablative Therapy and Bone Marrow Transplantation.” Journal of Clinical Laboratory Analysis (2005) 19:47-79].

TABLE 2
Indications for Myeoblative Therapy/Stem Cell Rescue

| Type of disease | Specific diseases |
|---|---|
| Primary and secondary | Aplastic anemia |
| bone marrow failure | Amegakaryocytosis/congenital |
| | thrombocytopenia |
| | Fanconi anemia |
| | Paroxysmal nocturnal |
| | hemoglobinuria (PNH) |
| Hematopoietic | Acute leukemias |
| malignancies | Chronic leukemia |
| | Myelodysplastic syndromes |
| | Myeloproliferative disorders |
| | Non-Hodgkin lymphoma |
| | Plasma cell diseases |
| | Histiocytic disorders |
| Solid tumors | Breast cancer |
| | Renal cell carcinoma |
| | Neuroblastoma |
| | Ewing sarcoma |
| | Testicular carcinoma |
| Congenital | Immunodeficiencies (severe combined |
| hematopoietic and | immunodeficiency, etc.) |
| immunodeficiency | Granulocyte deficiencies (chronic |
| states | granulomatous disease, etc.) |
| | Hemoglobulinopathies (beta-thalassemia |
| | major, sickle cell disease) |
| Inherited metabolic | Mucopolysaccharidoses |
| disorders | Hurler's syndrome |
| | Hunter's syndrome |
| | Niemann-Pick disease |
| | Mucolipidosis II |
| Other inherited | Osteopetrosis |
| diseases | Glanzmann thromboasthenia |
| | Lesch-Nyhan syndrome |
| | Cartilage-hair hypoplasia |

Generally, MBT regimens consist of HDC with alkylating agents (single agent types or multiple), and are delivered with or without TBI. Such regimens are expected to ablate marrow hematopoiesis, thereby not allowing autologous hematologic recovery. [Gyurkocza, Boglarka, and Brenda M Sandmaier. “Conditioning regimens for hematopoietic cell transplantation: one size does not fit all.” Blood (2014) vol. 124, 3: 344-53; for examples of specific MBT regimens see Atilla, E., Ataca Atilla, P., & Demirer, T. A Review of Myeloablative vs Reduced Intensity/Non-Myeloablative Regimens in Allogeneic Hematopoietic Stem Cell Transplantations. Balkan medical journal, (2017) 34(1), 1-9].

TABLE 3
Myeloablative Therapy Treatments and Dosages

| Myeloablative Therapy | Total Dosage (Days) | |
|---|---|---|
| Cy/TBI | | |
| Cy (mg/kg) | 120 | (−6, −5) |
| Total body irridation (GY) | 12-14 | (−3 to −1) |
| Bu/Cy | | |
| Bu (mg/kg) | 16 | (−7 to −4) |
| Cy (mg/kg) | 120 | (−3 to −2) |
| BACT | | |
| BCNU (mg/m²) | 200 | (−6) |
| ARA-C (mg/m²) | 800 | (−5 to −2) |
| Cy (mg/kg) | 50 | (−5 to −2) |
| 6-Thioguanine (mg/m²) | 800 | (−5 to −2) |
| BEAM | | |
| BCNU (mg/m²) | 300 | (−6) |
| Etoposide (mg/m²) | 800 | (−5 to −2) |
| ARA-C (mg/m²) | 800 | (−5 to −2) |
| Melphalan (mg/m²) | 140 | (−1) |
| TBI/VP | | |
| Total body irridation (Gy) | 12-13.2 | (−7 to −4) |
| Etoposide (mg/kg) | 60 | (−3) |
| AC/TBI | | |
| ARA-C (g/m²) | 36 | (−9 to −4) |
| Total body irridation (Gy) | 12 | (−3 to −1) |
| Mel/TBI | | |
| Melphalan (mg/m²) | 110-140 | |
| Total body irridation (Gy) | 10-14 | |
| Cy/VP/TBI | | |
| Cy (mg/kg) | 120 | (−6, −5) |
| Etoposide (mg/kg) | 30-60 | (−4) |
| Total body irridation (Gy) | 12-13.8 | (−3 to −1) |
| TBI/TT/Cy | | |
| Total body irridation (Gy) | 13.8 | (−9 to −6) |
| Thiotepa (mg/kg) | 10 | (−5, −4) |
| Cy (mg/kg) | 120 | (−3, −2) |
| Bu/Cy/Mel | | |
| Bu (mg/kg) | 16 | (−7 to −4) |
| Cy (mg/kg) | 120 | (−3, −2) |
| Mel (mg/m²) | 140 | (−1) |

ATG: anti-thymocyte globulin,
Bu: busulfan,
Cy: cyclophosphamide,
Mel: melphalan,
TBI: total body irridation,
TTP: thiotepa,
VP: etoposide

Total Body Irradation (TBI). TBI and high-dose TBI are widely used as part of the conditioning regimen due to its immunosuppressive properties, its effectiveness against most leukemias and lymphomas, and its ability to penetrate to sanctuary sites. The majority of regimens combined 12- to 16-Gy TBI, usually fractionated (meaning when the total dose of radiation is divided into several, smaller doses over a period of several days), with other chemotherapeutic agents, most commonly, cyclophosphamide, based on its antineoplastic and immunomodulatory properties. In general, higher doses of TBI, although reducing the relapse risk, resulted in increased, often fatal, gastrointestinal, hepatic, and pulmonary toxicities, secondary malignancies, and impaired growth and development in children. In addition to the delivered dose, other factors, such as dose rate, fractionation, interval between fractions, and the source of radiation (such as cobalt-60 vs linear accelerator) could also impact both the antineoplastic and toxic effects of TBI. Fractionation resulted in decreased organ toxicity but also sustained antineoplastic effects, due to a higher proportion of intact repair mechanisms retained in normal tissues as opposed to leukemic cells. Hyperfractionation (multiple fractions per day) with lung shielding resulted in a decreased incidence of interstitial pneumonitis of 4%, down from 50% observed with single-fraction TBI without lung shielding. The majority of TBI schedules in use today are either fractionated or hyperfractionated. In addition to cyclophosphamide, various agents, such as cytarabine (AraC), etoposide, melphalan, and busulfan, have been combined with high-dose TBI as conditioning regimens. (Gyurkocza, Boglarka, and Brenda M Sandmaier. “Conditioning regimens for hematopoietic cell transplantation: one size does not fit all.” Blood (2014) vol. 124, 3: 344-53).

The administration of high-dose TBI is associated with immediate and delayed toxicities, although it is not always possible to distinguish which component of the conditioning regimen is responsible for any given toxicity. Nausea, vomiting, transient acute parotiditis, xerostomia, mucositis, and diarrhea are commonly observed acute complications. Interstitial pneumonitis, idiopathic pulmonary fibrosis, and reduced lung pulmonary function can also be related to high-dose TBI. In addition, renal damage can occur and can be delayed (i.e., up to ˜2 years) after high-dose TBI. The occurrence of sinusoidal obstruction syndrome (SOS; formerly known as veno-occlusive disease of the liver) is more common in chemotherapy-based regimens described en infra. Long-term side effects of high-dose TBI include infertility, cataract formation, hyperthyroidism and thyroiditis, and secondary malignancies. (Gyurkocza, Boglarka, and Brenda M Sandmaier. “Conditioning regimens for hematopoietic cell transplantation: one size does not fit all.” Blood (2014) vol. 124, 3: 344-53).

High Dose Chemotherapy (HDC). The main component of HDC is the delivery of alkylating agents due to their favorable toxicity profile (marrow toxicity as dose-limiting toxicity) and their effect on nondividing tumor or malignant cells. Other agents that can be used include anthracyclines and taxanes. To avoid short- and long-term toxicities associated with high-dose TBI, especially in patients who received previous radiation therapy, high-dose chemotherapy-based regimens have been developed both in the autologous and allogeneic settings where TBI is replaced with additional chemotherapeutic agents. Alkalyating agents are often delivered with immunosuppressives; the treatment can include busulfan, cyclophosphamide, or fludarabine, melphalan, theiotepa, etoposide, and treosulfan, and the like, and combinations of such therapeutics. (Gyurkocza, Boglarka, and Brenda M Sandmaier. “Conditioning regimens for hematopoietic cell transplantation: one size does not fit all.” Blood (2014) vol. 124, 3: 344-53).

Morphological effects of MBT. The morphologic features of the bone marrow in a patient receiving MBT are determined by the overlapping processes of cell death and hematopoietic reconstitution. Aggressive chemotherapy alone or with TBI results in the obliteration of nearly all of the hematopoietic and immune cells over a period of several days. At the end of this period, the bone marrow is profoundly hypocellular, with an intact stroma containing a homogenous, periodic acid Schiff (PAS)-positive proteinaceous transudate resembling fibrinoid necrosis. A few residual plasma cells and macrophages are usually present, and vascular congestion, areas of nonspecific hemorrhage, small noncaseating granulomas, stromal edema, eosinophilia, mildreticulin fibrosis, sinus ectasia, osteocyte necrosis, and other anomalies may be found. A period of weeks is required for the restoration of normal levels of red blood cells, platelets, and granulocytes after MBT, with or without stem cell rescue, while complete functional reconstitution of the hematopoietic system takes place over a period of years. Further, in spite of the relatively rapid restoration of peripheral blood cell counts following myeloablative chemotherapy or bone marrow transplantation, there is a continuing severe cellular and humoral immunodeficiency for months to years. [Gyurkocza, Boglarka, and Brenda M Sandmaier. “Conditioning regimens for hematopoietic cell transplantation: one size does not fit all.” Blood vol. 124, 3 (2014): 344-53].

The myeloablative and conditioning regimens that purge malignant progenitors from the bone marrow may also remove or damage nonmalignant hematopoietic and stromal progenitor cells, resulting in a diminished capacity for transplanted and native stem cell renewal. This deficit is not always apparent from examination of a post transplant peripheral smear or marrow biopsy. A severe prolonged deficiency of erythroid and megakaryocyte marrow progenitors may persist for many years after bone marrow transplantation, even though peripheral blood cell counts and marrow cellularity may reach pre-transplant levels. Colony forming units-fibroblasts (CFU-f), the precursor stromal compartment for cells of the osteogenic lineage, are critical to hematopoietic cell survival, proliferation, and differentiation. CFU-f reconstitution may take as long as 12 years to reach pretransplant numbers and is solely of host origin.

### Stem Cell Rescue (SCR) Therapy

Stem cell rescue (or rescue transplant) is a method of replacing blood-forming stem cells that were destroyed by treatment with high doses of anticancer drugs or radiation therapy. It is usually done using the patient's own stem cells that were saved before treatment. The stem cells help the bone marrow recover and make healthy blood cells. Stem cell rescue may also allow more chemotherapy or radiation therapy to be given so that more cancer cells are killed.

Typically following MBT, patients will receive an infusion of hematopoietic stem cells isolated from either bone marrow or peripheral blood with the intent of curing a systemic malignancy, inherited metabolic disease, or potentially fatal disease of the hematopoietic or immune systems. The rationale for bone marrow transplantation in patients with bone marrow failure, malignancies, and congenital hematopoietic and immunodeficiency states is to supply normal stem cells for marrow repopulation after obliteration of the diseased marrow. Regeneration of new bone marrow (“bone marrow reconstitution”) occurs during recovery from myeloablative therapy, either from stem cell progenitors or, less often, from residual host progenitors. [Riley, et al., “Hematologic Aspects of Myeloablative Therapy and Bone Marrow Transplantation.” Journal of Clinical Laboratory Analysis (2005) 19:47-79].

Morphologic Features of Stem Cell Rescue. The immediate post-chemotherapy or post-transplant period is usually followed by 1 to 2 weeks of marked marrow aplasia (meaning incomplete, retarded or defective development). Fat cell regeneration provides the first morphologic evidence of bone marrow regeneration, followed at day 6 to day 14 by the appearance of minute clusters of immature, monotypic hematopoietic cells that gradually mature and enlarge. These colonies are comprised of cells of a single hematopoietic lineage (“monophyletic”), usually myeloid or erythroid, and presumably arise from committed stem cells in the bone marrow transplant patient. The regenerating colonies tend to be paratrabecular in patients receiving bone marrow transplant only and interstitial following stem cell transplantation. Very early post-transplant hematopoiesis is usually polyclonal but may be monoclonal. Early erythropoietic islands are usually dominated by large basophilic normoblasts that may exhibit dyserythropoietic features. As hematopoietic reconstitution continues, the distribution of hematopoietic cells in the bone marrow is often atypical, such that clusters of myeloid precursors are abnormally localized in the intratrabecular areas and erythroid precursors occur near the endosteum. Megakaryocytes are normally the last to engraft. They are usually localized in the central part of the intertrabecular areas, and may appear in clusters, rather than in their normal scattered distribution. Macrophages, pseudo-Gaucher cells, and sheets of regenerating promyelocytes may also appear. (Riley, et al., “Hematologic Aspects of Myeloablative Therapy and Bone Marrow Transplantation.” Journal of Clinical Laboratory Analysis (2005) 19:47-79).

The gradual return to normal bone marrow cellularity is accompanied by resolution of the edema, reticulin fibrosis (meaning increased reticulin staining), and fibrinoid necrosis (meaning a type of necrosis that occurs in the wall of a small artery or arteriole that is pink in color, and resembles fibrin (thus, fibrinoid); it represents actual death or necrosis of the cell wall). The marrow should be approximately 50% normocellular by the third posttransplantation week and normocellular by 8 to 12 weeks. The time course of the progression from early hematopoiesis to normal marrow cellularity is extremely variable; some patients may achieve normal cellularity in as little as 14 days, while others require several months. However, 28 days is typical. The kinetics of engraftment depend on the source of donor cells (e.g., peripheral blood stem cells, cord blood, bone marrow), the dose of infused CD34+ cells, the type and dose of exogenous hematopoietic growth factors (i.e., G-CSF, rhGM-CSF, erythropoietin), and HLA crossmatching. The rate of marrow recovery is affected by the homing efficiency and clonogenic potential of the transplanted cells and whether the infused cells were expanded in vitro prior to infusion. (Riley, et al., “Hematologic Aspects of Myeloablative Therapy and Bone Marrow Transplantation.” Journal of Clinical Laboratory Analysis (2005) 19:47-79).

In the peripheral blood, granulocyte colony stimulating factor (G-CSF) and granulocyte-macrophage colony stimulating factor (GM-CSF) cause an increase in the total white blood cell count and the absolute number of neutrophils, monocytes, and eosinophils. Their effects on the bone marrow include eosinophilic hyperplasia and increases in cellularity and the myeloid:erythroid (M:E) ratio. In addition, prominently granulated and/or vacuolated neutrophils and neutrophilic precursors appear in both the peripheral blood and bone marrow. (Riley, et al., “Hematologic Aspects of Myeloablative Therapy and Bone Marrow Transplantation.” Journal of Clinical Laboratory Analysis (2005) 19:47-79).

MBT and SCR Hematologic Consequences

Stem cell rescue via bone marrow transplantation is often compared to solid organ transplantation, but it is unique in several ways. Because bone marrow is a liquid “organ,” the problems of compatible organ size, biliary and ureteral obstruction, and other surgical problems are not encountered. Since bone marrow is rapidly replenished by normal individuals, cadaver organs are not required, and the living donor sustains no permanent organ insufficiency. Patients can even supply their own bone marrow for later transfusion (autologous SCR). However, transplant recipients receiving a marrow donation from another individual (allogeneic SCR) face the problem of graft rejection, as well as rejection of the host by the graft, known as graft-vs.-host-disease (GVHD). In addition, bone marrow transplant recipients are profoundly immunosuppressed until bone marrow reconstitution occurs, and they are extremely susceptible to opportunistic infections and other problems during this period. Therefore, SCR via bone marrow transplantation is a very expensive undertaking with a significant morbidity and mortality rate. Consequently, the indications for this procedure are limited and potential recipients undergo a thorough, and potentially lengthy, screening process. (Riley, et al., “Hematologic Aspects of Myeloablative Therapy and Bone Marrow Transplantation.” Journal of Clinical Laboratory Analysis (2005) 19:47-79). The below table briefly outlines adverse hematologic consequences of MBT and/or SCR.

TABLE 4
Adverse hematologic consequences of myeloabalative
therapy and/or stem cell rescue

| Problem | Diagnostic features |
|---|---|
| Engraftment failure and | Marked marrow hypocellularity |
| delayed engraftment | Diffuse histiocytic proliferation |
| Residual bone marrow | Careful review of bone marrow |
| disease | aspirate, biopsy, and clot section |
| | supplemental by flow cytometry |
| | and/or immunoperoxidase staining |
| Minimal residual disease | Advanced diagnostic technique with |
| | detection limit of at least 10⁻³ |
| | cells |
| Hematogones | Flow Cytometric immunophenotypic |
| | analysis |
| Graft-versus-host disease | Proliferation of macrophages, |
| | myelofibrosis, bone marrow hypoplasia |
| Myelofibrosis | Reticulin fibrosis, usually in |
| | patients with CML |
| Therapy-related acute | Morphology, immunophenotypic analysis, |
| leukemia (t-AML) | molecular analysis, EBV analysis |
| Post-transplant | Cytopenias with bone marrow |
| lymphoproliferative | hypocellularity, stromal damage, |
| disorders (PTLD) Toxic | edema, perivascular plasmacytosis, |
| myelopathy | necrobiosis of neutrophilic granulocytes, |
| | and cellular debris |

### Engraftment Failure, Acute Graft Rejection, and Delayed Engraftment

Many factors may cause poor outcome after SCR via bone marrow transplantation (“graft failure”) or the loss of recently engrafted marrow tissue (“graft rejection”). Generally, failure of initial engraftment (primary graft failure) is due to genetic differences between the donor and recipient, damaged stem cells or an insufficient numbers of stem cells, inadequate immunosuppression or pretransplant conditioning, alloimmunization due to previous multiple blood transfusions, excessive T-cell depletion of the engrafted material, an abnormal microenvironment in the bone marrow of the host, an abnormal donor marrow, drug toxicity, or viral infections. Failure of the graft after engraftment (secondary graft failure) occurs as a result of drug toxicity, infections, fibrosis, or cell-mediated immune reactions. [Riley, et al., “Hematologic Aspects of Myeloablative Therapy and Bone Marrow Transplantation.” Journal of Clinical Laboratory Analysis (2005) 19:47-79].

Immunologically-mediated bone marrow acute graft rejection is particularly common in three situations: 1) multiply-transfused patients with aplastic anemia, 2) patients receiving bone marrow from a major histocompatibility mismatched donor, and 3) patients receiving T-cell depleted bone marrow. The incidence of acute rejection is only about 1% in patients receiving an immunologically unmanipulated, HLA-matched graft from a sibling, but increases to 8-15% in patients receiving T-cell depleted-phenotypically matched grafts.

Graft rejection is primarily caused by host T lymphocytes that survive the pretransplant conditioning regimen, proliferate in the allografted bone marrow, then suppress the growth of donor cells, and initiate cell-mediated responses against donor targets. Slightly different mechanisms of rejection may be involved in different patient populations, since suppressor T lymphocytes (CD3+CD8+CD57+) predominate in HLA-matched siblings undergoing allograft rejection, while cytotoxic T lymphocytes (CD3+CD8+CD57−) are found in rejecting marrow transplant patients receiving an HLA-matched allograft from an unrelated donor. (Riley, et al., “Hematologic Aspects of Myeloablative Therapy and Bone Marrow Transplantation.” Journal of Clinical Laboratory Analysis (2005) 19:47-79).

Severe thrombocytopenia after primary post-transplant recovery of platelets (“secondary failure of platelet recovery” or “SFPR”) is associated with serious complications, a poor clinical outcome, or even death. Thrombocytopenia occurs in as many as 20% of patients undergoing allogeneic transplantation, but has a much lower incidence (8%) in autologous transplantation. Cytomegalovirus infection has been implicated as a significant risk factor for the development of SFPR by several groups of investigators. (Riley, et al., “Hematologic Aspects of Myeloablative Therapy and Bone Marrow Transplantation.” Journal of Clinical Laboratory Analysis (2005) 19:47-79).

Morphologically, bone marrow aspirate smears from patients with engraftment failure or delayed engraftment are markedly hypocellular, with a predominance of stromal cells, while core biopsies and clot sections often show diffuse proliferation of histiocytes (stationary phagocytic cells present in connective tissue). (Riley, et al., “Hematologic Aspects of Myeloablative Therapy and Bone Marrow Transplantation.” Journal of Clinical Laboratory Analysis (2005) 19:47-79).

Clinically, early graft failure (>50 days post transplantation) is manifested by host T lymphocytosis (CD3+, CD8+, DR+), while late graft failure (≥50 days post transplantation) is associated with a syndrome of delayed granulopoietic regeneration, fever of unknown origin, and abdominal symptomatology. Graft rejection is often heralded by progressive lymphocytosis (high lymphocyte count) and a sudden drop in the absolute neutrophil count. The prognosis for continued successful engraftment is poor once lymphocytosis occurs, although the infusion of donor lymphocytes has been successful in a few patients. Preventive therapy in high-risk patients is directed at the inclusion of increased total body irradiation, total lymphoid irradiation, or immunosuppressive agents into the preconditioning regimen. (Riley, et al., “Hematologic Aspects of Myeloablative Therapy and Bone Marrow Transplantation.” Journal of Clinical Laboratory Analysis (2005) 19:47-79).

### Minimal Residual Disease (MRD)

MRD is the persistence of leukemic cells in the bone marrow after remission induction therapy (meaning initial treatment with anticancer drugs) below the limit of detection by conventional morphologic assessment. It is believed that these residual leukemic cells are the possible source of disease relapse in many patients who achieve “complete” morphologic remission from various forms of leukemia, thereby leading to residual or relapse bone marrow disease. The clinically relevant level of sensitivity for MRD detection has not been established, nor has it been documented that additional therapy to eradicate very small numbers of residual cells improves survival for patients in clinical and morphologic remission. (Riley, et al., “Hematologic Aspects of Myeloablative Therapy and Bone Marrow Transplantation.” Journal of Clinical Laboratory Analysis (2005) 19:47-79).

### Graft-Versus-Dost Disease (GVHD)

GVHD is a major cause of morbidity and mortality following allogeneic bone marrow transplantation. It occurs in approximately 50% of cases with histocompatible marrow transfusions, and in nearly all cases of bone marrow transplantation with an HLA-mismatched marrow. When moderate to severe, GVHD has a significant mortality rate (40-80%). Although GVHD is a complex immunologic phenomenon that is poorly understood, it is usually a T-cell mediated process occurring in an environment of lymphocyte subset imbalance, alloantigen presentation, and abnormal production or increased responsiveness to cytokines. Both acute and chronic forms of GVHD are recognized.

Acute GVHD (aGVHD) follows lymphocyte reactivity to disparities of “minor” histocompatibility antigens in the skin, gastrointestinal tract, and liver. An increased likelihood of GVHD is associated with HLA disparity between donor and host, the older age of donor and host, allosensitization of the donor, sex mismatch between donor and recipient, increased intensity of the preparative regimen, and donor T cell dose. Clinical symptomatology varies from mild skin rashes, gastrointestinal (GI) disturbances (nausea, vomiting, diarrhea), and impaired liver function tests, to life-threatening disease, with skin destruction, liver failure, bloody diarrhea, and severe immunosuppression. Approximately 5-10% of marrow-transfused patients die of GVHD. (Riley, et al., “Hematologic Aspects of Myeloablative Therapy and Bone Marrow Transplantation.” Journal of Clinical Laboratory Analysis (2005) 19:47-79).

Chronic GVHD (cGVHD) may follow the acute process or develop de novo. It occurs in 25-65% of bone marrow transplant recipients. The platelet count is a predictor of survival; platelet counts <100,000/mL are associated with an overall mortality of >50%. cGVHD is believed to represent an immunodysregulatory state characterized by autoimmune phenomena, and the clinical picture resembles autoimmune disease, with involvement of the skin, GI tract, and liver. Circulating autoantibodies are present, and deposits of complement and immunoglobulins have been identified at the dermal-epidermal junction. Risk factors for cGVHD include prior aGVHD, older donor or recipient age, HLA mismatch, use of an unrelated donor, viral infection, splenectomy, donor lymphocyte infusion (DLI), and use of peripheral blood stem cells to treat cGVHD. (Riley, et al., “Hematologic Aspects of Myeloablative Therapy and Bone Marrow Transplantation.” Journal of Clinical Laboratory Analysis (2005) 19:47-79).

### Myelofibrosis

Mild transient reticulin fibrosis is not uncommon after chemotherapy, but severe collagen fibrosis is more typical of chronic myelogenous leukemia. Bone marrow fibrosis is marked by an increase in increased reticulin fiber density, and, in severe cases, an increased number of CD61+ megakaryopoiesis, increased CD68+ macrophages, a decreased number of erythroid precursors, and an elevated platelet count. There is usually an initial regression of myelofibrosis after transplantation, but it often recurs in areas of regenerating hematopoiesis, and is associated with the presence of atypical dwarf megakaryocytes, severe acute GVHD, and a significant delay in the time to achieve transfusion independence. (Riley, et al., “Hematologic Aspects of Myeloablative Therapy and Bone Marrow Transplantation.” Journal of Clinical Laboratory Analysis (2005) 19:47-79).

### Therapy-Related Acute Leukemia

Therapy-related acute myeloid leukemia (t-AML) is a form of secondary leukemia arising from cytotoxic chemotherapy and/or radiation therapy. The incidence of t-AML following high-dose chemotherapy for a prior malignancy is progressively increasing and t-AMLs are among the most common second malignancies in both pediatric and adult populations. Polymorphism or homozygous gene deletions of glutathione S-transferases P1, M1, and T1 may play a role in the increased incidence of t-AML due to insufficient detoxification of the chemotherapeutic drugs. Patients treated for Hodgkin's lymphoma, non-Hodgkin's lymphoma (NHL), myeloma, polycythemia vera, breast cancer, ovarian carcinoma, testicular carcinoma, or de novo acute lymphoblastic leukemia (ALL) are at the greatest risk of developing t-AML, and more than 50% of patients with secondary AML have breast cancer, NHL, and Hodgkin's lymphoma. In contrast to t-AML, the incidence of therapy-related ALL is rare, with limited indications of the use of previous drugs, such as those used in MBT, as being indicative. (Riley, et al., “Hematologic Aspects of Myeloablative Therapy and Bone Marrow Transplantation.” Journal of Clinical Laboratory Analysis (2005) 19:47-79).

### Post Transplant Lymphoproliferative Disorders (PTLDs)

PTLDs are lymphoid neoplasms that develop as a consequence of immunosuppressive therapy in patients receiving bone marrow or solid organ transplants. The spectrum of post transplant lymphoproliferative disease ranges from benign to malignant monoclonal or polyclonal lymphoid proliferations, and it occurs in about 2% of solid organ transplant recipients, approximately 1% of autologous bone marrow transplant recipients, and up to 20% of patients with multiple risk factors, including receipt of an HLA-mismatched allogeneic marrow transplant and immunosuppressive therapy for GVHD, such as anti-CD3 monoclonal OKT3, cyclosporine A, and FK506. Epstein-Barr virus, either primary or reactivated, is strongly associated with development of PTLD. Impaired immune surveillance, chronic antigenic stimulation from the allograft, and the oncogenic effects of immunosuppressive therapy are additional factors that lead to PTLDs. In contrast to the typical extranodal involvement of solid organ transplant recipients with PTLD, bone marrow allograft recipients with PTLD often have widespread disease involving both nodal and extranodal sites. (Riley, et al., “Hematologic Aspects of Myeloablative Therapy and Bone Marrow Transplantation.” Journal of Clinical Laboratory Analysis (2005) 19:47-79).

### Toxic Myelopathy

Toxic myelopathy is a rare bone marrow lesion caused by toxic damage to the stromal and mesenchymal components of the bone marrow. Persistent cytopenia is the clinical hallmark of toxic myelopathy; the bone marrow is hypocellular with prominent stromal damage, including edema, perivascular plasmacytosis, necrobiosis of neutrophilic granulocytes, and cellular debris. Toxic myelopathy occurs <1% of patients receiving chemotherapy or radiotherapy. (Riley, et al., “Hematologic Aspects of Myeloablative Therapy and Bone Marrow Transplantation.” Journal of Clinical Laboratory Analysis (2005) 19:47-79).

Many, if not all, of these conditions arise from incomplete removal of the disease/malignant cells through insufficient MBT therapy, improper stem cell rescue, use of immunosuppressives as typically required after MBT, or damage in the bone marrow environment. The prevention of successful SCR and/or associated conditions may be linked to the hematopoiesis system in the patient receiving MBT. It has been hypothesized that chronic inflammation within tissue specific microenvironments impairs the ability of supportive niche cells to appropriately nurture their cognate stem cells thereby preventing SCR and hematopoiesis reconstitution. (Wagers, A. J. The stem cell niche in regenerative medicine. Cell stem cell (2012) 10, 362-369, doi:10.1016/j.stem.2012.02.018; Lane, S. W., Williams, D. A. & Watt, F. M. Modulating the stem cell niche for tissue regeneration. Nature biotechnology (2014) 32, 795-803, doi:10.1038/nbt.2978; Schepers, K., Campbell, T. B. & Passegue, E. Normal and leukemic stem cell niches: insights and therapeutic opportunities. Cell stem cell (2015) 16, 254-267, doi:10.1016/j.stem.2015.02.014 911).

Recovery from Myelosuppression

Immune reconstitution follows a general pattern developing from immature to mature immune functions. [Carson K. et al., Chapter 35—Reimmunization after stem cell transplantation,” in Hematopoietic Stem Cell Transplantation in Clinical Practice (2009); Butler, J. M. et al. Endothelial cells are essential for the self-renewal and repopulation of Notch-dependent hematopoietic stem cells. Cell stem cell (2010) 6, 251-264, doi:10.1016/j.stem.2010.02.001; Kobayashi, H. et al. Angiocrine factors from Akt-activated endothelial cells balance self-renewal and differentiation of haematopoietic stem cells. Nature cell biology (2010) 12, 1046-1056, doi:10.1038/ncb2108; Winkler, I. G. et al. Vascular niche E-selectin regulates hematopoietic stem cell dormancy, self renewal and chemoresistance. Nature medicine (2012) 18, 1651-1657, doi:10.1038/nm.2969; Ding, L., et al., Endothelial and perivascular cells maintain haematopoietic stem cells. Nature (2012) 481, 457-462, doi:10.1038/nature10783; Poulos, M. G. et al. Endothelial jagged-1 is necessary for homeostatic and regenerative 947 hematopoiesis. Cell reports (2013) 4, 1022-1034, doi:10.1016/j.celrep.2013.07.048; Greenbaum, A. et al. CXCL12 in early mesenchymal progenitors is required for haematopoietic stem-cell maintenance. Nature (2013) 495, 227-230, doi:10.1038/nature11926; Doan, P. L. et al. Epidermal growth factor regulates hematopoietic regeneration after radiation injury. Nature medicine (2013) 19, 295-304, doi:10.1038/nm.3070]. Immune reactivity during the first month post graft is extremely low. Id. Innate immunity is the first to regain function. [Ogonek, J. et al., “Immune reconstitution after allogeneic hematopoietic stem cell transplantation” Front. Immunol. (2016) 7: 507. doi: 10.3389/fimmu.2016.00507]. The reappearance of hematopoietic lineages follows a reproducible order, with monocytoid cells emerging first in the peripheral blood, followed by granulocytes and then NK cells. The recovery of NK cells significantly precedes T cells and B cells, with respect to both cell number and functional maturation [Grzywacz, B. et al, Natural Killer Cell differentiation by myeloid progenitors, Blood (2011) 117 (13): 3548-58]. Cytotoxic and phagocytic functions recover by day 100, but the more specialized functions of T- and B-lymphocytes may remain impaired for a year or even longer. After a period of time, the various components of the immune systems of most healthy marrow recipients begin to work synchronously, whereas the immune systems of patients with chronic graft-versus-host disease (GvHD) remain suppressed. Delayed and incomplete immune reconstitution renders the patient susceptible to infections which are associated with high morbidity and mortality after allo-HCT.

It has long been known that hematopoietic regeneration and revascularization of the bone marrow cavity after radiation exposure are temporally related, and that there is no hematopoietic regeneration without vascular reconstitution of the bone marrow. It is now recognized that hematopoietic regeneration after myelosuppression with cytotoxic agents or whole-body irradiation is interdependent on the bone marrow sinusoidal network and hematopoietic cells as well as on megakaryocyte maturation. [Kopp, et. al. “The Bone Marrow Vascular Niche: Home of HSC Differentiation and Mobilization.” PHYSIOLOGY 20: 349-356, 2005; 10.1152/physiol.00025.2005].

Myelosuppression leads not only to apoptosis of cycling hematopoietic cells, but also to the destruction of the bone marrow vasculature. Because the intricate network of sinusoids lack a regular vessel wall, they are especially affected by ionizing radiation, and they display ultrastructural signs of necrosis, marked dilation, and overt breakdown with plasma and blood cell leakage. Bone marrow sinusoids seem to be supported by their neighboring hematopoietic cells themselves; losing this support means losing stability, leading to hemorrhage within the bone marrow cavity after radiation or myelosuppressive chemotherapy. In the process of hematopoietic regeneration, the sinusoids are reconstructed. The processes hematopoiesis and vasculogenesis/angiogenesis therefore are closely linked. [Id].

The vasculature provides a protective niche for HSCs following chemotherapy, promoting bone and haematopoietic regeneration. [Sivan U, et al., Role of angiocrine signals in bone development, homeostasis and disease. Open Biol. (2019) 9: 190144. http://dx.doi.org/10.1098/rsob.190144]. Long-term, quiescent HSCs (LT-HSCs) are associated with both sinusoids and type H blood vessels (endosteal vessels) [Id., citing Kiel M J, et al. (2005). SLAM family receptors distinguish hematopoietic stem and progenitor cells and reveal endothelial niches for stem cells. Cell 121, 1109-1121; Kunisaki Y, et al. (2013). Arteriolar niches maintain haematopoietic stem cell quiescence. Nature 502, 637-643]. The vascular niche is essential to regenerate the HSC population after irradiation. [Id., citing Hooper A T, et al. (2009). Engraftment and reconstitution of hematopoiesis is dependent on VEGFR2 mediated regeneration of sinusoidal endothelial cells. Cell Stem Cell 4, 263-274] Transplantation of bone marrow ECs following irradiation enhances haematopoiesis and protects radiosensitive tissue [Id., citing Poulos M G, et al, (2015) Vascular platform to define hematopoietic stem cell factors and enhance regenerative hematopoiesis. Stem Cell Rep. 5, 881-894, Chute J P, et al. (2007). Transplantation of vascular endothelial cells mediates the hematopoietic recovery and survival of lethally irradiated mice. Blood 109, 2365-2372]. Irradiated mice transplanted with bone marrow EC culture conditioned media showed increased survival, indicating that angiocrine factors can enhance survival but not compensate for a complete loss of HSCs. Endothelial-specific deletion of the Notch ligand JAG-1 leads to an impairment in HSC regeneration and increase lethality following irradiation. [Id., citing Poulos M G, et al. (2013). Endothelial Jagged-1 is necessary for homeostatic and regenerative hematopoiesis. Cell Rep. 4, 1022-1034] In addition to Notch signalling, ECs upregulate Fgf-2, Bmp4, Igfbp2 and Angiopoetin-1 to expand the hematopoietic stem progenitor cells (HSPCs) [Id., citing Arai F, et al (2004) Tie2/angiopoietin-1 signaling regulates hematopoietic stem cell quiescence in the bone marrow niche. Cell 118, 149-161, Kobayashi H, et al. (2010) Angiocrine factors from Akt-activated endothelial cells balance self-renewal and differentiation of haematopoietic stem cells. Nat. Cell Biol. 12, 1046-1056], indicating these angiocrine factors may be useful to protect HSC following irradiation. (Id.).

Aged bone marrow ECs impair HSCs and promote a myeloid bias, as demonstrated by transplantation of ECs from the bone marrow of the aged mice into the young recipients [Id., citing Chute J P, et al (2007) Transplantation of vascular endothelial cells mediates the hematopoietic recovery and survival of lethally irradiated mice. Blood 109, 2365-2372]. The aged bone marrow has a reduction in PDGFR-β expressing pericytes, which correlates with an expansion of disseminated tumour cells (DTCs). Furthermore, the aged bone marrow secretome promotes proliferation of breast cancer cells in bone. Type H ECs expand in response to radiation and chemotherapy and mediate the regenerative angiogenesis in the bone via blood flow-mediated secretion of PDGF-B, which promotes pericyte expansion [Id., citing Singh A, et al. (2019) Angiocrine signals regulate quiescence and therapy resistance in bone metastasis. JCI Insight 4, 125679 (10.1172/jci.insight.125679].

The number of elderly persons is increasing with unprecedented speed around the globe. The aging process is associated with an increased susceptibility to life-threatening diseases of the cardiovascular and hematopoietic systems.

While advances in non-ablative and reduced conditioning transplantation have dramatically increased the number of older individuals undergoing BM transplants, aging is associated with increased risk of negative outcomes/failures, because elderly patients respond poorly to myeloablative strategies that are necessary for the successful transplantation of HSCs, and also develop prolonged cytopenias following myelosuppressive therapies that are often used to treat hematopoietic malignancies and other cancers.

Currently, there is very little insight into how aging of the hematopoietic system, particularly the BM microenvironment, impacts engraftment or regeneration of the BM niche. Aging of the blood system is associated with loss of vascular integrity and dramatic changes in HSC function. In addition to increasing in number and losing self-renewal potential, old HSCs exhibit a myeloid bias and increasing propensity to develop hematologic malignancies. While some of these changes reflect cell-intrinsic alterations, emerging evidence suggests that some of these defects also may be regulated by the BM microenvironment, in particular the BM vascular niche.

The described invention has identified thrombospondin-1 as a candidate pro-aging factor. Experiments are ongoing to determine whether inhibiting this candidate factor can restore the functional capacity of an aging blood and vascular system and exploit the potential of bone marrow blood vessels to rejuvenate the aged blood system back to youthful levels. Pro-aging factor blockade may have one or more of the following benefits: preserve vascular function and integrity in multiple organ beds in the aged; enhance ex vivo expansion of aged HSCs for transplantation; promote bone regeneration in the aged; preserve perivascular stromal niche cell function; rejuvenate perivascular stromal niche cell function; or both; prevent premature aging of the hematopoietic and vascular system; rejuvenate an aged hematopoietic and vascular system; preserve stem cell function in multiple organs; rejuvenate stem cell function in multiple organs or both. Without being limited by theory, rejuvenating the blood and vascular system will be critical in reversing age-related hematopoietic deficiencies and in restoring overall healthspan.

### SUMMARY OF THE INVENTION

According to one aspect, the described invention provides a method for rejuvenating an aging blood and vascular system comprising aging-associated hematopoietic defects in an aging hematopoietic microenvironment of bone marrow including deteriorating vascular integrity, reduced hematopoietic stem cell function, or both, comprising: administering to a subject a pharmaceutical composition comprising an inhibitor of an angiocrine factor, a splice variant, or a fragment thereof, wherein the angiocrine factor is thrombospondin 1 (TSP1), and a pharmaceutically acceptable carrier; optionally administering a stem cell co-therapy comprising transplantation of a therapeutic amount of multipotent, self-renewing hematopoietic stem cells (HSCs) effective to regenerate the blood system and promote hematopoietic reconstitution of the bone marrow, and optionally administering a vascular endothelial co-therapy comprising transplantation of a therapeutic amount of endothelial cells (ECs) effective to regenerate the blood system and promote hematopoietic reconstitution of the bone marrow, and enhancing hematopoietic recovery in the hematopoietic bone marrow microenvironment by one or more of: reducing inflammation in the hematopoietic microenvironment of the bone marrow; preserving vascular integrity in the hematopoietic microenvironment of the bone marrow; or increasing frequency and numbers of cell types in the hematopoietic compartment to effect multi-lineage reconstitution.

According to some embodiments, inhibition of TSP1 is by an antibody, an siRNA, or TSP1 gene knockout by CRISPR-comprising a synthetic single guide RNA. According to some embodiments, the antibody is a non-neutralizing antibody to TSP1. According to some embodiments, the antibody is a neutralizing antibody to TSP1. According to some embodiments, the neutralizing antibody is commercially available as clone A4.1 (Thermofisher, Invitrogen RRID AB_10988669)). the HSC niche comprises hematopoietic stem cells (HSCs), hematopoietic progenitor cells (HPCs), resident niche cells comprising osteoblastic cells that regulate stem cell pool size during hematopoiesis, and secreted and membrane bound factors comprising chemokines, wherein at steady state, the HSCs are mostly quiescent, while HPCs are actively proliferating and contributing to daily hematopoiesis; and the vascular niche comprises an endothelial microniche comprising endothelial cells comprising bone marrow endothelial cells (BMECs), which, when activated, produce angiocrine factors that orchestrate a system of cellular crosstalk that results in differential production of the angiocrine factors.

According to some embodiments, the aged endothelial microenvironment within the aged bone marrow hematopoietic microenvironment of the HSC niche containing aged BMECs includes one or more of: a decrease in mTOR signaling, a reduced abundance of an mTOR subunit, reduced phosphorylation of mTOR catalytic subunits, reduced expression of mTOR transcription target genes; or reduced protein levels in mTOR catalytic subunit mTOR Complex 1 and mTOR Complex 2. According to some embodiments, the decrease in mTOR signaling by BMECs causes functional defects associated with aging in aged HSCs. According to some embodiments, expression levels of thrombospondin-1 (TSP1) are upregulated in aged BMECs when compared to a young control. According to some embodiments, top upregulated biological processes represented by changes in gene expression in aged BMECs, compared to a young control, which include changes in STAT3 pathway, TGF-b signaling, IGF-1 signaling or HMGB1 signaling, are regulated by TSP1. According to some embodiments, the deteriorating vascular integrity comprises increased vascular permeability including increased endothelial permeability, increased endothelial inflammation, or both. aging-associated hematopoietic defects in the HSC niche of the bone marrow hematopoietic microenvironment include one or more of: sustained inflammation; increased HSC cellularity; increased stem cell pool size; loss of HSC quiescence; increased HSC apoptosis; loss of HSC self-renewal potential; increased myeloid-biased differentiation of the HSCs, increased risk of failure of myeloablative strategies; or reduced engraftment and regeneration of the bone marrow niche after transplantation, compared to a young control. According to some embodiments, the sustained inflammation is derived from a myelosuppressive insult. According to some embodiments, the myelosuppressive insult comprises exposure to radiation, chemotherapy or both. According to some embodiments, the myelosuppressive insult comprises chemotherapy. According to some embodiments, the myelosuppressive insult is myeloablative. According to some embodiments, the increased myeloid-biased differentiation of the HSCs is at expense of lymphopoiesis. According to some embodiments, the loss of quiescence for HSCs leads to a transient increase in HSCs, long-term exhaustion of HSCs, and defects in long-term repopulation capacity of HSCs. According to some embodiments, overactivation of endothelial cell mTOR drives HSCs from quiescence into more active cell cycling. According to some embodiments, aging-associated hematopoietic defects in the HSC niche of the bone marrow hematopoietic microenvironment include changes in HSC gene expression. According to some embodiments, the changes in HSC gene expression associated with aging in aged HSCs comprise upregulation of one or more of SELP, NEO1, JAM2, SLAMF1, PLSCR2, CLU, SDPR, FYB, ITGA6 and downregulation of downregulation of one or more of RASSF4, FGF11, HSPA1B, HSPA1A, or NFKBIA.

According to another aspect, the described invention provides a method for preparing a hematopoietic stem cell product for hematopoietic stem cell transplantation comprising: (a) preparing ex vivo cultures of hematopoietic stem cells; (b) administering an antibody comprising anti-TSP1 antibodies to the cultures of hematopoietic stem cells in (a) to form a treated hematopoietic stem cell population; and (c) expanding the treated hematopoietic stem population in vitro to form a hematopoietic stem cell transplantation product comprising a therapeutic amount of treated hematopoietic stem cells, wherein engraftment potential of the hematopoietic stem cell transplantation product is enhanced compared to an untreated control.

According to some embodiments, the hematopoietic stem cells of step (a) are derived from a human subject. According to some embodiments, the hematopoietic stem cells of step (a) are derived from a mouse subject. According to some embodiments, the antibody comprising the anti-TSP1 antibodies are neutralizing antibodies. According to some embodiments, the anti-TSP1 antibodies further comprise antibodies to CD36, CD47 or both. According to some embodiments, the antibodies are humanized antibodies. According to some embodiments, the transplantation is autologous. According to some embodiments, the hematopoietic stem cell transplantation is allogeneic.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic of the mTOR signaling pathway showing key signaling nodes that regulate mTORC1 and MTorC2. FIG. 1B is a schematic showing the key outputs of the mTORC1 and mTORC2 pathways. (FIG. 1A, 1B, taken from Laplante, M., Sabatini, D M, Cell (2012) 149(2): 274-293). FIG. 1C is a schematic of the PI3K/Akt/mTOR signaling pathway. (Taken from Porta, C. et al, Frontiers in Oncol. (2014) doi 10.3389/fonc.2014.00064).

FIGS. 2A, 2B, 2C, and 2D show that aged bone marrow endothelial cells display impaired mTOR signaling. FIG. 2A illustrates abundance of PIK3CA/PIK3R1 complex in young and aged mice. The data show that mTOR subunit abundance is decreased in BMECs of aged mice. FIG. 2B shows quantification of mean fluorescent intensity of freshly-isolated BMECs in young and aged mice. The data demonstrate a decrease in the mTOR phosphor-Ser2448. FIG. 2C is an expression analysis of mTOR downstream transcriptional target genes by RT-PCR. Gene expression was normalized to the Actb gene, which encodes beta-actin. FIG. 2D shows Western blot analysis of pooled young and aged mice samples (N=5). In aged mice, a decrease in protein levels in the mTOR catalytic subunit (p-mTOR S2448), mTOR Complex 1 (p-S6K T389), and mTOR Complex 2 (p-AKT S473) was observed.

FIGS. 3A, 3B, 3C, 3D, 3E, 3F, 3G, 3H, and 3I show that EC-specific deletion of mTOR (mTOR^{(ECKO)}) caused alterations in HSCs reminiscent of those associated with aging. mTOR was specifically deleted from adult ECs by crossing an mTOR^{fl/fl} mouse to a tamoxifen-inducible cre transgenic mouse driven by the adult EC-specific VEcadherin promoter (mTOR^{(ECKO)}). Flow cytometric analysis was performed on young (12-16 weeks) mTOR^{(ECKO)} mice and young (12-16 weeks) control mice to determine the effect of EC-specific mTOR deletion on the regulation of HSCs and their progeny; 22-24 month old wild-type mice served as aged controls. mTOR^{(ECKO)} FIG. 3A is a plot of total hematopoietic cells/femur; FIG. 3B is a plot of phenotypic LT-HSCs/10⁶ whole bone marrow; FIG. 3C is a plot of % lineage+ cells from peripheral blood; FIG. 3D is a plot of CFUs/25,000 whole bone marrow cells; FIG. 3E is a plot of % polarized LT-HSCs; FIG. 3F shows representative images of αTUBULIN staining to demarcate cellular polarity; FIG. 3G, shows a plot of number of γH2AX foci per cell (x axis) versus percentage of cells scored (y axis); FIG. 3H, depicts representative images showing increased γH2AX foci; and FIG. 3I, shows transcriptional profiles.

FIGS. 4A, 4B, and 4C show that mTOR^{(ECKO)} HSCs express an aged HSC gene signature. FIG. 4A is a Venn Diagram comparing significant changes between young and aged HSC transcriptional datasets. FIG. 4B shows common aged HSC gene expression changes. Genes listed demonstrate shared changes in expression between the current study and published datasets whose expression was confirmed in HSCs (red—upregulated in aged HSCs; green—downregulated in aged HSCs). Genes in bold text comprise concordant expression changes between all datasets and represent an aged HSC expression signature. Ten (10) genes were identified that show significant upregulation in expression with aging, nine of which were confirmed by RT-qPCR analysis. mTOR^{(ECKO)} FIG. 4C depicts RT-qPCR confirmation of microarray-identified aged HSC gene expression signature in mTOR^{(ECKO)} and aged mice. Note that HSCs from mTOR^{(ECKO)} share an aged HSC gene expression signature.

FIG. 5 shows that mTOR^{(ECKO)} HSCs display aged hematopoietic defects following competitive transplantation of CD45.2+ HSCs from young mTOR^{(ECKO)} mice, young control mice, and aged control mice into lethally-irradiated CD45.1 mice. X axis, weeks post transplant. FIG. 5A, overall engraftment CD45.2 (y axis, % CD45.2+ engraftment); FIG. 5B, myeloid engraftment (y-axis, % CD45.2+GR1+(CD11B+) engraftment); FIG. 5C B cell engraftment (y-axis, % CD45.2+B220+ engraftment); FIG. 5D, T cell engraftment (y-axis % CD45.2+CD3+ engraftment)

FIG. 6 shows proteomics analysis on BMECs of young, mTOR^{(ECKO)}, and aged mice. FIG. 6A is a heatmap showing conserved gene changes in BMECs isolated from mTOR^{(ECKO)} and aged mice when compared to young mice. FIG. 6B shows volcano plots demonstrating that Thrombospondin-1 (TSP1) is the most significantly upregulated gene and has the greatest fold change in both mTOR^{(ECKO)} and aged BMECs when compared to young control BMECs. FIG. 6 C shows ingenuity pathway analysis of aged and mTOR^{(ECKO)} BMECs demonstrating that inhibition of Angiogenesis by TSP1 is the top upregulated biological process. FIG. 6D) shows relative TSP1 gene expression in Young=Y; Old=O; mTOR^{(ECKO)}=M BMECs. FIG. 6E shows TSP1 protein levels in Young=Y; Old=O; mTOR^{(ECKO)}=M BMECs.

FIG. 7 shows that inhibition of TSP1 in young mice increases HSC numbers and function. FIG. 7A shows steady state analysis of phenotypic LT-HSCs in control, TSP1−/− mice, and control mice that received infusions of a neutralizing antibody to TSP-1 (A4.1). FIG. 7B shows results of a progenitor colony-forming assay using WBM isolated from the aforementioned cohorts. FIG. 7C shows results when 100 LT-HSCs were infused in a competitive transplantation assay from the aforementioned cohorts. Note that TSP1−/− mice or mice treated with a TSP-1 inhibitor resulted in an increase in HSC function.

FIG. 8 shows that aged TSP1−/− mice have preserved HSC function. FIG. 8A shows a schematic of the experimental protocol whereby mice were aged for 12-months. It further shows steady state analysis of phenotypic LT-HSCs as determined in all 3 cohorts. FIG. 8B shows results of a progenitor colony-forming assay using WBM isolated from the aforementioned cohorts. FIG. 8C) shows results when 100 LT-HSCs were infused in a competitive transplantation assay from the aforementioned cohorts. Note that HSCs from aged TSP1−/− mice resemble HSCs isolated from young controls.

FIG. 9 shows that aged TSP1−/− mice have preserved HSC function. FIG. 9A is a depiction of the three cohorts (Young Controls, Aged Controls, and Aged TSP1 mice) used for HSC transplants and RNA sequencing. FIG. 9B is a bar graph of normalized mRNA expression (y-axis) vs. genes associated with HSC aging (x-axis). HSCs were isolated from the three cohorts depicted in FIG. 9A and subjected to RNA sequencing. Genes that are associated with HSC aging were decreased in aged HSCs from TSP1−/− mice. FIG. 9C is a bar graph of % CD45.2 engraftment (y axis) in the three cohorts of FIG. 9A (x-axis). FIG. 9D is a bar graph of % lineage+ cells (CD45.2) (y-axis) vs. myeloid peripheral blood cell type (CD11b+/GR1+), B cell (B220+) and T cell (CD3+) populations (x-axis) in the three cohorts. 100 LT-HSCs were infused in a competitive transplantation assay from the three cohorts. Note that HSCs from aged TSP-1−/− mice resemble HSCs isolated from young controls with enhanced long-term, multilineage engraftment.

FIG. 10 shows that TSP1 directly affects the expansion of young HSCs. FIG. 10A is a schematic demonstrating the ex vivo expansion protocol to test whether exogenous TSP1 can influence HSC expansion and function. FIG. 10B is a bar graph of % CD45.2 engraftment (y-axis) of cells treated (from left to right) with rTSP1 (500 ng/ml); uTSP1 neutralizing antibody clone 1 [ThermoFisher Scientific; MA5-13398]; uTSP1 neutralizing antibody clone 2 [ThermoFisher Scientific; MA5-13385; Ms IgG1k IgG control [ThermoFisher Scientific; MA5-13385; uTSP neutralizing antibody clone 3 ThermoFisher Scientific; MA5-13377; and Ms IgM control (x axis) [ThermoFisher Scientific; 14-4752-82]. Following an 11 day expansion, HSCs were competitively transplanted, and engraftment was assessed 24 months post-transplant. We found that exogenous TSP1 had profound, negative effects on HSC engraftment and that a neutralizing antibody to TSP1 (Clone #3) was able to override the defects of exogenous TSP, and also to enhance the functional output of the expanded HSCs. FIG. 10C is a bar graph of % lineage+ cells (CD45.2, y-axis) showing myeloid lineage (CD11b/GR1+), lymphoid [B220, B cell; CD3 T cell] lineage distributions 24 weeks post-transplant.

FIG. 11 shows that TSP1 directly affects the expansion of young HSCs. Ex-vivo expanded young HSCs were isolated from control and TSP1 global knockout (KO) mice in the PVA protocol and the HSCs competitively transplanted. As shown in FIG. 11A (bar graph of % CD45.2 engraftment (y-axis) vs. Control, TSP1−/−, uTSP1 antibody treated [ThermoFisher Scientific; MA5-13377] (x axis)), long-term, multilineage engraftment showed that HSCs treated with the TSP1 neutralization antibody engrafted similar to TSP1 knockout HSCs; both conditions out-performed control HSCs. Following an 11-day expansion, HSCs were competitively transplanted, and engraftment was assessed 24 months post-transplant. FIG. 11B is a bar graph of % CD45.2 engraftment (y-axis) in Young (control, uTSP1-treated), and aged (control, uTSP1-treated) HSCs (x-axis). We found that exogenous TSP1 had profound negative effects on HSC engraftment and that a neutralizing antibody to TSP1 (clone #3) was able to not only override the defects of exogenous TSP1, but also to enhance the functional output of the expanded HSCs. FIG. 11C is a bar graph of lineage composition (% of CD45.2+, y axis) vs. myeloid (CD11B+GR1+), lymphoid (B cell, B220+, T cell, CD3+) young (control, uTSP1-treated) and aged (control, α-TSP1-treated) (x-axis) HSCs 24 weeks post-transplant.

FIG. 12 shows that inhibition of TSP1 promotes healthy aging. FIG. 12A shows representative images of aged TSP1 mice alongside young controls and aged controls. Note the loss and graying of hair in aged controls, whereas aged TSP1 mice look similar to young controls. FIG. 12B is a bar graph showing body weight (g) (y-axis) vs. young control, aged control and aged TSP1 KO mice (x axis). FIG. 12C shows VE cadherin (red)/perilipin (green)/DAPI (blue) staining in the bone marrow microenvironment in young control, aged control and aged TSP1 KO mice (x-axis). FIG. 12D shows fat/body weight ratio (DEXAScan, y-axis) vs. control and TSP1 KO mice. FIGS. 12E, 12F, and 12G show blood chemistry for cholesterol (FIG. 12E), insulin (FIG. 12F), and fasted glucose levels (FIG. 12G) for control and TSP1KO mice. FIG. 12H shows DEXAScan used to determine bone mineralization to weight ratios in control and TSP1 KO mice. FIG. 12I shows forelimb/hindlimb grip strength in control and TSP1KO mice.

FIG. 13 shows downregulation of TSP1 gene expression via siRNA delivery in endothelial cells.

### DETAILED DESCRIPTION

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 20% of the numerical value of the number with which it is being used. Therefore, about 50% means in the range of 40%-60%, inclusive.

The term “adaptive immunity” as used herein refers to the protection of a host organism from a pathogen or toxin which is mediated by B cells and T cells, and is characterized by immunological memory. Adaptive immunity is highly specific to a given antigen and is highly adaptable.

“Administering” when used in conjunction with a therapeutic means to give or apply a therapeutic directly into or onto a target organ, tissue or cell, or to administer a therapeutic to a subject, whereby the therapeutic positively impacts the organ, tissue, cell, or subject to which it is targeted. Thus, as used herein, the term “administering”, when used in conjunction with compositions comprising an angiocrine factor, can include, but is not limited to, providing the composition into or onto the target organ, tissue or cell; or providing a composition systemically to a patient by, e.g., intravenous injection, so that the therapeutic reaches the target organ, tissue or cell. “Administering” may be accomplished by parenteral, oral or topical administration, by inhalation, or by such methods in combination with other known techniques.

The term “aging” as used herein refers to the process of growing or appearing older. The term “physiological aging” and its various grammatical forms as used herein is a measure of biological age in relation to changes that affect biological function and the ability to adapt to metabolic stress. Factors that play a role in determining physiological aging include, without limitation, chronological age, genetics, lifestyle, nutrition, diseases, and other conditions.

The term “angiogenesis” as used herein refers to the process by which new blood vessels take shape from existing blood vessels by “sprouting” of endothelial cells, thus expanding the vascular tree.

The term “amino acid” is used to refer to an organic molecule containing both an amino group and a carboxyl group; those that serve as the building blocks of naturally occurring proteins are alpha amino acids, in which both the amino and carboxyl groups are linked to the same carbon atom. The terms “amino acid residue” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Glutamic acid; G=Gly=Glycine; H=His=Histidine; I=Ile=lsoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

The following represent groups of amino acids that are conservative substitutions for one another:
Alanine (A), Serine (S), Threonine (T);
Aspartic Acid (D), Glutamic Acid (E);
Asparagine (N), Glutamine (Q);
Arginine (R), Lysine (K);
Isoleucine (I), Leucine (L), Methionine (M), Valine (V); and
Phenylalanine (F), Tyrosine (Y), Tryptophan (W).

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “angiogenesis” as used herein refers to the process by which new blood vessels take shape from existing blood vessels by “sprouting” of endothelial cells, thus expanding the vascular tree.

The term “angiocrine factor” as used herein refers to vascular niche-derived paracrine factors produced by endothelial cells that maintain organ homeostasis, balance the self-renewal and differentiation of stem cells, and orchestrate organ regeneration and tumor growth. Angiocrine factors comprise secreted and membrane-bound inhibitory and stimulatory growth factors, trophogens, chemokines, cytokines, extracellular matrix components, exosomes and other cellular products that are supplied by tissue-specific ECs to help regulate homeostatic and regenerative processes in a paracrine or juxtacrine manner. These factors also play a part in adaptive healing and fibrotic remodeling. Subsets of angiocrine factors can act as morphogens to determine the shape, architecture, size and patterning of regenerating organs. The angiocrine profile of each tissue-specific bed of ECs is different and reflects the diversity of cell types found adjacent to ECs in organs. Although subsets of angiocrine factors are produced constitutively, some angiogenic factors can modulate the production of other tissue-specific angiocrine factors. For example, VEGF-A induces the expression of defined angiocrine factors through interaction with VEGFR-1 and VEGFR-2. Similarly, FGF-2 (through the activation of FGFR-1) and the angiopoietins (through their interaction with the receptor Tie2) drive the expression of unique clusters of angiocrine factors. TSP-1 functions in a complex manner and can act as an inhibitory angiogenic factor as well as directly influence the differentiation of stem and progenitor cells. The molecular programmes that govern the production of context-dependent angiocrine factors from organ-specific ECs remain undefined. Rafii, S., et al, “Angiocrine functions of organ-specific endothelial cells,” Nature (2016) 529 (7586): 316-325).

Table 5 provides a glossary of exemplary angiocrine factors, with their reported cellular source, cellular target and function.

TABLE 5
Glossary of Angiocrine factors

| | Angiocrine | Cellular or | | |
|---|---|---|---|---|
| Abbreviation | Factor | Tissue Source | Target Cell | Function |
| Ang | Angiopoietin | endothelial | HSPC | protection |
| | | cells | | of HSPC |
| BDNF | Brain derived | | | |
| | nerve growth | | | |
| | factor | | | |
| BMP2, BMP4 | Bone | endothelial | chondrocytes | endochondral |
| | morphogeneic | cells, | | bone formation, |
| | protein 2 and 4 | nonspecific | | fracture repair, |
| | | | | organogenesis, |
| | | | | tumorigenesis |
| BTC | Betacellulin | | | |
| CTGF | Connective tissue | | | |
| | growth factor | | | |
| DKK1, DKK3 | Dickkopf WNT | | | |
| | signaling pathway | | | |
| | inhibitor 1 and 3 | | | |
| Dhh | Desert hedgehod | | | |
| EGFL7 | Epidermal growth | | | |
| | factor like-7 | | | |
| EFNB2 | Ephrin B2 | | | |
| | E-selectin | endothelial | osteoclasts, | trafficking |
| | | cells | leucocytes | leucocytes, |
| | | | | cancer |
| | | | | metastasis |
| FGF1 | Fibroblast growth | endothelial | osteoblast and | osteoprogenitor |
| | factor 1 | cells | osteoprogenitor | survival |
| FGF2 | Fibroblast growth | endothelial | HSPCs | HSPC expansion |
| | factor 2 | cells | | |
| GDF11 | Growth | | | |
| | differentiation | | | |
| | factor-11 | | | |
| GDNF | Glial cell line- | | | |
| | derived | | | |
| | neurotrophic | | | |
| | factor | | | |
| HB-EGF | Heparin binding- | | | |
| | epidermal growth | | | |
| | factor | | | |
| HGF | Hepatocyte | | | |
| | growth factor | | | |
| ICAM-1 | | endothelial | leucocytes and | leucocytes |
| | | cells | fibroblasts | trafficking |
| IGFBP | Insulin growth | endothelial | HSPC | expansion of |
| | factor binding | cells | | HSPCs |
| | protein | | | |
| Jag1, Jag2 | Jagged-1, Jagged | endothelial | HSCs | HSC |
| | 2 | cells | | regeneration, |
| | | | | haematopoiesis, |
| | | | | angiogenesis, and |
| | | | | tumorigenesis |
| IL1 | Interleukin-1 | | | |
| IL6 | Interleukin-6 | | | |
| IL7 | Interleukin-7 | endothelial | pro-8 cells | pro-B cell |
| | | cells and | | maintenance |
| | | perivascular | | |
| | | stromal cells | | |
| IL33 | Interleukin-13 | CD105+ | osteoblasts | osteogenesis, |
| | | endothelial | | haematopoiesis |
| | | cells | | |
| KL | Kit-ligand | endothelial | | |
| | | cells and | | |
| | | perivascular | | |
| | | stromal cells | | |
| LAMA4 | | | | |
| Mmp2, | Matrix | | | |
| Mmp9, | Metalloproteinases | | | |
| Mmp14 | 1, 9, and 14 | | | |
| | Noggin | endothelial | osteoblast and | bone growth, |
| | | cells | osteoprogenitor | mineralization |
| | | | | and chondrocyte |
| | | | | maturation |
| NRG | Neuregulin | | | |
| | nidogen-1 | sinusoidal and | pro-B cells | pro-B cell |
| | | perivascular | | maintenance |
| | | stromal cells | | |
| NO | Nitric oxide | | | |
| NOS2 | | endothelial | osteoblast | negative |
| | | cells | | regulation of |
| | | | | osteoblast |
| | | | | differentiation |
| NT-3 | Neurotrophin-3 | | | |
| OPG | | endothelial cell | osteoclasts | inhibit |
| | | | | osteoclastogenesis |
| PDGF | | endothelial | osteoprogenitor | osteoprogenitor |
| | | cells | | proliferation and |
| | | | | survival |
| PEDF | Pigment | | | |
| | epithelium- | | | |
| | derived factor | | | |
| PGE2 | Prostaglandin-E2 | | | |
| PIGF1 | PIGF2, Placental | | | |
| | growth factor-1 or | | | |
| | 2 | | | |
| SCF | | type H, arterial | HSCs | type H, arterial |
| | | and | | and sinuspoidal |
| | | sinuspoidal | | endothelial cells |
| | | endothelial | | |
| | | cells | | |
| SDF1 | Stromal derived | endothelial | HSCs | HSC maintenance |
| | factor-1 (Cxcll2) | cells and | | |
| | | mesenchymal | | |
| | | stem cells | | |
| SEMA-III | | endothelial | osteoclasts | bone remodeling |
| | | cells | | |
| | tenascin-C | endothelial | HSCs | HSC survival |
| | | cells | | |
| TGF | | endothelial | osteoprogenitor | osteoprogenitor |
| | | cells | | survival |
| Timp1-4 | | type H | chondrocytes | bone resorption |
| | | endothelial | | and remodeling |
| | | cells | | |
| TSP1 | Thrombospondin- | endothelial | disseminated | quiescence of |
| | 1 | cells | tumour cells | DTCs |
| TNF | Tumor necrosis | | | |
| | factor | | | |
| VCAM-1 | | endothelial | osteoclasts, | leucocytes |
| | | cells | leucocytes and | trafficking, |
| | | | fibroblasts | protection of |
| | | | | DTCs |
| VEGF | Vascular | | | |
| | endothelial | | | |
| | growth factor | | | |
| VEGFR1, | Vascular | | | |
| VEGFR2 | endothelial | | | |
| | growth factor | | | |
| | receptor-1 (Flt1), | | | |
| | and Vascular | | | |
| | endothelial | | | |
| | growth factor | | | |
| | Receptor-2 (KDR, | | | |
| | Flk1) | | | |
| | von Willebrand | endothelial | disseminated | protection of |
| | factor | cells | tumour cells | DTCs |
| Wls | Wntless | | | |
| Wnt2, Wnt9B | Wingless-type | | | |
| | MMTV | | | |
| | integration site | | | |
| | family | | | |

The terms “animal,” “patient,” and “subject” as used herein include, but are not limited to, humans and non-human vertebrates such as wild, domestic, and farm animals. According to some embodiments, the terms “animal,” “patient,” and “subject” may refer to mammals, including humans.

The term “antagonist” as used herein refers to a substance that counteracts the effects of another substance.

The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, κ and λ; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bispecific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies. Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage. The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

The term “antibody construct” as used herein refers to a polypeptide comprising one or more the antigen-binding portions of the invention linked to a linker polypeptide or an immunoglobulin constant domain. Linker polypeptides comprise two or more amino acid residues joined by peptide bonds and are used to link one or more antigen-binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

As used herein, the terms “antigen” or “immunogen” are used interchangeably to refer to a substance that elicits an immune response. An “antigenic determinant” or “epitope” is an antigenic site on a molecule. Sequential antigenic determinants/epitopes essentially are linear chains. In ordered structures, such as helical polymers or proteins, the antigenic determinants/epitopes essentially would be limited regions or patches in or on the surface of the structure involving amino acid side chains from different portions of the molecule which could come close to one another. These are conformational determinants.

Apoptotic Pathways. Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation, can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “autocrine signaling” as used herein refers to a type of cell signaling in which a cell secretes signal molecules that act on itself or on other adjacent cells of the same type.

The terms “autologous” or “autogeneic” as used interchangeably herein mean derived from the same organism.

The term “autophagy” as used herein refers to a self-degradative process that is important for balancing sources of energy at critical times in development and in response to nutrient stress, and also plays a housekeeping role in removing misfolded or aggregated proteins, clearing damaged organelles, such as mitochondria, endoplasmic reticulum and peroxisomes, as well as eliminating intracellular pathogens. Glick, D. et al., J. Pathol (2010) 221(1): 3-12). There are three defined types of autophagy: macro-autophagy, micro-autophagy, and chaperone-mediated autophagy, all of which promote proteolytic degradation of cytosolic components at the lysosome. Macro-autophagy delivers cytoplasmic cargo to the lysosome through the intermediary of a double membrane-bound vesicle, referred to as an autophagosome, that fuses with the lysosome to form an autolysosome. In micro-autophagy, by contrast, cytosolic components are directly taken up by the lysosome itself through invagination of the lysosomal membrane. Both macro- and micro-autophagy are able to engulf large structures through both selective and non-selective mechanisms. In chaperone-mediated autophagy (CMA), targeted proteins are translocated across the lysosomal membrane in a complex with chaperone proteins (such as Hsc-70) that are recognized by the lysosomal membrane receptor lysosomal-associated membrane protein 2A (LAMP-2A), resulting in their unfolding and degradation.

The term “binding” and its other grammatical forms as used herein means a lasting attraction between chemical substances.

“Binding fragments” of an antibody can be produced by recombinant DNA techniques, or by enzymatic or chemical cleavage of intact antibodies. Binding fragments include Fab, Fab′, F(ab′)₂, Fv, and single-chain antibodies.

A “bispecific” or “bifunctional antibody is an antibody in which each of its binding sites is not identical. A “bispecific” antibody construct or immunoglobulin is hence an artificial hybrid antibody or immunoglobulin having at least two distinct binding sites with different specificities. Bispecific antibody constructs can be produced by a variety of methods including fusion of hybridomas or linking of Fab′ fragments. See, e.g., Songsivilai & Lachmann, Clin. Exp. Immunol. 79:315-321 (1990).

An antibody other than a “bispecific” or “bifunctional” antibody is understood to have each of its binding sites identical.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bone marrow-derived endothelial cells” or BMECs as used herein refers to a functional component of the bone marrow stroma, which have been shown to release hematopoietic regulatory factors, as well as to selectively adhere and support the proliferation and differentiation of CD34+ hematopoietic progenitors.

Bone Cells. Four cell types in bone are involved in its formation and maintenance. These are 1) osteoprogenitor cells, 2) osteoblasts, 3) osteocytes, and 4) osteoclasts.

Osteoprogenitor Cells. Osteoprogenitor cells arise from mesenchymal cells, and occur in the inner portion of the periosteum and in the endosteum of mature bone. They are found in regions of the embryonic mesenchymal compartment where bone formation is beginning and in areas near the surfaces of growing bones. Structurally, osteoprogenitor cells differ from the mesenchymal cells from which they have arisen. They are irregularly shaped and elongated cells having pale-staining cytoplasm and pale-staining nuclei. Osteoprogenitor cells, which multiply by mitosis, are identified chiefly by their location and by their association with osteoblasts. Some osteoprogenitor cells differentiate into osteocytes. While osteoblasts and osteocytes are no longer mitotic, it has been shown that a population of osteoprogenitor cells persists throughout life.

Osteoblasts. Osteoblasts, which are located on the surface of osteoid seams (the narrow region on the surface of a bone of newly formed organic matrix not yet mineralized), are derived from osteoprogenitor cells. They are immature, mononucleate, bone-forming cells that synthesize collagen and control mineralization. Osteoblasts can be distinguished from osteoprogenitor cells morphologically; generally they are larger than osteoprogenitor cells, and have a more rounded nucleus, a more prominent nucleolus, and cytoplasm that is much more basophilic. Osteoblasts make a protein mixture known as osteoid, primarily composed of type I collagen, which mineralizes to become bone. Osteoblasts also manufacture hormones, such as prostaglandins, alkaline phosphatase, an enzyme that has a role in the mineralization of bone, and matrix proteins.

Osteocytes. Osteocytes, star-shaped mature bone cells derived from ostoblasts and the most abundant cell found in compact bone, maintain the structure of bone. Osteocytes, like osteoblasts, are not capable of mitotic division. They are actively involved in the routine turnover of bony matrix and reside in small spaces, cavities, gaps or depressions in the bone matrix called lacuna. Osteocytes maintain the bone matrix, regulate calcium homeostasis, and are thought to be part of the cellular feedback mechanism that directs bone to form in places where it is most needed. Bone adapts to applied forces by growing stronger in order to withstand them; osteocytes may detect mechanical deformation and mediate bone-formation by osteoblasts.

Osteoclasts. Osteoclasts, which are derived from a monocyte stem cell lineage and possess phagocytic-like mechanisms similar to macrophages, often are found in depressions in the bone referred to as Howship's lacunae. They are large multinucleated cells specialized in bone resorption. During resorption, osteoclasts seal off an area of bone surface; then, when activated, they pump out hydrogen ions to produce a very acid environment, which dissolves the hydroxyapatite component. The number and activity of osteoclasts increase when calcium resorption is stimulated by injection of parathyroid hormone (PTH), while osteoclastic activity is suppressed by injection of calcitonin, a hormone produced by thyroid parafollicular cells.

Bone Matrix. The bone matrix accounts for about 90% of the total weight of compact bone and is composed of microcrystalline calcium phosphate resembling hydroxyapatite (60%) and fibrillar type I collagen (27%). The remaining 3% consists of minor collagen types and other proteins including osteocalcin, osteonectin, osteopontin, bone sialoprotein, as well as proteoglycans, glycosaminoglycans, and lipids. Extracellular matrix glycoproteins and proteoglycans in bone bind a variety of growth factors and cytokines, and serve as a repository of stored signals that act on osteoblasts and osteoclasts. Examples of growth factors and cytokines found in bone matrix include, but are not limited to, Bone Morphogenic Proteins (BMPs), Epidermal Growth Factors (EGFs), Fibroblast Growth Factors (FGFs), Platelet-Derived Growth Factors (PDGFs), Insulin-like Growth Factor-1 (IGF-1), Transforming Growth Factors (TGFs), Bone-Derived Growth Factors (BDGFs), Cartilage-Derived Growth Factor (CDGF), Skeletal Growth Factor (hSGF), Interleukin-1 (IL-1), and macrophage-derived factors. There is an emerging understanding that extracellular matrix molecules themselves can serve regulatory roles, providing both direct biological effects on cells as well as key spatial and contextual information.

The Periosteum and Endosteum. The periosteum is a fibrous connective tissue investment of bone, except at the bone's articular surface. Its adherence to the bone varies by location and age. In young bone, the periosteum is stripped off easily. In adult bone, it is more firmly adherent, especially so at the insertion of tendons and ligaments, where more periosteal fibers penetrate into the bone as the perforating fibers of Sharpey (bundles of collagenous fibers that pass into the outer circumferential lamellae of bone). The periosteum consists of two layers, the outer of which is composed of coarse, fibrous connective tissue containing few cells but numerous blood vessels and nerves. The inner layer, which is less vascular but more cellular, contains many elastic fibers. During growth, an osteogenic layer of primitive connective tissue forms the inner layer of the periosteum. In the adult, this is represented only by a row of scattered, flattened cells closely applied to the bone. The periosteum serves as a supporting bed for the blood vessels and nerves going to the bone and for the anchorage of tendons and ligaments. The osteogenic layer, which is considered a part of the periosteum, is known to furnish osteoblasts for growth and repair, and acts as an important limiting layer controlling and restricting the extend of bone formation. Because both the periosteum and its contained bone are regions of the connective tissue compartment, they are not separated from each other or from other connective tissues by basal laminar material or basement membranes. Perosteal stem cells have been shown to be important in bone regeneration and repair. (Zhang et al., 2005, J. Musculoskelet. Neuronal. Interact. 5(4): 360-362).

The endosteum lines the surface of cavities within a bone (marrow cavity and central canals) and also the surface of trabeculae in the marrow cavity. In growing bone, it consists of a delicate striatum of myelogenous reticular connective tissue, beneath which is a layer of osteoblasts. In the adult, the osteogenic cells become flattened and are indistinguishable as a separate layer. They are capable of transforming into osteogenic cells when there is a stimulus to bone formation, as after a fracture.

Components of bone. Bone is composed of cells and an intercellular matrix of organic and inorganic substances. The organic fraction consists of collagen, glycosaminoglycans, proteoglycans, and glycoproteins. The protein matrix of bone largely is composed of collagen, a family of fibrous proteins that have the ability to form insoluble and rigid fibers. The main collagen in bone is type I collagen. The inorganic component of bone, which is responsible for its rigidity and may constitute up to two-thirds of its fat-free dry weight, is composed chiefly of calcium phosphate and calcium carbonate, in the form of calcium hydroxyapatite, with small amounts of magnesium hydroxide, fluoride, and sulfate. The composition varies with age and with a number of dietary factors. The bone minerals form long fine crystals that add strength and rigidity to the collagen fibers; the process by which it is laid down is termed mineralization.

The term “bone marrow” as used herein refers to soft blood-forming tissue that fills the cavities of bones and contains fat and immature and mature blood cells, including white blood cells, red blood cells, and platelets. Bone marrow contains a variety of precursor and mature cell types, including hematopoietic cells, which are precursor cells of mature blood cells, and mesenchymal stem cells, otherwise known as stromal cells, that are precursors of a broad spectrum of connective tissue cells, both of which are capable of differentiating into other cell types. Hematopoietic stem cells (HSCs) in the bone marrow give rise to two main types of cells: the myeloid lineage (including monocytes, macrophages, neutrophils, basophils, eosinophils, erythrocytes, dendritic cells, and megakaryocytes or platelets) and the lymphoid lineage (including T cells, B cells, and natural killer cells).

Bone Remodeling. Bone constantly is broken down by osteoclasts and re-formed by osteoblasts in the adult. It has been reported that as much as 18% of bone is recycled each year through the process of renewal, known as bone remodeling, which maintains bone's rigidity. The balance in this dynamic process shifts as people grow older: in youth, it favors the formation of bone, but in old age, it favors resorption. As new bone material is added peripherally from the internal surface of the periosteum, there is a hollowing out of the internal region to form the bone marrow cavity. This destruction of bone tissue is due to osteoclasts that enter the bone through the blood vessels. Osteoclasts dissolve both the inorganic and the protein portions of the bone matrix. Each osteoclast extends numerous cellular processes into the matrix and pumps out hydrogen ions onto the surrounding material, thereby acidifying and solubilizing it. The blood vessels also import the blood-forming cells that will reside in the marrow for the duration of the organism's life.

The number and activity of osteoclasts must be tightly regulated. If there are too many active osteoclasts, too much bone will be dissolved, and osteoporosis will result. Conversely, if not enough osteoclasts are produced, the bones are not hollowed out for the marrow, and osteopetrosis (known as stone bone disease, a disorder whereby the bones harden and become denser) will result.

The terms “bone marrow transplant” (BMT) or “hematopoietic stem cell transplant” (HSCT) are used interchangeably to refer to a procedure in which bone marrow stem cells are collected from one individual (the donor) and given to another (the recipient). The stem cells can be collected either directly from the bone marrow or from the blood by leukapheresis. A bone marrow transplant may be autologous (using a patient's own stem cells that were collected from the marrow and saved before treatment), allogeneic (using stem cells donated by someone who is not an identical twin), or syngeneic (using stem cells donated by an identical twin).

The term “cancellous bone tissue” refers to an open, cell-porous network also called trabecular or spongy bone, which fills the interior of bone, and is composed of a network of rod- and plate-like elements that make the overall structure lighter and allows room for blood vessels and marrow so that the blood supply surrounds bone. Cancellous bone accounts for 20% of total bone mass but has nearly ten times the surface area of cortical bone. It does not contain haversian sites and osteons and has a porosity of about 30% to about 90%. In cancellous bone, the marrow spaces are relatively large and irregularly arranged, and the bone substance is in the form of slender anastomosing trabeculae and pointed spicules. The head of a bone, termed the epiphysis, has a spongy appearance and consists of slender irregular bone trabeculae, or bars, which anastomose to form a lattice work, the interstices of which contain the marrow, while the thin outer shell appears dense. The irregular marrow spaces of the epiphysis become continuous with the central medullary cavity of the bone shaft, termed the diaphysis, whose wall is formed by a thin plate of cortical bone.

The term “CD31” as used herein refers to platelet endothelial cell adhesion molecule (PECAM-1). It is a six domain molecule that mediates both leukocyte and platelet/endothelial cell adhesion and transendothelial migration. CD31 is expressed on platelets and on most leukocytes and is constitutively present on endothelial linings in vivo.

The term “CD34” as used herein is a marker found on the surface of bone marrow stem cells.

The term “CD45” as used herein means the lymphocyte common antigen.

The term “complementary” as used herein refers to two nucleic acid sequences or strands that can form a perfect base-paired double helix with each other.

The term “complementary DNA” or “cDNA” as used herein refers to a DNA molecule obtained by reverse transcription of an RNA molecule (commonly an mRNA) and therefore lacking the introns that are presentin genomic DNA.

TABLE 6
Definitions of Cell Populations

| Cells | Cell Type | Surface marker phenotype |
|---|---|---|
| HSCs | Hematopoietic stem cells | Mouse: Lineage |
| | | (Ter119/CD11b/GR1/B220/CD3)-CD41- cKIT+ |
| Cells | Cell Type | Surface marker phenotype |
| | | SCA1+ CD48- CD150+ |
| | | Human: Lineage (CD2, CD3, CD11b, CD14, |
| | | CD15, CD16, CD19, CD56, CD123, and CD235a |
| | | (Glycophorin A))-CD45RA-CD38-CD34+ CD90+ |
| HSPC/KLS | Hematopoietic stem and | Mouse: Lineage- cKIT+ SCA1+ |
| | progenitor cells | Human: CD34+ |
| MPP | Multipotent progenitors that | Lineage- cKIT+ SCA1+ CD48- CD150- |
| | express the receptor tyrosine | |
| | kinase FLT3; can produce | |
| | both lymphoid and myeloid | |
| | cells | |
| HPC-1 | hematopoietic progenitor | Lineage- cKIT+ SCA1+ CD48+ CD150- |
| | cell subset 1 | |
| HPC−2 | hematopoietic progenitor | Lineage- cKIT+ SCA1+ CD48+ CD150+ |
| | cell subset 2 | |
| CLP* | Common lymphoid progenitor; | Lineage- cKITlowSCAllowFLT3+ IL7Rα+ |
| CMP | Common myeloid progenitor | Lineage- cKIT+ SCA1- CD34+ CD16/32- |
| GMP | Granulocyte-macrophage | Lineage- cKIT+ SCA1- CD34+ CD16/32+ |
| | progenitors | |
| MEP | Metakaryocyte/erythrocyte | Lineage- cKIT+ SCA1- CD34- CD16/32- |
| | progenitor | |
| Pre Pro B | A B cell progenitor subset | sIgM- B220+ CD43+ CD24- |
| Pro B | A B cell progenitor subset | sIgM- B220+ CD43+ CD24+ |
| Pre B | A B cell progenitor subset | sIgM- B220+ CD43- CD24+ |
| Myeloid | Peripheral blood cell type | CD45+ CD11B+ GR1+ |
| | (granulocytes and monocytes) | |
| B Cells | Antibody producing antigen | CD45+ B220+ |
| | specific lymphocyte | |
| | responsible for adaptive | |
| | immune responses | |
| T Cells | Antigen specific lymphocyte | CD45+ CD3+ |
| | responsible for cell-mediated | |
| | adaptive immune reactions | |
| BM ECs | Bone marrow endothelial cells | CD45- Ter119- CD31+ VEcadherin+ |
| BM Stromal | Nonlymphoid cell that provides | CD45- Ter119- CD31- VEcadherin- |
| Cells | soluble and cell-bound signals | |
| BM Lepr+ | Within the BM stromal | CD45- Ter119- CD31- Lepr+ |
| Cells | population. Include Nestin+ | |
| | and CXCL12 abundant | |
| | reticular cells; are an important | |
| | source of KitL and SDF1 for | |
| | HSC maintenance. | |
| Osteoblasts | immature, mononucleate, bone- | CD45- Ter119- CD31- SCA1- CD51+ |
| | forming cells that synthesize | |
| | collagen and control | |
| | mineralization derived from | |
| | osteoprogenitors, which arise | |
| | from MSCs | |

* HSCs differentiate into MPPs. Differentiation of MPPs into CLPs requires signaling through
the FLT3 receptor expressed on MPPs. (CLPs) derived from MPPS comprise a subset that can
generate B, T and NKcells; a second subset that can generate only Band T cells; and a third
subset that is committed exclusively to B cells. The B cell committed CLPs give rise to proB
cells. Developmental stages of the B cell lineage are: early pro-B cell, late pro-B cell, large
pre-B cell, small pre-B cell, immature B cell and Mature B cell.

The term “cell cycle” refers to the progress of cells through four phases: G1 (interphase), S (DNA synthesis phase), G2 (interphase) and M (mitosis phase). Nakamura-Ishizu, A., et al., Development (2014) 141: 4656-4666; citing Sisken, J E and Morasca, L., J. Cell Biol. (1965) 25: 179-189). Cells that proceed past the restriction point in the G1 phase enter the S phase, whereas those that do not pass the restriction point remain undivided. These undivided cells can withdraw from the cell cycle and enter the G0 phase, a state in which cells are termed quiescent or dormant (Id., citing Pardee, AB, Proc. Natl Acad. Sci. USA (1974) 71: 1286-90). Such non-cycling cells in the G0 phase can either reversibly re-enter the cell cycle and divide (Id., citing Cheung, T H and Rando, TA, Nat. Rev. Mol. Cell Biol. (2013) 14: 329-340) or remain dormant, losing the potential to cycle and, in some cases, becoming senescent (Id., citing Campisi, J. Cell (2005) 120: 513-22).

The term “cell lineage” or “lineage” as used herein refers to the developmental history of a differentiated cell as traced back to the cell from which it arises.

The term “chemokine” as used herein refers to a family of low molecular mass (8-11 kDa) structurally-related proteins with diverse immune and neural functions (Mackay C. R. Nat Immunol., Vol. 2: 95-101, (2001); Youn B. et al. Immunol Rev. (2000) Vol. 177: 150-174) that can be categorized into four subfamilies (C, CC, CXC and CX3C) based on the relative positions of conserved cysteine residues (Rossi D. et al. Annu Rev Immunol. (2000) 18: 217-242). Chemokines are essential molecules in directing leucocyte migration between blood, lymph nodes and tissues. They constitute a complex signaling network, because they are not always restricted to one type of receptor (Loetscher P. et al. J. Biol. Chem. (2001). 276: 2986-2991). Chemokines affect cells by activating surface receptors that are seven-transmembrane-domain G-protein-coupled receptors. Leukocyte responses to particular chemokines are determined by their expression of chemokine receptors. The binding of the chemokine to the receptor activates various signaling cascades, similar to the action of cytokines that culminate in the activation of a biological response. Secretion of the ligands for the CCR5 receptor, regulated upon activation normal T cell expressed and secreted (RANTES), macrophage inflammatory protein (MIP)-1α/and MIP-1β (Schrum S. et al. J Immunol. (1996) 157: 3598-3604) and the ligand for CXC chemokine receptor 3 (CXCR3), induced protein (IP)-10 (Taub D. D. et al. J Exp Med. (1993) 177:1809-1814) have been associated with unwanted heightened T_{H1} responses. Additionally, elevated damaging pro-inflammatory cytokine levels of IL-2 and IFN-γ correlate with type 1 diabetes (T1D) (Rabinovitch A. et al. Cell Biochem Biophys. (2007) 48 (2-3): 159-63). Chemokines have been observed in T_{H1} pancreatic infiltrates and other inflammatory lesions characterized by T cell infiltration (Bradley L. M. et al. J Immunol. (1999). 162:2511-2520).

The term “chemotherapy” as used herein refers to a treatment that uses drugs to destroy cancer cells, but is also used in bone marrow transplant patients without cancer in order to ensure successful engraftment.

The term “chronological age” as used herein refers to the time passed from birth to a given date. Chronological ages are commonly grouped into a small number of crude age ranges, reflecting the major stages of development and aging categories: According to Medical Subject Headings (MeSH), the age brackets for humans are: Young: from infant to young adult, i.e., Infant: 0-2; Preschood: 2-5; Child: 5-12; Adolescent: 12-19; Young adult: 19-24; adult: from 24-44; Middle aged: 44-65; and Aged: over 65 years. For the mouse, the chronological age categories by consensus are young (3 months); middle-aged (8-14 months) and old (18-24 months).

The term “competitive bone marrow transplantation” refers to an assay routinely used to determine hematopoietic stem and progenitor cells (HSPCs) functionality in vivo. The principle of the method is to transplant bone marrow donor cells derived from transgenic mice on C57BL6 background together with normal competitor bone marrow. Engraftment efficiency is evaluated in both blood and bone marrow in the irradiated transplant recipient mice.

The term “conditioning” as used herein refers to a combination of chemotherapy drugs, and sometimes radiation, given a few days prior to transplant that collectively prepare the body for transplant.

The term “contact” and its various grammatical forms as used herein refers to a state or condition of touching or of immediate or local proximity.

The term “cortical bone tissue” (also referred to as compact bone or dense bone), refers to the tissue of the hard outer layer of bones, so-called due to its minimal gaps and spaces. This tissue gives bones their smooth, white, and solid appearance. Cortical bone consists of haversian sites (the canals through which blood vessels and connective tissue pass in bone) and osteons (the basic units of structure of cortical bone comprising a haversian canal and its concentrically arranged lamellae), so that in cortical bone, bone surrounds the blood supply. Cortical bone has a porosity of about 5% to about 30%, inclusive and accounts for about 80% of the total bone mass of an adult skeleton. In cortical bone, the spaces or channels are narrow and the bone substance is densely packed.

The term “cytokine” as used herein refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (TNF)-related molecules, including TNFα and lymphotoxin; immunoglobulin super-family members, including interleukin 1 (IL-1); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “damage-associated molecule patterns” (DAMPs) as used herein refers to endogenous danger molecules that are released from damaged or dying cells, which activate the innate immune system by interacting with pattern recognition receptors (PRRs).

As used herein, the term “derived from” is meant to encompass any method for receiving, obtaining, or modifying something from a source of origin.

As used herein, the terms “detecting”, “determining”, and their other grammatical forms, are used to refer to methods performed for the identification or quantification of a biomarker, such as, for example, the presence or level of miRNA, or for the presence or absence of a condition in a biological sample. The amount of biomarker expression or activity detected in the sample can be none or below the level of detection of the assay or method.

The term “differentiation” as used herein refers to a process of development with an increase in the level of organization or complexity of a cell or tissue, accompanied by a more specialized function.

The term “differential” as used herein refers to of, relating to or constituting a difference. The term “differential production” with reference to angiocrine factors as used herein refers to differences in production between angiocrine factors.

The terms “disease” or “disorder” as used herein refer to an impairment of health or a condition of abnormal functioning.

The term “endogenous” as used herein refers to that which is naturally occurring, incorporated within, housed within, adherent to, attached to, or resident in.

The terms “endosteal niche” and “osteoblastic niche” are used interchangeably to describe a complex microenvironment that houses quiescent or long-term HSCs (LT-HSCs) that can be mobilized in response to tissue injury. (Guerrouahen, B. S., Al-Hijji, I., & Tabrizi, A. R. (2011). Osteoblastic and Vascular Endothelial Niches, Their Control on Normal Hematopoietic Stem Cells, and Their Consequences on the Development of Leukemia. Stem Cells International, 2011, 1-8).

The term “engraftment” as used herein refers to a process in which normal growth of transplanted (donor) stem cells and production of blood cells in the patient's (recipient's) marrow spaces resumes after transplant.

As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

The term “erythropoiesis” as used herein refers to the formation of red blood cells in blood-forming tissue. In the early development of a fetus, erythropoiesis takes place in the yolk sac, spleen, and liver. After birth, all erythropoiesis occurs in the bone marrow. The erythroid line of differentiation in bone marrow and spleen starts with the early progenitor pro-erythroblasts that are derived from pluripotent stem cells. In adult bone marrow, definitive erythropoiesis begins when an HSC-derived common myeloid progenitor (a multipotent stem cell) commits to the erythroid lineage. The appearance of a pronormoblast (also called proerythroblast or ribriblast) marks the first stage of differentiation. This is followed by early, intermediate and late normoblast (erythroblast) stages, at which time the nucleus is expelled and the cell becomes a reticulocyte. Upon exiting the bone marrow, reticulocytes enter the circulation to become fully mature RBCs.

The term “exogenous” as used herein refers to that which is non-naturally occurring, or that is originating or produced outside of a specific cell, organism, or species.

The term “expand” and its various grammatical forms as used herein refers to a process by which dispersed living cells propagate in vitro in a culture medium that results in an increase in the number or amount of viable cells.

As used herein, the term “expression” and its various grammatical forms refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Expression may also refer to the post-translational modification of a polypeptide or protein.

The term “extracellular matrix” (or “ECM”) as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached to one or more glycosaminoglycans.

The term “fragment” or “peptide fragment” as used herein refers to a small part derived, cut off, or broken from a larger antibody peptide, polypeptide or protein, which retains the desired biological activity of the larger antibody peptide, polypeptide or protein. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding fragment” or “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “gene” as used herein is the entire DNA sequence, including exons, introns, and noncoding transcription-control regions necessary for production of a functional protein or RNA.

The terms “gene expression” or “expression” are used interchangeably to refer to the process by which information encoded in a gene is converted into an observable phenotype.

The term “graft” as used herein refers to a tissue or organ infused or transplanted from a donor to a recipient. It includes, but is not limited to, a self tissue transferred from one body site to another in the same individual (“autologous graft”), a tissue transferred between genetically identical individuals or sufficiently immunologically compatible to allow tissue transplant (“syngeneic graft”), a tissue transferred between genetically different members of the same species (“allogeneic graft” or “allograft”), and a tissue transferred between different species (“xenograft”).

The term “growth factor” as used herein refers to extracellular polypeptide molecules that bind to a cell-surface receptor triggering an intracellular signaling pathway, leading to proliferation, differentiation, or other cellular response that stimulate the accumulation of proteins and other macromolecules, e.g., by increasing their rate of synthesis, decreasing their rate of degradation, or both. Exemplary growth factors include fibroblast growth factor (FGF), insulin-like growth factor (IGF-1), transforming growth factor beta (TGF-0), and vascular endothelial growth factor (VEGF)

Fibroblast Growth Factor (FGF). The fibroblast growth factor (FGF) family currently has over a dozen structurally related members. FGF1 is also known as acidic FGF; FGF2 is sometimes called basic FGF (bFGF); and FGF7 sometimes goes by the name keratinocyte growth factor. Over a dozen distinct FGF genes are known in vertebrates; they can generate hundreds of protein isoforms by varying their RNA splicing or initiation codons in different tissues. FGFs can activate a set of receptor tyrosine kinases called the fibroblast growth factor receptors (FGFRs). Receptor tyrosine kinases are proteins that extend through the cell membrane. The portion of the protein that binds the paracrine factor is on the extracellular side, while a dormant tyrosine kinase (i.e., a protein that can phosphorylate another protein by splitting ATP) is on the intracellular side. When the FGF receptor binds an FGF (and only when it binds an FGF), the dormant kinase is activated, and phosphorylates certain proteins within the responding cell, activating those proteins.

FGFs are associated with several developmental functions, including angiogenesis (blood vessel formation), mesoderm formation, and axon extension. While FGFs often can substitute for one another, their expression patterns give them separate functions. For example, FGF2 is especially important in angiogenesis, whereas FGF8 is involved in the development of the midbrain and limbs.

Insulin-Like Growth Factor (IGF-1). IGF-1, a hormone similar in molecular structure to insulin, has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 also can regulate cell growth and development, especially in nerve cells, as well as cellular DNA synthesis.

IGF-1 was shown to increase the expression levels of the chemokine receptor CXCR4 (receptor for stromal cell-derived factor-1, SDF-1) and to markedly increase the migratory response of MSCs to SDF-1 (Li, Y, et al. 2007 Biochem. Biophys. Res. Communic. 356(3): 780-784). The IGF-1-induced increase in MSC migration in response to SDF-1 was attenuated by PI3 kinase inhibitor (LY294002 and wortmannin) but not by mitogen-activated protein/ERK kinase inhibitor PD98059. Without being limited by any particular theory, the data indicate that IGF-1 increases MSC migratory responses via CXCR4 chemokine receptor signaling which is PI3/Akt dependent.

Transforming Growth Factor Beta (TGF-β). There are over 30 structurally related members of the TGF-β superfamily, and they regulate some of the most important interactions in development. The proteins encoded by TGF-β superfamily genes are processed such that the carboxy-terminal region contains the mature peptide. These peptides are dimerized into homodimers (with themselves) or heterodimers (with other TGF-β peptides) and are secreted from the cell. The TGF-β superfamily includes the TGF-β family, the activin family, the bone morphogenetic proteins (BMPs), the Vg-1 family, and other proteins, including glial-derived neurotrophic factor (GDNF, necessary for kidney and enteric neuron differentiation) and Millerian inhibitory factor, which is involved in mammalian sex determination. TGF-β family members TGF-β1, 2, 3, and 5 are important in regulating the formation of the extracellular matrix between cells and for regulating cell division (both positively and negatively). TGF-β1 increases the amount of extracellular matrix epithelial cells make both by stimulating collagen and fibronectin synthesis and by inhibiting matrix degradation. TGF-βs may be critical in controlling where and when epithelia can branch to form the ducts of kidneys, lungs, and salivary glands.

Vascular Endothelial Growth Factor (VEGF). VEGFs are growth factors that mediate numerous functions of endothelial cells including proliferation, migration, invasion, survival, and permeability. The VEGFs and their corresponding receptors are key regulators in a cascade of molecular and cellular events that ultimately lead to the development of the vascular system, either by vasculogenesis, angiogenesis, or in the formation of the lymphatic vascular system. VEGF is a critical regulator in physiological angiogenesis and also plays a significant role in skeletal growth and repair.

VEGF's normal function creates new blood vessels during embryonic development, after injury, and to bypass blocked vessels. In the mature established vasculature, the endothelium plays an important role in the maintenance of homeostasis of the surrounding tissue by providing the communicative network to neighboring tissues to respond to requirements as needed. Furthermore, the vasculature provides growth factors, hormones, cytokines, chemokines and metabolites, and the like, needed by the surrounding tissue and acts as a barrier to limit the movement of molecules and cells.

The VEGF family consists of number of secreted proteins: VEGF-A, VEGF-B, VEGF-C, VEGF-D, VEGF-E, and placental growth factor (PlGF), with VEGF-A being the most widely studied of the group. (Bazzazi, H. et al., “Computer Simulation of TSP1 inhibition of VEGF-Akt-eNOS: An angiogenesis triple threat. Front. Physiol. (2018) 9: 644). VEGF plays a crucial role in vasculogenesis and developmental angiogenesis (Id., citing Shalaby F., et al. Failure of blood-island formation and vasculogenesis in Flk-1-deficient mice. Nature (1995) 376: 62-66; Carmeliet P., et al. Abnormal blood vessel development and lethality in embryos lacking a single VEGF allele. (1996) Nature 380: 435-439) and adult vascular permeability and homeostasis. (Id., citing Ku D D, et al. Vascular endothelial growth factor induces EDRF-dependent relaxation in coronary arteries. Am. J. Physiol. (1993) 265: H586-H592; Lee et al, Vascular endothelial growth factor induces EDRF-dependent relaxation in coronary arteries. Am. J. Physiol. (1993) 265: H586-H592; Curwen J. O., et al. Inhibition of vascular endothelial growth factor-a signaling induces hypertension: examining the effect of cediranib (recentin; AZD2171) treatment on blood pressure in rat and the use of concomitant antihypertensive therapy. Clin. Cancer Res. (2008) 14: 3124-3131) Dysregulation in VEGF signaling contributes to a wide array of diseases including cancer (Id., citing Kieran, M. et al, The VEGF pathway in cancer and disease: responses, resistance, and the path forward. Cold Spring Harb. Perspect. Med. (2012) 2:a006593. 10.1101/cshperspect.a006593; Claesson-Welsh L., Welsh M. VEGFA and tumour angiogenesis. J. Intern. Med. (2013) 273: 114-127), wound healing (Id., citing Bao, P. et al., The role of vascular endothelial growth factor in wound healing. J. Surg. Res. (2009) 153: 347-358), age-related macular degeneration (Id., citing Ferrara, N, Vascular endothelial growth factor and age-related macular degeneration: from basic science to therapy. Nat. Med. (2010) 16: 1107-1111), and peripheral arterial disease (PAD) (Id., citing MacGabhaan, F. et al Systems biology of pro-angiogenic therapies targeting the VEGF system. Wiley Interdiscip. Rev. Syst. Biol. Med. (2010) 2: 694-707; Boucher J. M., Bautch V. L. Antiangiogenic VEGF-A in peripheral artery disease. Nat. Med. (2014) 20: 1383-1385; Clegg L. E., et al. Systems pharmacology of VEGF165b in peripheral artery disease. CPT Pharmacometrics Syst. Pharmacol. (2017) 6: 833-844; Clegg L. E., Mac Gabhann F. A computational analysis of pro-angiogenic therapies for peripheral artery disease. Integr. Biol. (2018) 10: 18-33). The response to VEGF is mediated by its binding to multiple receptors and co-receptors on endothelial cells such as VEGF receptor 2 (VEGFR2) and neuropilin-1 (NRP1).

VEGF binding to receptor tyrosine kinase VEGFR2 leads to the activation of downstream signaling pathways including ERK1/2 and PI3K/Akt that induce cellular proliferation, survival, motility, and enhanced vascular permeability (Id., citing Olsson A. K., et al. VEGF receptor signalling—in control of vascular function. Nat. Rev. Mol. Cell Biol. (2006) 7: 359-371; Dellinger M. T., Brekken R. A. Phosphorylation of Akt and ERK1/2 is required for VEGF-A/VEGFR2-induced proliferation and migration of lymphatic endothelium. PLoS One (2011) 6: e28947; Simons M., et al. Mechanisms and regulation of endothelial VEGF receptor signalling. Nat. Rev. Mol. Cell Biol. (2016) 17: 611-625), the dominant pathway in post-natal angiogenesis. VEGF-VEGFR2 activation also induces nitric oxide (NO) release as a result of the activation of endothelial nitric oxide synthase (eNOS), substantially contributing to the angiogenic response. (Id., citing Papapetropoulos, A. et al. Nitric oxide production contributes to the angiogenic properties of vascular endothelial growth factor in human endothelial cells. J. Clin. Invest. (1997) 100: 3131-3139; Fukumura, D. et al. Predominant role of endothelial nitric oxide synthase in vascular endothelial growth factor-induced angiogenesis and vascular permeability. Proc. Natl. Acad. Sci. U.S.A. (2001) 98: 2604-2609.

Physiological VEGF signaling is tightly regulated by a balance of promoters and inhibitors of angiogenesis. (Id., citing Folkman J. Endogenous angiogenesis inhibitors. APMIS (2004) 112: 496-507). The matricellular protein thrombospondin-1 (TSP1) was among the first identified endogenous inhibitors of angiogenesis. (Id., citing Bagavandoss P., Wilks J. W. Specific inhibition of endothelial cell proliferation by thrombospondin. Biochem. Biophys. Res. Commun. (1990). 170: 867-872); Good, D J et al. A tumor suppressor-dependent inhibitor of angiogenesis is immunologically and functionally indistinguishable from a fragment of thrombospondin. Proc. Natl. Acad. Sci. U.S.A. (1990) 87: 6624-6628; Taraboletti, G et al. Platelet thrombospondin modulates endothelial cell adhesion, motility, and growth: a potential angiogenesis regulatory factor. J. Cell Biol. (1990) 111 765-772. TSP1 potently inhibits VEGF signaling at multiple levels. At nanomolar concentrations, TSP1 can directly bind and sequester VEGF (Id., citing Gupta, K et al Binding and displacement of vascular endothelial growth factor (VEGF) by thrombospondin: effect on human microvascular endothelial cell proliferation and angiogenesis. Angiogenesis (1999) 3: 147-1589) or lead to the internalization of TSP1-VEGF complex via binding to the TSP1 receptor LDL-related receptor protein 1 (LRP1). (Id., citing Greenaway, J. et al −1 inhibits VEGF levels in the ovary directly by binding and internalization via the low density lipoprotein receptor-related protein-1 (LRP-1). J. Cell. Physiol. (2007) 210: 807-818). At these concentrations, TSP1 may also inhibit Akt/eNOS/NO signaling by binding to the cell surface receptor CD36. (Id., citing Isenberg, J S et al, Thrombospondin-1 inhibits nitric oxide signaling via CD36 by inhibiting myristic acid uptake. J. Biol. Chem. (2007) 282: 15404-15415). Binding of TSP1 to CD36, a fatty acid translocase, also inhibits its ability to uptake myristate into endothelial cells inhibiting activation of Src kinases and cGMP signaling. (Id., citing Isenberg, J S, et al, Thrombospondin-1 inhibits nitric oxide signaling via CD36 by inhibiting myristic acid uptake. J. Biol. Chem. (2007) 282: 15404-15415) At picomolar concentrations, TSP1 potently inhibits angiogenesis by binding to CD47, an integrin associate glycoprotein membrane receptor. (Id., citing Kaur, S. et al Thrombospondin-1 inhibits VEGF receptor-2 signaling by disrupting its association with CD47. J. Biol. Chem. (2010) 285: 38923-38932). CD47 is the necessary TSP1 receptor for the inhibition of signals downstream of NO namely soluble guanylate cyclase (sGC) and cGMP-dependent protein kinase. Id, citing Isenberg, J S, et al CD47 is necessary for inhibition of nitric oxide-stimulated vascular cell responses by thrombospondin-1. J. Biol. Chem. 281: 26069-26080; Isenberg, J S et al. Thrombospondin-1 stimulates platelet aggregation by blocking the antithrombotic activity of nitric oxide/cGMP signaling. Blood (2008) 111: 613-623. TSP1-CD47 interaction also inhibits eNOS activation and eNOS-dependent endothelial cell vasorelaxation. (Id., citing Bauer E M et al Thrombospondin-1 supports blood pressure by limiting eNOS activation and endothelial-dependent vasorelaxation. Cardiovasc. Res. (2010) 88: 471-481). Mice deficient in CD47 or TSP1 show enhanced angiogenesis in models of wound healing. (Id., citing Isenberg, J S, et al Blockade of thrombospondin-1-CD47 interactions prevents necrosis of full thickness skin grafts. (2008) Ann. Surg. 247: 180-190.

Further, TSP1-CD47 interaction has been demonstrated to potently inhibit VEGFR2 phosphorylation and Akt activation. (Id., citing Kaur, S. et al Thrombospondin-1 supports blood pressure by limiting eNOS activation and endothelial-dependent vasorelaxation. Cardiovasc. Res. (2010) 88: 471-481. Suppression of CD47 or downregulation of its expression rescued VEGFR2 phosphorylation, indicating that the anti-angiogenic phenotype initiated by TSP1-CD47 interaction goes beyond mere inhibition of NO signaling, pointing toward a role in a more global inhibitory effect. (Id., citing Kaur, S. et al Thrombospondin-1 supports blood pressure by limiting eNOS activation and endothelial-dependent vasorelaxation. Cardiovasc. Res. (2010) 88: 471-481).

The term “healthspan” as used herein refers to the length of time in one's life during which an individual is in reasonably good health.

The term “HPCs” as used herein refers to hematopoietic progenitor cells.

The term “HSPCs” as used herein refers to hematopoietic stem and progenitor cells, a rare population of precursor cells that possess the capacity for self-renewal and multilineage differentiation.

The term “heterotypic” as used herein refers to two different cell types. The term “heterotypic signaling” as used herein refers to communication between dissimilar cell types.

The term “homotypic” as used herein refers to identical cell types.

The terms “immune reconstitution” or “reconstitution” as used herein refers to a process of rebuilding the immune system from transplanted HSCs after HSCT.

The terms “immune response” and “immune-mediated” are used interchangeably herein to refer to any functional expression of a subject's immune system, against either foreign or self-antigens, whether the consequences of these reactions are beneficial or harmful to the subject.

The term “immune system” as used herein refers to the body's system of defenses against disease. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens.

The term “innate immunity” as used herein refers to the various innate resistance mechanisms that are encountered first by a pathogen, before adaptive immunity is induced, such as anatomical barriers, antimicrobial peptides, the complement system and macrophages and neutrophils carrying nonspecific pathogen-recognition receptors. Innate immunity is present in all individuals at all times, does not increase with repeated exposure to a given pathogen, and discriminates between groups of similar pathogens, rather than responding to a particular pathogen.

The terms “immunomodulatory”, “immune modulator” and “immune modulatory” are used interchangeably herein to refer to a substance, agent, or cell that is capable of augmenting or diminishing immune responses directly or indirectly, e.g., by expressing chemokines, cytokines and other mediators of immune responses.

The term “immunosuppressive agent” as used herein refers to an agent that decreases the body's immune responses.

The term “immunosuppression” as used herein refers to a state of decreased immunity or a lowering of the body's immune response. The term “immunosuppressive therapy” as used herein refers to a treatment that lowers the activity of the body's immune system.

The term “inflammation” as used herein refers to the physiologic process by which vascularized tissues respond to injury. See, e.g., Fundamental Immunology, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses. The term “acute inflammation” as used herein refers to the rapid, short-lived (minutes to days), relatively uniform response to acute injury characterized by accumulations of fluid, plasma proteins, and neutrophilic leukocytes. The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory mediators” or “inflammatory cytokines” as used herein refers to molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1-beta (IL-10), interleukin-4 (IL-4), interleukin-6 (IL-6), interleukin-8 (IL-8), tumor necrosis factor-alpha (TNF-α), interferon-gamma (IF-γ), and interleukin-12 (IL-12).

The term “infuse” and its other grammatical forms as used herein refers to introduction of a fluid other than blood into a vein.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

The term “inhibitor” as used herein refers to a second molecule that binds to, contacts or otherwise interferes with activity of a first molecule thereby decreasing the first molecule's activity.

The term “insult,” as used herein, refers to damage or harm to a structure or function of the body caused by an outside agent or force, which may be physical or chemical, or an interior condition.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “knock-in” as used herein refers to a genetic engineering method that involves the insertion of a protein coding cDNA sequence at a particular locus in a target organism's chromosome (Gibson, Greg (2009). A Primer of Genome Science 3rd ed. Sunderland, Mass.: Sinauer. pp. 301-302).

The terms “knockout” or “KO” or “knockdown” are used interchangeably herein to refer to a genetic engineering method n which specific gene(s) have been disrupted or deleted such that the corresponding gene product(s) are not synthesized in active form or are absent.

The term “Lineage-positive (Lin+) cells as used herein refers to a mix of all cells expressing mature cell lineage markers. The rest of the cells are lineage-negative (Lin−), meaning they are not stained by the lineage antibodies. All step and progenitor cell activity was identified within the Lin− population.

The term “lymphocyte common antigen” or CD45, means a receptor-linked protein tyrosine phosphatase expressed on all leukocytes.

The term “lymphoid lineage cells” as used herein refers to all cells that are derived from the common lymphoid progenitor (CLP) cell, which differentiates from bone marrow hematopoietic stem cells. They include T lymphocytes, B lymphocytes and natural killer (NK) cells.

The terms “major histocompatibility complex” and “MHC” is used herein to refer to cell-surface molecules that display a molecular fraction known as an epitope or an antigen and mediate interactions of leukocytes with other leukocyte or body cells. MHCs are encoded by a large gene group and can be organized into three subgroups—class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP1:TAP2 peptide transporter, the PSMB (or LMP) genes that encode proteasome subunits, the genes encoding the DMα and BMβ chains (DMA and DMB), the genes enclosing the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. Janeways Immunobiology. 9th ed., GS, Garland Science, Taylor & Francis Group, 2017. pps. 232-233.

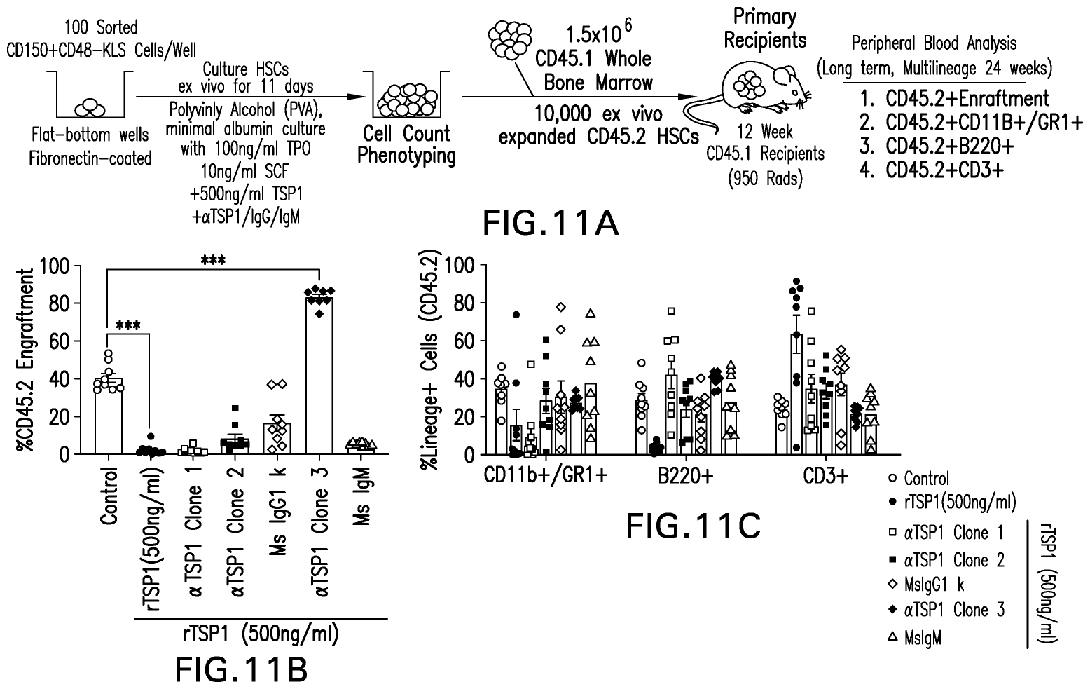

The abbreviation “MAPK” as used herein refers to Mitogen-Activated Protein Kinase (MAPK) signaling, which activates a three-tiered cascade with MAPK kinase kinases (MAP3K) activating MAPAK kinases (MAP2K) and finally MAPK. MAPKs are protein Ser/Thr kinases that convert extracellular stimuli into a wide range of cellular responses. (Cargnello, M. and Roux, PP, Microbiol. Mol. Biol. Rev. (2011) 75(1): 50-83). The major MAPK pathways involved in inflammatory diseases are extracellular regulating kinase (ERK), p38 MAPK, and c-Jun NH2-terminal kinase (JNK). Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). (See FIG. 11, taken from Barnes, PJ (2016) Pharmacological Revs. 68: 788-815).

The term “matrix metalloproteinases” as used herein refers to a collection of zinc-dependent proteases involved in the breakdown and the remodeling of extracellular matrix components (Guiot, J. et al. Lung (2017) 195(3): 273-280, citing Oikonomidi et al. Curr Med Chem. 2009; 16(10): 1214-1228). For example, the MMP2 gene provides instructions for making matrix metallopeptidase 2. This enzyme is produced in cells throughout the body and becomes part of the extracellular matrix, which is an intricate lattice of proteins and other molecules that forms in the spaces between cells. One of the major known functions of MMP-2 is to cleave type IV collagen, which is a major structural component of basement membranes, the thin, sheet-like structures that separate and support cells as part of the extracellular matrix.

The term “mimic” as used herein refers to a compound or substance that chemically resembles a parent compound or substance and retains at least a degree of the desired function of the parent compound or substance. The term “mimic” may be used interchangeably with “mimetic”, which refers to chemicals containing chemical moieties that mimic the function of a peptide. For example, if a peptide contains two charged chemical moieties having functional activity, a mimetic places two charged chemical moieties in a spatial orientation and constrained structure so that the charged chemical function is maintained in three-dimensional space.

The terms “modify” or “modulate” as used herein means to regulate, alter, adapt, or adjust to a certain measure or proportion. The terms “modified” or “modulated” as used herein in the context of cell types refers to changing the form or character of the cell type.

The term “myeloid lineage cells” refers collectively to granulocytes and monocytes, which are differentiated descendants from common myeloid progenitors (CMPs) derived from hematopoietic stem cells in the bone marrow. Commitment to either lineage of myeloid cells is controlled by distinct transcription factors followed by terminal differentiation in response to specific colony-stimulating factors and release into the circulation. [Kawamoto, H., Minato, N. Intl J. Biochem. Cell Biol. (2004) 36 (8): 1374-70].

The term “myeloablative therapy” as used herein refers to a therapeutic regimen (such as high dose chemotherapy or high doses of irradiation) used to kill cells that live in the bone marrow, including cancer cells, which lowers the number of normal blood-forming cells in the bone marrow, resulting in fewer red blood cells, white blood cells, and platelets. The term “non-myeloablative” as used herein refers to the conditioning regimen prior to transplant in which limited amounts of chemotherapy are administered in order to prevent rejection of the donor bone marrow stem cells without destroying the recipient's bone marrow.

The term “myelosuppression” as used herein refers to a condition in which bone marrow activity is decreased, resulting in fewer red blood cells, white blood cells, and platelets. When myelosuppression is severe, it is called myeloablation. Myelosuppression leads not only to apoptosis of cycling hematopoietic cells, but also to the destruction of the bone marrow vasculature. (Kopp, et. al. “The Bone Marrow Vascular Niche: Home of HSC Differentiation and Mobilization.” PHYSIOLOGY 20: 349-356, 2005; 10.1152/physiol.00025.2005)

The term “neutralizing antibody” as used herein refers to an antibody that reduces the biological activity of its target. The term “non-neutralizing antibodies” as used herein refers to functional antibodies with low or no neutralization activity in vitro. Non-neutralizating binding antibodies function in multiple different ways, including, without limitation, by binding to and sterically inhibiting activity of proteins.

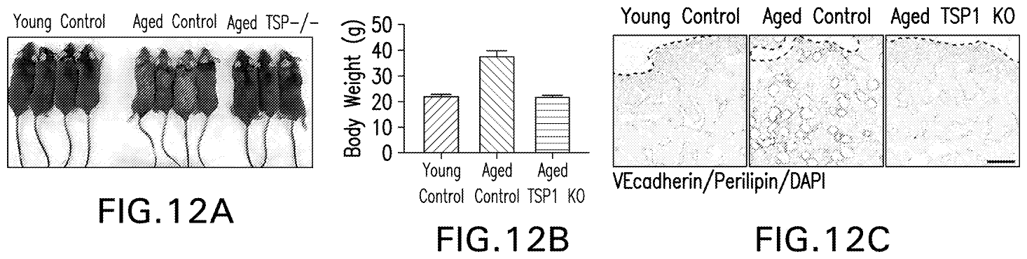

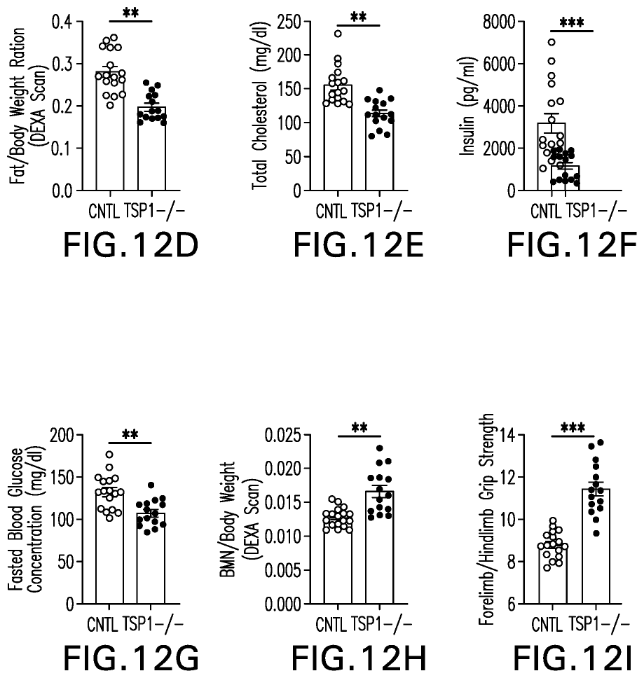

The abbreviation “NFκB” as used herein refers to which is a proinflammatory transcription factor. It switches on multiple inflammatory genes, including cytokines, chemokines, proteases, and inhibitors of apoptosis, resulting in amplification of the inflammatory response (Barnes, P J, (2016) Pharmacol. Rev. 68: 788-815). The molecular pathways involved in NF-κB activation include several kinases. The classic (canonical) pathway for inflammatory stimuli and infections to activate NF-κB signaling involve the IKK (inhibitor of κB kinase) complex, which is composed of two catalytic subunit, IKK-α and IKK-β, and a regulatory subunit IKK-γ (or NFκB essential modulator (Id., citing Hayden, M S and Ghosh, S (2012) Genes Dev. 26: 203-234). The IKK complex phosphorylates Nf-κB-bound IκBs, targeting them for degradation by the proteasome and thereby releasing NF-κB dimers that are composed of p65 and p50 subunits, which translocate to the nucleus where they bind to κB recognition sites in the promoter regions o inflammatory and immune genes, resulting in their transcriptional activation (FIG. 12). This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β (Id., citing Sun, S C. (2012) Immunol. Rev. 246: 125-140). This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes (Id., citing Lombardi, V et al. (2010) Int. Arch. Allergy Immnol. 151: 179-89).

The term “nucleic acid” is used herein to refer to a deoxyribonucleotide or ribonucleotide polymer in either single- or double-stranded form, and, unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The term “oligonucleotide” as used herein refers to relatively short (13-25 nucleotides) unmodified or chemically modified single-stranded DNA molecules.

The term “organ” as used herein refers to a differentiated structure consisting of cells and tissues that performs some specific function in an organism.

The term “organotypic” as used herein refers to that which is typical or characteristic of an organ or type of tissue.

The term “osteogenesis” as used herein refers to the process by which osseous or bony tissue is formed. Osseous tissue is a rigid form of connective tissue normally organized into definite structures, the bones. There are two major modes of osteogenesis, both of which involve the transformation of a preexisting mesenchymal tissue into bone tissue. The direct conversion of mesenchymal tissue into bone is called intramembranous ossification. This process occurs primarily in the bones of the skull. In other cases, mesenchymal cells differentiate into cartilage, which is later replaced by bone. The process by which a cartilage intermediate is formed and replaced by bone cells is called endochondral ossification.

Intramembranous ossification is the characteristic way in which the flat bones of the scapula, the skull and the turtle shell are formed. In intramembranous ossification, bones develop sheets of fibrous connective tissue. During intramembranous ossification in the skull, neural crest-derived mesenchymal cells proliferate and condense into compact nodules. Some of these cells develop into capillaries; others change their shape to become osteoblasts, committed bone precursor cells. The osteoblasts secrete a collagen-proteoglycan matrix that is able to bind calcium salts. Through this binding, the prebone (osteoid) matrix becomes calcified. In most cases, osteoblasts are separated from the region of calcification by a layer of the osteoid matrix they secrete. Occasionally, osteoblasts become trapped in the calcified matrix and become osteocytes. As calcification proceeds, bony spicules radiate out from the region where ossification began, the entire region of calcified spicules becomes surrounded by compact mesenchymal cells that form the periosteum, and the cells on the inner surface of the periosteum also become osteoblasts and deposit osteoid matrix parallel to that of the existing spicules. In this manner, many layers of bone are formed.

Intramembranous ossification is characterized by invasion of capillaries into the mesenchymal zone, and the emergence and differentiation of mesenchymal cells into mature osteoblasts, which constitutively deposit bone matrix leading to the formation of bone spicules, which grow and develop, eventually fusing with other spicules to form trabeculae. As the trabeculae increase in size and number they become interconnected forming woven bone (a disorganized weak structure with a high proportion of osteocytes), which eventually is replaced by more organized, stronger, lamellar bone.

The molecular mechanism of intramembranous ossification involves bone morphogenetic proteins (BMPs) and the activation of a transcription factor called CBFA1. Bone morphogenetic proteins, for example, BMP2, BMP4, and BMP7, from the head epidermis are thought to instruct the neural crest-derived mesenchymal cells to become bone cells directly. BMPs activate the Cbfa1 gene in mesenchymal cells. The CBFA1 transcription factor is known to transform mesenchymal cells into osteoblasts. Studies have shown that the mRNA for mouse CBFA1 is largely restricted to the mesenchymal condensations that form bone, and is limited to the osteoblast lineage. CBFA1 is known to activate the genes for osteocalcin, osteopontin, and other bone-specific extracellular matrix proteins.

Endochondral Ossification (Intracartilaginous Ossification). Endochondral ossification, which involves the in vivo formation of cartilage tissue from aggregated mesenchymal cells, and the subsequent replacement of cartilage tissue by bone, can be divided into five stages. The skeletal components of the vertebral column, the pelvis, and the limbs are first formed of cartilage and later become bone.

First, the mesenchymal cells are committed to become cartilage cells. This commitment is caused by paracrine factors that induce the nearby mesodermal cells to express two transcription factors, Pax1 and Scleraxis. These transcription factors are known to activate cartilage-specific genes. For example, Scleraxis is expressed in the mesenchyme from the sclerotome, in the facial mesenchyme that forms cartilaginous precursors to bone, and in the limb mesenchyme.

During the second phase of endochondral ossification, the committed mesenchyme cells condense into compact nodules and differentiate into chondrocytes (cartilage cells that produce and maintain the cartilaginous matrix, which consists mainly of collagen and proteoglycans). Studies have shown that N-cadherin is important in the initiation of these condensations, and N-CAM is important for maintaining them. In humans, the SOX9 gene, which encodes a DNA-binding protein, is expressed in the precartilaginous condensations.

During the third phase of endochondral ossification, the chondrocytes proliferate rapidly to form the model for bone. As they divide, the chondrocytes secrete a cartilage-specific extracellular matrix.

In the fourth phase, the chondrocytes stop dividing and increase their volume dramatically, becoming hypertrophic chondrocytes. These large chondrocytes alter the matrix they produce (by adding collagen X and more fibronectin) to enable it to become mineralized by calcium carbonate.

The fifth phase involves the invasion of the cartilage model by blood vessels. The hypertrophic chondrocytes die by apoptosis, and this space becomes bone marrow. As the cartilage cells die, a group of cells that have surrounded the cartilage model differentiate into osteoblasts, which begin forming bone matrix on the partially degraded cartilage. Eventually, all the cartilage is replaced by bone. Thus, the cartilage tissue serves as a model for the bone that follows.

The replacement of chondrocytes by bone cells is dependent on the mineralization of the extracellular matrix. A number of events lead to the hypertrophy and mineralization of the chondrocytes, including an initial switch from aerobic to anaerobic respiration, which alters their cell metabolism and mitochondrial energy potential. Hypertrophic chondrocytes secrete numerous small membrane-bound vesicles into the extracellular matrix. These vesicles contain enzymes that are active in the generation of calcium and phosphate ions and initiate the mineralization process within the cartilaginous matrix. The hypertrophic chondrocytes, their metabolism and mitochondrial membranes altered, then die by apoptosis.

In the long bones of many mammals (including humans), endochondral ossification spreads outward in both directions from the center of the bone. As the ossification front nears the ends of the cartilage model, the chondrocytes near the ossification front proliferate prior to undergoing hypertrophy, pushing out the cartilaginous ends of the bone. The cartilaginous areas at the ends of the long bones are called epiphyseal growth plates. These plates contain three regions: a region of chondrocyte proliferation, a region of mature chondrocytes, and a region of hypertrophic chondrocytes. As the inner cartilage hypertrophies and the ossification front extends farther outward, the remaining cartilage in the epiphyseal growth plate proliferates. As long as the epiphyseal growth plates are able to produce chondrocytes, the bone continues to grow.

The term “osteopenia” as used herein refers to a reduced bone mass of less severity than osteoporosis. It is defined by bone densitometry as a T score of −1 to −2.5.

The term “osteoporosis” as used herein refers to a decrease in bone density in which the bones become more porous and fragile, with an increased risk of fracture. It is defined as a T score of ≤−2.5.

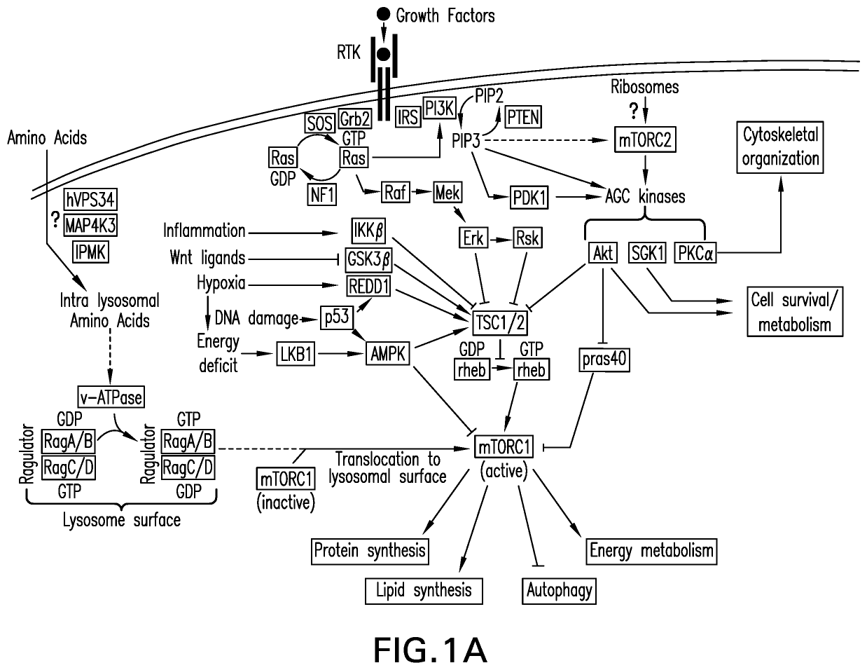

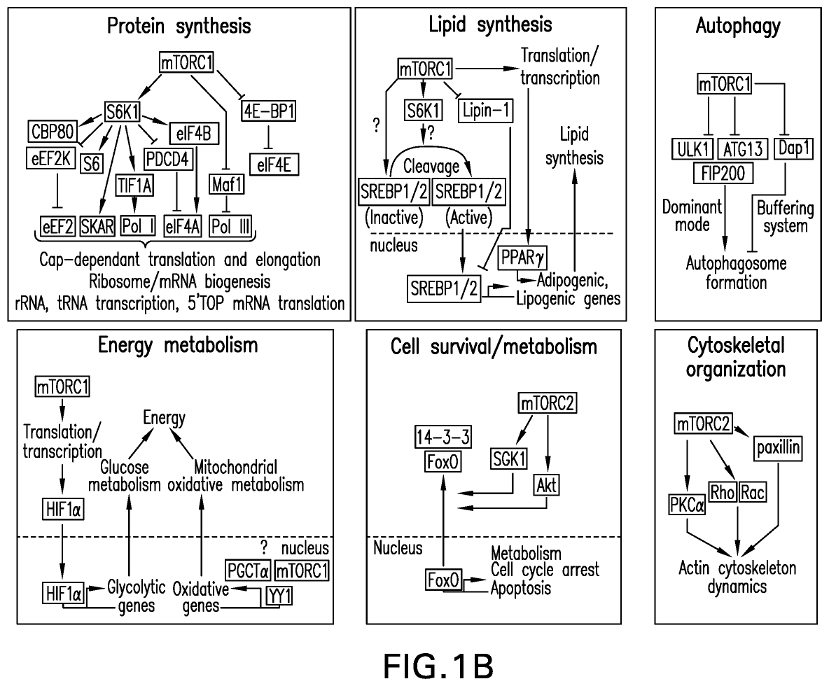

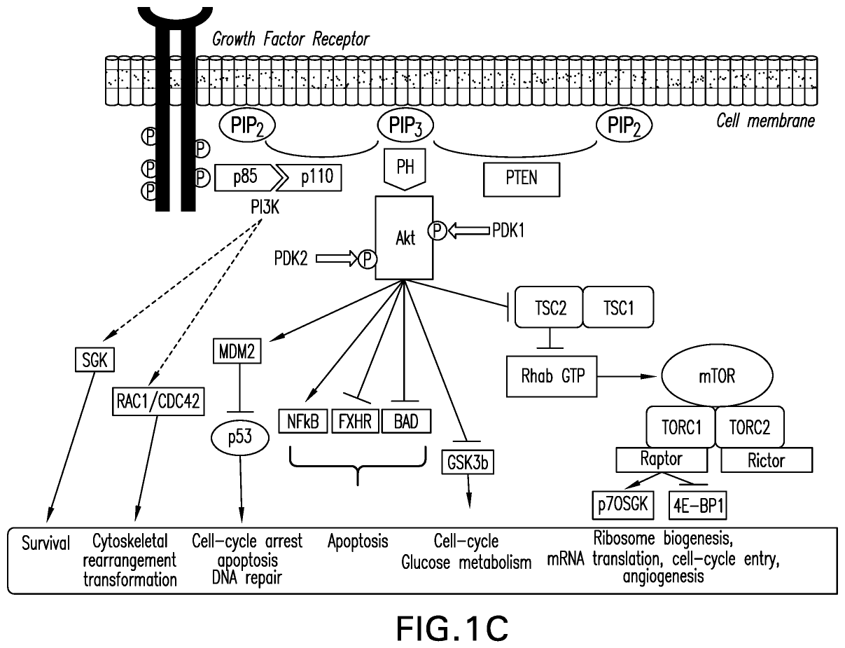

PI3K/Akt/mTOR Signaling Pathway. A schematic representation of the PI3K/Akt/mTor pathway is shown in FIG. 1.

The phosphatidylinositol-3-kinase (PI3K)/Akt and the mammalian target of rapamycin (mTor) signaling pathways are crucial to many aspects of cell growth and survival. Porta, C. et al., “Targeting PI2K/Akt/mTor signaling in cancer. Frontiers in Oncology (2014) doi.10.3389/fpmc.2014.00064). They are so interconnected that they could be regarded as a single pathway that, in turn, heavily interacts with many other pathways, including that of hypoxia inducible factors (HIFs).

PI3Ks constitute a lipid kinase family characterized by the capability to phosphorylate inositol ring 3′-OH group in inositol phospholipids. (Id., citing Fruman, D A et al., Phosphoinositide kinases. Annu. Rev. Biochem. (1998) 67: 481-507). Class I PI3Ks are heterodimers composed of a catalytic (CAT) subunit (i.e., p110) and an adaptor/regulatory subunit (i.e., p85). This classis further divided into two subclasses: subclass IA (PI3Kα, β, and δ), which is activated by receptors with protein tyrosine kinase activity, and subclass IB (PI3Kγ), which is activated by receptors coupled with G proteins (Id., citing Fruman, D A et al., Phosphoinositide kinases. Annu. Rev. Biochem. (1998) 67: 481-507).

Activation of growth factor receptor protein tyrosine kinases results in autophosphorylation on tyrosine residues. PI3K is then recruited to the membrane by directly binding to phosphotyrosine consensus residues of growth factor receptors or adaptors through one of the two SH2 domains In the adaptor subunit. This leads to allosteric activation of the CAT subunit. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) (Id., citing Fruman, D A et al., Phosphoinositide kinases. Annu. Rev. Biochem. (1998) 67: 481-507, Fresno-Vara, J A, et al., PI3K/Akt signaling pathway and cancer. Cancer Treat. Rev. (2004) 30: 193-204). Akt/PKB, on its own, regulates several cell processes involved in cell survival and cell cycle progression.

Akt. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor (Shimamura, H, et al., J. Am. Soc. Nephrol. 14: 1427-1434, 2003; Datta K, Franke T F, Chan T O, Makris A, Yang S I, Kaplan D R, Morrison D K, Golemis E A, Tsichlis P N, Mol Cell Biol 15: 2304-2310, 1995; Kulik G, Klippel A, Weber M J, Mol Cell Biol 17: 1595-1606, 1997; Yao R, Cooper G M, Science 267: 2003-2006, 1995). Stimulation of Akt has been shown to be dependent on phosphatidylinositol 3-kinase (PI3-kinase) activity (Fruman D A, Meyers R E, Cantley L C, Annu Rev Biochem 67: 481-507, 1998; Choudhury G G, Karamitsos C, Hemandez J, Gentilini A, Bardgette J, Abboud H E, Am J Physiol 273: F931-938, 1997, Franke T F, Yang S I, Chan T O, Datta K, Kazlauskas A, Morrison D K, Kaplan D R, Tsichlis P N, Cell 81: 727-736, 1995; Franke T F, Kaplan D R, Cantley L C, Cell 88: 435-437, 1997).

Akt has been shown to act as a mediator of survival signals that protect cells from apoptosis in multiple cell lines (Brunet A, Bonni A, Zigmond M J, Lin M Z, Juo P, Hu L S, Anderson M J, Arden K C, Blenis J, Greenberg M E, Cell 96: 857-868, 1999; Downward J, Curr Opin Cell Biol 10: 262-267, 1998). For example, phosphorylation of the pro-apoptotic Bad protein by Akt was found to decrease apoptosis by preventing Bad from binding to the anti-apoptotic protein Bcl-XL (Dudek H, Datta S R, Franke T F, Bimbaum M J, Yao R, Cooper G M, Segal R A, Kaplan D R, Greenberg M E, Science 275: 661-665, 1997; Datta S R, Dudek H, Tao X, Masters S, Fu H, Gotoh Y, Greenberg M E, Cell 91: 231-241, 1997). Akt was also shown to promote cell survival by activating nuclear factor-kB (NF-kB) (Cardone M H, Roy N, Stennicke H R, Salvesen G S, Franke T F, Stanbridge E, Frisch S, Reed J C, Science 282: 1318-1321, 1998; Khwaja A, Nature 401: 33-34, 1999) and inhibiting the activity of the cell death protease caspase-9 (Kennedy S G, Kandel E S, Cross T K, Hay N, Mol Cell Biol 19: 5800-5810, 1999).

mTOR signaling pathway: The mTOR signaling pathway is shown in FIG. 1A and FIG. 1B (taken from Laplante, M., Sabatini, DM, Cell (2012) 149(2): 274-293). Mechanistic target of rapamycin (mTOR) is an atypical serine/threonine kinase that is present in two distinct complexes. The first, mTOR complex 1 (mTORC1), is composed of mTOR, Raptor, GβL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis, or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumour-suppressor genes mutated in the tumour syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1). (Huang, J. Manning B D, Biochem J. (2008) 412(2): 179-90). Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GOL, Sin1, PRR5/Protor-1, and DEPTOR. mTORC2 promotes cellular survival by activating Akt, regulates cytoskeletal dynamics by activating PKCα, and controls ion transport and growth via SGK1 phosphorylation. Aberrant mTOR signaling is involved in many disease states

As used herein, the term “paracrine signaling” refers to short range cell-cell communication via secreted signal molecules that act on adjacent cells.

The term “pathogen associated molecular patterns” (PAMPs) as used herein refer to molecules specifically associated with groups of pathogens that are recognized by cells of the innate immune system.

The term “phenotype” as used herein refers to the observable characteristics of a cell, for example, expression of a protein.

The terms “polypeptide” and “protein” are used herein in their broadest sense to refer to a sequence of subunit amino acids, amino acid analogs, or peptidomimetics. The subunits are linked by peptide bonds, except where noted. These terms also apply to amino acid polymers in which one or more amino acid residue is an artificial chemical analogue of a corresponding naturally occurring amino acid, as well as to naturally occurring amino acid polymers. The terms also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, or they may be circular, with or without branching, generally as a result of posttranslational events, whether by natural processing or by events brought about by human manipulation, which do not occur naturally. Circular, branched and branched circular polypeptides may be synthesized by entirely synthetic methods

The term “pharmaceutical composition” is used herein to refer to a composition that is employed to prevent, reduce in intensity, cure or otherwise treat a target condition or disease. The terms “formulation” and “composition” are used interchangeably herein to refer to a product of the described invention that comprises all active and inert ingredients.

The term “pharmaceutically acceptable,” is used to refer to a carrier, diluent or excipient being compatible with the other ingredients of the formulation or composition (meaning capable of being combined with each other in a manner such that there is no interaction that would substantially reduce the efficacy of the composition under ordinary use conditions) and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use.

Polarization capacity. Specifying an axis of directionality is essential for most living cells. In the repertoire of cells that move individually, determining the cell front and back is a prerequisite for organizing the machinery that powers cell motility Early observations of single cells using conventional microscopy defined polarization according to cell shapes with elongated cells being more polarized than round cells. For moving cells, the migration direction is typically in the direction of the polarity axis, defined as the long-axis of the cell. [Rappel, W J, Edelstein-Keshet, L., Mechanisms of cell polarization. Curr. Opin. Syst. Biol. (2017) 3: 43-53].

The term “primer” refers to a nucleic acid which, when hybridized to a strand of DNA, is capable of initiating the synthesis of an extension product in the presence of a suitable polymerization agent. The primer is sufficiently long to uniquely hybridize to a specific region of the DNA strand. A primer also may be used on RNA, for example, to synthesize the first strand of cDNA.

The term “progenitor cell” as used herein refers to an immature cell in the bone marrow that may be isolated by growing suspensions of marrow cells in culture dishes with added growth factors. Progenitor cells mature into precursor cells that mature into blood cells. Progenitor cells are referred to as colony-forming units (CFU) or colony-forming cells (CFC). The specific lineage of a progenitor cell is indicated by a suffix, such as, but not limited to, CFU-E (erythrocytic), CFU-GM (granulocytic/macrophage), and CFU-GEMM (pluripotent hematopoietic progenitor).

The term “purification” and its various grammatical forms as used herein refers to the process of isolating or freeing from foreign, extraneous, or objectionable elements.

The term “quantitative real-time reverse transcription PCR” or “real-time quantitative reverse transcription PCR” (Real-Time qRT-PCR) refers a PCR technology that enables reliable detection and measurement of products generated during each cycle of the PCR process. RNA is used as the starting material, which is transcribed into complementary DNA (cDNA) by reverse transcriptase; the cDNA is used as the template for the quantitative PCR reaction.

The term “quiescence” as used herein is a property that often characterizes tissue-resident stem cells and allows them to act as a dormant reserve that can replenish tissues during homeostasis. Quiescence is thought to be a fundamental characteristic of hematopoietic stem cells (HSCs), which possess multi-lineage differentiation and self-renewal potential, and are able to give rise to all cell types within the blood lineage (Nakamura-Ischizu, A. et al., Development (2014) 141: 4656-66, citing Pietras, E M. et al., J. Cell Biol. (2011) 195: 709-720). Precise regulation of the cell cycle of quiescent HSCs is required for the effective production of mature hematopoietic cells with minimal stem cell exhaustion (Id., citing Orford, K W and Scadden, DT, Nature Rev. Genet. (2008) 9: 115-128). Since proliferating cells are more susceptible to genetic mutations and become senescent once their turnovers reach their maximum, a limit known as the Hayflick limit (Id., citing Hayflick, L. and Moorhead, P S, Expl Cell Res., (1961) 25: 585-621), quiescence supposedly protects HSCs from malignant transformation and malfunction (Id., citing Wang, J C Y and Dick, JE, Trends Cell Biol. (2005) 15: 494-501). Both cell-intrinsic and -extrinsic signals induced in response to various stresses, such as inflammation or blood loss, permit quiescent HSCs to re-enter the cell cycle, proliferate and differentiate (Id., citing Morrison, S J and Weissman, IL Immunity (1994) 1: 661-673; Suda, T. et al., Proc. Nat. Acad. Sci. USA (1983) 80: 6689-93).

The term “reference sequence” refers to a sequence used as a basis for sequence comparison. A reference sequence may be a subset or the entirety of a specified sequence.

The terms “regenerate”, “restore”, and “rejuvenate” are used interchangeably herein to refer to bringing back to a former youthful functional state; to make new again.

RNA interference (RNAi), or Post-Transcriptional Gene Silencing (PTGS) is a conserved biological response to double-stranded RNA that mediates resistance to both endogenous parasitic and exogenous pathogenic nucleic acids, and regulates the expression of protein-coding genes. It is a natural process by which double-stranded RNAs initiate the degradation of homologous RNA; researchers can take advantage of this process to study gene expression. A simplified model for the RNAi pathway is based on two steps, each involving ribonuclease enzyme. In the first step, the trigger RNA (either dsRNA or miRNA primary transcript) is processed into a short, interfering RNA (siRNA) by the RNase II enzymes Dicer and Drosha. In the second step, siRNAs are loaded into the effector complex RNA-induced silencing complex (RISC). The siRNA is unwound during RISC assembly and the single-stranded RNA hybridizes with a mRNA target. Gene silencing is a result of nucleolytic degradation of the targeted mRNA by the RNase H enzyme Argonaute (Slicer).

Gene silencing, however, can also occur not via siRNA-mediated cleavage of targeted mRNA, but rather, via translational inhibition. If the siRNA/mRNA duplex contains mismatches the mRNA is not cleaved; in these cases, direct translational inhibition may occur, especially when high concentrations of siRNA are present. The mechanism of this translation inhibition is not known.

As a result, siRNA can elicit two distinct modes of post-transcriptional repression. Because the requirement for target complementarity is less stringent for direct translational inhibition than for target mRNA cleavage, siRNAs designed for the latter may inadvertently trigger the former in another gene. Therefore, siRNAs designed against one gene may trigger silencing of an unrelated gene.

shRNA (short hairpin RNA) sequences offer the possibility of prolonged gene silencing. shRNAs are usually encoded in a DNA vector that can be introduced into cells via plasmid transfection or viral transduction. There are two main categories of shRNA molecules based on their design: simple stem-loop and microRNA-adapted shRNA. A simple stem-loop shRNA is often transcribed under the control of an RNA Polymerase III (Pol III) promoter [Bartel, D P, MicroRNAs: genomics, biogenesis, mechanism, and function. Cell 116(2):281-297 (2004), Kim, V. N. MicroRNA biogenesis: coordinated cropping and dicing. Nature Reviews, Molecular Cell Biology 6(5):376-385 (2005)]. The 50-70 nucleotide transcript forms a stem-loop structure consisting of a 19 to 29 bp region of double stranded RNA (the stem) bridged by a region of predominantly single-stranded RNA (the loop) and a dinucleotide 3′ overhang [Brummelkamp, T. R. et al. (2002) A system for stable expression of short interfering RNAs in mammalian cells. Science 296(5567):550-553; Paddison, P. J. et al. (2002) Stable suppression of gene expression by RNAi in mammalian cells. PNAS 99(3):1443-1448; Paul, C. P. et al. (2002) Effective expression of small interfering RNA in human cells. Nature Biotechnology 20(5):505-508]. The simple stem-loop shRNA is transcribed in the nucleus and enters the RNAi pathway similar to a pre-microRNA. The longer (>250 nucleotide) microRNA-adapted shRNA is a design that more closely resembles native pri-microRNA molecules, and consists of a shRNA stem structure which may include microRNA-like mismatches, bridged by a loop and flanked by 5′ and 3′ endogenous microRNA sequences [Silva, J. M. et al. (2005) Second-generation shRNA libraries covering the mouse and human genomes. Nature Genetics 37(11):1281-1288.]. The microRNA-adapted shRNA, like the simple stem-loop hairpin, is also transcribed in the nucleus but is thought to enter the RNAi pathway earlier similar to an endogenous pri-microRNA.

The term “small interfering RNAs,” which comprises both microRNA (miRNA) and small interfering RNA (siRNA), are small noncoding RNA molecules that play a role in RNA interference. siRNAs are synthesized from double-stranded segments of matched mRNA via RNA-dependent RNA polymerase, and siRNAs regulate the degradation of mRNA molecules identical in sequence to that of the corresponding siRNA, resulting in the silencing of the corresponding gene and the shutting down of protein synthesis. The main mechanism of action of siRNA is the mRNA cleavage function. There are no genes that encode for siRNAs. siRNAs can also silence gene expression by triggering promoter gene methylation and chromatin condensation. miRNAs are synthesized from an unmatched segment of RNA precursor featuring a hairpin turn, and miRNAs are encoded by specific miRNA genes as short hairpin pri-miRNAs in the nucleus. miRNAS are also small noncoding RNAs, but they seem to require only a 7- to 8-base-pair “seed” match between the 5′ region of the miRNA and the 3′UTR of the target. While the majority of miRNA targets are translationally repressed, degradation of the target mRNA can also occur. The main mechanism of action of miRNA may be the inhibition of mRNA translation, although the cleavage of mRNA is also an important role (Ross et al. Am J Clin Pathol. 2007; 128(5): 830-36).

The term “specifically hybridizes” as used herein refers to a process whereby a nucleic acid distinctively or definitively forms base pairs with complementary regions of at least one strand of the nucleic acid target sequence that was not originally paired to the nucleic acid. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 85% sequence identity, at least 90% sequence identity, at least 95% sequence identity, or at least 100% sequence identity (i.e., complementary) with each other.

The term “splice-site variant” as used herein refers to a genetic alteration in the DNA sequence that occurs at the boundary of an exon and an intron (splice site) that can result in an altered protein-coding sequence.

The term “steady state” as used herein refers to a state of dynamic equilibrium, where rate of loss quals the rate of gain.

The term “stem cells” as used herein refers to undifferentiated cells having high proliferative potential with the ability to self-renew that can generate daughter cells that can undergo terminal differentiation into more than one distinct cell phenotype. Stem cells are distinguished from other cell types by two characteristics. First, they are unspecialized cells capable of renewing themselves through cell division, sometimes after long periods of inactivity. Second, under certain physiologic or experimental conditions, they can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

Adult (somatic) stem cells are undifferentiated cells found among differentiated cells in a tissue or organ. Their primary role in vivo is to maintain and repair the tissue in which they are found. Adult stem cells have been identified in many organs and tissues, including brain, bone marrow, peripheral blood, blood vessels, skeletal muscles, skin, teeth, gastrointestinal tract, liver, ovarian epithelium, and testis. Adult stem cells are thought to reside in a specific area of each tissue, known as a stem cell niche, where they may remain quiescent (non-dividing) for long periods of time until they are activated by a normal need for more cells to maintain tissue, or by disease or tissue injury.

Bone Marrow Stem Cells. The term “bone marrow stem cells” as used herein refers to stem cells derived from the bone marrow and include HSCs and MSCs. The mononuclear fraction of bone marrow contains stromal cells, hematopoietic precursors, and endothelial precursors.

Peripheral Blood Stem Cells. The term “peripheral blood stem cells” as used herein refers to stem cells derived from peripheral blood. Peripheral blood houses adult (somatic) stem cells which are undifferentiated cells found among differentiated cells in a tissue or organ. Examples of peripheral blood stem cells include, but not limited to, hematopoietic stem cells, and mesenchymal stem cells [Dzierzak E. et al., “Of lineage and legacy: the development of mammalian hematopoietic stem cells,” Nature Immunol., Vol. 9(2): 129-136, (2008)].

Hematopoietic Stem Cells. As used herein, the term “hematopoietic stem cells” (also known as the colony-forming unit of the myeloid and lymphoid cells (CFU-M,L), or CD34⁺ cells) are rare pluripotent cells within the blood-forming organs that are responsible for the continued production of blood cells during life [Li Y. et al., “Inflammatory signaling regulates embryonic hematopoietic stem and progenitor cell production”, Genes Dev., Vol. 28(23): 2596-2612, (2014)]. HSCs can generate a variety of cell types, including erythrocytes, neutrophils, basophils, eosinophils, platelets, mast cells, monocytes, tissue macrophages, osteoclasts, and the T and B lymphocytes. The regulation of hematopoietic stem cells is a complex process involving self-renewal, survival and proliferation, lineage commitment and differentiation and is coordinated by diverse mechanisms including intrinsic cellular programming and external stimuli, such as adhesive interactions with the microenvironmental stroma and the actions of cytokines.

Different paracrine factors (cytokines) are important in causing hematopoietic stem cells to differentiate along particular pathways. The cytokines can be made by several cell types, but they are collected and concentrated by the extracellular matrix of the stromal (mesenchymal) cells at the sites of hematopoiesis. For example, granulocyte-macrophage colony-stimulating factor (GM-CSF) and the multilineage growth factor IL-3 both bind to the heparan sulfate glycosaminoglycan of the bone marrow stroma. The extracellular matrix then presents these factors to the stem cells in concentrations high enough to bind to their receptors [Alvarez S. et al., “GM-CSF and IL-3 activities in schistosomal liver granulomas are controlled by stroma-associated heparan sulfate proteoglycans,” J Leukoc Biol., Vol. 59(3): 435-441, (1996)].

Mesenchymal Stem Cells. Mesenchymal stem cells (MSCs) (also known as bone marrow stromal stem cells or skeletal stem cells) are non-blood adult stem cells found in a variety of tissues. They are characterized by their spindle-shape morphologically; by the expression of specific markers on their cell surface; and by their ability, under appropriate conditions, to differentiates along a minimum of three lineages (osteogenic, chondrogenic, and adipogenic) [Najar M. et al., “Mesenchymal stromal cells and immunomodulation: A gathering of regulatory immune cells”, Cytotherapy, Vol. 18(2): 160-171, (2016)]. No single marker that definitely delineates MSCs in vivo has been identified due to the lack of consensus regarding the MSC phenotype, but it generally is considered that MSCs are positive for cell surface markers CD105, CD166, CD90, and CD44 and that MSCs are negative for typical hematopoietic antigens, such as CD45, CD34, and CD14. As for the differentiation potential of MSCs, studies have reported that populations of bone marrow-derived MSCs have the capacity to develop into terminally differentiated mesenchymal phenotypes both in vitro and in vivo, including bone, cartilage, tendon, muscle, adipose tissue, and hematopoietic supporting stroma. Studies using transgenic and knockout mice and human musculoskeletal disorders have reported that MSC differentiate into multiple lineages during embryonic development and adult homeostasis [Najar M. et al., “Mesenchymal stromal cells and immunomodulation: A gathering of regulatory immune cells”, Cytotherapy, Vol. 18(2): 160-171, (2016)].

Analysis of the in vitro differentiation of MSCs under appropriate conditions that recapitulate the in vivo process have led to the identification of various factors essential for stem cell commitment. Among them, secreted molecules and their receptors (e.g., transforming growth factor-(P), extracellular matrix molecules (e.g., collagens and proteoglycans), the actin cytoskeleton, and intracellular transcription factors (e.g., Cbfa1/Runx2, PPARy, Sox9, and MEF2) have been shown to play important roles in driving the commitment of multipotent MSCs into specific lineages, and maintaining their differentiated phenotypes [Davis L. A. et al., “Mesodermal fate decisions of a stem cell: the Wnt switch”, Cell Mol Life Sci., Vol. 65(17): 2568-2574, (2008)].

The term “stem cell niche” as used herein refers to the specific area of each tissue within which adult stem cells reside, where they may remain quiescent (non-dividing) for long periods of time until they are activated by a normal need for more cells to maintain tissue, or by disease or tissue injury. Cells of the stem-cell niche interact with the stem cells to maintain them or promote their differentiation.

The term “stem cell rescue” or “rescue transplant” as used herein refers to a method of replacing blood-forming stem cells that were destroyed by treatment with high doses of anticancer drugs or radiation therapy. It is usually done using the patient's own stem cells that were saved before treatment. The stem cells help the bone marrow recover and make healthy blood cells. A stem cell rescue may allow more chemotherapy or radiation therapy to be given so that more cancer cells are killed.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one composition of the described invention, unless the context and usage of the phrase indicates otherwise.

The term “suspension” as used herein refers to a dispersion (mixture) in which a finely-divided species is combined with another species, with the former being so finely divided and mixed that it doesn't rapidly settle out.

The term “target” as used herein refers to a biological entity, such as, for example, but not limited to, a protein, cell, organ, or nucleic acid, whose activity can be modified by an external stimulus. Depending upon the nature of the stimulus, there may be no direct change in the target, or a conformational change in the target may be induced.

As used herein, the term “therapeutic agent” or “active agent” refers to refers to the ingredient, component or constituent of the compositions of the described invention responsible for the intended therapeutic effect.

The term “therapeutic component” as used herein refers to a therapeutically effective dosage (i.e., dose and frequency of administration) that eliminates, reduces, or prevents the progression of a particular disease manifestation in a percentage of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

As used herein, the term “tissue” refers to a collection of similar cells and the intercellular substances surrounding them. For example, connective tissue is the supporting or framework tissue of the body formed of fibrous and ground substance with numerous cells of various kinds. It is derived from the mesenchyme, and this in turn from the mesoderm. The varieties of connective tissue include, without limitation, areolar or loose; adipose; sense, regular or irregular, white fibrous; elastic; mucous; lymphoid tissue; cartilage and bone.

Thrombospondins. The thrombospondins (TSPs) are a family of five matricellular proteins that function during a wide range of physiological and pathological processes, including development, inflammation, angiogenesis and neoplasia (Duquette, M. et al., “Members of the thrombospondin gene family bind stromal interaction molecule 1 and regulate calcium channel activity,” Matrix Biol. (2014) 37: 15-24, citing Adams J C, Lawler J. The thrombospondins. Cold Spring Harb. Perspect. Biol. 2011; 3:a009712). They are transiently associated with the cell surface where they interact with a variety of membrane proteins, including proteoglycans, integrins, CD36, and CD47 (Id., citing Adams J C, Lawler J. The thrombospondins. Cold Spring Harb. Perspect. Biol. (2011) 3:a009712). Through these varied interactions, TSPs regulate extracellular matrix structure and cellular phenotype during tissue development and remodeling. For example, TSP-1 increases the association of CD36 with vascular endothelial growth factor receptor-2 (VEGFR-2) while decreasing the association of CD47 with VEGFR-2 in endothelial cells (Id., citing Kaur, S. et al. Thrombospondin-1 inhibits VEGF receptor-2 signaling by disrupting its association with CD47. J. Biol. Chem. (2010) 285:38923-38932; Kazerounian, S. et al., Priming of the vascular endothelial growth factor signaling pathway by thrombospondin-1, CD36, and spleen tyrosine kinase. Blood. (2011) 117:4658-4666). As a result, TSP-1 orchestrates fundamental changes in the way that endothelial cells respond to VEGF (Id., citing Kaur, S. et al. Thrombospondin-1 inhibits VEGF receptor-2 signaling by disrupting its association with CD47. J. Biol. Chem. (2010) 285:38923-38932; Kazerounian, S. et al., Priming of the vascular endothelial growth factor signaling pathway by thrombospondin-1, CD36, and spleen tyrosine kinase. Blood. (2011) 117:4658-4666); Chu, Y F et al., Thrombospondin-1 modulates VEGF signaling via CD36 by recruiting SHP-1 to VEGFR2 complex in microvascular endothelial cells. Blood. (2013) 122:1822-1832).

Each subunit of the TSP-1 trimer consists of multiple domains: amino- and carboxyl-terminal globular domains, a region of sequence homology to procollagen (PHR), and three types of repeated sequence motifs, designated type 1, type 2, and type 3 repeats (Id., citing Lawler J, Hynes R O. The structure of human thrombospondin, an adhesive glycoprotein with multiple calcium binding sites and homologies with several different proteins. J. Cell Biol. (1986) 103:1635-1648). Since the type 1 repeats were first identified in TSP-1 as a distinct structural motif, they have been designated thrombospondin repeats or TSRs (Id., citing Lawler J, Hynes R O. The structure of human thrombospondin, an adhesive glycoprotein with multiple calcium binding sites and homologies with several different proteins. J. Cell Biol. (1986) 103:1635-1648; Tucker R P. The thrombospondin type 1 repeat superfamily. Int. J. Biochem. Cell Biol. (2004) 36:969-974). The five members of the thrombospondin gene family can be divided into two subgroups, based on their structures (Bornstein, P et al., A second, expressed thrombospondin gene (Thbs2) exists in the mouse genome. J. Biol. Chem. (1991) 266:12821-128241; Oldberg, A. et al., COMP is structurally related to the thrombospondins. J. Biol. Chem. (1992) 267:22346-223502; Vos, H L et al., Thrombospondin-3 (Thbs3), a new member of the thrombospondin gene family. J. Biol. Chem. (1992) 267:12192-121962; Lawler, J. et al., evolution of the thrombospondin gene family. J. Mol. Evol. (1993a) 36:509-516; Lawler, J. et al, Identification and characterization of thrombospondin-4, a new member of the thrombospondin gene family. J. Cell Biol. (1993b) 120:1059-1067; Efimov, V P et al., The thrombospondin-like chains of cartilage oligomeric matrix protein are assembled by a five-stranded a-helical bundle between residues 20 and 83. FEBS Lett. (1994) 341:54-58; Newton, G et al., Characterization of human and mouse cartilage oligomeric matrix protein. Genomics. (1994) 24:435-439). TSP-1 and -2 (subgroup A) have the complete set of structural domains described above and are trimeric. By contrast, the subgroup B TSPs, TSP-3, and -4, and cartilage oligomeric matrix protein (COMP), lack both the TSRs and the PHR but contain an additional type 2 repeat (Id., citing Oldberg, A. et al., J. Biol. Chem. (1992) 267:22346-223502; Vos, H L et al., J. Biol. Chem. (1992) 267:12192-12196; Lawler, J et al., J. Cell Biol. (1993b) 120:1059-1067). The subgroup B proteins are also different from the subgroup A members in that they form pentamers instead of trimers (Id., citing Vos, H L et al., J. Biol. Chem. (1992) 267:12192-12196; Efimov, V P et al., FEBS Lett. (1994) 341:54-58). The type 2 repeats, the type 3 repeats and the carboxyl-terminal domains have the highest level of conservation amongst the TSPs and are collectively known as the signature domain. The structure of all or part of the signature domains of TSP-1 and -2, and COMP have been determined by X-ray crystallography revealing that the C-terminal domain forms a β-sandwich and that the type 3 repeats and portions of the type 2 repeats are closely associated with the surfaces of the 0-sandwich (Id., citing Kvansakul, M et al., Structure of a thrombospondin C-terminal fragment reveals a novel calcium core in the type 3 repeats. EMBO J. (2004) 23:1223-12334; Carlson, C B et al., Structure of the calcium-rich signature domain of human thrombospondin-2. Nat. Struct. Mol. Biol. (2005) 12:910-914; Tan, K et al., The crystal structure of the signature domain of cartilage oligomeric matrix protein: implications for collagen, glycosaminoglycan and integrin binding. FASEB J. (2009) 23:2490-2501). Binding sites for about 30 calcium ions are included in this structure. These sites are primarily located in the type 3 repeats which fold to form a contiguous series of calcium-binding sites, but calcium-binding sites are also present in the type 2 repeats and the C-terminal β-sandwich.

The term “transplantation” and its various grammatical forms as used herein refers to a surgical procedure in which tissue or an organ is transferred from one area of a person's body to another area, or from one person (the donor) to another person (the recipient).

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “type H vessels” as used herein refer to blood vessels characterized by high expression of CD31 (CD31^{hi} and endomucin (emcn^{hi}), which connect to arterioles and are surrounded by osteoprogenitors and release factors promoting osteogenesis. The term “L vessels” as used herein refers to vessels that are CD31^{lo}Emcn^{lo}, which correspond to BM sinusoids, and lack arteriolar connections and osteoprogenitor association. [Kusumbe, A. et al., Age-dependent modulation of vascular niches for haematopoietic stem cells. Nature (2016) 532 (7599): 380-84].

The term “vasculogenesis” as used herein refers to the process of new blood vessel formation.

The term “volume/volume percentage is a measure of the concentration of a substance in a solution. It is expressed as the ratio of the volume of the solute to the total volume of the solution multiplied by 100. Volume percent (vol/vol % or v/v %) should be used whenever a solution is prepared by mixing pure liquid solutions.

The abbreviation “WBM” stands for whole bone marrow.

The term “weight by weight percentage” or wt/wt % is used herein to refer to the ratio of weight of a solute to the total weight of the solution.

As used herein, the terms “wild type,” “naturally occurring,” or grammatical equivalents thereof, are meant to refer to an amino acid sequence or a nucleotide sequence that is found in nature and includes allelic variations; that is, an amino acid sequence or a nucleotide sequence that usually has not been intentionally modified. Accordingly, the term “non-naturally occurring,” “synthetic,” “recombinant,” or grammatical equivalents thereof, are used interchangeably to refer to an amino acid sequence or a nucleotide sequence that is not found in nature; that is, an amino acid sequence or a nucleotide sequence that usually has been intentionally modified. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purpose of the described invention.

### Methods

According to one aspect, the described invention provides a method for rejuvenating an aging blood and vascular system comprising aging-associated hematopoietic defects in a hematopoietic microenvironment of bone marrow including deteriorating vascular integrity, reduced hematopoietic stem cell function, or both, comprising
administering to a subject a pharmaceutical composition comprising an inhibitor of an angiocrine factor, a splice variant, or a fragment thereof, and a pharmaceutically acceptable carrier;
optionally administering a stem cell co-therapy comprising transplantation of a therapeutic amount of multipotent, self-renewing hematopoietic stem cells (HSCs) effective to regenerate the blood system and promote hematopoietic reconstitution of the bone marrow, and
optionally administering a vascular endothelial co-therapy comprising transplantation of a therapeutic amount of endothelial cells (ECs) effective to regenerate the blood system and promote hematopoietic reconstitution of the bone marrow, and
enhancing hematopoietic recovery in the hematopoietic bone marrow microenvironment by one or more of: reducing inflammation in the hematopoietic microenvironment of the bone marrow; preserving vascular integrity in the hematopoietic microenvironment of the bone marrow; or increasing frequency and numbers of cell types in the hematopoietic compartment to effect multi-lineage reconstitution.

According to some embodiments, the bone marrow microenvironment comprises a hematopoietic microenvironment comprising a hematopoietic stem cell (HSC) niche and a HSC-associated vascular niche comprising an endothelial microniche, and a perivascular niche comprising a mesenchymal cell.

According to some embodiments, the bone marrow (BM) microenvironment comprises BMECs, BM stromal cells, BM Lepr+ cells, and BM osteoblasts. According to some embodiments, the BMECs are sinusoidal and arteriole BMECs. According to some embodiments, the immunophenotype of BMECs is CD45−Ter119−CD31+VEcadherin+. According to some embodiments, the immunophenotype of BM stromal cells is CD45−Ter119−CD31−VEcadherin−. According to some embodiments, the immunophenotype of BM Lepr+ cells within the BM stromal population is CD45−Ter119−CD31−Lepr+. According to some embodiments, the immunophenotype of murine HSCs comprises lin−Ter119−CD11b−GR1−B220−CD3−CD41−ckit+SCA1+CD48−CD150+. According to some embodiments, the immunophenotype of human HSCs comprises Lineage−CD45RA−CD38−CD34+CD90+.

According to some embodiments, the HSC niche comprises one or more of hematopoietic stem cells (HSCs), hematopoietic stem and progenitor cells (HPSCs), multipotent progenitor cells (MPPs), and hematopoietic progenitor cell subsets.

According to some embodiments, the HSC niche further comprises a cell component. According to some embodiments, the cell component of the HSC niche comprises hematopoietic stem cells (HSCs), hematopoietic progenitor cells (HPCs), and resident cells of the HSC niche. According to some embodiments, at steady state, HSCs are mostly quiescent, while HPCs are actively proliferating and contributing to daily hematopoiesis. According to some embodiments, the HSC niche comprises secreted and membrane bound factors. According to some embodiments, the secreted and membrane factors bind surface receptors on the HSCs and HPCs. According to some embodiments, the secreted and membrane bound factors that bind surface receptors are chemokines. According to some embodiments, the secreted and membrane bound factors include Wnt, SCF, Cxcl12 and Jagged-1. According to some embodiments, the angiocrine factors generate angiocrine signals which balance self-renewal and differentiation of HSCs and HPCs. According to some embodiments, the resident niche cells of the HSC niche comprise endothelial and perivascular stromal cells.

According to some embodiments, the endothelial microniche comprises endothelial cells. According to some embodiments, the endothelial cells of the endothelial microniche comprise bone marrow endothelial cells (BMECs). According to some embodiments, the BMECs of the vascular niche of the hematopoietic microenvironment of bone marrow, when activated, produce angiocrine factors. According to some embodiments, the angiocrine factors produced by BMECs include at least one of CXCL-12, CXCR-4; bone morphogeneic protein 2 (BMP2) and bone morphogeneic protein 4 (BMP4), E-selectin, fibroblast growth factor 1 (FGF1) fibroblast growth factor 2 (FGF2), insulin growth factor binding protein (IGFBP), Jagged 1 (Jag 1), Jagged 2 (Jagged 2), interleukin 7 (IL-7), IL33, Noggin, stromal derived factor-1 (SDF1), SEMA-III, tenascin-C, TGF, thrombospondin-1 (TSP1), or tumor necrosis factor (TNF).

According to some embodiments, vasculogenesis in the vascular niche of the hematopoietic microenvironment of bone marrow comprises communication paths between HSCs and BMECs that create effective cellular crosstalk. According to some embodiments, the communication paths comprise one or more of SDF-1-CXCR-4 signaling, VEGF signaling, Notch signaling, Hedgehog signaling, or Wnt signaling.

According to some embodiments, the communication paths activated within the BMECs in the endothelial niche orchestrate a system of cellular crosstalk that results in differential production of the angiocrine factors. According to some embodiments, a communication path between HSCs and endothelial progenitor cells that create the cellular crosstalk during vasculogenesis includes one or more of SDF-1 (CXCL12)-CXCR-4 signaling; VEGF signaling, Notch signaling, Hedgehog signaling, or Wnt signaling. According to some embodiments, a communication path between HSCs and endothelial progenitor cells that create the cellular crosstalk during vasculogenesis includes SDF-1 (CXCL12)-CXCR-4 signaling. According to some embodiments, a communication path between HSCs and endothelial progenitor cells that create the cellular crosstalk during vasculogenesis includes VEGF signaling. According to some embodiments, a communication path between HSCs and endothelial progenitor cells that create the cellular crosstalk during vasculogenesis includes Notch signaling. According to some embodiments, a communication path between HSCs and endothelial progenitor cells that create the cellular crosstalk during vasculogenesis includes Hedgehog signaling. According to some embodiments, a communication path between HSCs and endothelial progenitor cells that create the cellular crosstalk during vasculogenesis includes Wnt signaling. According to some embodiments, the hematopoietic microenvironment comprises an osteoblastic or endosteal niche and an osteoblastic niche-associated vascular niche. According to some embodiments, the osteoblastic or endosteal niche comprises a cell component and growth factors.

According to some embodiments, the aging process is chronological aging. According to some embodiments, the aging process is physiological aging. According to some embodiments, the subject is a human subject. According to some embodiments, the subject is a mouse.

According to some embodiments, the aged hematopoietic microenvironment comprises one or more of sustained inflammation; increased stem cell pool size; myeloid biased differentiation of the HSCs, or reduced engraftment and regeneration of the bone marrow niche.

According to some embodiments, the aged hematopoietic microenvironment comprises sustained inflammation. According to some embodiments, the sustained inflammation in the hematopoietic microenvironment of the bone marrow includes vascular inflammation. According to some embodiments, the sustained inflammation in the hematopoietic microenvironment of the bone marrow includes inflammation of BM stromal cells. According to some embodiments, the sustained inflammation in the hematopoietic microenvironment of the bone marrow includes inflammation of hematopoietic cells. According to some embodiments, the sustained inflammation is derived from a myelosuppressive insult. According to some embodiments, the myelosuppressive insult comprises exposure to radiation, chemotherapy or both. According to some embodiments, the radiation is sublethal radiation, total body irradiation, or total lymphoid irradiation. According to some embodiments, the myelosuppressive insult comprises chemotherapy. According to some embodiments, the myelosuppressive insult is myeloablative.

According to some embodiments, the aged hematopoietic microenvironment comprises increased stem cell pool size. According to some embodiments, the aged hematopoietic microenvironment comprises myeloid biased differentiation of the HSCs.

According to some embodiments, the aged hematopoietic microenvironment comprises reduced engraftment and regeneration of the bone marrow niche after transplantation into the aged hematopoietic environment. According to some embodiments, the reduced engraftment after transplantation into the aged hematopoietic environment comprises a hematopoietic repopulation that is biased toward production of myeloid cells. According to some embodiments, the biased production of myeloid cells is at the expense of lymphopoiesis.

According to some embodiments, the deteriorating vascular integrity comprises increased vascular permeability. According to some embodiments, the deteriorating vascular integrity comprises increased endothelial permeability, increased endothelial inflammation, or both.

According to some embodiments, the aging associated hematopoietic defects in the HSC niche of the BM hematopoietic microenvironment include one or more of: increased HSC cellularity; changes to HSC pool size, loss of HSC self-renewal potential; increased HSC myeloid-biased differentiation, increased risk of failure of myeloablative strategies; or reduced engraftment after transplantation. According to some embodiments, the aging associated hematopoietic defects in the HSC niche of the BM hematopoietic microenvironment include increased HSC cellularity. According to some embodiments, the aging associated hematopoietic defects in the HSC niche of the BM hematopoietic microenvironment include changes to HSC pool size. According to some embodiments, the aging associated hematopoietic defects in the HSC niche of the BM hematopoietic microenvironment include loss of HSC self-renewal potential. According to some embodiments, the aging associated hematopoietic defects in the HSC niche of the BM hematopoietic microenvironment include expansion of HSC myeloid biased differentiation. According to some embodiments, the aging associated hematopoietic defects in the HSC niche of the BM hematopoietic microenvironment include increased risk of failure of myeloablative strategies According to some embodiments, the aging associated hematopoietic defects in the HSC niche of the BM hematopoietic microenvironment include reduced engraftment after transplantation.

According to some embodiments, the aging associated hematopoietic defects in the HSC microenvironment of the BM hematopoietic microenvironment include impaired HSC quiescence, increased HSC apoptosis or both. According to some embodiments, the aging associated hematopoietic defects in the HSC microenvironment of the BM hematopoietic microenvironment include impaired HSC quiescence. According to some embodiments, the aging associated hematopoietic defects in the HSC microenvironment of the BM hematopoietic microenvironment include increased HSC apoptosis.

According to some embodiments, aged HSCs exhibit one or more of activation of mTOR, autophagy-dependent survival, dysregulated DNA methylation, impaired histone modification, or disturbed cell polarity. According to some embodiments, overactivation of mTOR drives HSCs from quiescence into more active cell cycling.

According to some embodiments, the changes in HSC gene expression associated with aging in aged HSCs comprise upregulation of one or more of SELP, NEO1, JAM2, SLAMF1, PLSCR2, CLU, SDPR, FYB, ITGA6. According to some embodiments, the changes in HSC gene expression associated with aging in aged HSCs comprise upregulation of SELP. According to some embodiments, the changes in HSC gene expression associated with aging in aged HSCs comprise upregulation of NEO1. According to some embodiments, the changes in HSC gene expression associated with aging in aged HSCs comprise upregulation of JAM2. According to some embodiments, the changes in HSC gene expression associated with aging in aged HSCs comprise upregulation of SLAMF1. According to some embodiments, the changes in HSC gene expression associated with aging in aged HSCs comprise upregulation of PLSCR2. According to some embodiments, the changes in HSC gene expression associated with aging in aged HSCs comprise upregulation of CLU. According to some embodiments, the changes in HSC gene expression associated with aging in aged HSCs comprise upregulation of SDPR. According to some embodiments, the changes in HSC gene expression associated with aging in aged HSCs comprise upregulation of FYB. According to some embodiments, the changes in HSC gene expression associated with aging in aged HSCs comprise upregulation of ITGA6.

According to some embodiments, the changes in HSC gene expression associated with aging in aged HSCs comprise downregulation of one or more of RASSF4, FGF11, HSPA1B, HSPA1A, or NFKBIA. According to some embodiments, the changes in HSC gene expression associated with aging in aged HSCs comprise downregulation of RASSF4. According to some embodiments, the changes in HSC gene expression associated with aging in aged HSCs comprise downregulation of FGF11. According to some embodiments, the changes in HSC gene expression associated with aging in aged HSCs comprise downregulation of HSPA1B. According to some embodiments, the changes in HSC gene expression associated with aging in aged HSCs comprise downregulation of HSPA1A. According to some embodiments, the changes in HSC gene expression associated with aging in aged HSCs comprise downregulation of NFKB1A.

SELP is the gene encoding Selectin P, which redistributes to the plasma membrane during platelet activation and degranulation and mediates the interaction of activated endothelial cells or platelets with leukocytes.

NEO1 is the gene encoding neogenin 1, a cell surface protein that is a member of the immunoglobulin superfamily. The encoded protein may be involved in cell growth and differentiation and in cell-cell adhesion. Defects in this gene are associated with cell proliferation in certain cancers. Alternate splicing results in multiple transcript variants.

JAM2 is the gene encoding junctional adhesion molecule 2, which belongs to the immunoglobulin superfamily, and the junctional adhesion molecule (JAM) family. The protein encoded by this gene is a type I membrane protein that is localized at the tight junctions of both epithelial and endothelial cells. It acts as an adhesive ligand for interacting with a variety of immune cell types, and may play a role in lymphocyte homing to secondary lymphoid organs. Alternatively spliced transcript variants have been found for this gene.

SLAMF1 is the gene encoding self-ligand receptor of the signaling lymphocytic activation molecule family. SLAM receptors triggered by homo- or heterotypic cell-cell interactions modulate the activation and differentiation of a wide variety of immune cells and thus are involved in the regulation and interconnection of both innate and adaptive immune response. Activities are controlled by presence or absence of small cytoplasmic adapter proteins, SH2D1A/SAP and/or SH2D1B/EAT-2.

PLSCR2, is the gene encoding Phospholipid Scramblase 2, which may mediate accelerated ATP-independent bidirectional transbilayer migration of phospholipids upon binding calcium ions that results in a loss of phospholipid asymmetry in the plasma membrane.

CLU is the gene encoding Clusterin, a secreted chaperone that can under some stress conditions also be found in the cell cytosol.

SDPR is the gene that encodes caveeolae associated protein 1, which a calcium-independent phospholipid-binding protein whose expression increases in serum-starved cells. This protein is a substrate for protein kinase C (PKC) phosphorylation and recruits polymerase I and transcript release factor (PTRF) to caveolae.

FYB (FYN binding protein 1) is the gene encoding FYN binding protein 1, which is an adapter for the FYN protein and LCP2 signaling cascades in T-cells. The encoded protein is involved in platelet activation and controls the expression of interleukin-2. Three transcript variants encoding different isoforms have been found for this gene.

ITGA6 is the gene encoding integrin subunit alpha 6, which a member of the integrin alpha chain family of proteins. Integrins are heterodimeric integral membrane proteins composed of an alpha chain and a beta chain that function in cell surface adhesion and signaling.

RASSF4 is the gene encoding a potential tumor suppressor, which may promote apoptosis and cell cycle arrest.

FGF11 is the gene encoding a member of the fibroblast growth factor (FGF) family. FGF family members possess broad mitogenic and cell survival activities, and are involved in a variety of biological processes, including embryonic development, cell growth, morphogenesis, tissue repair, tumor growth and invasion. The function of this gene has not yet been determined. The expression pattern of the mouse homolog implies a role in nervous system development. Alternative splicing results in multiple transcript variants.

HSPA1B and HSPA1A are genes encoding molecular chaperones implicated in a wide variety of cellular processes, including protection of the proteome from stress, folding and transport of newly synthesized polypeptides, activation of proteolysis of misfolded proteins and the formation and dissociation of protein complexes. They encode 70 kDa heat shock proteins which are members of the heat shock protein 70 family.

NFKBIA is the gene encoding nuclear factor kappa B subunit 1. NF-kappa-B is a pleiotropic transcription factor present in almost all cell types and is the endpoint of a series of signal transduction events that are initiated by a vast array of stimuli related to many biological processes such as inflammation, immunity, differentiation, cell growth, tumorigenesis and apoptosis. NF-kappa-B is a homo- or heterodimeric complex formed by the Rel-like domain-containing proteins RELA/p65, RELB, NFKB1/p105, NFKB1/p50, REL and NFKB2/p52 and the heterodimeric p65-p50 complex appears to be most abundant one. The dimers bind at kappa-B sites in the DNA of their target genes and the individual dimers have distinct preferences for different kappa-B sites that they can bind with distinguishable affinity and specificity. Different dimer combinations act as transcriptional activators or repressors, respectively.

According to some embodiments, the aged endothelial microenvironment within the aged bone marrow hematopoietic microenvironment containing aged BMECs comprising one or more of a decrease in mTOR signaling, a reduced abundance of an mTOR subunit, reduced phosphorylation of mTOR catalytic subunits, or reduced expression of mTOR transcription target genes; or reduced protein levels in mTOR catalytic subunit mTOR Complex 1 and mTOR Complex 2. According to some embodiments, the aged endothelial microenvironment within the aged bone marrow hematopoietic microenvironment comprises BMECs comprising a decrease in mTOR signaling. According to some embodiments, decrease of mTOR signaling comprises at least one of: a decreased level of phosphatidylinositol 3-kinase/rapamycin (PI3k-mTOR) pathway signaling; a decreased level of PI3k-mTOR subunit abundance; a decreased expression of mTOR transcriptional target genes; or a decrease in the protein levels of mTOR subunits.

According to some embodiments, the aged endothelial microenvironment within the aged bone marrow hematopoietic microenvironment comprises BMECs comprising a reduced abundance of an mTOR subunit.

According to some embodiments, the aged endothelial microenvironment within the aged bone marrow hematopoietic microenvironment comprises aged BMECs with a decline in mTOR signaling. According to some embodiments, phosphorylation status of mTOR catalytic units mTOR signaling is reduced in BMECs of aged subjects, compared to young subjects. According to some embodiments, expression of mTOR downstream transcriptional target genes in aged BMECs is reduced compared to young BMEC controls. According to some embodiments, protein levels in mTOR catalytic subunit mTOR Complex 1 and mTOR Complex 2 are reduced in aged subjects.

According to some embodiments, the decline in mTOR signaling by BMECs causes functional defects associated with aging in aged HSCs. According to some embodiments, the functional defects associated with aging include one or more of a significant increase in total hematopoietic cells, an increase frequency of phenotypic LT-HSCs; a significant myeloid bias; reduced HPC activity; reduced polarization capacity; an increase in double strand DNA breaks; or changes in HSC gene expression similar to those of aged controls.

According to some embodiments, the functional defects associated with aging include a significant increase in total hematopoietic cells compared to a young control. According to some embodiments, the functional defects associated with aging include an increase frequency of phenotypic LT-HSCs, compared to a young control. According to some embodiments, the functional defects associated with aging include a significant myeloid bias compared to a young control. According to some embodiments, the functional defects associated with aging include reduced HPC activity compared to a young control. According to some embodiments, the functional defects associated with aging include reduced polarization capacity compared to a young control. According to some embodiments, the functional defects associated with aging include an increase in double strand DNA breaks, compared to a young control. According to some embodiments, the functional defects associated with aging include changes in HSC gene expression similar to those of aged controls, compared to a young control.

According to some embodiments the impaired (mTOR) signaling results in loss of quiescence for HSCs. According to some embodiments the loss of quiescence for HSCs leads to a transient increase in HSCs. According to some embodiments the loss of quiescence for HSCs leads to long-term exhaustion of HSCs. According to some embodiments the impaired mTOR signaling leads to defects in long-term repopulation capacity of HSCs. According to some embodiments the defects in long-term repopulation capacity of HSCs comprises a decreased potential for long-term engraftment. According to some embodiments, the defects in long term repopulation capacity of HSCs comprise a decreased capacity for multi-lineage repopulation. According to some embodiments, the defects in long term repopulation capacity of HSCs comprise a decreased potential for long-term engraftment potential, a decreased capacity for multi-lineage repopulation, and defective engraftment of HSCs.

According to some embodiments, top upregulated biological processes represented by changes in gene expression in aged BMECs, compared to a young control include one or more of inhibition of angiogenesis by TSP1, STAT3 pathway, TGF-b signaling, IGF-1 signaling or HMGB1 signaling. According to some embodiments, a top upregulated biological process represented by changes in gene expression in aged BMECs includes STAT3 pathway signaling. According to some embodiments, a top upregulated biological process represented by changes in gene expression in aged BMECs includes TGF-b signaling. According to some embodiments, a top upregulated biological process represented by changes in gene expression in aged BMECs includes IGF-1 signaling. According to some embodiments, a top upregulated biological process represented by changes in gene expression in aged BMECs includes HBGB1 signaling. According to some embodiments, each of STAT3 pathway signaling, TGF-b signaling, IGF-1 signaling and HMGB1 signaling is regulated by thrombospondin 1. According to some embodiments, inhibition of angiogenesis by TSP1 is the top upregulated biological process represented by changes in gene expression in aged subjects, compared to a young control.

According to some embodiments, expression levels of thrombospondin-1 (TSP1) are upregulated in aged BMECs when compared to young control subjects.

According to some embodiments, aged BMECs display impaired mTOR signaling. According to some embodiments, the impaired mTOR signaling comprises overactivation of mammalian target of rapamycin (mTOR), compared to a young control.

According to some embodiments, TSP1 activity includes regulation of platelet aggregation and anti-angiogenic activity. According to some embodiments, TSP1 is expressed by mature hematopoietic cells comprising megakaryocytes. According to some embodiments, TSP1 is expressed by BMECs. According to some embodiments, TSP1 activity includes regulation of platelet aggregation and anti-angiogenic activity in the vascular niche. According to some embodiments, TSP1 activity includes binding to and neutralizing vascular endothelial growth factor (VEGF). According to some embodiments, TSP1 activity comprises engaging CD47 and blocking VEGF receptor-2 (VEGFR2) signaling in the endothelial microniche. According to some embodiments, TSP1 activity comprises destabilizing adhesive contacts in the endothelial microniche.

According to some embodiments the angiocrine inhibitor is an inhibitor of thrombospondin 1 (TSP1). According to some embodiments, inhibition of thrombospondin 1 (TSP1) rejuvenates the aged hemopoietic microenvironment. According to some embodiments, engraftment potential is increased by inhibition of TSP1 in BMECs in aged subjects. According to some embodiments, lineage composition of HSC function is increased by inhibition of TSP1 in BMECs in aged subjects. According to some embodiments, both engraftment potential and lineage composition of HSC function are increased by inhibition of TSP1 in BMECs in aged subjects. According to some embodiments, HSC engraftment potential comprises percent change in CD45.2 engraftment in a competitive transplantation assay.

According to some embodiments inhibition of TSP1 is by binding of an antibody specific for TSP1, e.g., without limitation, uTSP1 neutralizing antibody clone 1 [ThermoFisher Scientific; MA5-13398]; uTSP1 neutralizing antibody clone 2 [ThermoFisher Scientific; MA5-13385]; Ms IgG1k IgG control [ThermoFisher Scientific; 16-4714-82]; uTSP neutralizing antibody clone 3[ThermoFisher Scientific; MA5-13377]; and Ms IgM control (x axis) [ThermoFisher Scientific; 14-4752-82]. According to some embodiments, inhibition of TSP1 is by binding of a nonneutralizing antibody to TSP1. According to some embodiments, inhibition of TSP1 is by binding of a neutralizing antibody to TSP1. According to some embodiments, the neutralizing antibody is commercially available as clone A4.1 (Thermofisher, Invitrogen RRID AB_10988669)). According to some embodiments, inhibition of TSP1 comprises administering the neutralizing antibody to TSP1 (uTSP1) by infusion. According to some embodiments, chimeric immunoglobulins having variable regions from one species (e.g., mouse) and constant regions from another species (e.g., human) can be prepared by linking DNA sequences encoding for the variable regions of the light and heavy chains from one species to the constant regions of the light and heavy chains respectively from a different species. Introduction of the resulting genes into mammalian host cells under conditions for expression as described in U.S. Pat. No. 5,807,715, which is incorporated herein by reference, provides for production of chimeric immunoglobulins having the specificity of the variable region derived from the mouse and the physiological functions of the constant region from the human. According to some embodiments, fully human monoclonal antibodies can be produced. In one approach, a conventional mouse hybridoma is made from an ordinary hyperimmunized BALB/c mouse, and the antibody-coding genes are then manipulated so that the constant regions are of human rather than murine origin. A further modification is to also ‘humanize’ the framework regions of the mouse antibody leaving only the CDRs (complementarity determining regions) of murine origin. Such antibodies elicit little or no immune response in humans. In another approach, according to some embodiments, a highly immune deficient NSG™ mouse (The Jackson Laboratory) can be reconstituted with a human immune system and hyperimmunized. Such mice produce murine B lymphocytes making human antibodies, which can then be used in a normal mouse fusion yielding a murine hybridoma making human antibodies.

Other techniques for knocking down gene expression are known. These include, without limitation, siRNA and miRNA based RNAi¹⁻⁴, anti-sense oligonucleotides⁵ and CRISPR/TALEN/zinc finger endonuclease⁶⁻¹⁰ based gene editing. According to some embodiments, the inhibitor of TSP1 is a nucleic acid inhibitor that knocks down gene expression both in vitro and in vivo.

According to some embodiments, the nucleic acid inhibitor is a siRNA. According to some embodiments, the siRNA can be modified to increase stability of the RNA. According to some embodiments, the siRNA is an LNA™-modified siRNA to increase its thermal stability. According to some embodiments, the nucleic acid inhibitor is an antisense oligonucleotide. An antisense oligonucleotide (ASO) is a short strand of deoxyribonucleotide analogue that hybridizes with the complementary mRNA in a sequence-specific manner via Watson-Crick base pairing. Formation of the ASO-mRNA heteroduplex either triggers RNase H activity, leading to mRNA degradation, induces translational arrest by steric hindrance of ribosomal activity, interferes with mRNA maturation by inhibiting splicing, or destabilizes pre-mRNA in the nucleus, resulting in downregulation of target protein expression. Chan, J H, Wong, L S, “Clin. Exp. Pharmacol. Physiol. 2006, 33 (5-6): 533-40.

According to some embodiments, the antisense oligonucleotide is a DNA antisense oligonucleotide. According to some embodiments, the antisense oligonucleotide is an RNA antisense oligonucleotide. According to some embodiments, the RNA antisense oligonucleotide is phosphorothioate modified to increase its stability and half-life.

According to some embodiment, the nucleic acid inhibitor is an oligodeoxynucleotide (ODN) decoy. A decoy oligonucleotide is a synthesized short DNA sequence that has the same sequence as that found on the portion of the promoter region of a gene where a transcription factor lands. Normally when a transcription factor lands on the promoter region of a gene, transcription of the gene is switched on leading to its expression. However, the decoy oligonucleotide acts as the promoter's “lure”, binds with the specific transcription factor in the cell so that the transcription factor cannot land on the genome, and the gene expression is suppressed.

According to some embodiments, inhibition of TSP1 accelerates recovery of the hematopoietic system. According to some embodiments, inhibition of TSP1 accelerates recovery of the hematopoietic system in the bone marrow of a subject. According to some embodiments, inhibition of TSP1 accelerates recovery of the hematopoietic system in the bone marrow of a subject subjected to a myelosuppressive insult. According to some embodiments, the myelosuppressive insult comprises sublethal radiation, chemotherapy, or both. According to some embodiments, the myelosuppressive insult comprises sublethal irradiation. According to some embodiments, the myelosuppressive insult comprises total body irradiation. According to some embodiments, the myelosuppressive insult comprises total lymphoid irradiation. According to some embodiments, the myelosuppressive insult comprises chemotherapy. According to some embodiments, the myelosuppressive insult comprises high-dose chemotherapy. According to some embodiments, the myelosuppressive insult is myeloablative. According to some embodiments, inhibition of TSP1 recues inflammation in the BM microenvironment.

According to some embodiments, recovery of the hematopoietic system comprises revascularization of the BM vascular niche. According to some embodiments, revascularization of the BM vascular niche is effective to establish regeneration of the BM vascular niche, restabilization of the BM vascular niche, or both. According to some embodiments, recovery of the hematopoietic system comprises restabilization of the BM vascular niche. According to some embodiments, inhibition of TSP1 is effective to regenerate the BM vascular niche, to restabilize the BM vascular niche, or both. According to some embodiments, inhibition of TSP1 is effective to regenerate the BM vascular niche. According to some embodiments, inhibition of TSP1 is effective to restabilize the BM vascular niche. According to some embodiments, inhibition of TSP1 in the endothelial microniche of a subject is effective to regenerate the BM vascular niche and to restabilize the BM vascular niche, or both.

According to some embodiments, inhibition of TSP1 is effective to increase HSC niche function in a BM hematopoietic microenvironment of an aged subject, compared to a young control. According to some embodiments, inhibition of TSP1 is effective to restore HSC function in the BM hematopoietic microenvironment of an aged subject, compared to a young control. According to some embodiments, inhibition of TSP1 is effective to restore multi-lineage capacity of the HSC niche in the BM hematopoietic microenvironment of an aged subject, compared to a young control. According to some embodiments, inhibition of TSP1 is effective to restore vascular integrity of the vascular niche in the BM hematopoietic microenvironment of an aged subject, compared to a young control. According to some embodiments, inhibition of TSP1 is effective to restore long-term engraftment potential of the HSC niche in the BM hematopoietic microenvironment of an aged subject, compared to a young control.

According to another aspect, the described invention provides a method for preparing a hematopoietic stem cell product for hematopoietic stem cell transplantation comprising (a) preparing ex vivo cultures of hematopoietic stem cells; (b) administering an antibody comprising anti-TSP1 antibodies to the cultures of (a) form a treated hematopoietic stem cell population; and (c) expanding the treated hematopoietic stem population in vitro to form a hematopoietic stem cell transplantation product comprising a therapeutic amount of treated hematopoietic stem cells, wherein engraftment potential of the hematopoietic stem cell transplantation product is enhanced compared to an untreated control. According to some embodiments, administering step (b) inhibits TSP1 in the treated hematopoietic stem cell population. According to some embodiments, the hematopoietic stem cells of step (a) are derived from a human subject. According to some embodiments, the hematopoietic stem cells of step (a) are derived from a mouse subject. According to some embodiments, the anti-TSP1 antibodies are neutralizing antibodies. According to some embodiments, the anti-TSP1 antibodies further comprise antibodies to CD36, CD47 or both, e.g., uTSP1 neutralizing antibody clone 1 [ThermoFisher Scientific; MA5-13398]; uTSP1 neutralizing antibody clone 2 [ThermoFisher Scientific; MA5-13385]; Ms IgG1k IgG control [ThermoFisher Scientific; 16-4714-82]; uTSP neutralizing antibody clone 3[ThermoFisher Scientific; MA5-13377]; and Ms IgM control (x axis) [ThermoFisher Scientific; 14-4752-82]. According to some embodiments, the antibodies are humanized antibodies. According to some embodiments, the anti-TSP1 neutralizing antibodies are commercially available as clone A4.1 (Thermofisher, Invitrogen RRID AB_10988669)). According to some embodiments, the transplantation is autologous. According to some embodiments, the hematopoietic stem cell transplantation is allogeneic.

According to some embodiments, the methods described are effective to increase HSC functionality in an aged HSC niche. According to some embodiments, the methods described herein are effective to increase HSC functionality in an aged HSC niche by at least 1%, by at least 2%, by at least 3%, by at least 4%, by at least 5%, by at least 6%, by at least 7%, by at least 8%, by at least 9%, by at least 10%, by at least 11%, by at least 12%, by at least 13%, by at least 14%, by at least 15%, by at least 16%, by at least 17%, by at least 18%, by at by at least 19%, by at least 20%, by at least 21%, by at least 22%, by at least 23%, by at least 24%, by at least 25%, by at least 26%, by at least 27%, by at least 28%, by at least 29%, by at least 30%, by at least 31%, by at least 32%, by at least 33%, by at least 34%, by at least 35%, by at least 36%, by at least 37%, by at least 38%, by at least 39%, by at least 40%, by at least 41%, by at least 42%, by at least 43%, by at least 44%, by at least 45%, by at least 46%, by at least 47%, by at least 48%, by at least 49%, by at least 50%, %, by at least 51%, by at least 52%, by at least 53%, by at least 54%, by at least 55%, by at least 56%, by at least 57%, by at least 58%, by at least 59%, by at least 60%, by at least 61%, by at least 62%, by at least 63%, by at least 64%, by at least 65%, by at least 66%, by at least 67%, by at least 68%, by at least 69%, by at least 70%, by at least 71%, by at least 72%, by at least 73%, by at least 74%, by at least 75%, by at least 76%, by at least 77%, by at least 78%, by at least 79%, by at least 80%, by at least 81%, by at least 82%, by at least 83%, by at least 84%, by at least 85%, by at least 86%, by at least 87%, by at least 88%, by at least 89%, by at least 90%, by at least 91%, by at least 92%, by at least 93%, by at least 94%, by at least 95%, by at least 96%, by at least 97%, by at least 98%, by at least 99%, or by at least 100%, compared to an untreated aged control.

According to some embodiments, the methods described herein are effective to enhance long-term engraftment potential of aged HSCs in the aged hematopoietic microenvironment. According to some embodiments, the methods described herein are effective to enhance long-term engraftment potential of aged HSCs in the aged hematopoietic microenvironment. by at least 1%, by at least 2%, by at least 3%, by at least 4%, by at least 5%, by at least 6%, by at least 7%, by at least 8%, by at least 9%, by at least 10%, by at least 11%, by at least 12%, by at least 13%, by at least 14%, by at least 15%, by at least 16%, by at least 17%, by at least 18%, by at by at least 19%, by at least 20%, by at least 21%, by at least 22%, by at least 23%, by at least 24%, by at least 25%, by at least 26%, by at least 27%, by at least 28%, by at least 29%, by at least 30%, by at least 31%, by at least 32%, by at least 33%, by at least 34%, by at least 35%, by at least 36%, by at least 37%, by at least 38%, by at least 39%, by at least 40%, by at least 41%, by at least 42%, by at least 43%, by at least 44%, by at least 45%, by at least 46%, by at least 47%, by at least 48%, by at least 49%, by at least 50%, %, by at least 51%, by at least 52%, by at least 53%, by at least 54%, by at least 55%, by at least 56%, by at least 57%, by at least 58%, by at least 59%, by at least 60%, by at least 61%, by at least 62%, by at least 63%, by at least 64%, by at least 65%, by at least 66%, by at least 67%, by at least 68%, by at least 69%, by at least 70%, by at least 71%, by at least 72%, by at least 73%, by at least 74%, by at least 75%, by at least 76%, by at least 77%, by at least 78%, by at least 79%, by at least 80%, by at least 81%, by at least 82%, by at least 83%, by at least 84%, by at least 85%, by at least 86%, by at least 87%, by at least 88%, by at least 89%, by at least 90%, by at least 91%, by at least 92%, by at least 93%, by at least 94%, by at least 95%, by at least 96%, by at least 97%, by at least 98%, by at least 99%, or by at least 100%, compared to an untreated aged control.

According to some embodiments, the methods described are effective to effect multi-lineage reconstitution of the aged hematopoietic microenvironment. According to some embodiments, the methods described herein are effective to effect multi-lineage reconstitution of the aged hematopoietic microenvironment by at least 1%, by at least 2%, by at least 3%, by at least 4%, by at least 5%, by at least 6%, by at least 7%, by at least 8%, by at least 9%, by at least 10%, by at least 11%, by at least 12%, by at least 13%, by at least 14%, by at least 15%, by at least 16%, by at least 17%, by at least 18%, by at by at least 19%, by at least 20%, by at least 21%, by at least 22%, by at least 23%, by at least 24%, by at least 25%, by at least 26%, by at least 27%, by at least 28%, by at least 29%, by at least 30%, by at least 31%, by at least 32%, by at least 33%, by at least 34%, by at least 35%, by at least 36%, by at least 37%, by at least 38%, by at least 39%, by at least 40%, by at least 41%, by at least 42%, by at least 43%, by at least 44%, by at least 45%, by at least 46%, by at least 47%, by at least 48%, by at least 49%, by at least 50%, by at least 51%, by at least 52%, by at least 53%, by at least 54%, by at least 55%, by at least 56%, by at least 57%, by at least 58%, by at least 59%, by at least 60%, by at least 61%, by at least 62%, by at least 63%, by at least 64%, by at least 65%, by at least 66%, by at least 67%, by at least 68%, by at least 69%, by at least 70%, by at least 71%, by at least 72%, by at least 73%, by at least 74%, by at least 75%, by at least 76%, by at least 77%, by at least 78%, by at least 79%, by at least 80%, by at least 81%, by at least 82%, by at least 83%, by at least 84%, by at least 85%, by at least 86%, by at least 87%, by at least 88%, by at least 89%, by at least 90%, by at least 91%, by at least 92%, by at least 93%, by at least 94%, by at least 95%, by at least 96%, by at least 97%, by at least 98%, by at least 99%, or by at least 100% compared to an untreated aged control.

### Formulations/Administration

According to some embodiments, the inhibitor of an angiocrine factor, splice variant, or fragment may be formulated as a composition. According to some embodiments, the angiocrine factor is TSP1. According to some embodiments, the inhibitor is an antibody or antigen-binding fragment thereof. The antibodies and antigen binding fragments of the described invention can be formulated as a pharmaceutical composition suitable for parenteral administration. The injectable solution can be composed of either a liquid or lyophilized dosage form.

According to some embodiments, if the pharmaceutical composition is formulated for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. According to some embodiments, sterile aqueous media that can be employed will be known to those of skill in the art in light of the present disclosure. According to some embodiments, formulations should meet appropriate sterility, pyrogenicity, general safety and purity standards as required The buffer can be L-histidine (1-50 mM), optimally 5-10 mM, at pH 5.0 to 7.0 (optimally pH 6.0). The term “buffer” as used herein refers to a solution or liquid whose chemical makeup neutralizes acids or bases without a significant change in pH. Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). According to some embodiments, the infusion solution is isotonic to subject tissues. According to some embodiments, the infusion solution is hypertonic to subject tissues.

Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, for example 1-10% mannitol (optimally 2-4%). Stabilizers can be used in both liquid and lyophilized dosage forms, for example 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the antibodies and antibody-portions of the described invention prepared as an injectable solution for parenteral administration can further comprise an agent useful as an adjuvant, such as those used to increase the absorption, or dispersion of a therapeutic protein (e.g., antibody). An exemplary adjuvant is hyaluronidase, such as HYLENEX (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions. (see WO2004078140, US2006104968 incorporated herein by reference).

The compositions of the described invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application. Typical exemplary compositions are in the form of injectable or infusible solutions, such as compositions similar to those used for passive immunization of humans with other antibodies. The exemplary mode of administration is parenteral (e.g., intravenous, subcutaneous, intraperitoneal, intramuscular). According to a one embodiment, the antibody is administered by intravenous infusion or injection. According to another embodiment, the antibody is administered by intramuscular or subcutaneous injection.

Therapeutic compositions typically must be sterile and stable under the conditions of manufacture and storage. According to some embodiments, the composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies and antigen-binding fragments of the described invention can be administered by a variety of methods known in the art, for example, subcutaneous injection, intravenous injection or infusion. As will be appreciated by the skilled artisan, the route and/or mode of administration will vary depending upon the desired results.

According to some embodiments, the composition is a pharmaceutical composition comprising a pharmaceutically acceptable carrier. According to some embodiments, the active compound may be prepared with a carrier that will protect the active against rapid release, such as a controlled release formulation, including implants, transdermal patches, and microencapsulated delivery systems. According to some embodiments, the carrier of the composition of the present invention may include a release agent such as sustained release or delayed release carrier. Biodegradable, biocompatible polymers can be used, such as ethylene vinyl acetate, polyanhydrides, polyglycolic acid, collagen, polyorthoesters, and polylactic acid. According to some such embodiments, the carrier can be any material capable of sustained or delayed release of the active to provide a more efficient administration, e.g., resulting in less frequent and/or decreased dosage of the composition, improve ease of handling, and extend or delay effects on diseases, disorders, conditions, syndromes, and the like, being treated, prevented or promoted. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

According to other embodiments, an antibody or antibody portion of the described invention may be conjugated to a polymer-based species such that the polymer-based species may confer a sufficient size upon said antibody or antigen binding antibody fragment of the described invention such that the antibody or antigen-binding portion of the described invention benefits from the enhanced permeability and retention effect (EPR effect) (See also PCT Publication No. WO2006/042146A2 and U.S. Publication Nos. 2004/0028687A1, 2009/0285757A1, and 2011/0217363A1, and U.S. Pat. No. 7,695,719 (each of which is incorporated by reference herein in its entirety and for all purposes).

Supplementary active compounds can also be incorporated into the compositions. In certain embodiments, an antibody or antibody fragment of the described invention is formulated with and/or co-administered with one or more additional therapeutic agents. For example, the antibody or antibody fragment may be formulated and/or co-administered with one or more additional antibodies that bind other targets (e.g., antibodies that bind cytokines or that bind cell surface molecules). Furthermore, the antibody or antibody fragment of the described invention may be used in combination with two or more therapeutic agents. Such combination therapies may advantageously utilize lower dosages of the administered therapeutic agents, thus avoiding possible toxicities or complications associated with the various monotherapies.

According to some embodiments, an antibody, or fragment thereof is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044, which are hereby incorporated by reference for any purpose.

According to some embodiments, the pharmaceutical composition is administered with a co-therapy. According to some embodiments, the pharmaceutical composition is administered with a therapeutic amount of a co-therapy. According to some embodiments, the pharmaceutical composition herein is administered before the co-therapy. According to some embodiments, the pharmaceutical composition herein is administered after a co-therapy. According to some embodiments, the pharmaceutical composition herein is administered concurrently with the co-therapy.

According to some embodiments, the adjunct therapy is a stem cell therapy. According to some embodiments, the pharmaceutical composition is administered with a therapeutic amount of the stem cell therapy, wherein the therapeutic amount is effective to promote or induce stem cell rescue.

According to some embodiments, a stem cell transplant may be formulated by any appropriate methods. In brief, stem cell therapy comprises the steps of isolating hematopoietic stem cells from a population of mononuclear cells isolated from a tissue source, enriching the isolated population of mononuclear cells for hematopoietic stem cells by positive or negative selection, and infusing the enriched isolated population of hematopoietic stem cells to the subject. According to some embodiments, the tissue source is autologous. According to some embodiments, the tissue source is allogeneic. The specificities of the above described method depends on the tissue source of the stem cells.

Autologous Tissue. According to some embodiments, the tissue source comprises autologous tissue. According to some embodiments, the autologous tissue is harvested prior to myeloablative insult. According to some embodiments, the harvested autologous tissue comprising stem cells further undergoes purging to deplete contaminating tumor cells. According to some embodiments, if malignant cells exist in the harvested tissue, the stem cells are enriched through the use of anti-CD34 specific monoclonal antibodies and immunobeads (“positive selection”) and/or the malignant cells are removed through the use of antitumor monoclonal antibodies (“negative selection”).

Allogeneic Tissue. According to some embodiments, the tissue source comprises allogeneic tissue. According to some embodiments, the donor allogeneic tissue is screened for histocompatibility with the recipient subject. According to some embodiments, histocompability is screened through histocompatibility matching wherein the donor and the recipient subject are human leukocyte antigen (HLA) identical or nearly identical or similar. According to some embodiments, if malignant cells exist in the harvested tissue, the harvested tissue is purged as described above. According to some embodiments, histo-incompatible material may be removed from the harvested material. According to some embodiments, the allogeneic harvested tissue may also undergo ex-vivo T cell depletion (TCD).

Bone marrow tissue. According to some embodiments, the tissue source comprises bone marrow wherein the tissue is either allogeneic or autologous. According to some embodiments, any known method to harvest bone marrow tissue may be used. For example, bone marrow for transplantation may be obtained (“harvested”) by multiple aspirations of the iliac crest over 2-3 hours under general or spinal anesthesia. Approximately 10-40×10⁹ nucleated cells (2×10⁸/kg of recipient weight), up to a maximum of 20 mL/kg of donor weight, will be obtained. The marrow aspirate will primarily consist of stromal cells, undifferentiated stem cells, early committed progenitor cells, T lymphocytes and erythroid, myeloid, monocytic, megakaryocytic, and lymphoid cell lines in various stages of development. Particulate material in the marrow will be removed by filtration. If an ABO blood group incompatibility exists, plasmapheresis may be utilized to remove isohemagglutinins, while differential centrifugation can be utilized to remove incompatible erythrocytes. Special processing (“purging”) may also be performed to reduce the marrow burden of tumor cells, T lymphocytes, or other specific components that may have a deleterious effect on the recipient subject. After processing, harvested, processed tissue comprising the stem cells will be immediately administered to the recipient via intravenous infusion or will be cryopreserved and stored for later transfusion.

Peripheral blood. According to some embodiments, the tissue source is peripheral blood wherein the tissue is either allogeneic or autologous. According to some embodiments, any known method to harvest peripheral blood may be used. According to some embodiments, the population of mononuclear cells is obtained after treatment with a hematopoietic stem cell mobilizing agent. According to some such embodiments, the hematopoietic stem cell mobilizing agent comprises G-CSF, GM-CSF (e.g., Sargramostim (LEUKINE®)), or a pharmaceutically acceptable analog or derivative thereof. According to some embodiments, the hematopoietic stem cell mobilizing agent is a recombinant analog or derivative of a colony stimulating factor. According to some embodiments, the hematopoietic stem cell mobilizing agent is filgrastim (NEUPOGEN®). According to some embodiments, the hematopoietic stem cell mobilizing agent is one or more of plerixafor (MOZOBIL®), eltrombopag (PROMACTA®), Romiplostim (NPLATE®), pegfilgrastim (NEULASTA®), darbepoietin alfa (ARANESP®). Then, the donor's buffy coat comprising stem cells then may be isolated by leukapheresis. After processing, the enriched population of hematopoietic stem cells will be immediately administered to the recipient via intravenous infusion or will be cryopreserved frozen and stored for later transfusion.

### Doses/Dosage Regimes

According to some embodiments, the amount of antibody or antigen-binding antibody fragment can be prepared so that a suitable dosage is e contained in a unit dose of the pharmaceutical composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

According to some embodiments, the actual dosage amount of a composition of the present disclosure administered to a subject can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject.

### Subjects

The compositions and methods described herein are intended for use with any subject that may experience the described benefits. Thus, “subjects,” “patients,” and “individuals” (used interchangeably) include humans as well as non-human subjects, particularly domesticated animals.

According to some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell.

According to some embodiments, the subject and/or animal is a human. According to some embodiments, the human is an adult human. According to some embodiments, the human is a geriatric human. In other embodiments, the human may be referred to as a patient.

According to some embodiments, the subject is a non-human animal, and therefore the described invention pertains to veterinary use. According to some embodiments, the non-human animal is a household pet. According to some embodiments, the non-human animal is a livestock animal.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Loss of Endothelial mTOR Drives Hematopoietic Stem Cell Aging

### Example 1A: Aging Results in a Decrease in mTOR Signaling in BMECs

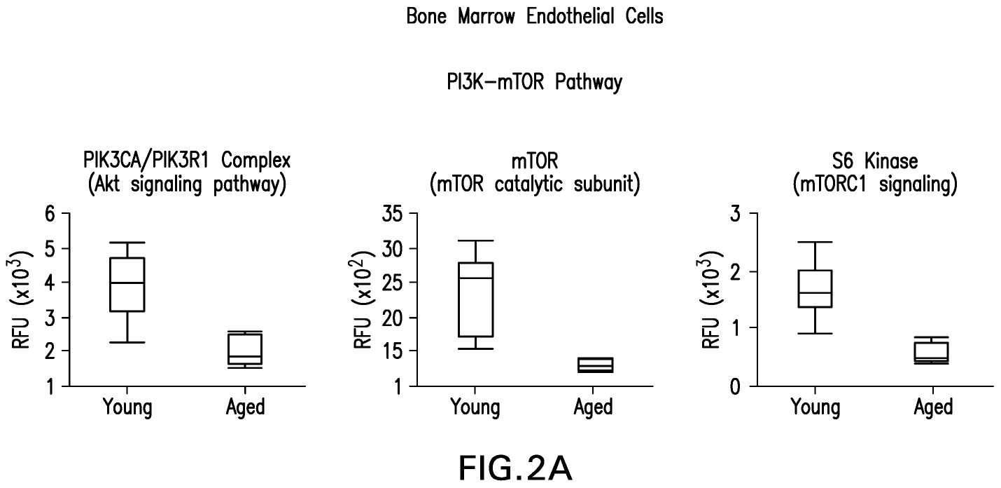

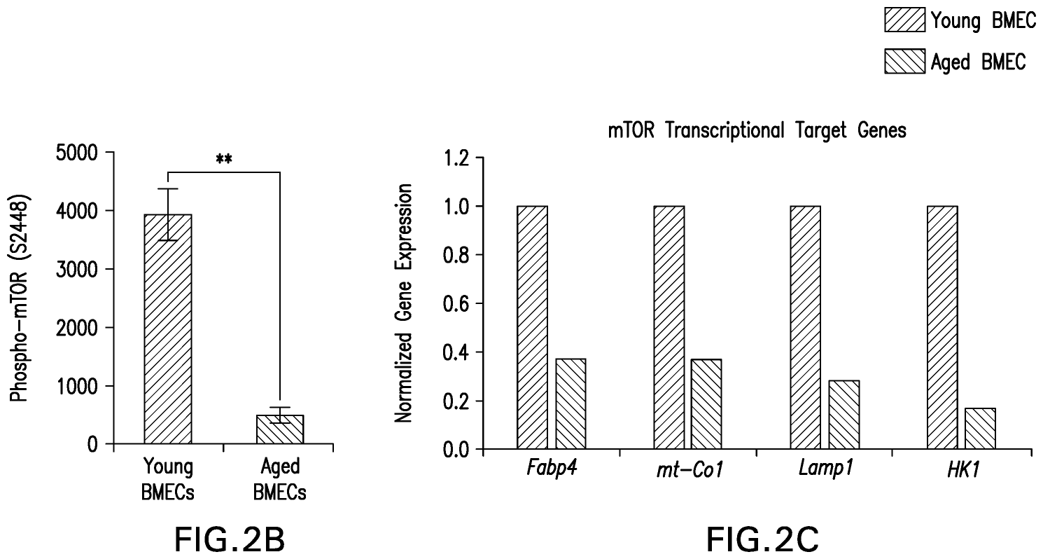

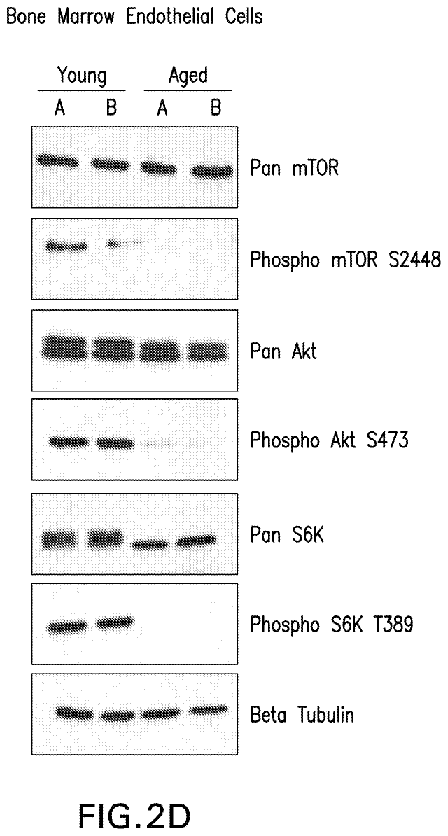

To better understand how physiological aging could potentially lead to the functional defects observed in the hematopoietic system, proteomic analysis (Somalogic) was performed on bone marrow microvascular endothelial cells (BMECs) isolated from young (12 weeks) and aged (24 months) mice. 154 candidate factors were identified using cutoffs for the false discovery rate (FDR) at q=0.02 and p-values of 0.05, and screening for changes in proteins in aged BMECs when compared to young controls. The list was further refined to 86 candidate proteins by excluding proteins discovered with low confidence. Many proteins within the data set are found to be associated with the PI3K/AKT/mTOR signaling axis. FIG. 2A illustrates abundance of PIK3CA/PIK3R1 complex in young and aged mice. The data show that mTOR subunit abundance is decreased in BMECs of aged mice. Consistent with these data it has been demonstrated that BMEC signaling through the mTOR pathway is critical for expanding HSCs [Kobayashi, H., et al., Angiocrine factors from Akt-activated endothelial cells balance self-renewal and differentiation of haematopoietic stem cells. Nat Cell Biol, 2010. 12(11): p. 1046-56]

Next, the effects of physiological aging on mTOR signaling within BMECs were examined. Phospho-Flow cytometry was to measure the phosphorylation status of mTOR catalytic subunits. To assess mTOR phosphorylation, young (12-16 weeks) and aged (24 months) C57BL6 mice were injected retro-orbitally with 25 μg of a fluorophore-conjugated antibody raised again VE-cadherin (BV13; Biolegend) 15 minutes prior to sacrifice. Long bones were isolated, crushed, and enzymatically disassociated in Digestion buffer for 15 minutes at 37° C. Resulting cell suspensions were filtered (40 μm), washed using MACS buffer. Single-cell WBM suspensions were depleted of lineage-committed hematopoietic cells using a Lineage Cell Depletion Kit (Miltenyi) according to the manufacturer's suggestions. Resulting lineage⁻ cells were stained with fluorophore-conjugated antibodies raised against CD31 (390; Biolegend) and CD45 (30-F11; Biolegend). Stained cells were washed using MACS buffer, fixed, permeabilized using Phosphoflow Fix Buffer 1 and Perm Buffer 3 (BD Biosciences) and stained with antibodies raised against phosphorylated mTOR (Ser2448) (BD Biosciences 563489), phosphorylated AKT (S473) (BD Biosciences 560404) and phosphorylated S6 (S235/236) (BD Biosciences 560434) for 30 minutes at room temperature according to the manufacturer's recommendations. Cells were washed in MACS buffer. Appropriate concentration matched isotype controls were utilized for gating and analysis by Flow cytometry. mTOR signaling was found to be significantly decreased in BMECs of aged (22 month) mice compared to young (12 weeks) mice. FIG. 2B shows quantification of mean fluorescent intensity of freshly-isolated BMECs in young and aged mice. The data demonstrate a decrease in the mROT phosphor-Ser2448.A The data also show a decrease in the expression of mTOR transcriptional targets. FIG. 2C is an expression analysis of mTOR downstream transcriptional target genes by RT-PCR. Gene expression was normalized to the Actb gene, which encodes beta-actin. The results demonstrate a reduction in mTOR-dependent gene expression in aged BMECs compared to young BMC controls, [Pradeep Ramalingam, et al. Endothelial mTOR maintains hematopoiesis during aging. (2020) https://doi.org/10.1084/jem.20191212], further demonstrating a decline in mTOR signaling within the aged endothelial niche. To further confirm these findings, samples (N=5) of young and aged mice were pooled and BMECs were isolated for Western analysis in 2 independent cohorts; A and B. WBM from long bones (femur and tibia) were flushed using a 26.5-gauge needle with ice-cold PBS (pH 7.2) containing 2 mM EDTA. WBM was depleted of red blood cells (RBC Lysis Buffer; Biolegend) according to the manufacturer's recommendations. Briefly, flushed marrow cells were pelleted by centrifugation (500 g for 5 minutes at 40 C) and the cells were resuspended in 3 mL of ice-cold 1×RBC lysis buffer, vortexed briefly and incubated for 5 minutes on ice. Cells were pelleted by centrifugation (500 g for 5 minutes at 40 C), supernatant was discarded, and cells were washed with 3 mL of ice-cold PBS (pH 7.2). Cell pellets were lysed in RIPA buffer (107 cells in 0.5 mL RIPA buffer; Thermo Cat #89900) containing 2× Phosphatase Inhibitor (Thermo Cat #78428) and 2× Protease Inhibitor Cocktail (Thermo Cat #78430) for 1 hour at 4° C. with gentle agitation, sonicated, and centrifuged for 10 minutes at 21,000×g at 4° C. to remove insoluble debris. Protein concentrations were determined using the DC Protein Assay (BioRad 5000111) and 20 μg total protein was denatured for 5 min at 70° C. in 1× Laemmli Buffer (Sigma Cat #53401-10VL), resolved on SDS-acrylamide gels and electroblotted to nitrocellulose. Transferred blots were blocked for 1 hour in 5% w/v non-fat dry milk in 1×TBST (Cell Signaling Cat #9997). Blots were washed 3× for 5 minutes in 1×TBST and incubated overnight at 4° C. in 5% BSA w/v in 1×TBST with primary antibodies raised against phospho-S6 (Cell Signaling 4858), S6 (Cell Signaling 2217), phospho 4EBP-1 (Cell Signaling 2855), 4EBP-1 (Cell Signaling 9644) and Actb (Cell Signaling 4970), at the manufacturer recommended dilutions. Blots were washed 3×5 mins in 1×TBST and incubated in 5% non-fat dry milk in 1×TBST containing anti-rabbit (H+L) horseradish peroxidase (Jackson ImmunoResearch Laboratories) secondary antibodies at a dilution of 1:20,000 for 1 hour at room temp. Blots were rinsed twice and washed 4×5 minutes in 1×TBST and developed using Amersham ECL Prime Western Blotting Detection Reagent (GE Healthcare RPN2232), according to the manufacturer's suggestions. All blots were developed using Carestream Kodak BioMax Light Film (Sigma-Aldrich). FIG. 2D shows the resulting Western blot analysis of the BMEC samples. In aged mice, a decrease in protein levels in the mTOR catalytic subunit (p-mTOR S2448), mTOR Complex 1 (p-S6K T389), and mTOR Complex 2 (p-AKT S473) was observed.

Together, the data support the conclusion that aging is associated with a strong reduction of mTOR signaling in BMECs.

### Example 1B: Endothelial-Specific Deletion of mTOR Results in Premature Aging of the HSC

We previously reported that EC-specific AKT/mTOR activation supports HSC maintenance and self-renewal ex vivo [Kobayashi, H., et al., Angiocrine factors from Akt-activated endothelial cells balance self-renewal and differentiation of haematopoietic stem cells. Nat Cell Biol, 2010. 12(11): p. 1046-56.] The observed decrease in mTOR signaling within aged BMECs (FIG. 2A, 2B, 2C, 2D) may underlie the age associated HSC functional defects in vivo.

To test this hypothesis, mTOR was specifically deleted from adult ECs by crossing an mTOR^{fl/fl} mouse to a tamoxifen-inducible cre transgenic mouse driven by the adult EC-specific VEcadherin promoter (mTOR^{(ECKO)})[Pradeep Ramalingam, et al. Endothelial mTOR maintains hematopoiesis during aging. (2020) https://doi.org/10.1084/jem.20191212]. Flow cytometric analysis was performed on young (12-16 weeks) mTOR^{(ECKO)} mice and young (12-16 weeks) control mice to determine the effect of EC-specific mTOR deletion on the regulation of HSCs and their progeny; 22-24 month old wild-type mice served as aged controls.

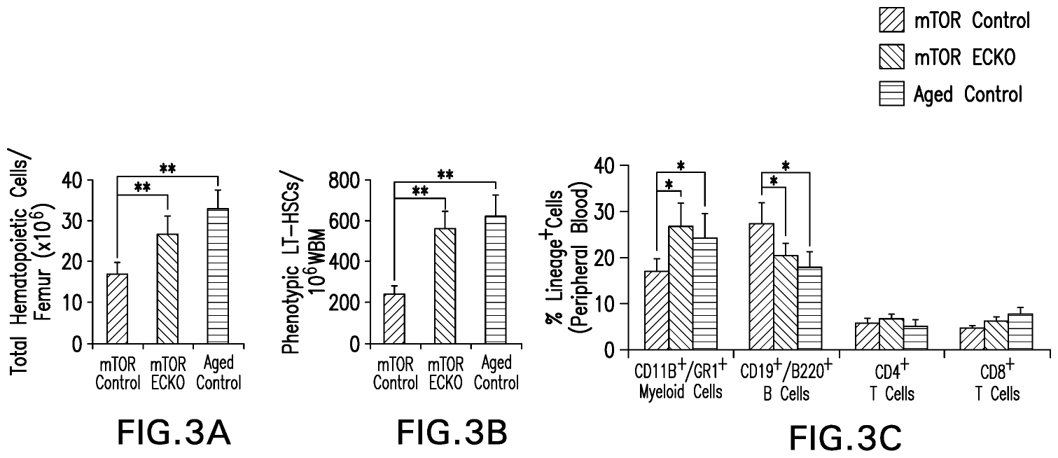

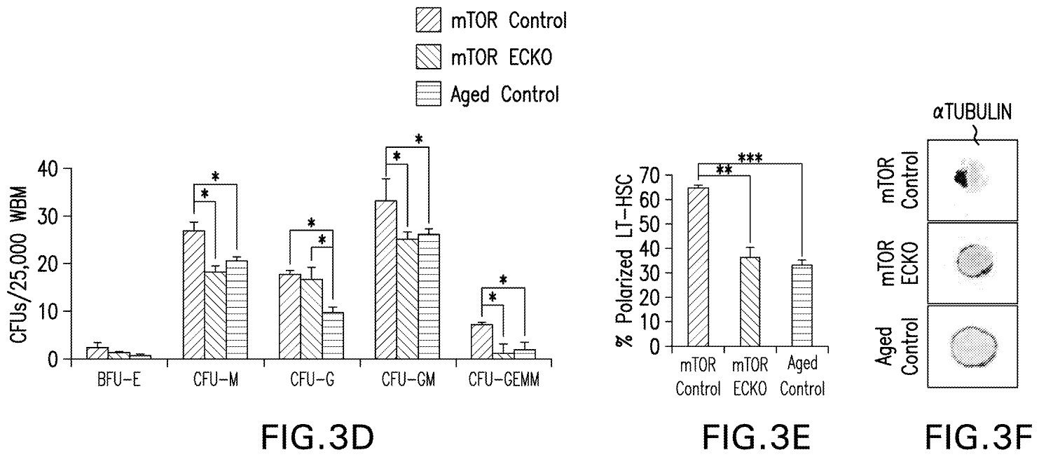

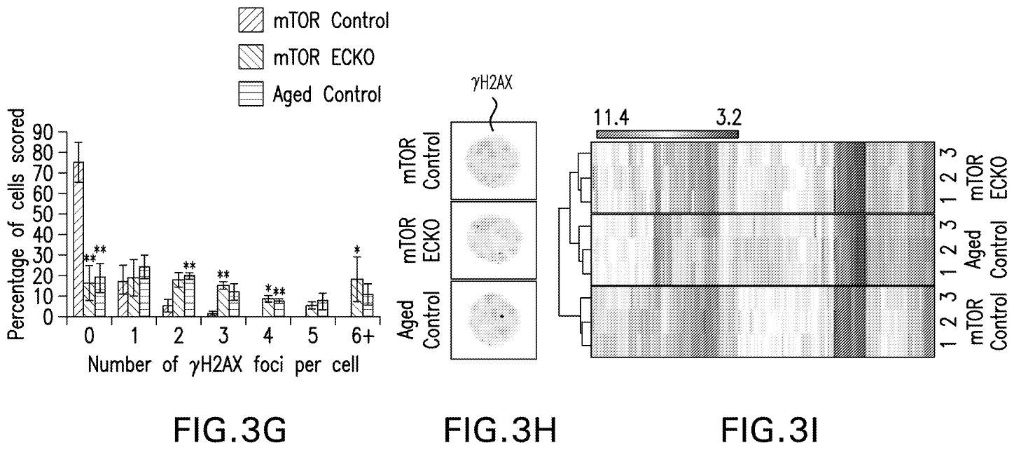

FIGS. 3A, 3B, 3C, 3D, 3E, 3F, 3G, 3H, and 3I show that EC-specific deletion of mTOR (mTOR(ECKO)) caused alterations in HSCs reminiscent of those associated with aging. mTOR^{(ECKO)} resulted in FIG. 3A) a significant increase in total hematopoietic cells;

FIG. 3B, an increased frequency of phenotypic LT-HSCs per femur; FIG. 3C, a significant myeloid bias; FIG. 3D, reduced hematopoietic progenitor activity as assessed by quantifying colony-forming units; FIG. 3F, reduced polarization capacity (quantification); FIG. 3F, reduced polarization capacity (showing representative images of αTUBULIN staining to demarcate cellular polarity; FIG. 3G, increased γH2AX foci (quantification); FIG. 3H, increased γH2AX foci (representative images); and FIG. 3I, transcriptional profiles similar to those of aged controls.

As depicted in FIG. 3A and FIG. 3B mTOR^{(ECKO)} mice displayed a significant increase in both total BM hematopoietic cells and the frequency of phenotypic HSCs, similar to aged controls. As depicted in FIG. 3C, peripheral blood analysis for lineage composition revealed a significant increase in myeloid cells in young mTOR^{(ECKO)} mice and aged controls, with decreased levels of B and T cells as compared to young control mice. FIG. 3D shows that in a methylcellulose colony forming unit (CFU) assay, whole bone marrow (WBM) isolated from mTOR^{(ECKO)} and aged mice displayed a drastic loss of progenitor activity.

HSCs from mTOR^{(ECKO)} mice were further analyzed for levels of γH2AX foci [Flach, J., et al., Replication stress is a potent driver of functional decline in ageing haematopoietic stem cells. Nature, 2014. 512(7513): p. 198-202.] and their polarity status [Florian, M. C., et al., Cdc42 activity regulates hematopoietic stem cell aging and rejuvenation. Cell Stem Cell, 2012. 10(5): p. 520-30]. The γH2AX focus assay represents a fast and sensitive approach for detection of double-strand DNA breaks (DSB); it exploits the phosphorylation of histone variant H2AX (resulting in γH2AX) in response to the induction of DNA double stranded breaks. The phosphorylation is initiated at a site of DSB but extends to the adjacent chromatin area. This event can be visualized microscopically as a distinct focus within a cell using a fluorescent antibody specific for γH2AX. (Ivashkevich, A N, et al., Mutat. Res. (2011) 711 (1-2): 49-60).

As shown in FIG. 3E, FIG. 3F, FIG. 3G, and FIG. 3H, HSCs from mTOR^{(ECKO)} mice and aged controls displayed a significant increase in γH2AX foci and a striking loss of αTUBULIN polarity compared to young control mice.

As depicted in FIG. 3I, transcriptional analysis revealed that EC-specific mTOR deletion leads to changes in HSC gene expression that cluster with aged HSCs).

Next, a specific gene expression signature that characterizes an aged HSC was defined and its presence in the mTOR^{(ECKO)} model was tested. Current microarray data was compared with prior published datasets by Rossi et al [Rossi, D. J., et al., Cell intrinsic alterations underlie hematopoietic stem cell aging. Proc Natl Acad Sci USA, 2005. 102(26): p. 9194-9] and Chambers et al [Chambers, S. M., et al., Aging hematopoietic stem cells decline in function and exhibit epigenetic dysregulation. PLoS Biol, 2007. 5(8): p. e201].

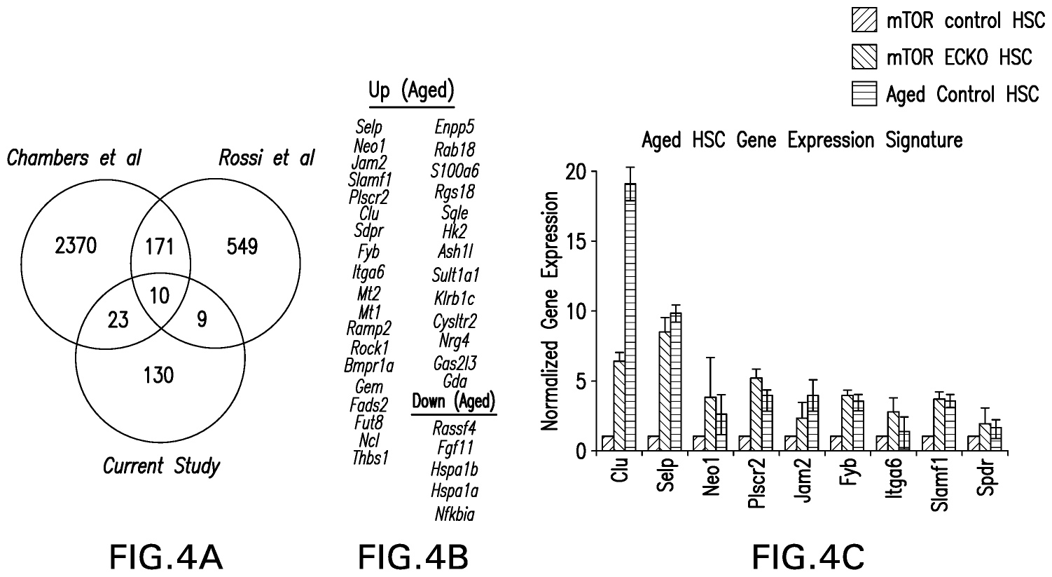

FIG. 4A, 4B, 4C show that mTOR^{(ECKO)} HSCs express an aged HSC gene signature. FIG. 4A is a Venn Diagram comparing significant changes between young and aged HSC transcriptional datasets. FIG. 4B shows common aged HSC gene expression changes. Genes listed demonstrate shared changes in expression between the current study and published datasets whose expression was confirmed in HSCs (red—upregulated in aged HSCs; green—downregulated in aged HSCs). Genes in bold text comprise concordant expression changes between all datasets and represent an aged HSC expression signature. Ten (10) genes were identified that show significant upregulation in expression with aging, nine of which were confirmed by RT-qPCR analysis. mTOR^{(ECKO)}; including SELP, NEO1, JAM2, SLAMF1, PLSCR2, CLU, SDPR, FYB, ITGA6. RASSF4, FGF11, HSPA1B, HSPA1A, and NFKBIA were downregulated. FIG. 4C depicts RT-qPCR confirmation of microarray-identified aged HSC gene expression signature in mTOR^{(ECKO)} and aged mice. Note that HSCs from mTOR^{(ECKO)} share an aged HSC gene expression signature. HSCs also displayed a similar upregulation of the ‘aging-signature’ genes, as observed in aged HSCs

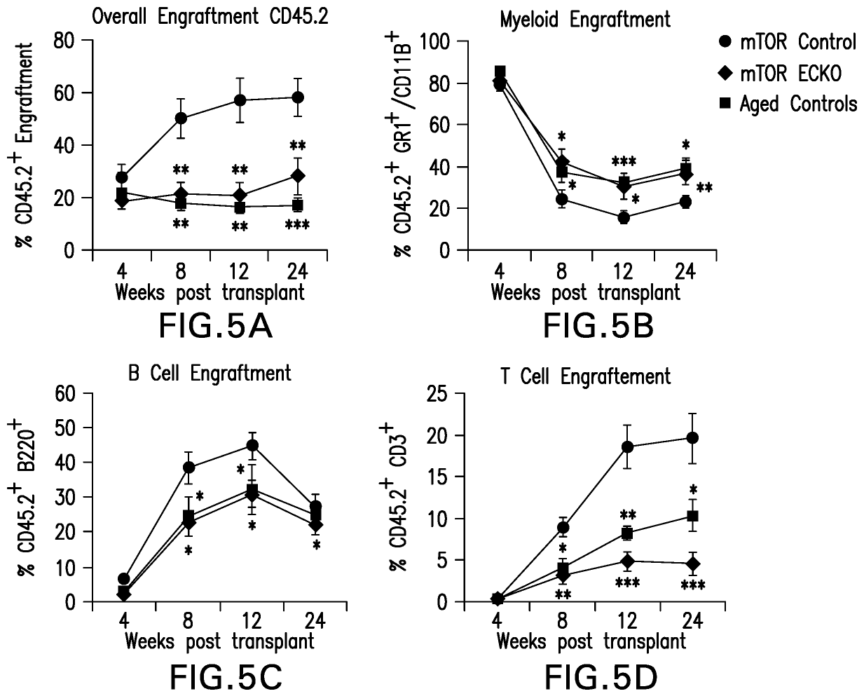

To determine whether these age-related alterations are due to direct effects on the HSCs, the long-term repopulation capacity of HSCs isolated from mTOR^{(ECKO)} mice was examined in a BM transplantation assay. One hundred (100) CD45.2+ HSCs from young mTOR^{(ECKO)} mice, young control mice, and aged control mice were competitively transplanted into lethally-irradiated CD45.1 mice. The data in FIG. 5 show that mTOR^{(ECKO)} HSCs display aged hematopoietic defects following transplantation. x axis, weeks post transplant. FIG. 5A, overall engraftment CD45.2 (y axis, % CD45.2+ engraftment); FIG. 5B, myeloid engraftment (y-axis, % CD45.2+GR1+(CD11B+) engraftment); FIG. 5C, B cell engraftment (y-axis, % CD45.2+B220+ engraftment); FIG. 5D, T cell engraftment (y-axis % CD45.2+CD3+ engraftment). The results showed that similar to aged HSCs, HSCs from young mTOR^{(ECKO)} mice displayed diminished engraftment and a significant myeloid bias at the expense of lymphopoiesis compared to HSCs from young control mice.

Taken together, these observations show that EC-specific mTOR deletion in mice is sufficient to induce transcriptional, phenotypic, and functional premature aging of the HSCs at steady state.

### Example 2 Discovery of a Candidate Pro-HSC-Aging Factor

### Example 2A: Endothelial-Specific Deletion of mTOR or Physiological Aging is Associated with Increased Thrombospondin-1

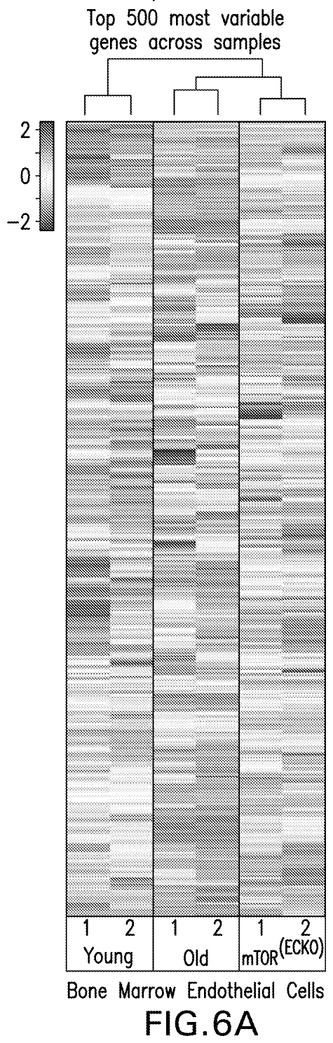

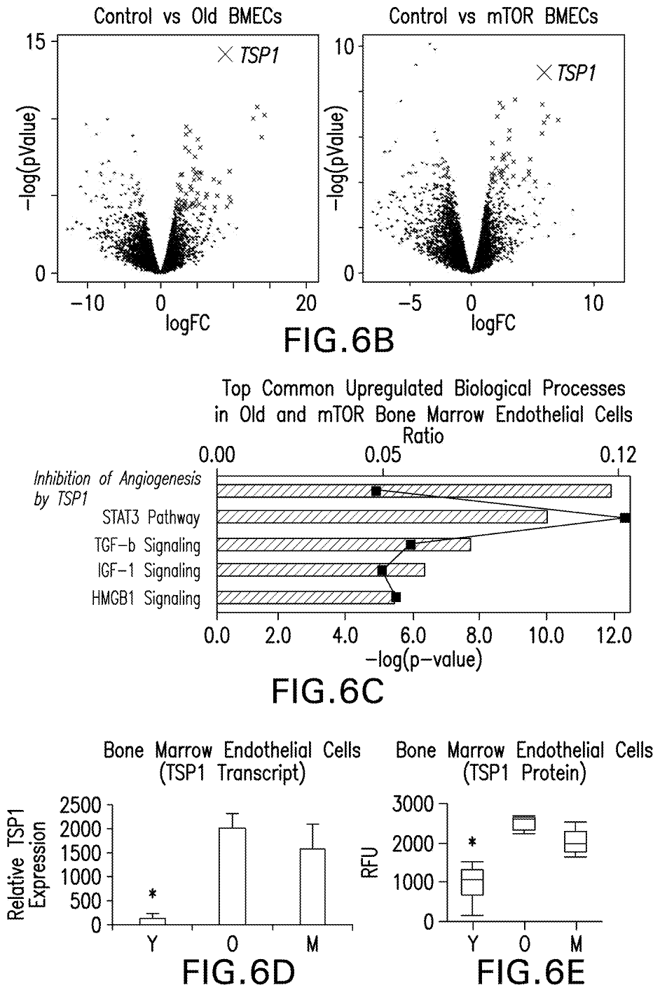

To identify BMEC factors that promote HSC aging, transcriptomes of young mTOR(ECKO) and aged wild type mice were analyzed and compared to those of young wild type controls. The focus was significant changes in gene expression that were common to both mTOR(ECKO) and aged mice relative to young controls. FIG. 6 depicts proteomics analysis on BMECs of young, mTOR^{(ECKO)}, and aged mice. FIG. 6A, a heatmap of conserved gene changes in BMECs isolated from mTOR^{(ECKO)} and aged mice when compared to young mice, shows the top 500 most variable genes across samples. FIG. 6B depicts volcano plots of BMECs isolated from both mTOR^{(ECKO)} and aged mice showing that Thrombospondin-1 (TSP1) was the most significant upregulated gene and had the highest fold change in both cohorts when compared to young control BMECs. FIG. 6C depicts the results of ingenuity pathway analysis, which demonstrated that inhibition of angiogenesis by TSP1 is the top upregulated biological process represented by the transcriptional changes. The other four top processes (STAT3 pathway; TGF-b signaling; IGF-1 signaling, and HMGB1 signaling) are all regulated by Thrombospondin-1

Next, the expression changes of TSP1 in aged and mTOR(ECKO) BMECs were confirmed by transcriptional and protein analysis. Fresh BMECs were isolated from young (12 weeks; “Y”), young mTOR(ECKO) (12 weeks' “M”), and aged (24 months; “O”) mice (n=3; ˜1,500 BMECs/mouse). FIG. 6D shows relative TSP1 gene expression in Y, O, and M BMECs. The data confirmed that the relative gene expression of TSP1 was indeed upregulated in mTOR(ECKO) and aged BMECs Next, BMECs were again isolated from young (12 weeks, “Y”), young mTOR(ECKO) (12 weeks, “M”), and aged (24 months, “O”) mice (n=3; each N was 5 mice pooled in order to achieve optimal protein concentrations). FIG. 6E shows TSP-1 protein levels in Y, O, and M BMECs using an aptamer-based proteomic system (Somalogic). The results show that TSP1 protein levels were elevated in M (mTOR(ECKO) and O (aged) mice. Thus, transcriptome and protein analysis identified Thrombospondin 1 (TSP1) as a pro-HSC-aging factor.

TSP1 is a secreted, matrix-bound glycoprotein that plays major roles in regulating cellular interactions between cells and the surrounding matrix (i.e. laminin, fibronectin, and fibrinogen). TSP1 binds and neutralizes VEGF, blocks VEGFR2 signaling on EC, and destabilizes adhesive contacts between ECs [Gupta, K., et al., Binding and displacement of vascular endothelial growth factor (VEGF) by thrombospondin: effect on human microvascular endothelial cell proliferation and angiogenesis. Angiogenesis, (1999) 3(2): p. 147-58; Kaur, S., et al., Thrombospondin-1 inhibits VEGF receptor-2 signaling by disrupting its association with CD47. J Biol Chem, (2010) 285(50): p. 38923-32; Garg, P., et al., Thrombospondin-1 opens the paracellular pathway in pulmonary microvascular endothelia through EGFR/ErbB2 activation. Am J Physiol Lung Cell Mol Physiol, (2011) 301(1): p. L79-90.]. TSP1 has also been shown to regulate platelet aggregation and is a potent anti-angiogenic factor that is expressed in the BM microenvironment [Agah, A., et al., The lack of thrombospondin-1 (TSP1) dictates the course of wound healing in double-TSP1/TSP2-null mice. Am J Pathol, (2002) 161(3): p. 831-9; Agah, A., et al., Thrombospondin 2 levels are increased in aged mice: consequences for cutaneous wound healing and angiogenesis. Matrix Biol, (2004) 22(7): p. 539-47; Iruela-Arispe, M. L., et al., Thrombospondin-1, an inhibitor of angiogenesis, is regulated by progesterone in the human endometrium. J Clin Invest. (1996) 97(2): p. 403-12]. TSP1 is expressed by mature hematopoietic cells, such as megakaryocytes [Long, M. W. and V. M. Dixit, Thrombospondin functions as a cytoadhesion molecule for human hematopoietic progenitor cells. Blood (1990) 75(12): p. 2311-8; Kyriakides, T. R., et al., Mice that lack thrombospondin 2 display connective tissue abnormalities that are associated with disordered collagen fibrillogenesis, an increased vascular density, and a bleeding diathesis. J Cell Biol. (1998) 140(2): p. 419-30], as well as BMECs (FIG. 6, panel B) [Reed, M. J., et al., Expression of thrombospondins by endothelial cells. Injury is correlated with TSP-1. Am J Pathol, 1995. 147(4): p. 1068-80; DiPietro, L. A., D. R. Nebgen, and P. J. Polverini, Downregulation of endothelial cell thrombospondin 1 enhances in vitro angiogenesis. J Vasc Res, 1994. 31(3): p. 178-85]. It has been demonstrated that hematopoietic recovery after myelosuppression is accelerated in TSP1 knockout mice (TSP1−/−) [Kopp, H. G., et al., Thrombospondins deployed by thrombopoietic cells determine angiogenic switch and extent of revascularization. J Clin Invest, (2006) 116(12): p. 3277-91.]. Without being limited by theory, these data suggest that one mechanism by which recovery of the BM revascularization is regulated is through the inhibition of TSP1 by BMECs, thereby allowing for the regeneration and restabilization of the BM vascular niche [Agah, A., et al., Thrombospondin 2 levels are increased in aged mice: consequences for cutaneous wound healing and angiogenesis. Matrix Biol, (2004) 22(7): p. 539-47; Kyriakides, T. R., et al., Megakaryocytes require thrombospondin-2 for normal platelet formation and function. Blood, (2003) 101(10): p. 3915-23; Huh, H. Y., et al., CD36 induction on human monocytes upon adhesion to tumor necrosis factor-activated endothelial cells. J Biol Chem. (1995) 270(11): p. 6267-71; Bornstein, P., et al. Thrombospondin 2, a matricellular protein with diverse functions. Matrix Biol, (2000) 19(7): p. 557-68; Bornstein, P., et al., Thrombospondin 2 modulates collagen fibrillogenesis and angiogenesis. J Investig Dermatol Symp Proc, (2000) 5(1): p. 61-6.].

### Example 2B: TSP1 Knockout Mice have Increased HSC and Progenitor Function

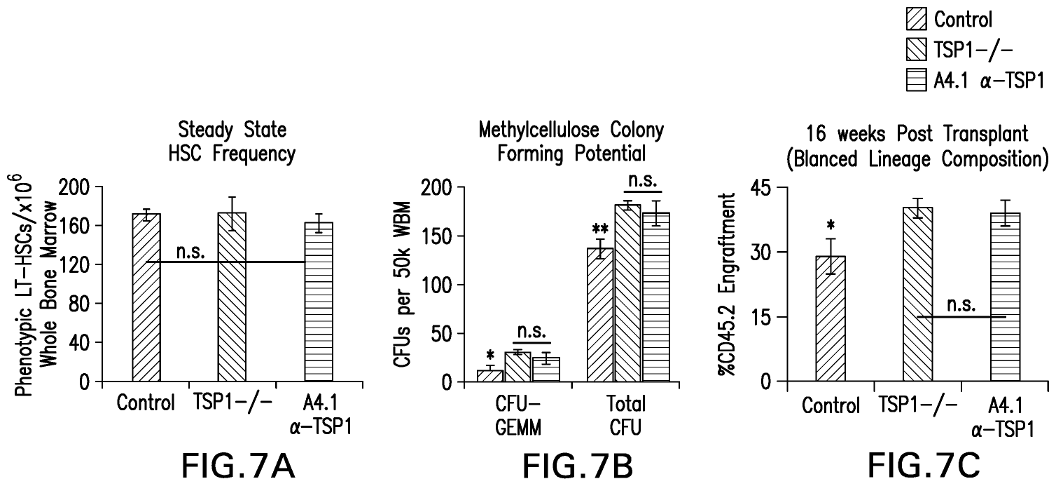

To date, most data generated on TSP1−/− mice has been done in the context of injury and regeneration. Very little is known about the role of TSP1 in steady state hematopoiesis. To address this gap in knowledge, hematopoietic cells were isolated from TSP1−/− (Jax Lab: 006141) mice and the frequency and function of HSCs was assessed. FIG. 7 shows that inhibition of TSP1 in young mice increases HSC numbers and function. FIG. 7A is a steady state analysis of phenotypic LT-HSCs in control, TSP-1−/− mice, and control mice that received infusions of a neutralizing antibody to TSP-1. In young TSP1−/− mice there were no differences in the number of phenotypic HSCs. Next, whole BM was isolated from TSP1−/− mice and a methylcellulose-based progenitor colony-forming colony assay was performed. The data is shown in FIG. 7B; TSP1−/− mice had significantly more primitive CFU-GEMMs and overall more total CFUs; other colony types were not changed). Additionally, 100 phenotypic HSCs were infused in a competitive transplantation assay from control, TSP-1−/− mice, and control mice that received infusions of a neutralizing antibody to TSP-1. The results showed that TSP1−/− mice harbored HSCs that were more robust in their engraftment potential without any alterations in lineage-specific reconstitution (data not shown). Furthermore, the activity of commercially available neutralizing antibody to TSP1 (clone A4.1, Thermofisher, Invitrogen RRID AB_10988669)) was measured by treating control, young C57BL6 mice with 4 μg of uTSP1 every day for 3 days. This dosing concentration and regimen were determined by performing a dose response experiment, which showed that 4 μg was the maximal concentration to increase HSC numbers and that after 3 days there was no additional benefit. Antibody treatment elicited responses similar to those seen in the TSP1 knockout mice as shown in FIGS. 7A, 7B and 7C. The results showed that TSP1−/− mice or mice treated with a TSP1 inhibitor resulted in an increase in HSC function. These data support the potential of TSP1 inhibition as a therapeutic modality.

In addition to neutralizing antibodies, other techniques for knocking down gene expression are known. These include, without limitation, siRNA and miRNA based RNAi¹⁻⁴, anti-sense oligonucleotides⁵ and CRISPR/TALEN/zinc finger endonuclease⁶⁻¹⁰ based gene editing. Accordingly, these additional techniques can be used to knowkdown TSP1 gene expression both in vitro and in vivo.

The CRISPR-Cas system relies on two main components: a guide RNA (gRNA) and CRISPR-associated (Cas) nuclease.

The guide RNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA, which serves as a binding scaffold for the Cas nuclease.

The CRISPR-associated protein is a non-specific endonuclease. It is directed to the specific DNA locus by a gRNA, where it makes a double-strand break. There are several versions of Cas nucleases isolated from different bacteria. The most commonly used one is the Cas9 nuclease from Streptococcus pyogenes. Single guide RNA (sgRNA) is a single RNA molecule that contains both the custom-designed short crRNA sequence fused to the scaffold tracrRNA sequence. sgRNA can be synthetically generated or made in vitro or in vivo from a DNA template.

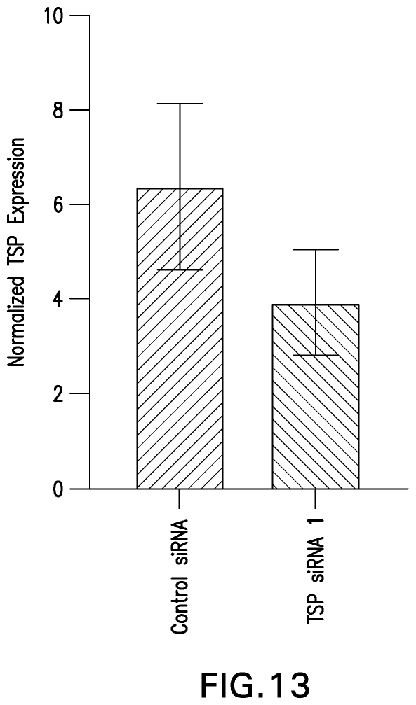

Efficient knockdown of TSP1 gene expression via siRNA delivery. FIG. 13 is a bar graph of normalized TSP expression (y-axis) vs. sample (control siRNA, TSP siRNA 1). 10 nM siRNA [Thbs1 siRNA #1, ID S75095, Thermofisher] was transfected utilizing Lipofectamine RNAiMax, and total RNA was purified 48 hours following transfection utilizing TRIZOL Reagent. cDNA was synthesized from the purified RNA (superscript 3) and qPCT (Applied Biosystems) performed utilizing primers targeting Thbs1. Expression was normalized to beta-actin. The data demonstrate a decrease in expression of TSP1 mRNA in endothelial cells following transfection of siRNA targeting TSP1.

Commercially available sequences are as follows:

Thbs1 siRNA #1

| | |
|---|---|
| SIRNA ID | s75095 |
| Catalog # | 4390771 (www.thermofisher.com) |

Sequence (5′-3′)
Sense strand:
(SEQ ID NO: 1)
GAACUUGUCCAGACUGUAAtt
Antisense strand:
(SEQ ID NO: 2)
UUACAGUCUGGACAAGUUCtt

| Thbs1 siRNA #2 | |
|---|---|
| SIRNA ID | s75096 |
| Catalog # | 4390771 |

Sequence (5′-3′)
Sense strand:
(SEQ ID NO: 3)
CAACGAGGAGUGGACUGUAtt
Antisense strand:
(SEQ ID NO: 4)
UACAGUCCACUCCUCGUUGtt

Negative Control siRNA:
Sense strand:
(SEQ ID NO: 5)
UUCUCCGAACGUGUCACGUtt
Antisense strand:
(SEQ ID NO: 6)
ttAAGAGGCUUGCACAGUGCA

https://www.thermofisher.com/crispr/invitrogen/query/thbs1:

TrueGuide ™ Synthetic sgRNA

| | |
|---|---|
| 1. Cat # A35533 | ID: CRISPR573571_SGM |
| Target DNA Sequence: | GGCATTCTCAATGCGGAAGG |
| | (SEQ ID NO: 7) |
| Target locus | Chr.2: 118113072- |
| | 118113094 on GRCm38 |
| Strand | Forward |
| Application | Gene Knockout |
| 2. Cat # A35533 | ID: CRISPR573574_SGM |
| Target DNA Sequence | AACTCATTGGAGGTGCACGA |
| | (SEQ ID NO: 8) |
| Target locus | Chr.2: 118113006- |
| | 118113028 on GRCm38 |
| Strand | Forward |
| Application | Gene Knockout |

Negative Control gRNA (Mus musculus)

| | |
|---|---|
| gRNA sequence | GCGAGGTATTCGGCTCCGCG |
| | (SEQ ID NO: 9) |

Source:
https://www.addgene.org/66895/
https://www.ncbi.nlm.nih.gov/pmc/articles/
PMC4486245/

### CITED REFERENCES

1 Fire, A. et al. Potent and specific genetic interference by double-stranded RNA in Caenorhabditis elegans. Nature 391, 806-811, doi:10.1038/35888 (1998).
2 Carthew, R. W. & Sontheimer, E. J. Origins and Mechanisms of miRNAs and siRNAs. Cell 136, 642-655, doi:10.1016/j.cell.2009.01.035 (2009).
3 Sheridan, C. With Alnylam's amyloidosis success, RNAi approval hopes soar. Nature biotechnology 35, 995-997, doi:10.1038/nbt1117-995 (2017).
4 Setten, R. L., Rossi, J. J. & Han, S. P. The current state and future directions of RNAi-based therapeutics. Nat Rev Drug Discov 18, 421-446, doi:10.1038/s41573-019-0017-4 (2019).
5 Rinaldi, C. & Wood, M. J. A. Antisense oligonucleotides: the next frontier for treatment of neurological disorders. Nat Rev Neurol 14, 9-21, doi:10.1038/nrneurol.2017.148 (2018).
6 Doudna, J. A. & Charpentier, E. Genome editing. The new frontier of genome engineering with CRISPR-Cas9. Science 346, 1258096, doi:10.1126/science.1258096 (2014).
7 Zipkin, M. CRISPR's “magnificent moment” in the clinic. Nature biotechnology, doi:10.1038/d41587-019-00035-2 (2019).
8 CRISPR's powers unleashed for disease detection. Nature 554, 406, doi:10.1038/d41586-018-02200-0 (2018).
9 Joung, J. K. & Sander, J. D. TALENs: a widely applicable technology for targeted genome editing. Nat Rev Mol Cell Biol 14, 49-55, doi:10.1038/nrm3486 (2013).
10 Urnov, F. D., Rebar, E. J., Holmes, M. C., Zhang, H. S. & Gregory, P. D. Genome editing with engineered zinc finger nucleases. Nat Rev Genet 11, 636-646, doi:10.1038/nrg2842 (2010).

### Example 2C: Inhibition of TSP1 Preserves HSC Function in Aged Mice

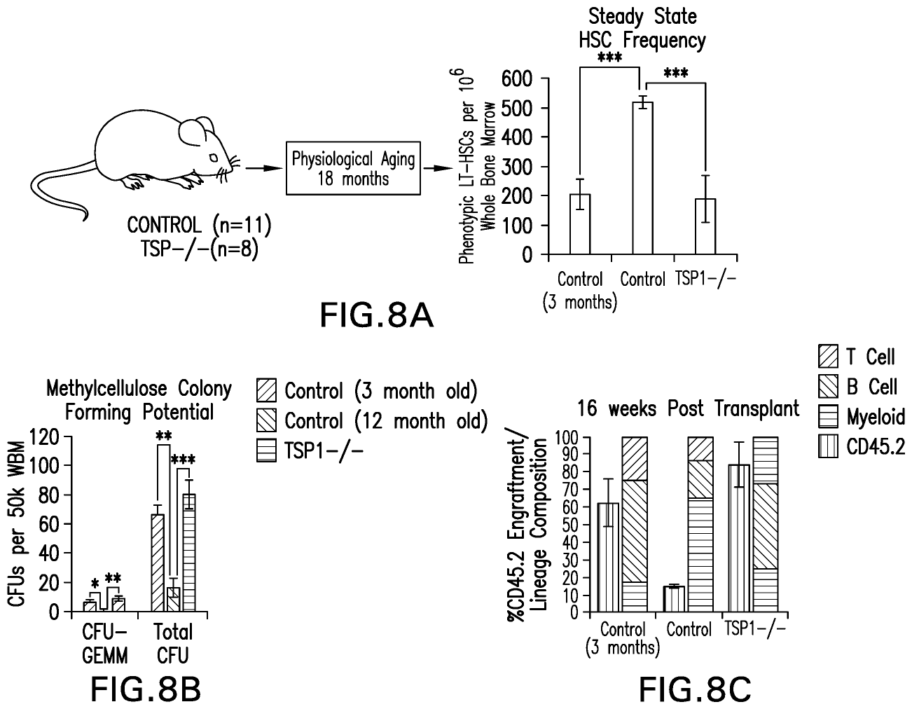

To determine if loss/inhibition of TSP1 can prevent/suppress aging phenotypes observed in the hematopoietic system, control and TSP1−/− mice were aged for 18 months Young 3-month old mice served as controls. FIG. 8A is a schematic of the protocol, and provides a steady state analysis of phenotypic LT-HSCs in all three cohorts. 18-month old control mice had a significant and robust increase in the frequency of phenotypic HSCs, whereas, TSP−/− mice had HSC numbers similar to young control mice (FIG. 8A). Further, WBM was isolated from the three cohorts, and subjected to a progenitor colony-forming assay. Results are shown in FIG. 8B. WBM from aged TSP−/− mice preserved their colony forming potential when put in a methylcellulose. Finally, 100 phenotypic HSCs were infused in a competitive transplantation assay from the three cohorts. Results are shown in FIG. 8C. The results showed that HSCs from aged TSP1−/− mice resemble HSCs isolated from young controls. The engraftment potential and lineage composition of young 3-month old control HSCs were indistinguishable from HSCs transplanted from aged TSP−/− mice while aged control HSCs had decreased engraftment levels and developed a myeloid bias at the expense of lymphopoiesis. Together, the data suggest that TSP1 loss has rejuvenative effects on the hematopoietic system.

### Example 3A. Inhibition of Thrombospondin-1 (TSP1) Preserves Hematopoietic Stem Cell (HSC) Function in Aged Mice

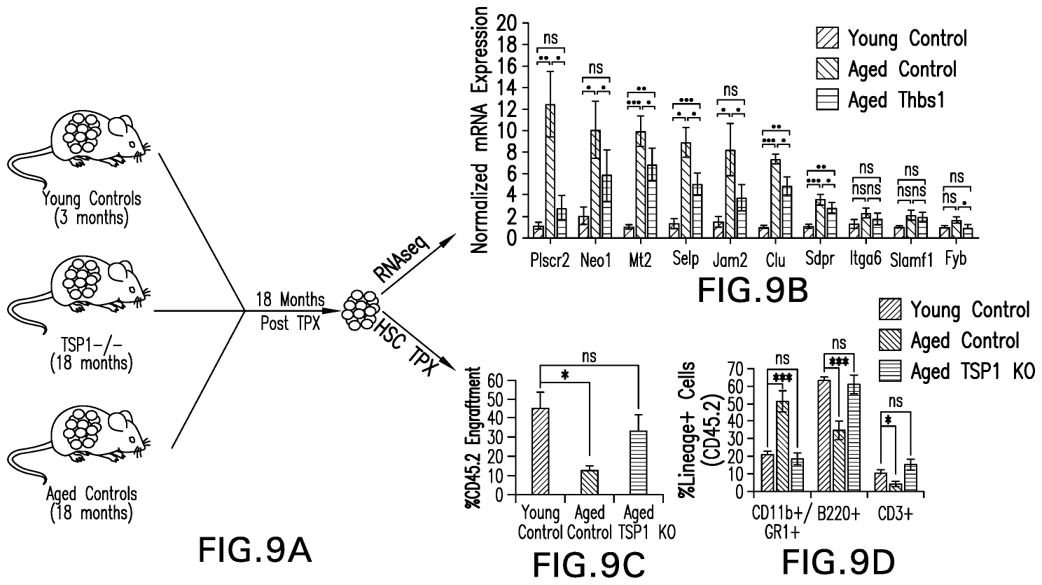

FIG. 9 shows that aged TSP1−/− mice have preserved HSC function. To determine if loss/inhibition of TSP1 can prevent/suppress aging phenotypes observed in the hematopoietic system, we physiologically aged control and global TSP1 knockout mice (TSP1−/−) for 18 months; FIG. 9A is a depiction of the three cohorts (Young Controls, Aged Controls, and Aged TSP1 mice) used for HSC transplants and RNA sequencing. Young 3-month old mice served as controls.

To examine the functional potency of aged HSCs from TSP1−/− mice, we transplanted 100 phenotypic HSCs from young control and aged control and aged TSP1−/− mice and found that the engraftment potential and lineage composition of young 3-month old control HSCs were indistinguishable from HSCs transplanted from aged TSP1−/− mice. FIG. 9C is a bar graph of % CD45.2 engraftment (y axis) in the three cohorts of FIG. 9A (x-axis). FIG. 9D is a bar graph of % lineage+ cells (CD45.2) (y-axis) vs. myeloid peripheral blood cell type (CD11 b+/GR1+), B cell (B220+) and T cell (CD3+) populations (x-axis) in the three cohorts. As shown in FIG. 9C and FIG. 9D, aged control HSCs had decreased engraftment levels and developed a myeloid bias at the expense of lymphopoiesis. Utilizing the same HSC pool that was used for the transplantation, we subjected the HSCs to RNA sequencing and determined that the HSCs isolated from aged TSP1−/− mice were transcriptionally identical to young HSCs. FIG. 9B is a bar graph of normalized mRNA expression (y-axis) vs. genes associated with HSC aging (x-axis). HSCs were isolated from the three cohorts depicted in FIG. 9A and subjected to RNA sequencing. Genes that are associated with HSC aging were decreased in aged HSCs from TSP1−/− mice. Together, these data suggest that TSP1 loss has rejuvenative effects on the hematopoietic system.

### Example 3B. TSP1 Directly Affects the Expansion of Young HSCs

Myelosuppression sets off a remarkable adaptation in hematopoiesis that sacrifices HSC quiescence to meet an urgent need for new blood cell production. Under these conditions, HSCs undergo a significant increase in their self-renewal, proliferation, and lineage-directed differentiation. This process has been difficult to reproduce in an ex vivo setting, with current ex vivo HSC expansion strategies inevitably leading to HSC exhaustion and inducing differentiation to progenitors incapable of long-term engraftment. Another major obstacle to the safe and effective expansion of HSCs has been a lack of methodologies that recapitulate the complexity of hematopoiesis ex vivo. The development of ex vivo expansion strategies of bona fide HSCs will help alleviate the morbidity and mortality associated with prolonged cytopenias bone marrow (BM) transplants and expand the pool of potential donors for allogeneic BM transplants. Recently, we have been successful in co-opting an in vitro system to deliver compounds (e.g., neutralizing antibodies and recombinant proteins) that provide the physiological signals necessary to orchestrate HSC homeostasis in a way that preserves HSC self-renewal capacity and allows for the expansion of engraftable HSCs with balance lineage distribution [1-4]. Utilizing this system, we have been successful in validating critical signaling molecules that are required for HSC expansion and maintenance [1-6]. Utilizing this system, we have tested whether inhibition of TSP1 signaling could directly promote HSC expansion and functional output, significantly advancing the development of strategies for accelerating hematopoietic recovery. T

To this end, we ex vivo expanded HSCs utilizing polyvinyl alcohol (PVA)[7] in a BSA-free, low dose KitL (10 ng/ml) and TPO (100 ng/ml) to determine whether exogenous TSP1 directly or indirectly supports HSC function. We first set out to test if exogenous TSP1 could directly affect the function of young HSCs. Using the PVA expansion protocol, we cultured 300 sorted phenotypic HSCs in a fibronectin-coated 96-well format with and without 500 ng/ml of TSP1. Additionally, we included cohorts that received 3 independent TSP1 neutralizing antibodies and their IgG controls.

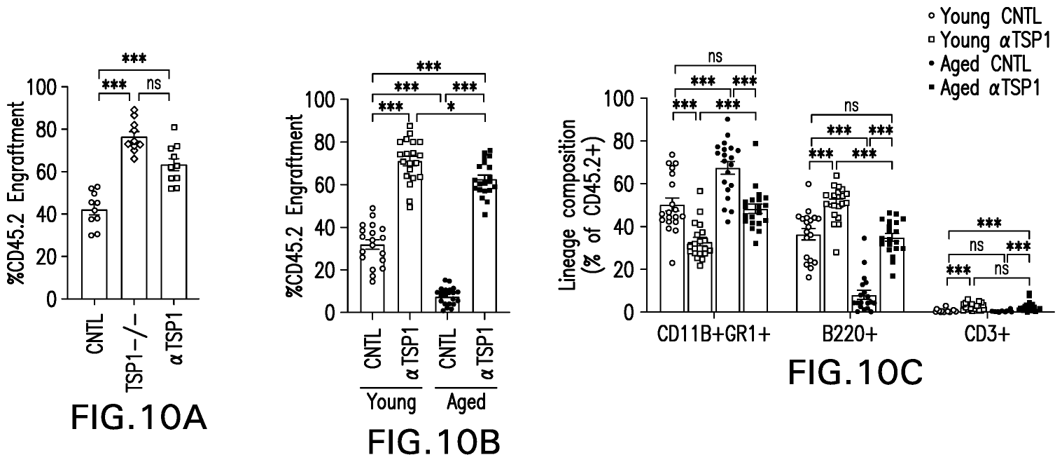

FIG. 10 shows that TSP1 directly affects the expansion of young HSCs. FIG. 10A is a schematic demonstrating the ex vivo expansion protocol to test whether exogenous TSP1 can influence HSC expansion and function. FIG. 10B is a bar graph of % CD45.2 engraftment (y-axis) of cells treated (from left to right) with rTSP1 (500 ng/ml); uTSP1 neutralizing antibody clone 1 [ThermoFisher Scientific; MA5-13398]; uTSP1 neutralizing antibody clone 2[ThermoFisher Scientific; MA5-13385]; Ms IgG1k IgG control [ThermoFisher Scientific; 16-4714-82]; uTSP neutralizing antibody clone 3; and Ms IgM control (x axis) [ThermoFisher Scientific; 14-4752-82]. FIG. 10C is a bar graph of % lineage+ cells (CD45.2, y-axis) showing myeloid lineage (CD11b/GR1+), lymphoid [B220, B cell; CD3 T cell] lineage distributions 24 weeks post-transplant.

Following an 11 day expansion, HSCs were competitively transplanted, and engraftment was assessed 24 months post-transplant. We did not see any significant differences in the expansion frequency of phenotypic HSCs, but upon transplantation of 104 total expanded cells with 10⁶ CD45.1 competitors, we found that hematopoietic cells treated with TSP1 led to a significant decrease in the engraftment potential with very little differences in lineage reconstitution (FIG. 10A, 10B, 10C). Two of the three neutralizing antibodies (Clone 1 and 2) did not block the deleterious effects of exogenous TSP1 to the functional output of the expanded HSCs. However, Clone 3 resulted in the expansion of HSCs that gave rise to robust hematopoietic engraftment with balanced lineage distribution (FIG. 10A, 10B, 10C).

### Example 3C. Neutralizing Antibody to TSP1 can Rejuvenate and Ex Vivo Expand Aged HSCs

We first set out to test how efficient our neutralizing antibody was at inhibiting TSP1 signaling in treated HSCs. We ex vivo expanded young HSCs isolated from control and TSP1 global knockout mice in our PVA protocol and competitively transplanted the HSCs.

FIG. 11 shows that TSP1 directly affects the expansion of young HSCs. Ex-vivo expanded young HSCs were isolated from control and TSP1 global knockout (KO) mice in the PVA protocol and the HSCs competitively transplanted. Following an 11-day expansion, HSCs were competitively transplanted, and engraftment was assessed 24 months post-transplant.

As shown in FIG. 11A (bar graph of % CD45.2 engraftment (y-axis) vs. Control, TSP1−/−, uTSP1 antibody treated [ThermoFisher Scientific; MA5-13377] (x axis)), long-term, multilineage engraftment showed that HSCs treated with the TSP1 neutralization antibody engrafted similar to TSP1 knockout HSCs; both conditions out-performed control HSCs.

We next set out to test if inhibiting TSP1 signaling in aged HSCs could rejuvenate their function. We isolated HSCs from young and aged (18-month old) mice and subjected them to our ex vivo expansion protocol with and without the TSP1 neutralizing antibody. FIG. 11B is a bar graph of % CD45.2 engraftment (y-axis) in Young (control, uTSP1-treated), and aged (control, uTSP1-treated) HSCs (x-axis). FIG. 11C is a bar graph of lineage composition (% of CD45.2+, y axis) vs. myeloid (CD11B+GR1+), lymphoid (B cell, B220+, T cell, CD3+) young (control, uTSP1-treated) and aged (control, α-TSP1-treated) (x-axis) HSCs 24 weeks post-transplant.

We found that aged HSCs treated with the antibody were able to achieve long-term engraftment, superior to both young and aged non-antibody treated HSCs (FIG. 11B, 11C). Furthermore, aged HSCs treated with the neutralizing antibody were able to provide balanced lineage engraftment, unlike their control counterparts which manifested a myeloid bias at the expense of lymphopoiesis (FIG. 11B,C).

As shown in FIG. 12, in addition to preserving and rejuvenating the functional output of aged HSCs, inhibition of TSP1 improves many indicators of aging and frailty.

FIG. 12A shows representative images of aged TSP1 mice alongside young controls and aged controls. Note the loss and graying of hair in aged controls, whereas aged TSP1 mice look similar to young controls. FIG. 12B is a bar graph showing body weight (g) (y-axis) vs. young control, aged control and aged TSP1 KO mice (x axis). When comparing coat color and body size of aged (18 months) TSP1 mice to young and aged controls, we found that aged TSP1 mice have smooth, shiny coats and have lower body weights that are similar to young controls (FIG. 12A, 12B).

FIG. 12C shows VE cadherin (red)/perilipin (green)/DAPI (blue) staining in the bone marrow microenvironment in young control, aged control and aged TSP1 KO mice (x-axis). Infiltration and accumulation of perilipin+ adipocytes within the BM microenvironment is common in aged mice. However, aged TSP1 do not manifest an increase in adipocytes and look similar to young controls (FIG. 12C).

To confirm that the lack of weight gain in aged TSP1 was not specific to the BM but the whole body, we subjected TSP1 mice and controls to a DEXAScan. FIG. 12D shows fat/body weight ratio (DEXAScan, y-axis) vs. control and TSP1 KO mice. FIG. 12H shows DEXAScan used to determine bone mineralization to weight ratios in control and TSP1 KO mice. We found that indeed TSP1 mice had significantly less fat accumulation. Additionally, we found that TSP1 mice had an increase in bone mineralization, suggesting that they do not manifest a loss in bone mass or strength (FIG. 12D, 12H).

Based on the lack of weight gain in aged TSP1 mice, we performed blood chemistry analysis. FIGS. 12E, 12F, and 12G show blood chemistry for cholesterol (FIG. 12E), insulin (FIG. 12F), and fasted glucose levels (FIG. 12G) for control and TSP1KO mice. We found that TSP1 mice have reduced total cholesterol and triglycerides, with an increase in good, HDL cholesterol (FIG. 12E). Furthermore, they had lower insulin levels and a decrease in fasted blood glucose levels (FIG. 12F,G).

Lastly, TSP1 mice were subjected to a grip strength test. FIG. 12I shows forelimb/hindlimb grip strength in control and TSP1KO mice. We found that TSP1 mice had an increase in forelimb/hindlimb grip strength (FIG. 12I).

Taken together, these data indicate that inhibition of TSP1 can improve overall healthspan by reducing the risk of cardiovascular disease and obesity, as well as preserving indicators of frailty.

### REFERENCES FOR EXAMPLE 3

1. Butler, J. M., et al., Development of a vascular niche platform for expansion of repopulating human cord blood stem and progenitor cells. Blood, 2012. 120(6): p. 1344-7.
2. Poulos, M. G., et al., Vascular Platform to Define Hematopoietic Stem Cell Factors and Enhance Regenerative Hematopoiesis. Stem Cell Reports, 2015. 5(5): p. 881-94.
3. Poulos, M. G., et al., Endothelial-specific inhibition of NF-kappaB enhances functional haematopoiesis. Nat Commun, 2016. 7: p. 13829.
4. Poulos, M. G., et al., Endothelial transplantation rejuvenates aged hematopoietic stem cell function. J Clin Invest, 2017. 127(11): p. 4163-4178.
5. Butler, J. M., et al., Endothelial cells are essential for the self-renewal and repopulation of Notch-dependent hematopoietic stem cells. Cell Stem Cell, 2010. 6(3): p. 251-64.
6. Poulos, M. G., et al., Endothelial jagged-1 is necessary for homeostatic and regenerative hematopoiesis. Cell Rep, 2013. 4(5): p. 1022-34.
7. Wilkinson, A. C., et al., Long-term ex vivo expansion of mouse hematopoietic stem cells. Nat Protoc, 2020. 15(2): p. 628-648.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. A method for rejuvenating an aging blood and vascular system comprising aging-associated hematopoietic defects in an aging hematopoietic microenvironment of bone marrow including deteriorating vascular integrity, reduced hematopoietic stem cell function, or both, comprising
administering to a subject a pharmaceutical composition comprising an inhibitor of an angiocrine factor, a splice variant, or a fragment thereof, wherein the angiocrine factor is thrombospondin 1 (TSP1), and a pharmaceutically acceptable carrier;
optionally administering a stem cell co-therapy comprising transplantation of a therapeutic amount of multipotent, self-renewing hematopoietic stem cells (HSCs) effective to regenerate the blood system and promote hematopoietic reconstitution of the bone marrow, and
optionally administering a vascular endothelial co-therapy comprising transplantation of a therapeutic amount of endothelial cells (ECs) effective to regenerate the blood system and promote hematopoietic reconstitution of the bone marrow, and
enhancing hematopoietic recovery in the hematopoietic bone marrow microenvironment by one or more of:
reducing inflammation in the hematopoietic microenvironment of the bone marrow; preserving vascular integrity in the hematopoietic microenvironment of the bone marrow; or
increasing frequency and numbers of cell types in the hematopoietic compartment to effect multi-lineage reconstitution.

2. The method according to claim 1, wherein the inhibitor of TSP1 is an antibody, an siRNA, or TSP1 gene knockout by CRISPR-comprising a synthetic single guide RNA.

3. The method according to claim 2,
(a) wherein the antibody is a non-neutralizing antibody to TSP1; or
(b) wherein the antibody is a neutralizing antibody to TSP1.

4. (canceled)

5. The method according to claim 3, wherein the neutralizing antibody is commercially available as clone A4.1 (Thermofisher, Invitrogen RRID AB_10988669)).

6. The method according to claim 1, wherein
a. the HSC niche comprises hematopoietic stem cells (HSCs), hematopoietic progenitor cells (HPCs), resident niche cells comprising osteoblastic cells that regulate stem cell pool size during hematopoiesis, and secreted and membrane bound factors comprising chemokines, wherein at steady state, the HSCs are mostly quiescent, while HPCs are actively proliferating and contributing to daily hematopoiesis; and
b. the vascular niche comprises an endothelial microniche comprising endothelial cells comprising bone marrow endothelial cells (BMECs), which, when activated, produce angiocrine factors that orchestrate a system of cellular crosstalk that results in differential production of the angiocrine factors.

7. The method according to claim 6, wherein the aged endothelial microenvironment within the aged bone marrow hematopoietic microenvironment of the HSC niche containing aged BMECs includes one or more of
a decrease in mTOR signaling,
a reduced abundance of an mTOR subunit,
reduced phosphorylation of mTOR catalytic subunits,
reduced expression of mTOR transcription target genes; or
reduced protein levels in mTOR catalytic subunit mTOR Complex 1 and mTOR Complex 2.

8. The method according to claim 7,
(a) wherein the decrease in mTOR signaling by BMECs causes functional defects associated with aging in aged HSCs; or
(b) wherein expression levels of thrombospondin-1 (TSP1) are upregulated in aged BMECs when compared to a young control.

9. (canceled)

10. The method according to claim 8, wherein top upregulated biological processes represented by changes in gene expression in aged BMECs, compared to a young control, which include changes in STAT3 pathway, TGF-b signaling, IGF-1 signaling or HMGB1 signaling, are regulated by TSP1.

11. The method according to claim 1, wherein the deteriorating vascular integrity comprises increased vascular permeability including increased endothelial permeability, increased endothelial inflammation, or both.

12. The method according to claim 1, wherein aging-associated hematopoietic defects in the HSC niche of the bone marrow hematopoietic microenvironment include one or more of:
sustained inflammation;
increased HSC cellularity
increased stem cell pool size;
loss of HSC quiescence;
increased HSC apoptosis
loss of HSC self-renewal potential;
increased myeloid-biased differentiation of the HSCs,
increased risk of failure of myeloablative strategies; or
reduced engraftment and regeneration of the bone marrow niche after transplantation, compared to a young control.

13. The method according to claim 12, wherein the sustained inflammation is derived from a myelosuppressive insult.

14. The method according to claim 13,
(a) wherein the myelosuppressive insult comprises exposure to radiation, chemotherapy or both; or
(b) wherein the myelosuppressive insult comprises chemotherapy; or
(c) wherein the myelosuppressive insult is myeloablative.

15. (canceled)

16. (canceled)

17. The method according to claim 12,
(a) wherein the increased myeloid-biased differentiation of the HSCs is at expense of lymphopoiesis; or
(b) wherein the loss of quiescence for HSCs leads to a transient increase in HSCs, long-term exhaustion of HSCs, and defects in long-term repopulation capacity of HSCs; or
(c) wherein aging-associated hematopoietic defects in the HSC niche of the bone marrow hematopoietic microenvironment include changes in HSC gene expression.

18. (canceled)

19. The method according to claim 17, wherein overactivation of mTOR drives HSCs from quiescence into more active cell cycling.

20. (canceled)

21. The method according to claim 19, wherein the changes in HSC gene expression associated with aging in aged HSCs comprise upregulation of one or more of SELP, NEO1, JAM2, SLAMF1, PLSCR2, CLU, SDPR, FYB, ITGA6 and downregulation of downregulation of one or more of RASSF4, FGF11, HSPA1B, HSPA1A, or NFKBIA.

22. A method for preparing a hematopoietic stem cell product for hematopoietic stem cell transplantation comprising
(a) preparing ex vivo cultures of hematopoietic stem cells;
(b) administering an antibody comprising anti-TSP1 antibodies to the cultures of hematopoietic stem cells in (a) to form a treated hematopoietic stem cell population; and
(c) expanding the treated hematopoietic stem population in vitro to form a hematopoietic stem cell transplantation product comprising a therapeutic amount of treated hematopoietic stem cells, wherein engraftment potential of the hematopoietic stem cell transplantation product is enhanced compared to an untreated control.

23. The method according to claim 22,
(a) wherein the hematopoietic stem cells of step (a) are derived from a human subject; or
(b) wherein the hematopoietic stem cells of step (a) are derived from a mouse subject; or
(c) wherein the antibody comprising the anti-TSP1 antibodies are neutralizing antibodies; or
(d) wherein the anti-TSP1 antibodies further comprise antibodies to CD36, CD47 or both; or
(e) wherein the antibodies are humanized antibodies; or
(f) wherein the hematopoietic stem cell transplantation is allogeneic.

24. (canceled)

25. (canceled)

26. (canceled)

27. (canceled)

28. (canceled)

29. (canceled)


SEQUENCE LISTING (9 sequences)

SEQ ID NO: 1 - DNA, 21 bp, Artificial Sequence
  Description of Artificial Sequence Synthetic oligonucleotide
  Description of Combined DNA/RNA Molecule Synthetic oligonucleotide
gaacuuguccagacuguaatt

SEQ ID NO: 2 - DNA, 21 bp, Artificial Sequence
  Description of Artificial Sequence Synthetic oligonucleotide
  Description of Combined DNA/RNA Molecule Synthetic oligonucleotide
uuacagucuggacaaguuctt

SEQ ID NO: 3 - DNA, 21 bp, Artificial Sequence
  Description of Artificial Sequence Synthetic oligonucleotide
  Description of Combined DNA/RNA Molecule Synthetic oligonucleotide
caacgaggaguggacuguatt

SEQ ID NO: 4 - DNA, 21 bp, Artificial Sequence
  Description of Artificial Sequence Synthetic oligonucleotide
  Description of Combined DNA/RNA Molecule Synthetic oligonucleotide
uacaguccacuccucguugtt

SEQ ID NO: 5 - DNA, 21 bp, Artificial Sequence
  Description of Artificial Sequence Synthetic oligonucleotide
  Description of Combined DNA/RNA Molecule Synthetic oligonucleotide
uucuccgaacgugucacgutt

SEQ ID NO: 6 - DNA, 21 bp, Artificial Sequence
  Description of Artificial Sequence Synthetic oligonucleotide
  Description of Combined DNA/RNA Molecule Synthetic oligonucleotide
acgugacacguucggagaatt

SEQ ID NO: 7 - DNA, 20 bp, Mus musculus
ggcattctcaatgcggaagg

SEQ ID NO: 8 - DNA, 20 bp, Mus musculus
aactcattggaggtgcacga

SEQ ID NO: 9 - DNA, 20 bp, Artificial Sequence
  Description of Artificial Sequence Synthetic oligonucleotide
gcgaggtattcggctccgcg